(19) United States
(12) Patent Application Publication
(10) Pub. No.: US 2006/0079453 A1
(43) Pub. Date: Apr. 13, 2006

(54) Hla binding peptides and their uses

(76) Inventors: John Sidney, San Diego, CA (US); Scott Southwood, Santee, CA (US); Alessandro Sette, La Jolla, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/530,061

(22) PCT Filed: Oct. 3, 2003
(86) PCT No.: PCT/US03/31308

§ 371 Date: Apr. 4, 2005

Related U.S. Application Data
(60) Provisional application No. 60/416,207, filed on Oct. 3, 2002.
(60) Provisional application No. 60/417,269, filed on Oct. 8, 2002.

(51) Int. Cl. A61K 48/00; A61K 38/16; A61K 38/17; A61K 9/127

(52) U.S. Cl. 514/12; 514/44; 424/450

(57) ABSTRACT

Provided herein are peptides in certian pathogens and/or human or murine proteins that are identified as capable of binding one or more MHC molecules and inducing an immume response in a system. Also provided are compositions that include one or more of the peptides and methods for inducing an immune reponse in a system by administering the compositions to the system.

### FIELD OF THE INVENTION

The invention relates to peptides that bind major histocompatibility (MHC) molecules and the use of these peptides to induce and modulate an immune response.

### BACKGROUND

The recognition of foreign pathogens, foreign cells (e.g., tumor), or one's own cells by the immune system occurs largely through major histocompatibility (MHC) molecules. MHC molecules present unique molecular fragments of foreign and self molecules that permit recognition and, when appropriate, stimulation of various immune effectors, namely B and T lymphocytes. MHC molecules are classified as either class I or class II. Class II MHC molecules are expressed primarily on activated lymphocytes and antigen-presenting cells. CD4+ T lymphocytes are activated with recognition of a unique peptide fragment presented by a class II MHC molecule, usually found on an antigen presenting cell like a macrophage or dendritic cell. Often known as helper T lymphocytes (HTL), CD4+ lymphocytes proliferate and secrete cytokines that either support a antibody-mediated response through the production of IL-4 and IL-10 or support a cell-mediated response through the production of IL-2 and IFN-γ. Class I MHC molecules, on the other hand, are expressed on virtually all nucleated cells. Peptide fragments presented in the context of Class I MHC molecules are recognized by CD8+ T lymphocytes. CD8+ T lymphocytes frequently mature into cytotoxic effector which can lyse cells bearing the stimulating antigen. Otherwise known as cytotoxic T lymphocytes (CTLs), CTLs are particularly effective in eliminating tumor cells and in fighting viral infections.

T lymphocytes recognize an antigen in the form of a peptide fragment bound to the MHC class I or class II molecule rather than the intact foreign antigen itself. An antigen presented by a MHC class I molecule is typically one that is endogenously synthesized by the cell (e.g., an intracellular pathogen). The resulting cytoplasmic antigens are degraded into small fragments in the cytoplasm, usually by the proteosome (Niedermann et al., Immunity, 2: 289-99(1995)). Some of these small fragments are transported into the endoplasmic reticulum where the fragment interacts with class I heavy chains to facilitate proper folding and association with the subunit β2 microglobulin to result in a stable complex formation between the fragment, MHC class I chain and β2 microglobulin. This complex is then transported to the cell surface for expression and potential recognition by specific CTLs. Antigens presented by MHC class II molecules are usually soluble antigens that enter the antigen presenting cell via phagocytosis, pinocytosis, or receptor-mediated endocytosis. Once in the cell, the antigen is partially degraded by acid-dependent proteases in endosomes. The resulting fragments or peptide associate with the MHC class II molecule after the release of the CLIP fragment to form a stable complex that is then transported to the surface for potential recognition by specific HTLs. See Blum et al., Crit. Rev. Immunol., 17: 411-17 (1997); Arndt et al., Immuno.l Res., 16: 261-72 (1997).

Peptides that bind some MHC complexes have been identified by acid elution methods (Buus et al., Science 242: 1065 (1988)), chromatography methods (Jardetzky, et al., Nature 353: 326 (1991) and Falk et al., Nature 351: 290 (1991)), and by mass spectrometry methods (Hunt, et al., Science 225: 1261 (1992)). A review of naturally processed peptides that bind MHC class I molecules is set forth in Rötzschke and Falk, Immunol. Today 12: 447 (1991).

Peptides that bind a particular MHC allele frequently will fit within a motif and have amino acid residues with particular biochemical properties at specific positions within the peptide. Such residues are usually dictated by the biochemical properties of the MHC allele. Peptide sequence motifs have been utilized to screen peptides capable of binding MHC molecules (Sette et al., Proc. Natl. Acad. Sci. USA 86:3296 (1989)), and it has been reported that class I binding motifs identified potential immunogenic peptides in animal models (De Bruijn et al., Eur. J. Immunol. 21: 2963-2970 (1991); Pamer et al., Nature 353: 852-955 (1991)). Also, binding of a particular peptide to a MHC molecule has been correlated with immunogenicity of that peptide (Schaeffer et al., Proc. Natl. Acad. Sci. USA 86:4649 (1989)).

Of the many thousand possible peptides that are encoded by a complex foreign pathogen, only a small fraction ends up in a peptide form capable of binding to MHC class I or class II antigens and thus of being recognized by T cells. This phenomenon is known as immunodominance (Yewdell et al., Ann. Rev. Immunol., 17: 51-88 (1997)). More simply, immunodominance describes the phenomenon whereby immunization or exposure to a whole native antigen results in an immune response directed to one or a few “dominant” epitopes of the antigen rather than every epitope that the native antigen contains. Immunodominance is influenced by a variety of factors that include MHC-peptide affinity, antigen processing, and antigen availability.

Accordingly, while some MHC binding peptides have been identified, there is a need in the art to identify novel MHC binding peptides from pathogens that can be utilized to generate an immune response in vaccines against the pathogens from which they originate. Further, there is a need in the art to identify peptides capable of binding a wide array of different types of MHC molecules such they are immunogenic in a large fraction a human outbred population.

### SUMMARY

The present invention relates to compositions and methods for preventing, treating or diagnosing a number of pathological states such as viral diseases and cancers. Thus, provided herein are novel peptides capable of binding selected major histocompatibility complex (MHC) molecules and inducing or modulating an immune response. Some of the peptides disclosed are capable of binding human class II MHC (HLA) molecules, including HLA-DR and HLA-DQ alleles. Other peptides disclosed herein are capable of binding to human class I molecules, including one or more of the following: HLA-A1, HLA-A2.1, HLA-A3.2, HLA-A11, HLA-A24.1, HLA-B7, and HLA-B44 molecules. Other peptides disclosed are capable of binding to murine class I molecules. Also provided are compositions that include immunogenic peptides having binding motifs specific for MHC molecules. The peptides and compositions disclosed can be utilized in methods for inducing an immune response, a cytotoxic T lymphocyte (CTL) response or helper T lymphocyte (HTL) response in particular, when administered to a system. The peptides and compositions disclosed herein are also useful as diagnostic reagents (e.g., tetramer reagents; Beckman Coulter).

### BRIEF DESCRIPTION OF THE DRAWINGS

FIG. 1. Preferred Motif Table.

FIG. 2. HLA superfamilies for HLA-A and HLA-B alleles. Various alleles of HLA-A and HLA-B are classified according to superfamily based on sequencing analysis or binding assays (verified supertype members) or on the basis of B and F pocket structure (predicted supertype members).

### DEFINITIONS

The following definitions are provided to enable one of ordinary skill in the art to understand some of the preferred embodiments of invention disclosed herein. It is understood, however, that these definitions are exemplary only and should not be used to limit the scope of the invention as set forth in the claims. Those of ordinary skill in the art will be able to construct slight modifications to the definitions below and utilize such modified definitions to understand and practice the invention disclosed herein. Such modifications, which would be obvious to one of ordinary skill in the art, as they may be applicable to the claims set forth below, are considered to be within the scope of the present invention. If a definition set forth in this section is contrary to or otherwise inconsistent with a definition set forth in patents, published, patent applications and other publications and sequences from GenBank and other data bases that are herein incorporated by reference, the definition set forth in this section prevails over the definition that is incorporated herein by reference.

As used herein, the term “HLA supertype or HLA family,” refers to sets of HLA molecules grouped based on shared peptide-binding specificities. The terms HLA superfamily, HLA supertype family, HLA family, and HLA xx-like molecules (where xx denotes a particular HLA type), are synonyms.

As used herein, the term “IC₅₀” refers to the concentration of peptide in a binding assay at which 50% inhibition of binding of a reference peptide is observed. Depending on the conditions in which the assays are run (e.g., limiting MHC proteins and labeled peptide concentrations), these values may approximate K_{D} values.

As used herein, the term “peptide” is used interchangeably with “epitope” in the present specification to designate a series of residues, typically L-amino acids, connected one to the other, typically by peptide bonds between the α-amino and carboxyl groups of adjacent amino acids, that binds to a designated MHC allele.

As used herein, the term “pharmaceutically acceptable” refers to a generally non-toxic, inert, and/or physiologically compatible composition.

As used herein, the term “protective immune response” or “therapeutic immune response” refers to a CTL and/or an HTL response to an antigen derived from an infectious agent or a tumor antigen, which in some way prevents or at least partially arrests disease symptoms, side effects or progression. The immune response may also include an antibody response that has been facilitated by the stimulation of helper T cells.

As used herein, the term “residue” refers to an amino acid or amino acid mimetic incorporated in a peptide by an amide bond or amide bond mimetic.

As used herein, the term “motif” refers to the pattern of residues in a peptide of defined length, usually a peptide of from about 8 to about 13 amino acids for a class I MHC motif and from about 6 to about 25 amino acids for a class II MHC motif, which is recognized by a particular MHC molecule. Peptide motifs are typically different for each protein encoded by each MHC allele and differ in the pattern of the highly conserved and negative residues.

As used herein, the term “supermotif” refers to an amino acid sequence for a peptide that provides binding specificity shared by MHC molecules encoded by two or more MHC alleles. Preferably, a supermotif-bearing peptide is recognized with high or intermediate affinity (as defined herein) by two or more MHC antigens.

As used herein, the term “conserved residue” refers to an amino acid which occurs in a significantly higher frequency than would be expected by random distribution at a particular position in a peptide. Typically a conserved residue is one where the MHC structure may provide a contact point with the immunogenic peptide. At least one to three or more, preferably two, conserved residues within a peptide of defined length defines a motif for an immunogenic peptide. These residues are typically in close contact with the peptide binding groove, with their side chains buried in specific pockets of the groove itself Typically, an immunogenic peptide will comprise up to three conserved residues, more usually two conserved residues.

As used herein, “negative binding residues” are amino acids which if present at certain positions (for example, positions 1, 3, 6 and/or 7 of a 9-mer) will result in a peptide being a nonbinder or poor binder and in turn fail to be immunogenic, e.g., induce a CTL response.

As used herein, the term “synthetic peptide” refers to a peptide that is not naturally occurring, but is man-made using such methods as chemical synthesis or recombinant DNA technology.

As used herein, the term “immunogenic peptide” refers to a peptide which comprises an allele-specific motif such that the peptide will bind an MHC molecule and induce a CTL or HTL response. An immunogenic response includes one that stimulates a CTL and/or HTL response in vitro and/or in vivo as well as modulates an ongoing immune response through directed induction of cell death (or apoptosis) in specific T cell populations.

As used herein, the phrases “isolated” or “biologically pure” refer to material which is substantially or essentially free from components which normally accompany it as found in its native state. Thus, the peptides of this invention do not contain materials normally associated with their in situ environment, e.g., MHC I molecules on antigen presenting cells. Even where a protein has been isolated to a homogeneous or dominant band, there are trace contaminants in the range of 5-10% of native protein which co-purify with the desired protein. Isolated peptides of this invention do not contain such endogenous co-purified protein.

Nomenclature used to describe peptide compounds follows the conventional practice wherein the amino group is presented to the left (the N-terminus) and the carboxyl group to the right (the C-terminus) of each amino acid residue. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G.

### DETAILED DESCRIPTION

A. Peptide and Motif Identification

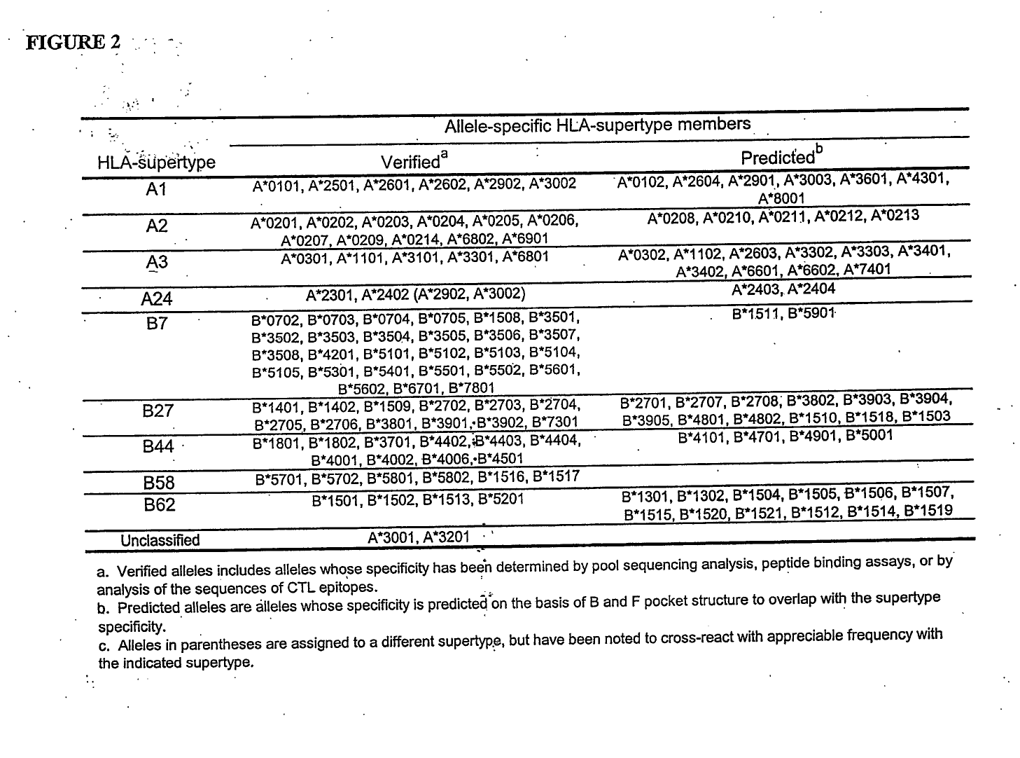

The present invention relates to allele-specific peptide motifs and binding peptides for human and murine MHC allele. It is contemplated that the peptide binding motifs of the invention are relatively specific for each allele. In an embodiment of the invention, the allele-specific motifs and binding peptides are for human class I MHC (or HLA) alleles. HLA alleles include HLA-A, HLA-B, and HLA-C alleles. In another embodiment of the invention the allele-specific motifs and binding peptides are for human class II MHC (or HLA) alleles. Such HLA alleles include HLA-DR and HLA-DQ alleles. HLA molecules that share similar binding affinity for peptides bearing certain amino acid motifs are grouped into HLA supertypes. See, e.g., Stites, et al., IMMUNOLOGY, 8^{TH} ED., Lange Publishing, Los Altos, Calif. (1994). Peptides that bind one or more alleles in one or more supertypes are contemplated as part of the invention. Examples of the supertypes within HLA-A and HLA-B molecules are shown in FIG. 2. In yet another embodiment, the allele-specific motifs and binding peptides are for murine class I (or H-2) MHC alleles. Such H-2 alleles include H-2Dd, H-2 Kb, H-2 Kd, H-2 Db, H-2Ld, and H-2Kk. Exemplary tables describing allele-specific motifs are presented below. Binding within a particular supertype for murine MHC alleles is also contemplated.

To identify peptides of the invention, MHC-peptide complex isolation, and isolation and sequencing of naturally processed peptides was carried out as described in the related applications. This application may be relevant to U.S. Ser. No. 09/189,702 filed Nov. 10, 1998, which is a CIP of U.S. Ser. No. 08/205,713 filed Mar. 4, 1994, which is a CIP of Ser. No. 08/159,184 filed Nov. 29, 1993 and now abandoned, which is a CIP of Ser. No. 08/073,205 filed Jun. 4, 1993 and now abandoned, which is a CIP of Ser. No. 08/027,146 filed Mar. 5, 1993 and now abandoned. The present application is also related to U.S. Ser. No. 09/226,775, which is a CIP of U.S. Ser. No. 08/815,396, which claims the benefit of U.S. Ser. No. 60/013,113, now abandoned. Furthermore, the present application is related to U.S. Ser. No. 09/017,735, which is a CIP of abandoned U.S. Ser. No. 08/589,108; U.S. Ser. No. 08/53,622, U.S. Ser. No. 08/822,382, abandoned U.S. Ser. No. 60/013,980, U.S. Ser. No. 08/454,033, U.S. Ser. No. 09/116,424, and U.S. Ser. No. 08/349,177. The present application is also related to U.S. Ser. No. 09/017,524, U.S. Ser. No. 08/821,739, abandoned U.S. Ser. No. 60/013,833, U.S. Ser. No. 08/758,409, U.S. Ser. No. 08/589,107, U.S. Ser. No. 08/451,913, U.S. Ser. No. 08/186,266, U.S. Ser. No. 09/116,061, and U.S. Ser. No. 08/347,610, which is a CIP of U.S. Ser. No. 08/159,339, which is a CIP of abandoned U.S. Ser. No. 08/103,396, which is a CIP of abandoned U.S. Ser. No. 08/027,746, which is a CIP of abandoned U.S. Ser. No. 07/926,666. The present application may also be relevant to U.S. Ser. No. 09/017,743, U.S. Ser. No. 08/753,615; U.S. Ser. No. 08/590,298, U.S. Ser. No. 09/115,400, and U.S. Ser. No. 08/452,843, which is a CIP of U.S. Ser. No. 08/344,824, which is a CIP of abandoned U.S. Ser. No. 08/278,634. The present application may also be related to provisional U.S. Ser. No. 60/087,192 and U.S. Ser. No. 09/009,953, which is a CIP of abandoned U.S. Ser. No. 60/036,713 and abandoned U.S. Ser. No. 60/037,432. In addition, the present application may be relevant to U.S. Ser. No. 09/098,584, and U.S. Ser. No. 09/239,043. The present application may also be relevant to co-pending U.S. Ser. No. 09/583,200 filed May 30, 2000, U.S. Ser. No. 09/260,714 filed Mar. 1, 1999, and U.S. Provisional Application “Heteroclitic Analogs And Related Methods”, Attorney Docket Number 018623-015810US filed Oct. 6, 2000. All of the above applications are incorporated herein by reference.

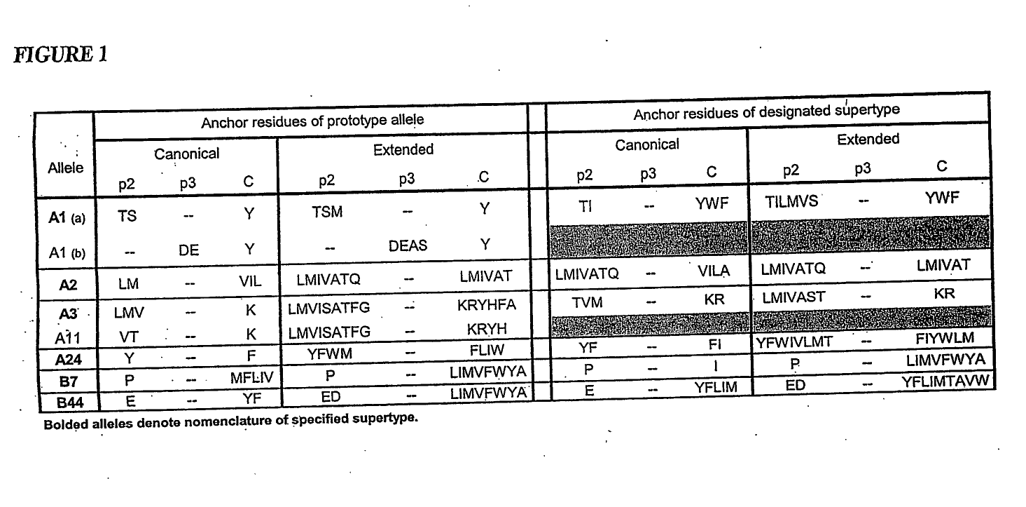

These peptides were then used to define specific binding motifs for each of the following alleles A3.2, A1, A11, and A24.1. These motifs are described previously. The motifs described in Tables 1-4, below, are defined from pool sequencing data of naturally processed peptides as described in the related applications. Preferred (i.e., canonical) and tolerated (i.e., extended) residues associated with anchor positions of the indicated HLA supertypes are presented in FIG. 1 and Table 5.

In one embodiment, the motif for HLA-A3.2 comprises from the N-terminus to C-terminus a first conserved residue of L, M, I, V, S, A, T and F at position 2 and a second conserved residue of K, R or Y at the C-terminal end. Other first conserved residues are C, G or D and alternatively E. Other second conserved residues are H or F. The first and second conserved residues are preferably separated by 6 to 7 residues. In another embodiment, the motif for HLA-A1 comprises from the N-terminus to the C-terminus a first conserved residue of T, S or M, a second conserved residue of D or E, and a third conserved residue of Y. Other second conserved residues are A, S or T. The first and second conserved residues are adjacent and are preferably separated from the third conserved residue by 6 to 7 residues. A second motif consists of a first conserved residue of E or D and a second conserved residue of Y where the first and second conserved residues are separated by 5 to 6 residues.

In yet another embodiment, the motif for HLA-A11 comprises from the N-terminus to the C-terminus a first conserved residue of T, V, M, L, I, S, A, G, N, C D, or F at position 2 and a C-terminal conserved residue of K, R, Y or H. The first and second conserved residues are preferably separated by 6 or 7 residues. In one embodiment, the motif for HLA-A24.1 comprises from the N-terminus to the C-terminus a first conserved residue of Y, F or W at position 2 and a C terminal conserved residue of F, I, W, M or L. The first and second conserved residues are preferably separated by 6 to 7 residues.

TABLE 1
Summary
HLA-A3, 2 Allele-Specific Motif

| | Conserved |
|---|---|
| Position | Residues |
| 1 | — |
| 2 | V, L, M |
| 3 | Y, D |
| 4 | — |
| 5 | — |
| 6 | — |
| 7 | I |
| 8 | Q, N |
| 9 | K |
| 10 | K |

TABLE 2
Summary
HLA-A1 Allele-Specific Motif

| | Conserved |
|---|---|
| Position | Residues |
| 1 | — |
| 2 | S, T |
| 3 | D, E |
| 4 | P |
| 5 | — |
| 6 | — |
| 7 | L |
| 8 | — |
| 9 | Y |
| 10 | K |

TABLE 3
Summary
HLA-A11 Allele-Specific Motif

| | Conserved |
|---|---|
| Position | Residues |
| 1 | — |
| 2 | T, V |
| 3 | M, F |
| 4 | — |
| 5 | — |
| 6 | — |
| 7 | — |
| 8 | Q |
| 9 | K |
| 10 | K |

TABLE 4
Summary
HLA-A24.1 Allele-Specific Motif

| | Conserved |
|---|---|
| Position | Residues |
| 1 | — |
| 2 | Y |
| 3 | I, M |
| 4 | D, E, G, K, P |
| 5 | L, M, N |
| 6 | V |
| 7 | N, V |
| 8 | A, E, K, Q, S |
| 9 | F, L |
| 10 | F, A |

The MHC-binding peptides identified herein represent epitopes of a native antigen. With regard to a particular amino acid sequence, an epitope is a set of amino acid residues which is recognized by a particular antibody or T cell receptor. Such epitopes are usually presented to lymphocytes via the MHC-peptide complex. An epitope retains the collective features of a molecule, such as primary, secondary and tertiary peptide structure, and charge, that together form a site recognized by an antibody, T cell receptor or MHC molecule. It is to be appreciated, however, that isolated or purified protein or peptide molecules larger than and comprising an epitope of the invention are still within the bounds of the invention. Moreover, it is contemplated that synthesized peptides can incorporate various biochemical changes that enhance their immunological effectiveness.

The epitopes present in the invention can be dominant, sub-dominant, or cryptic. A dominant epitope is an epitope that induces an immune response upon immunization with a whole native antigen. See, e.g., Sercarz, et al., Ann. Rev. Immunol. 11: 729-766 (1993). Such a peptide is considered immunogenic because it elicits a response against the whole antigen. A subdominant epitope, on the other hand, is one that evokes little or no response upon immunization with whole antigen that contains the epitope, but for which a response can be obtained by immunization with an isolated epitope. Immunization with a sub-dominant epitope will prime for a secondary response to the intact native antigen. A cryptic epitope elicits a response by immunization with an isolated peptide, but fails to prime a secondary response to a subsequent challenge with whole antigen.

An epitope present in the invention can be cross-reactive or non-cross-reactive in its interactions with MHC alleles and alleles subtypes. Cross-reactive binding of an epitope (or peptide) permits an epitope to be bound by more than one HLA molecule. Such cross-reactivity is also known as degenerate binding. A non-cross-reactive epitope would be restricted to binding a particular MHC allele or allele subtype.

The epitopes of the present invention can be any suitable length. Class I molecule binding peptides typically are about 8 to 13 amino acids in length, and often 9, 10, 11, or 12 amino acids in length. These peptides include conserved amino acids at certain positions such as the second position from the N-terminus and the C-terminal position. Also, the peptides often do not include amino acids at certain positions that negatively affect binding of the peptide to the HLA molecules. For example, the peptides often do not include amino acids at positions 1, 3, 6 and/or 7 for peptides 9 amino acid peptides in length or positions 1, 3, 4, 5, 7, 8 and/or 9 for peptides 10 amino acids in length. Further, defined herein are positions within a peptide sequence that can be utilized as criteria for selecting HLA-binding peptide. These defined positions are often referred to herein as a binding “motif.”

Definition of motifs specific for different MHC alleles allows the identification of potential peptide epitopes from an antigenic protein whose amino acid sequence is known. Typically, identification of potential peptide epitopes is initially carried out using a computer to scan the amino acid sequence of a desired antigen for the presence of motifs. The epitopic sequences are then synthesized.

In general, class I peptide binding motifs generally include a first conserved residue at position two from the N-terminus (wherein the N-terminal residue is position one) and a second conserved residue at the C-terminal position (often position 9 or 10). As a specific example, the HLA A*0201 class I peptide binding motifs include a first conserved residue at position two from the N-terminus (wherein the N-terminal residue is position one) selected from the group consisting of I, V, A and T and a second conserved residue at the C-terminal position selected from the group consisting of V, L, I, A and M. Alternatively, the peptide may have a first conserved residue at the second position from the N-terminus (wherein the N-terminal residue is position one) selected from the group consisting of L, M, I, V, A and T; and a second conserved residue at the C-terminal position selected from the group consisting of A and M. If the peptide has 10 residues it will contain a first conserved residue at the second position from the N-terminus (wherein the N-terminal residue is position one) selected from the group consisting of L, M, I, V, A, and T; and a second conserved residue at the C-terminal position selected from the group consisting of V, I, L, A and M; wherein the first and second conserved residues are separated by 7 residues.

One embodiment of an HTL-inducing peptide is less than about 50 residues in length and usually consist of between about 6 and about 30 residues, more usually between about 12 and 25, and often between about 15 and 20 residues, for example 15, 16, 17, 18, 19, or 20 residues. One embodiment of an CTL-inducing peptide is 13 residues or less in length and usually consists of about 8, 9, 10 or 11 residues, preferably 9 or 10 residues. In one embodiment, HLA-DR3 a binding is characterized by an L, I, V, M, F or Y residue at position 1 and a D or E residue at position 4. In another embodiment, HLA-DR3 b binding is characterized by an L, I, V, M, F, Y or A residue at position 1, a D, E, N, Q, S or T residue at position 4, and a K, R or H residue at position 6. In another embodiment, key anchor residues of a DR supertype binding motif are an L, I, V, M, F, W or Y residue at position 1 and an L, I, V, M, S, T, P, C or A residue at position 6. See table 5.

TABLE 5
HLA-DR motifs

Anchor residues of HLA-DR core motifs

| | p1 | p4 | p6 |
|---|---|---|---|
| DR supertype | LIVMFWY | — | LIVMSTPCA |
| DR3 a | LIVMFY | DE | — |
| DR3 b | LIVMFYA | DENQST | KRH |

Moreover, in another embodiment, murine Db binding is characterized by an N residue at position 5 and L, I, V or M residue at the C-terminal position. In yet another embodiment, murine Kb binding is characterized by a Y or F residue at position 5 and an L, I, V or M residue at the C-terminal position. In an additional embodiment, murine Kd binding is characterized a Y or F residue at position 2 and an L, I, V, or M residue at the C-terminal position. In a further embodiment, murine Kk binding is characterized by an E or D residue at position 2 and an L, I, M, V, F, W, Y or A residue at the C-terminal position. In a further embodiment, murine Ld binding is characterized by a P residue at position 2 and an L, I, M, V, F, W or Y residue at the C-terminal position. See Table 6.

TABLE 6
Murine Class I Motifs

Anchor residues of mouse class I motifs

| Allele | p2 | p3 | p5 | C terminus |
|---|---|---|---|---|
| Db | — | — | N | LIVM |
| Dd | G | P | — | LVI |
| Kb | — | — | YF | LIVM |
| Kd | YF | — | — | LIVM |
| Kk | ED | — | — | LIMVA |
| Ld | P | — | — | LIMVFWY |

The peptides present in the invention can be identified by any suitable method. For example, peptides are conveniently identified using the algorithms of the invention described in the co-pending U.S. patent application Ser. No. 09/894,018. These algorithms are mathematical procedures that produce a score which enables the selection of immunogenic peptides. Typically one uses the algorithmic score with a binding threshold to enable selection of peptides that have a high probability of binding at a certain affinity and will in turn be immunogenic. The algorithm are based upon either the effects on MHC binding of a particular amino acid at a particular position of a peptide or the effects on binding MHC of a particular substitution in a motif containing peptide.

Peptide sequences characterized in molecular binding assays and capture assays have been and can be identified utilizing various technologies. Motif-positive sequences are identified using a customized application created at Epimmune. Sequences are also identified utilizing matrix-based algorithms, and have been used in conjunction with a “power” module that generates a predicted 50% inhibitory concentration (PIC) value. These latter methods are operational on Epimmune's HTML-based Epitope Information System (EIS) database. All of the described methods are viable options in peptide sequence selection for IC₅₀ determination using binding assays.

Additional procedures useful in identifying the peptides of the present invention generally follow the methods disclosed in Falk et al., Nature 351:290 (1991). Briefly, the methods involve large-scale isolation of MHC class I molecules, typically by immunoprecipitation or affinity chromatography, from the appropriate cell or cell line. Examples of other methods for isolation of the desired MHC molecule equally well known to the artisan include ion exchange chromatography, lectin chromatography, size exclusion, high performance liquid chromatography, and a combination of some or all of the above techniques.

For example, isolation of peptides bound to MHC class I molecules include lowering the culture temperature from 37° C. to 26° C. overnight to destabilize β₂ microglobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which may slow the cell's metabolic rate. It is also likely that cells not actively synthesizing MHC molecules (e.g., resting PBMC) would not produce high amounts of empty surface MHC molecules by the cold temperature procedure.

Immunoprecipitation is also used to isolate the desired allele. A number of protocols can be used, depending upon the specificity of the antibodies used. For example, allele-specific mAb reagents can be used for the affinity purification of the HLA-A, HLA-B, and HLA-C molecules. Several mAb reagents for the isolation of HLA-A molecules are available (Table 5). Monoclonal antibody BB7.2 is suitable for isolating HLA-A2 molecules. Thus, for each of the targeted HLA-A alleles, reagents are available that may be used for the direct isolation of the HLA-A molecules. Affinity columns prepared with these mAbs using standard techniques are successfully used to purify the respective HLA-A allele products.

In addition to allele-specific mAbs, broadly reactive anti-HLA-A, B, C mAbs, such as W6/32 and B9.12.1, and one anti-HLA-B, C mAb, B1.23.2, could be used in alternative affinity purification protocols as described in patents and patent applications described herein.

TABLE 7
HLA CLASS I MHC MOLECULES

| | | Ab utilized |
|---|---|---|
| HLA-A, B | | for Capture |
| Allele | Cell Lines | assay |
| A*0101 | Steinlin, MAT | W6/32 |
| A*2601 | Pure Protein, QBL | W6/32 |
| A*2902 | Sweig, Pure Protein, Pitout | W6/32 |
| A*3002 | DUCAF, Pure Protein | W6/32 |
| A*2301 | Pure Protein, WT51 | W6/32 |
| A*2402 | KT3, Pure Protein, KAS116 | W6/32 |
| A*0201 | JY, OMW | W6/32 |
| A*0202 | M7B | W6/32 |
| A*0203 | FUN | W6/32 |
| A*0205 | DAH | W6/32 |
| A*0206 | CLA | W6/32 |
| A*0207 | AP | W6/32 |
| A*6802 | AMAI | W6/32 |
| A*0301 | GM3107 | W6/32 |
| A*1101 | BVR | W6/32 |
| A*3101 | SPACH, OLL | W6/32 |
| A*3301 | LWAGS | W6/32 |
| A*6801 | CIR, 2F7 | W6/32 |
| B*0702 | GM3107, JY | W6/32 |
| B*3501 | CIR, BVR | W6/32 |
| B*5101 | KAS116 | W6/32 |
| B*5301 | AMAI | W6/32 |
| B*5401 | KT3 | W6/32 |
| B*1801 | DUCAF | W6/32 |
| B*4001 | 2F7 | W6/32 |
| B*4002 | Sweig | W6/32 |
| B*4402 | WT47 | B1.23.1 |
| B*4403 | Pitout | B1.23.1 |
| B*4501 | OMW | W6/32 |
| A*3201 | Pure Protein, WT47 | W6/32 |

The peptides bound to the peptide binding groove of the isolated MHC molecules are typically eluted using acid treatment. Peptides can also be dissociated from MHC molecules by a variety of standard denaturing means, such as, for example, heat, pH, detergents, salts, chaotropic agents, or a combination acid treatment and/or more standard denaturing means.

Peptide fractions are further separated from the MHC molecules by reversed-phase high performance liquid chromatography (HPLC) and sequenced. Peptides can be separated by a variety of other standard means well known to the artisan, including filtration, ultrafiltration, electrophoresis, size chromatography, precipitation with specific antibodies, ion exchange chromatography, isoelectrofocusing, and the like.

Sequencing of the isolated peptides can be performed according to standard techniques such as Edman degradation (Hunkapiller, M. W., et al., Methods Enzymol. 91, 399 (1983)). Other methods suitable for sequencing include mass spectrometry sequencing of individual peptides as previously described (Hunt, et al., Science 225:1261 (1992)). Amino acid sequencing of bulk heterogeneous peptides (e.g., pooled HPLC fractions) from different MHC molecules typically reveals a characteristic sequence motif for each MHC allele. A large number of cells with defined MHC molecules, particularly MHC Class I molecules, are known and readily available. For example, human EBV-transformed B cell lines have been shown to be excellent sources for the preparative isolation of class I and class II MHC molecules. Well-characterized cell lines are available from private and commercial sources, such as American Type Culture Collection (“Catalogue of Cell Lines and Hybridomas,” 6th edition (1988) Manassas, Va., U.S.A.); National Institute of General Medical Sciences 1990/1991 Catalog of Cell Lines (NIGMS) Human Genetic Mutant Cell Repository, Camden, N.J.; and ASHI Repository, Brigham and Women's Hospital, 75 Francis Street, Boston, Mass. 02115. Table 5 lists some B cell lines suitable for use as sources for HLA alleles. All of these cell lines can be grown in large batches and are therefore useful for large scale production of MHC molecules. One of skill will recognize that these are merely exemplary cell lines and that many other cell sources can be employed. Specific cell lines and antibodies used to determine class II and murine peptides disclosed herein are set forth in Tables 8 and 9.

TABLE 8
HLA Class II MHC molecules

| | HLA-DR, DQ | | Ab utilized for |
|---|---|---|---|
| Antigen | Allele | Cell Line | Capture assay |
| DR1 | DRB1*0101 | LG2 | LB3.1 |
| DR3 | DRB1*0301 | MAT | LB3.1 |
| DR4 | DRB1*0401 | PREISS | LB3.1 |
| DR4 | DRB1*0404 | BIN40 | LB3.1 |
| DR4 | DRB1*0405 | KT3 | LB3.1 |
| DR7 | DRB1*0701 | PITOUT, DBB | LB3.1 |
| DR8 | DRB1*0802 | OLL | LB3.1 |
| DR9 | DRB1*0901 | HID | LB3.1 |
| DR11 | DRB1*1101 | SWEIG | LB3.1 |
| DR12 | DRB1*1201 | HERLUF | LB3.1 |
| DR13 | DRB1*1302 | H0301 | LB3.1 |
| DR15 | DRB1*1501 | L466.1 | LB3.1 |
| DR52 | DRB3*0101 | MAT | LB3.1 |
| DR53 | DRB4*0101 | L257.6 | LB3.1 |
| DR51 | DRB5*0101 | GM3107, L416.3 | LB3.1 |
| DQ7 | DQA1*0301/B*0301 | PF | |
| DQ2 | DQA1*0501/B*0201 | MAT, STEINLIN | |
| DQ8 | DQA1*0301/B*0302 | 145b, PREISS, YAR | |

TABLE 9
Murine MHC molecules

| MHC | | | Ab utilized for |
|---|---|---|---|
| class | Allele | Cell Line | Capture Assay |
| I | Db | EL4 | |
| I | Db | P815 | |
| I | Kb | EL4 | |
| I | Kd | P815 | |
| I | Kk | CH27 | Y3 |
| I | Ld | P815 | |
| II | IAb | DB27.4 | |
| II | IAd | A20 | |
| II | IAk | CH12 | |
| II | IAs | LS102.9 | |
| II | IAu | 91.7 | |
| II | IEd | A20 | |
| II | IEk | CH12 | |

The peptides of the invention can be prepared synthetically, or by recombinant DNA technology or from natural sources such as whole viruses or tumors. Although the peptide will preferably be substantially free of other naturally occurring host cell proteins and fragments thereof, in some embodiments the peptides can be synthetically or naturally conjugated to native protein fragments or particles. The peptides of the invention can be prepared in a wide variety of ways. Because of their relatively short size, the peptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984), supra.

B. MHC Binding Assays

The capacity to bind MHC molecules is measured in a variety of different ways. One means is a MHC binding assay as described in the related applications, noted above. Other alternatives described in the literature include inhibition of antigen presentation (Sette, et al., J. Immunol. 141:3893 (1991), in vitro assembly assays (Townsend, et al., Cell 62:285 (1990), and FACS based assays using mutated cells, such as RMA. S (Melief, et al., Eur. J. Immunol. 21:2963 (1991)).

Capture Assay: Unlike the HPLC-based molecular binding assay, noted above, the high throughput screening (“HTS”) Capture assay does not utilize a size-exclusion silica column for separation of bound from unbound radioactive marker. Instead, wells of an opaque white 96-well Optiplate (Packard) are coated with 3 μg (100 μl @ 30 μg/ml) of HLA-specific antibody (Ab) that “capture” complexes of radiolabeled MHC and unlabeled peptide transferred from the molecular binding assay plate in 100 μl of 0.05% NP40/PBS. After a 3-hour incubation period, the supernatant is decanted and scintillation fluid (Microscint 20) added. Captured complexes are then measured on a microplate scintillation and luminescence counter (TopCount NXTTM; Packard).

Additional assays for determining binding are described in detail, e.g., in PCT publications WO 94/20127 and WO 94/03205. Binding data results are often expressed in terms of IC₅₀ value. IC₅₀ is the concentration of peptide in a binding assay at which 50% inhibition of binding of a reference peptide occurs. Given the conditions in which the assays are performed (e.g., limiting MHC proteins and labeled peptide concentrations), these values approximate K_{D} values. It should be rioted that IC₅₀ values can change, often dramatically, if the assay conditions are varied, and depending on the particular reagents used (e.g., MHC preparation, etc.). For example, excessive concentrations of MHC molecules will increase the apparent measured IC₅₀ of a given ligand. Alternatively, binding is expressed relative to a reference peptide. Although as a particular assay becomes more, or less, sensitive, the IC₅₀'s of the peptides tested may change somewhat, the binding relative to the reference peptide will not significantly change. For example, in an assay preformed under conditions such that the IC₅₀ of the reference peptide increases 10-fold, the IC₅₀ values of the test peptides will also increase approximately 10-fold. Therefore, to avoid ambiguities, the assessment of whether a peptide is a good, intermediate, weak, or negative binder is generally based on its IC₅₀, relative to the IC₅₀ of a standard peptide.

Binding may also be determined using other assay systems including those using: live cells (e.g., Ceppellini et al., Nature 339:392, 1989; Christnick et al., Nature 352:67, 1991; Busch et al., Int. Immunol. 2:443, 19990; Hill et al., J. Immunol. 147:189, 1991; del Guercio et al., J. Immunol. 154:685, 1995), cell free systems using detergent lysates (e.g., Cerundolo et al., J. Immunol. 21:2069, 1991), immobilized purified MHC (e.g. Hill et al., J. Immunol. 152, 2890, 1994; Marshall et al., J. Immunol. 152:4946, 1994), ELISA systems (e.g., Reay et al., EMBO J. 11:2829, 1992), surface plasmon resonance (e.g., Khilko et al., J. Biol. Cheni. 268:15425, 1993); high flux soluble phase assays (e.g., Hammer et al., J. Exp. Med. 180:2353, 1994), and measurement of class I MHC stabilization or assembly (e.g., Ljunggren et al., Nature 346:476, 1990; Schumacher et al., Cell 62:563, 1990; Townsend et al., Cell 62:285, 1990; Parker et al., J. Immunol. 149:1896, 1992).

High affinity with respect to HLA class I molecules is defined as binding with an IC₅₀, or K_{D} value, of 50 nM or less; intermediate affinity with respect to HLA class I molecules is defined as binding with an IC₅₀ or K_{D} value of between about 50 and about 500 nM. High affinity with respect to binding to HLA class II molecules is defined as binding with an IC₅₀ or K_{D} value of 100 nM or less; intermediate affinity with respect to binding to HLA class II molecules is defined as binding with an IC₅₀ or K_{D} value of between about 100 and about 1000 nM. These values are as previously defined in the related patents and applications cited above.

C. Peptide Compositions

The polypeptides or peptides of the invention can be a variety of lengths, either in their neutral (uncharged) forms or in forms which are salts, and either free of modifications such as glycosylation, side chain oxidation, or phosphorylation or containing one or more of these modifications, subject to the condition that the modification not destroy the biological activity of the polypeptides as herein described.

Desirably, the peptide will be as small as possible while still maintaining substantially all of the biological activity of the large peptide. In one embodiment, it may be desirable to optimize peptides of the invention to a length of 9 or 10 amino acid residues, commensurate in size with endogenously processed viral peptides or tumor cell peptides that are bound to MHC class I molecules on the cell surface. In another embodiment, it may be desirable to optimize peptides of the invention to about 15 to 20 amino acid residues, commensurate with peptides that are bound to MHC class II molecules on the cell surface.

Peptides having the desired activity may be modified as necessary to provide certain desired attributes, e.g., improved pharmacological characteristics, while increasing or at least retaining substantially all of the biological activity of the unmodified peptide to bind the desired MHC molecule and activate the appropriate T cell. For instance, the peptides may be subject to various changes, such as substitutions, either conservative or non-conservative, where such changes might provide for certain advantages in their use, such as improved MHC binding. “Conservative substitution” refers to the replacement of an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany and Merrifield, The Peptides, Gross and Meienhofer, eds. (N.Y., Academic Press), pp. 1-284 (1979); and Stewart and Young, Solid Phase Peptide Synthesis, (Rockford, Ill., Pierce), 2d Ed. (1984).

The peptides of the invention can also be modified by extending or decreasing the compound's amino acid sequence, e.g., by the addition or deletion of amino acids. The peptides or analogs of the invention can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-natural amino acids as well, such as β-γ-δ-amino acids, as well as many derivatives of L-α-amino acids.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

Substitutions, deletions, insertions or any combination thereof may be combined to arrive at a final peptide. Substitutional variants are those in which at least one residue of a peptide has been removed and a different residue inserted in its place. Such substitutions generally are made in accordance with the following Table 10 when it is desired to finely modulate the characteristics of the peptide.

TABLE 10

| Original | Exemplary |
|---|---|
| Residue | Substitution |
| Ala | Ser |
| Arg | Lys, His |
| Asn | Gln |
| Asp | Glu |
| Cys | Ser |
| Gln | Asn |
| Glu | Asp |
| Gly | Pro |
| His | Lys; Arg |
| Ile | Leu; Val |
| Leu | Ile; Val |
| Lys | Arg; His |
| Met | Leu; Ile |
| Phe | Tyr; Trp |
| Ser | Thr |
| Thr | Ser |
| Trp | Tyr; Phe |
| Tyr | Trp; Phe |
| Val | Ile; Leu |
| Pro | Gly |

The peptides may also comprise isosteres of two or more residues in the MHC-binding peptide. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

Modifications of peptides with various amino acid mimetics or unnatural amino acids are particularly useful in increasing the stability of the peptide in vivo. Stability can be assayed in a number of ways. For instance, peptidases and various biological media, such as human plasma and serum, have been used to test stability. See, e.g., Verhoef et al., Eur. J. Drug Metab. Pharmacokin. 11:291-302 (1986). Half life of the peptides of the present invention is conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

The peptides of the present invention or analogs thereof which have CTL and/or HTL stimulating activity may be modified to provide desired attributes other than improved serum half life. For instance, the ability of the peptides to induce CTL activity can be enhanced by linkage to a sequence which contains at least one epitope that is capable of inducing a HTL response. Particularly preferred immunogenic peptides/T helper conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues, for example, 3, 4, 5 or 6 residues. Alternatively, the CTL peptide may be linked to the HTL peptide without a spacer. The immunogenic peptide may be linked to the HTL peptide either directly or via a spacer either at the amino or carboxy terminus of the CTL peptide. The amino terminus of either the immunogenic peptide or the HTL peptide may be acylated. Exemplary HTL peptides include tetanus toxoid 830-843, influenza 307-319, malaria circumsporozoite 382-398 and 378-389.

In addition, additional amino acids can be added to the termini of a peptide to provide for ease of linking peptides one to another, for coupling to a carrier support, or larger peptide, for modifying the physical or chemical properties of the peptide or oligopeptide, or the like. Amino acids such as tyrosine, cysteine, lysine, glutamic or aspartic acid, or the like, can be introduced at the C- or N-terminus of the peptide or oligopeptide. Modification at the C-terminus in some cases may alter binding characteristics of the peptide. In addition, the peptide or oligopeptide sequences can differ from the natural sequence by being modified by terminal-NH₂ acylation, e.g., by alkanoyl (C₁-C₂₀) or thioglycolyl acetylation, terminal-carboxylamidation, e.g., ammonia, methylamine, etc. In some instances these modifications may provide sites for linking to a support or other molecule.

Alternatively, recombinant DNA technology may be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1982). Thus, fusion proteins which comprise one or more peptide sequences of the invention can be used to present the appropriate T cell epitope.

As the coding sequence for peptides of the length contemplated herein can be synthesized by chemical techniques, for example, using the phosphotriester method of Matteucci et al., J. Am. Chem. Soc. 103:3185 (1981), with modification made simply by substituting the appropriate base(s) for those encoding the native peptide sequence. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts may also be used, employing suitable vectors and control sequences that are well-known in the art.

The peptide compositions of this invention may encode an MHC epitope operably linked to a MHC targeting sequence. The use of a MHC targeting sequence enhances the immune response to an antigen, relative to delivery of antigen alone, by directing the peptide epitope to the site of MHC molecule assembly and transport to the cell surface, thereby providing an increased number of MHC molecule-peptide epitope complexes available for binding to and activation of T cells. MHC Class I targeting sequences can be used in the present invention, e.g., those sequences that target an MHC Class I epitope peptide to a cytosolic pathway or to the endoplasmic reticulum (see, e.g., Rammensee et al., Immunogenetics 41:178-228 (1995)). Such MHC Class I targeting sequences are well known in the art, and include, e.g., signal sequences such as those from Ig, tissue plasminogen activator or insulin. See, e.g., Bonnerot et al., Immunity 3:335-347 (1995). A preferred signal peptide is the human Ig kappa chain sequence. Endoplasmic reticulum signal sequences can also be used to target MHC Class II epitopes to the endoplasmic reticulum, the site of MHC Class I molecule assembly. MHC Class II targeting sequences can also be used in the invention, e.g., those that target a peptide to the endocytic pathway. These targeting sequences typically direct extracellular antigens to enter the endocytic pathway, which results in the antigen being transferred to the lysosomal compartment where the antigen is proteolytically cleaved into antigen peptides for binding to MHC Class II molecules. For example, a group of MHC Class II targeting sequences useful in the invention are lysosomal targeting sequences, which localize polypeptides to lysosomes. Lysosomal targeting sequences are well known in the art and include exemplary sequences as described in U.S. Pat. No. 5,633,234 and Copier et al., J. Immunol. 157:1017-1027 (1996).

Substantial changes in function (e.g., affinity for MHC molecules or T cell receptors) are made by selecting substitutions that are less conservative than those in Table 10, e.g., selecting residues that differ more significantly in their effect on maintaining (a) the structure of the peptide backbone in the area of the substitution, for example as a sheet or helical conformation, (b) the charge or hydrophobicity of the molecule at the target site or (c) the bulk of the side chain. The substitutions which in general are expected to produce the greatest changes in peptide properties will be those in which (a) a hydrophilic residue, e.g. seryl, is substituted for (or by) a hydrophobic residue, e.g. leucyl, isoleucyl, phenylalanyl, valyl or alanyl; (b) a residue having an electropositive side chain, e.g., lys1, arginyl, or histidyl, is substituted for (or by) an electronegative residue, e.g. glutamyl or aspartyl; or (c) a residue having a bulky side chain, e.g. phenylalanine, is substituted for (or by) one not having a side chain, e.g., glycine.

Epitopes on any number of potential target proteins can be identified. Examples of suitable antigens include prostate specific antigen (PSA), prostate specific membrane antigen (PSM) hepatitis B virus core and surface antigens (HBVc, HBVs), hepatitis C antigens, malignant melanoma antigens (MAGE-1, MAGE-2, MAGE-3), Epstein-Barr virus antigens, human immunodeficiency type-1 virus (HIV-1), human immunodeficiency virus type-2 (HIV-2), papilloma virus antigens, Lassa virus, mycobacterium tuberculosis (MT) antigens, p53 and murine p53 (mp53) antigens, CEA, HER2/neu, and members of the tyrosine kinase related protein families (TKP). The peptides are thus useful in pharmaceutical compositions for both in vivo and ex vivo therapeutic and diagnostic applications.

D. Peptide Immunogenicity In Vitro and In Vivo

Peptides comprising the epitopes from these antigens are synthesized and then tested for their ability to bind to the appropriate MHC molecules in assays using, for example, purified MHC molecules and radioiodonated peptides and/or cells expressing empty MHC molecules by, for instance, immunofluorescent staining and flow microfluorometry, peptide-dependent class I assembly assays, and inhibition of CTL or HTL recognition by peptide competition. Those peptides that bind to the MHC molecule are further evaluated for their ability to serve as targets for CTLs and/or HTLs derived from infected or immunized individuals, as well as for their capacity to induce primary in vitro or in vivo T cell responses that can give rise to CTL and/or HTL populations capable of reacting with virally infected target cells or tumor cells as potential therapeutic agents.

Since mutant cell lines do not exist for every human MHC allele, it is advantageous to use various techniques to remove endogenous MHC-associated peptides from the surface of antigen presenting cell (APC) (e.g., mild acid treatment) followed by loading the resulting empty MHC molecules with the immunogenic peptides of interest. Antigen-presenting cells can be normal cells such as peripheral blood mononuclear cells or dendritic cells (Inaba, et al., J. Exp. Med. 166:182 (1987); Boog, Eur. J. Immunol. 18:219 (1988)). The use of non-transformed (non-tumorigenic), non-infected cells, and preferably, autologous cells of patients as the source of APC is desirable for the design of T cell induction protocols directed towards development of ex vivo CTL and/or HTL therapies.

Alternatively, mutant mammalian cell lines that are deficient in their ability to load class I molecules with internally processed peptides, such as the mouse cell lines RMA-S (Karre, et al., Nature, 319:675 (1986); Ljunggren, et al., Eur. J. Immunol. 21:2963-2970 (1991)), and the human somatic T cell hybrid, T-2 (Cerundolo, et al., Nature 345:449-452 (1990)) and which have been transfected with the appropriate human class I genes are conveniently used, when peptide is added to them, to test for the capacity of the peptide to induce in vitro primary CTL responses. Other eukaryotic cell lines which could be used include various insect cell lines such as mosquito larvae (e.g., ATCC cell lines CCL 125, 126, 1660, 1591, 6585, 6586), silkworm (e.g., ATTC CRL 8851), armyworm (e.g., ATCC CRL 1711), moth (e.g., ATCC CCL 80) and Drosophila cell lines (e.g., a Schneider cell line (see Schneider, J. Embryol. Exp. Morphol., 27:353-365 (1927))).

Specificity and MHC restriction of the CTL or HTL is determined by testing against different peptide target cells expressing appropriate or inappropriate MHC molecules. The peptides that test positive in the MHC binding assays and give rise to specific CTL and/or HTL responses are referred to herein as immunogenic peptides.

Analyses of CTL and HTL responses against the immunogen, as well as against common recall antigens are commonly used and are known in the art. Assays employed included chromium release, lymphokine secretion and lymphoproliferation assays. Assays useful in these determinations are described in Current Protocols in Immunology, J. E. Coligan, et al., eds., John Wiley & Sons Press (2000), chapters 3, 4, 6, and 7.

In one embodiment, the appropriate antigen-presenting cells are incubated with 10-100 μM of peptide in serum-free media for 4 hours under appropriate culture conditions. The peptide-loaded antigen-presenting cells are then incubated with the responder cell populations in vitro for 7 to 10 days under optimized culture conditions. If screening for MHC class I presented peptides, positive CTL activation can be determined by assaying the cultures for the presence of CTLs that kill radiolabeled target cells, both specific peptide-pulsed targets as well as target cells expressing the endogenously processed form of the relevant virus or tumor antigen from which the peptide sequence was derived. If screening for MHC class II-presented peptides, positive HTL activation can be determined by assaying cultures for cytokine production or proliferation.

In one embodiment, prior to incubation of the stimulator cells with the cells to be activated, e.g., precursor CD8+ cells, an amount of antigenic peptide is added to the stimulator cell culture, of sufficient quantity to become loaded onto the human Class I molecules to be expressed on the surface of the stimulator cells. In the present invention, a sufficient amount of peptide is an amount that will allow about 200, and preferably 200 or more, human Class I MHC molecules loaded with peptide to be expressed on the surface of each stimulator cell. Preferably, the stimulator cells are incubated with >20 μg/ml peptide.

Resting or precursor CD8+ cells are then incubated in culture with the appropriate stimulator cells for a time period sufficient to activate the CD8+ cells. Preferably, the CD8+ cells are activated in an antigen-specific manner. The ratio of resting or precursor CD8+ (effector) cells to stimulator cells may vary from individual to individual and may further depend upon variables such as the amenability of an individual's lymphocytes to culturing conditions and the nature and severity of the disease condition or other condition for which the within-described treatment modality is used. Preferably, however, the lymphocyte:stimulator cell ratio is in the range of about 30:1 to 300:1. The effector/stimulator culture may be maintained for as long a time as is necessary to stimulate a therapeutically useable or effective number of CD8+ cells.

The peptides of the invention can be identified and tested for in vivo immunogenicity using HLA transgenic mice. The utility of HLA transgenic mice for the purpose of epitope identification (Sette et al., J Immunol, 153:5586-92 (1994); Wentworth et al., Int Immunol, 8:651-9 (1996); Engelhard et al., J Immunol, 146:1226-32 (1991); Man et al., Int Immunol, 7:597-605 (1995); Shirai et al., J Immunol, 154:2733-42 (1995)), and vaccine development (Ishioka et al., J Immunol, 162:3915-25 (1999)) has been established. Most of the published reports have investigated the use of HLA A2.1/K^{b} mice but it should be noted that B*27, and B*3501 mice are also available. Furthermore, HLA A*11/K^{b} mice (Alexander et al., J. Immunol., 159:4753-61 (1997)), and HLA B7/K^{b} and HLA A1/K^{b} mice have also been generated. Data from 38 different potential epitopes was analyzed to determine the level of overlap between the A2.1-restricted CTL repertoire of A2.1/K^{b}-transgenic mice and A2.1+humans (Wentworth et al., Eur J Immunzol, 26:97-101 (1996)). In both humans and mice, an MHC peptide binding affinity threshold of approximately 500 nM correlates with the capacity of a peptide to elicit a CTL response in vivo. A high level of concordance between the human data in vivo and mouse data in vivo was observed for 85% of the high-binding peptides, 58% of the intermediate binders, and 83% of the low/negative binders. Similar results were also obtained with HLA A11 and HLA B7 transgenic mice (Alexander et al., J Immunol, Vol. 159(10):4753-61 (1997)). Thus, because of the extensive overlap that exists between T cell receptor repertoires of HLA transgenic mouse and human CTLs, transgenic mice are valuable for assessing immunogenicity of the multi-epitope constructs described herein. Peptides binding to MHC class II alleles can be examined using HLA-DR transgenic mice. See, e.g., Taneja V., David C. S., Immunol Rev, 169:67-79 (1999)).

More sensitive techniques such as the ELISPOT assay, intracellular cytokine staining, and tetramer staining have become available in the art to determine lymphocyte antigen responsiveness. It is estimated that these newer methods are 10- to 100-fold more sensitive than the common CTL and HTL assays (Murali-Krishna et al., Immunity, 8:177-87 (1998)), because the traditional methods measure only the subset of T cells that can proliferate in vitro, and may, in fact, be representative of only a fraction of the memory T cell compartment (Ogg G. S., McMichael A. J., Curr Opin Immunol, 10:393-6 (1998)). Specifically in the case of HIV, these techniques have been used to measure antigen-specific CTL responses from patients that would have been undetectable with previous techniques (Ogg et al., Science, 279:2103-6 (1998); Gray et al., J Immunol, 162:1780-8 (1999); Ogg et al., J Virol, 73:9153-60 (1999); Kalams et al., J Virol, 73:6721-8 (1999); Larsson et al., AIDS, 13:767-77 (1999); Come et al., J Acquir Immune Defic Syndr Hum Retrovirol, 20:442-7 (1999)).

The peptides of the present invention and pharmaceutical and vaccine compositions thereof are useful for administration to mammals, particularly humans, to treat and/or prevent viral infection and cancer. Examples of diseases which can be treated using the immunogenic peptides of the invention include prostate cancer, hepatitis B, hepatitis C, AIDS, renal carcinoma, cervical carcinoma, lymphoma, CMV and chondyloma acuminatum. A protective (or prophylatic) vaccine includes one that will protect against future exposure to pathogen or cancer. A therapeutic vaccine includes one that will ameliorate, attenuate, or ablate symptoms or disease state induced by or related to a pathogen or malignancy.

In circumstances in which efficacy of a prophylactic vaccine is primarily correlated with the induction of a long-lasting memory response, restimulation assays can be the most appropriate and sensitive measures to monitor vaccine-induced immunological responses. Conversely, in the case of therapeutic vaccines, the main immunological correlate of activity can be the induction of effector T cell function, most aptly measured by primary assays. Thus, the use of sensitive assays allows for the most appropriate testing strategy for immunological monitoring of vaccine efficacy.

In some embodiments it may be desirable to include in the pharmaceutical compositions of the invention at least one component which primes CTL. Lipids have been identified as agents capable of priming CTL in vivo against viral antigens. The lipidated peptide can then be injected directly in a micellar form, incorporated into a liposome or emulsified in an adjuvant, e.g., incomplete Freund's adjuvant.

For pharmaceutical compositions, the immunogenic peptides of the invention are administered to an individual already suffering from cancer or infected with the virus of interest. Those in the incubation phase or the acute phase of infection can be treated with the immunogenic peptides separately or in conjunction with other treatments, as appropriate. In therapeutic applications, compositions are administered to a patient in an amount sufficient to elicit an effective CTL and/or HTL response to the virus or tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 5000 μg of peptide for a 70 kg patient, (e.g., 1.0 μg, 1.5 μg, 2.0 μg, 2.5 μg, 3.0 μg, 3.5 μg, 4.0 μg, 4.5 μg, 5.0 μg, 7.5 μg, 10 μg, 12.5 μg, 15 μg, 17.5 μg, 20 μg, 25 μg, 30 μg, 35 μg, 40 μg, 45 μg, 50 μg, 75 μg, 100 μg, 250 μg, 500 μg, 750 μg, 1000 μg, 1500 μg, 2000 μg, 2500 μg, 3000 μg, 3500 μg, 4000 μg, 4500 μg or 5000 μg), followed by boosting dosages of from about 1.0 μg to about 1000 μg of peptide (e.g., 1.0 μg, 2.0 μg, 2.5 μg, 3.0 μg, 3.5 μg, 4.0 μg, 4.5 μg, 5.0 μg, 7.5 μg, 10 μg, 12.5 μg, 15 μg, 17.5 μg, 20 μg, 25 μg, 30 μg, 35 μg, 40 μg, 45 μg, 50 μg, 75 μg, 100 μg, 250 μg, 500 μg, 750 μg, 1000 μg, 1500 μg, 2000 μg, 2500 μg, 3000 μg, 3500 μg, 4000 μg, 4500 μg or 5000 μg) pursuant to a boosting regimen over weeks to months depending upon the patient's response and condition by measuring specific T cell activity in the patient's blood. It must be kept in mind that the peptides and compositions of the present invention may generally be employed in serious disease states, that is, life-threatening or potentially life threatening situations. In such cases, in view of the minimization of extraneous substances and the relative nontoxic nature of the peptides, it is possible and may be felt desirable by the treating physician to administer substantial excesses of these peptide compositions.

The peptide compositions can also be used for the treatment of chronic infection and to stimulate the immune system to eliminate virus-infected cells in carriers. It is important to provide an amount of immuno-potentiating peptide in a formulation and mode of administration sufficient to effectively stimulate an appropriate response. Thus, for treatment of chronic infection, a representative dose is in the range of about 1.0 μg to about 5000 μg, preferably about 5 μg to 1000 μg (e.g., 5.0 μg, 7.5 μg, 10 μg, 12.5 μg, 15 μg, 17.5 μg, 20 μg, 25 μg, 30 μg, 35 μg, 40 μg, 45 μg, 50 μg, 75 μg, 100 μg, 250 μg, 300 μg, 350 μg, 400 μg, 450 μg, 500 μg, 550 μg, 600 μg, 650 μg, 700 μg, 750 μg, 800 μg, 900 μg, 950 μg, or 1000 μg,) for a 70 kg patient per dose. Immunizing doses followed by boosting doses at established intervals, e.g., from one to four weeks, may be required, possibly for a prolonged period of time to effectively immunize an individual. In the case of chronic infection, administration should continue until at least clinical symptoms or laboratory tests indicate that the viral infection has been eliminated or substantially abated and for a period thereafter.

The pharmaceutical compositions for therapeutic treatment are intended for parenteral, topical, oral or local administration. Preferably, the pharmaceutical compositions are administered parenterally, e.g., intravenously, subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

A pharmaceutical composition of the invention may comprise one or more T cell stimulatory peptides of the invention. For example, a pharmaceutical composition may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more T cell stimulatory peptides of the invention. Moreover, a pharmaceutical composition of the invention may comprise one or more T cell stimulatory peptides of the invention in combination with one or more other T cell stimulatory peptides. The concentration of each unique T cell stimulatory peptide of the invention in the pharmaceutical formulations can vary widely, e.g., from less than about 0.001%, about 0.002%, about 0.003%, about 0.004%, about 0.005%, about 0.006%, 0.007%, 0.008%, 0.009%, about 0.01%, about 0.02%, about 0.025%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 6%, about 7%, about 8%, about 9%, about 10%, about 20%, to about 50% or more by weight, and will be selected primarily by fluid volumes, viscosities, etc., in accordance with the particular mode of administration selected. In a preferred embodiment, the concentration of each unique T cell stimulatory peptide of the invention in the pharmaceutical formulations is about 0.001%, about 0.002%, about 0.003%, about 0.004%, about 0.005%, about 0.006%, 0.007%, 0.008%, 0.009%, about 0.01%, about 0.02%, about 0.025%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1% by weight. In a more preferred embodiment, the concentration of each unique T cell stimulatory peptide of the invention in the pharmaceutical formulations is about 0.01%, about 0.02%, about 0.025%, about 0.03%, about 0.04%, about 0.05%, about 0.06%, about 0.07%, about 0.08%, about 0.09%, about 0.1% by weight.

The peptides of the invention may also be administered via liposomes, which serve to target the peptides to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes either filled or decorated with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728, 4,837,028, and 5,019,369, each of which is incorporated herein by reference.

For targeting to the immune cells, a ligand to be incorporated into the liposome can include, e.g., antibodies or fragrnents thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

In another aspect the present invention is directed to vaccines which contain as an active ingredient an immunogenically effective amount of an immunogenic peptide as described herein. The peptide(s) may be introduced into a host, including humans, linked to its own carrier or as a homopolymer or heteropolymer of active peptide units. Such a polymer has the advantage of increased immunological reaction and, where different peptides are used to make up the polymer, the additional ability to induce antibodies and/or CTLs that react with different antigenic determinants of the virus or tumor cells. Useful carriers are well known in the art, and include, e.g., thyroglobulin, albumins such as human serum albumin, tetanus toxoid, polyamino acids such as poly(lysine:glutamic acid), influenza, hepatitis B virus core protein, hepatitis B virus recombinant vaccine and the like. The vaccines can also contain a physiologically tolerable (acceptable) diluent such as water, phosphate buffered saline, or saline, and further typically include an adjuvant. Adjuvants such as incomplete Freund's adjuvant (“IFA”), aluminum phosphate, aluminum hydroxide, or alum are materials well known in the art. And, as mentioned above, CTL responses can be primed by conjugating peptides of the invention to lipids, such as P₃CSS. Upon immunization with a peptide composition as described herein, via injection, aerosol, oral, transdermal or other route, the immune system of the host responds to the vaccine by producing large amounts of CTLs specific for the desired antigen, and the host becomes at least partially immune to later infection, or resistant to developing chronic infection.

Vaccine compositions containing the peptides of the invention are administered to a patient susceptible to or otherwise at risk of viral infection or cancer to elicit an immune response against the antigen and thus enhance the patient's own immune response capabilities. Such an amount is defined to be an “immunogenically effective dose.” In this use, the precise amounts again depend on the patient's state of health and weight, the mode of administration, the nature of the formulation, etc., but generally range from about 1.0 μg to about 5000 μg per 70 kilogram patient, more commonly from about 10 μg to about 500 μg per 70 kg of body weight (e.g., 10 μg, 15 μg, 20 μg, 25 μg, 30 μg, 35 μg, 40 μg, 45 μg, 50 μg, 60 μg, 70 μg, 80 μg, 90 μg, 100 μg, 125 μg, 150 μg, 175 μg, 200 μg, 225 μg, 250 μg, 275 μg, 300 μg, 325 μg, 375 μg, 400 μg, 425 μg, 450 μg, 475 μg or 500 μg per 70 kg of body weight).

For therapeutic or immunization purposes, nucleic acids encoding one or more of the peptides of the invention can also be administered to the patient. A number of methods are conveniently used to deliver the nucleic acids to the patient. For instance, the nucleic acid can be delivered directly, as “naked DNA”. This approach is described, for instance, in Wolff et. al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. The nucleic acids can also be delivered complexed to cationic compounds, such as cationic lipids. Lipid-mediated gene delivery methods are described, for instance, in WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414. The peptides of the invention can also be expressed by attenuated viral hosts, such as vaccinia or fowlpox. This approach involves the use of vaccinia virus as a vector to express nucleotide sequences that encode the peptides of the invention. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848, incorporated herein by reference. Another suitable vector is BCG (Bacille Calmette Guerin). BCG vectors are described, e.g., in Stover, et al., (Nature 351:456-460 (1991)). A wide variety of other vectors useful for therapeutic administration or immunization of the peptides of the invention, e.g., Salmonella typhi vectors and the like, will be apparent to those skilled in the art from the description herein.

A preferred means of administering nucleic acids encoding the peptides of the invention uses minigene constructs encoding multiple epitopes of the invention. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences, including DNA sequence encoding a variety of spacers between none, some or all DNA sequence encoding peptides, are adjoined to create, a continuous polypeptide sequence. To optimize expression and/or immunogenicity, additional elements can be incorporated into the minigene design. Examples of amino acid sequence that could be reverse translated and included in the minigene sequence include: helper T lymphocyte epitopes, a leader (signal) sequence, and an endoplasmic reticulum retention signal. In addition, MHC presentation of CTL epitopes may be improved by including synthetic (e.g. poly-alanine) or naturally-occurring flanking sequences adjacent to the CTL epitopes.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, IL12, GM-CSF), cytokine-inducing molecules (e.g., LeIF) or costimulatory molecules. Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g., TGF-β) may be beneficial in certain diseases.

The immunogenic peptides of this invention may also be used to make monoclonal antibodies. Such antibodies may be useful as potential diagnostic or therapeutic agents.

The peptides are also useful as diagnostic reagents (e.g., tetramer reagents; Beckman Coulter, San Diego, Calif.). For example, a peptide of the invention may be used to determine the susceptibility of a particular individual to a treatment regimen which employs the peptide or related peptides, and thus may be helpful in modifying an existing treatment protocol or in determining a prognosis for an affected individual. In addition, the peptides may also be used to predict which individuals will be at substantial risk for developing chronic infection.

The present invention relates to the determination of allele-specific peptide motifs for human and murine MHC allele subtypes. These motifs are then used to define T cell epitopes from any desired antigen, particularly those associated with human viral diseases, cancers or autoimmune diseases, for which the amino acid sequence of the potential antigen or autoantigen targets is known. The contents of all documents cited above are expressly incorporated herein by reference.

Brief Description of Tables 11-29

Table 11. Identified HLA-A1 allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 12. Binding affinity of HLA-A1 binding peptides. Peptides are identified by amino acid sequence, SEQ ID NO., and binding affinity to the designated HLA-A1 alleles (expressed as an IC₅₀).

Table 13. Identified HLA-A2 allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 14. Binding affinity of HLA-A2 binding peptides. Peptides are identified by amino acid sequence, SEQ ID NO., and binding affinity to the designated HLA-A2 alleles (expressed as an IC₅₀).

Table 15. Identified HLA-A3 allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 16. Binding affinity of HLA-A3 binding peptides. Peptides are identified by amino acid sequence, SEQ ID NO., and binding affinity to the designated HLA-A3 alleles (expressed as an IC₅₀).

Table 17. Identified HLA-A24 allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 18. Binding affinity of HLA-A24 binding peptides. Peptides are identified by amino acid sequence, SEQ ID NO., and binding affinity to the designated HLA-A24 alleles (expressed as an IC₅₀).

Table 19. Identified HLA-B7 allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 20. Binding affinity of HLA-B7 binding peptides. Peptides are identified by amino acid sequence, SEQ ID NO., and binding affinity to the designated HLA-B7 alleles (expressed as an IC₅₀).

Table 21. Identified HLA-B44 allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 22. Binding affinity of HLA-B44 binding peptides. Peptides are identified by amino acid sequence, SEQ ID NO., and binding affinity to the designated HLA-B44 alleles (expressed as an IC₅₀).

Table 23. Identified HLA-DQ allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 24. Binding affinity of HLA-DQ binding peptides. Peptides are identified by amino acid sequence, SEQ ID NO., and binding affinity to the designated HLA-DQ alleles (expressed as an IC₅₀).

Table 25. Identified HLA-DR allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 26. Binding affinity of HLA-DR binding peptides. Peptides are identified by amino acid sequence, SEQ ID NO., and binding affinity to the designated HLA-DR alleles (expressed as an IC₅₀).

Table 27. Binding affinity of HLA-DR binding peptides. Peptides are identified by amino acid sequence, SEQ ID NO., and binding affinity to the designated HLA-DR alleles (expressed as an IC₅₀).

Table 28. Identified murine MHC class I allele-binding peptides. Peptides are identified by amino acid sequence, SEQ ID NO., number of amino acids in peptide (AA), origin of peptide (organism), identity of originating protein, position of peptide within protein sequence, and analog status, wherein an analog is a peptide of the invention where the amino acid sequence of any naturally-occurring peptide sequence has been modified by substitution of one or more amino acid residues.

Table 29. Binding affinity of murine MHC class I-binding peptides. Peptides are identified by amino acid sequence, SEQ ID NO., and binding affinity to the designated murine MHC class I alleles (expressed as an IC₅₀).

TABLE 11
HLA-A1 SUPERTYPE

| | | | | | | |
|---|---|---|---|---|---|---|
| | SEQ ID | | | | | |
| Sequence | NO. | AA | Organism | Protein | Position | Analog |

| | | | | | |
|---|---|---|---|---|---|
| AYGPGPGKF | 9 | Artificial | Consensus | | A |
| | | sequence | | | |
| AEIPYLAKY | 9 | Artificial | pool | | A |
| | | sequence | consensus | | |
| AADAAAAKY | 9 | Artificial | | | PolyA |
| | | sequence | | | |
| AYSSWMYSY | 9 | EBV | EBNA3 | 176 | |
| LAEKTMKEY | 9 | FluA | POL2 | 16 | |
| GTYDYWAGY | 9 | Gonorrhea | | | |
| LSVHSIQNDY | 10 | Gonorrhea | | | |
| DTGQCPELVY | 10 | Gonorrhea | | | |
| DLLDTASALY | 10 | HBV | Core | 419 | |
| WFHISCLTF | 9 | HBV | NUC | 102 | |
| LSLDVSAAFY | 10 | HBV | pol | 426 | |
| LSGPGPGAFY | 10 | HBV | pol | 426 | A |
| LSLGPGPGFY | 10 | HBV | pol | 426 | A |
| LSLDGPGPGY | 10 | HBV | pol | 426 | A |
| LTYGRKLHLY | 10 | HBV | pol | 1098 | |
| KTGPGPGHLY | 10 | HBV | pol | 1098 | A |
| KTYGPGPGLY | 10 | HBV | pol | 1098 | A |
| KTYGGPGPGY | 10 | HBV | pol | 1098 | A |
| KYTSFPWL | 8 | HBV | pol | 745 | |
| FAAPFTQCGY | 10 | HBV | pol | 631 | |
| SYQHFRKLLL | 10 | HBV | pol | 4 | |
| LYSHPIILGF | 10 | HBV | pol | 492 | |
| MS1TDLEAY | 9 | HBV | X | 103 | |
| MYVGGPGPGVF | 11 | HCV | E1 | 275 | A |
| VMGSSYGF | 8 | HCV | NS5 | 2639 | |
| EVDGVRLHRY | 10 | HCV | NS5 | 2129 | |
| RTEILDLWVY | 10 | HIV | NEF | 182 | A |
| RQDILDLWVY | 10 | HIV | NEF | 182 | A |
| RTDILDLWYY | 10 | HIV | NEF | 182 | A |
| YTDGPGIRY | 9 | HIV | NEF | 207 | A |
| ATELHPEYY | 9 | HIV | NEF | 322 | A |
| DLWVYHTQGYY | 11 | HIV | NEF | 188 | A |
| WVYHTQGYY | 9 | HIV | NEF | 191 | A |
| FFLKEKGGF | 9 | HIV | NEF | 116 | A |
| LYVYHTQGY | 9 | HIV | NEF | 190 | A |
| ITKILYQSNPY | 11 | HIV | REV | 20 | A |
| KTLYQSNPY | 9 | HIV | REV | 22 | A |
| PVDPNLEPY | 9 | HIV | TAT | 3 | A |
| STVKHHMY | 8 | HIV | VIF | 23 | A |
| LSKISEYRHY | 10 | HPV | E6 | 70 | |
| ISEYRHYNY | 9 | HPV | E6 | 73 | |
| RFHNIRGRW | 9 | HPV | E6 | 131 | |
| RPLSKISEY | 9 | HPV | E6 | 68 | |
| RFHNISGRW | 9 | HPV | E6 | 124 | |
| TLEKLTNTGLY | 11 | HPV | E6 | 89 | |
| TLGPGPGTGLY | 11 | HPV | E6 | 89 | A |
| TLEGPGPGGLY | 11 | HPV | E6 | 89 | A |
| TLEKGPGPGLY | 11 | HPV | E6 | 89 | A |
| TLEKLGPGPGY | 11 | HPV | E6 | 89 | A |
| TLEKLTNTGLY | 11 | HPV | E6 | 89 | |
| TLEKITNTELY | 11 | HPV | E6 | 89 | |
| PYGVCIMCLRF | 11 | HPV | E6 | 59 | |
| ITDIILECVY | 10 | HPV | E6 | 30 | A |
| YSDISEYRHY | 10 | HPV | E6 | 77 | A |
| LThIEITCVY | 10 | HPV | E6 | 25 | A |
| YSDIRELRHY | 10 | HPV | E6 | 72 | A |
| ELSSALEIPY | 10 | HPV | E6 | 14 | |
| ETSSALEIPY | 10 | HPV | E6 | 14 | A |
| ELDSALEIPY | 10 | HPV | E6 | 14 | A |
| YTKVSEFRWY | 10 | HPV | E6 | 70 | A |
| YSDVSEFRWY | 10 | HPV | E6 | 70 | A |
| LThVSIACVY | 10 | HPV | E6 | 25 | A |
| FTSRIRELRY | 10 | HPV | E6 | 71 | A |
| YSDIRELRYY | 10 | HPV | E6 | 72 | A |
| LTDLRLSCVY | 10 | HPV | E6 | 26 | A |
| FTSKVRKYRY | 10 | HPV | E6 | 72 | A |
| YSDVRKYRYY | 10 | HPV | E6 | 73 | A |
| FYSKVSEFRF | 10 | HPV | E6 | 69 | A |
| FYSRIRELRF | 10 | HPV | E6 | 71 | A |
| PYAVCRVCLF | 10 | HPV | E6 | 62 | A |
| ITEYRHYNY | 9 | HPV | E6 | 73 | A |
| ISDYRHYNY | 9 | HPV | E6 | 73 | A |
| ITEYRHYQY | 9 | HPV | E6 | 73 | A |
| ISDYRHYQY | 9 | HPV | E6 | 73 | A |
| LTDLLIRCY | 9 | HPV | E6 | 99 | A |
| KTDQRSEVY | 9 | HPV | E6 | 35 | A |
| AYRDLCIVY | 9 | HPV | E6 | 53 | A |
| KYYSKISEY | 9 | HPV | E6 | 75 | A |
| KFYSKISEF | 9 | HPV | E6 | 75 | A |
| RYHNIRGRW | 9 | HPV | E6 | 131 | A |
| RFHNTRGRF | 9 | HPV | E6 | 131 | A |
| AYKDLFVVY | 9 | HPV | E6 | 48 | A |
| LFVVYRDSF | 9 | HPV | E6 | 52 | A |
| RYNNIAGHY | 9 | HPV | E6 | 126 | A |
| RFHNIAGHF | 9 | HPV | E6 | 126 | A |
| VYGTTLEKF | 9 | HPV | E6 | 83 | A |
| AYADLTVVY | 9 | HPV | E6 | 46 | A |
| AFADLTVVF | 9 | HPV | E6 | 46 | A |
| RYLSKISEY | 9 | HPV | E6 | 68 | A |
| RYHNISGRW | 9 | HPV | E6 | 124 | A |
| AYKDLCIVY | 9 | HPV | E6 | 48 | A |
| RYHSIAGQY | 9 | HPV | E6 | 126 | A |
| RFHSIAGQF | 9 | HPV | E6 | 126 | A |
| KYLFTDLRI | 9 | HPV | E6 | 44 | A |
| KFLFTDLRF | 9 | HPV | E6 | 44 | A |
| LYTDLRIVY | 9 | HPV | E6 | 46 | A |
| LFTDLRIVF | 9 | HPV | E6 | 46 | A |
| RFLSKISEF | 9 | HPV | E6 | 68 | A |
| EYRHYQYSF | 9 | HPV | E6 | 75 | A |
| RYHNIMGRW | 9 | HPV | E6 | 124 | A |
| RFHNIMGRF | 9 | HPV | E6 | 124 | A |
| NFACTELKF | 9 | HPV | E6 | 47 | A |
| PYAVCRVCF | 9 | HPV | E6 | 62 | A |
| LYYSKVRKY | 9 | HPV | E6 | 71 | A |
| VYADLRIVY | 9 | HPV | E6 | 46 | A |
| VFADLRIVF | 9 | HPV | E6 | 46 | A |
| NYSLYGDTF | 9 | HPV | E6 | 80 | A |
| RFHNISGRF | 9 | HPV | E6 | 124 | A |
| FTDLTIVY | 8 | HPV | E6 | 47 | |
| FTDLRIVY | 8 | HPV | E6 | 47 | |
| TLEKLTNTGLY | 11 | HPV | E6 | 89 | |
| LTDIEITGVY | 10 | HPV | E6 | 25 | A |
| LTDVSIACVY | 10 | HPV | E6 | 25 | A |
| ITDIILECVY | 10 | HPV | E6 | 30 | |
| KTDQRSEVY | 9 | HPV | B6 | 35 | |
| FTDLTIVY | 8 | HPV | E6 | 47 | |
| YSDTRELRYY | 10 | HPV | E6 | 72 | A |
| YTKVSEFRWY | 10 | HPV | E6 | 70 | A |
| FTSRIRELRY | 10 | HPV | E6 | 71 | A |
| FTSKVRKYRY | 10 | HPV | E6 | 72 | A |
| ISDYRHYNY | 9 | HPV | E6 | 73 | A |
| ISEYRHYQY | 9 | HPV | E6 | 73 | |
| ISDYRHYQY | 9 | HPV | E6 | 73 | A |
| EYRHYCYSLY | 10 | HPV | E6 | 82 | |
| EYRHYNYSLY | 10 | HPV | E6 | 75 | |
| LTDLLIRCY | 9 | HPV | E6 | 99 | |
| ETRITYCYSLY | 10 | HPV | E6 | 82 | A |
| EYDHYCYSLY | 10 | HPV | E6 | 82 | A |
| KTRYYDYSVY | 10 | HPV | E6 | 78 | A |
| KYDYYDYSVY | 10 | HPV | E6 | 78 | A |
| ETRHYNYSLY | 10 | HPV | E6 | 75 | A |
| EYDHYNYSLY | 10 | HPV | E6 | 75 | A |
| PTLKEYVLDLY | 11 | HPV | E7 | 6 | |
| HTDTPTLHEY | 10 | HPV | E7 | 2 | A |
| RTETPTLQDY | 10 | HPV | E7 | 2 | A |
| ETDPVDLLCY | 10 | HPV | E7 | 20 | A |
| QTEQATSNYY | 10 | HPV | E7 | 46 | A |
| ATDNYYIVTY | 10 | HPV | E7 | 50 | A |
| LTEYVLDLY | 9 | HPV | E7 | 8 | A |
| QTEQATSNY | 9 | HPV | E7 | 46 | A |
| RQAKQHTCY | 9 | HPV | E7 | 51 | |
| RTAKQHTCY | 9 | HPV | E7 | 51 | A |
| HTDTPTLHEY | 10 | HPV | E7 | 2 | A |
| RTETPTLQDY | 10 | HPV | E7 | 2 | A |
| PTLKEYVLDLY | 11 | HPV | E7 | 6 | |
| LTEYVLDLY | 9 | HPV | E7 | 8 | A |
| QAEQATSNY | 9 | HPV | E7 | 46 | |
| ATSNYYIVTY | 10 | HPV | E7 | 50 | |
| ATDNYYIVTY | 10 | HPV | E7 | 50 | A |
| RVLPPNWKY | 9 | Human | 40s | 132 | |
| | | | riboprot | | |
| | | | S13 | | |
| RLAHEVGWKY | 10 | Human | 60s | 139 | |
| | | | ribo prot | | |
| | | | L13 A | | |
| AYKKQFSQY | 9 | Human | 60s | 217 | |
| | | | ribo prot | | |
| | | | L5 | | |
| AADNPPAQY | 9 | Human | CEA | 261 | A |
| RSGPGPGNVLY | 11 | Human | CEA | 225 | A |
| RSDGPGPGVLY | 11 | Human | CEA | 225 | A |
| RSDSGPGPGLY | 11 | Human | CEA | 225 | A |
| RSDSVGPGPGY | 11 | Human | CEA | 225 | A |
| SLFVSNHAY | 9 | Human | fructose | 355 | |
| | | | biphosphate- | | |
| | | | aldolase | | |
| RWGLLLALL | 9 | Human | Her2/neu | 8 | |
| YTGPGPGVY | 9 | Human | Jchain | 102 | A |
| YTAGPGPGY | 9 | Human | Jchain | 102 | A |
| TQDLVQEKY | 9 | Human | MAGE1 | 240 | |
| TQGPGPGKY | 9 | Human | MAGE1 | 240 | A |
| TQDGPGPGY | 9 | Human | MAGE1 | 240 | A |
| EVGPGPGLY | 9 | Human | MAGE3 | 161 | A |
| EVDGPGPGY | 9 | Human | MAGE3 | 161 | A |
| IYGPGPGLIF | 10 | Human | MAGE3 | 195 | A |
| RISGVDRYY | 9 | Human | NADH | 53 | |
| | | | ubiqoxido- | | |
| | | | reductase | | |
| IMVLSFLF | 8 | Pf | CSP | 427 | |
| ALFQEYQCY | 9 | Pf | CSP | 18 | |
| LSEYYDXDIY | 10 | Pf | | 347 | |
| FQAAESNERY | 10 | Pf | | 13 | |
| ELEASISGKY | 10 | Pf | | 81 | |
| FVSSIFISFY | 10 | Pf | | 255 | |
| KVSDEIWNY | 9 | Pf | | 182 | |
| IMNHLMTLY | 9 | Pf | | 38 | |
| LIENELMNY | 9 | Pf | | 149 | |
| NVDQQNDMY | 9 | Pf | | 182 | |
| SSFFMNRFY | 9 | Pf | | 309 | |
| QAAESNERY | 9 | Pf | | 14 | |
| LEASISGKY | 9 | Pf | | 82 | |
| NLALLYGEY | 9 | Pf | | 188 | |
| SSPLFNNFY | 9 | Pf | | 14 | |
| QNADKNFLY | 9 | Pf | | 145 | |
| VSSIFISFY | 9 | Pf | | 256 | |
| SYKSSKRDKF | 10 | Pf | | 225 | |
| RYQDPQNYEL | 10 | Pf | | 21 | |
| DFFLKSKFNI | 10 | Pf | | 3 | |
| NYMKIMNHL | 9 | Pf | | 34 | |
| TYKKKNNHI | 9 | Pf | | 264 | |
| SFFMNRFYI | 9 | Pf | | 310 | |
| FYITTRYKY | 9 | Pf | | 316 | |
| KYINFINFI | 9 | Pf | | 328 | |
| TWKPTIFLL | 9 | Pf | | 135 | |
| KYNYFIHFF | 9 | Pf | | 216 | |
| HFFTWGTMF | 9 | Pf | | 222 | |
| RMTSLKNEL | 9 | Pf | | 61 | |
| YYNNFNNNY | 9 | Pf | | 77 | |
| GTDEXRNXY | 9 | Unknown | Naturally | | A |
| | | | processed | | |
| ETDXXXDRSEY | 11 | Unknown | Naturally | | A |
| | | | processed | | |
| FTDVNSXXRY | 10 | Unknown | Naturally | | A |
| | | | processed | | |
| VXDPYNXKY | 9 | Unknown | Naturally | | A |
| | | | processed | | |
| VADKVHXMY | 9 | Unknown | Naturally | | A |
| | | | processed | | |
| ETXXPDWSY | 9 | Unknown | Naturally | | A |
| | | | processed | | |
| XTHNXVDXY | 9 | Unknown | Naturally | | A |
| | | | processed | | |

TABLE 12

HLA-A1 SUPERTYPE

| | | | | |
|---|---|---|---|---|
| | SEQ ID | | | |
| Sequence | NO. | A*0101 | A*2902 | A*3002 |

| | | | |
|---|---|---|---|
| AYGPGPGKF | | 44854 | 3.2 |
| AEIPYLAKY | | | 144 |
| AADAAAAKY | 20 | | |
| AYSSWMYSY | | | 4.9 |
| LAEKTMKEY | 174 | | |
| GTYDYWAGY | 141 | | |
| LSVHSIQNDY | 279 | | |
| DTGQCPELVY | 129 | | |
| DLLDTASALY | | 74 | 37 |
| WFHISCLTF | 85324 | 95 | 75094 |
| LSLDVSAAFY | 267 | 12 | 7.1 |
| LSGPGPGAIFY | 25 | 1383 | 6.6 |
| LSLGPGPGFY | 21 | 132 | 8.2 |
| LSLDGPGPGY | 266 | 274 | 181 |
| KTYGRKLHLY | 171 | 27 | 1.5 |
| KTGPGPGHLY | 29 | 192 | 1.3 |
| KTYGPGPGLY | 5.7 | 227 | 0.96 |
| KTYGGPGPGY | 282 | 228 | 1.7 |
| KYTSFPWL | | >172413 | 346 |
| FAAPFTQCGY | | 461 | 1364 |
| SYQHFRIKLLL | >83333 | 28 | 3768 |
| LYSHPIILGF | 3166 | 109 | 1116 |
| MSTTDLEAY | | 2565 | 396 |
| MYVGGPGPGVF | | 89 | 2870 |
| XTMGSSYGF | | 145 | 41967 |
| EVDGVRLLTRY | | 14940 | 113 |
| RTEJLDLWVY | 99 | 10204 | 315 |
| RQDILDLWvY | 8995 | 13928 | 95 |
| RTDILDLWVY | 85 | 13424 | 360 |
| YTDGPGIRY | 11 | 562 | 7911 |
| ATELHPEYY | 43 | 6608 | 1734 |
| DLWYYHTQGYY | 5880 | 852 | 16 |
| WVYHTQGYY | 703 | 215 | 5.6 |
| FFLKEKGGF | | 3015 | 141 |
| LYVYHTQGY | | 216 | 258 |
| ITKILYQSNPY | >10060 | 64908 | 298 |
| KTLYQSNPY | 6912 | 1703 | 35 |
| PVDPNLEPY | 195 | 13193 | 7121 |
| STVKHHMY | 8132 | 1760 | 68 |
| LSKISEYRIHY | 14306 | 55190 | 186 |
| ISEYRHYNY | 25 | 1329 | 32 |
| RFHNIRGRW | 52917 | 18 | 58 |
| RFLSKISEY | >40322 | 34623 | 23 |
| RFHNISGRW | 48564 | 174 | 37 |
| TLEKLTNTGLY | 23 | 991 | 92 |
| TLGPGPGTGLY | 350 | 1320 | 7.4 |
| TLEGPGPGGLY | 11 | 2320 | 40 |
| TLEKGPGPGLY | 13 | 2036 | 40 |
| TLEKLGPGPGY | 269 | 4473 | 1962 |
| TLEKLTNTGLY | 77 | 5500 | 154 |
| TLEKITNTELY | 17 | 8402 | 3897 |
| PYGVCIMCLRF | | 69 | 43722 |
| ITDIILECVY | 1.8 | 7660 | 505 |
| YSDISEYRHY | 3.8 | 1350 | 514 |
| LTDIEITCVY | 12 | 540 | 80 |
| YSDIRELRHY | 14 | 1137 | 740 |
| ELSSALEIPY | 171 | 6031 | 4472 |
| ETSSALEIPY | 19 | 12026 | 7144 |
| ELDSALEIPY | 38 | 82189 | 38284 |
| YTKVSEFRWY | 276 | 3308 | 420 |
| YSDVSEFRWY | 3.9 | 1842 | 1026 |
| LTDVSIACVY | 2.9 | 764 | 72 |
| FTSRTRELRY | 4.4 | 77 | 50 |
| YSDIRELRYY | 9.4 | 733 | 456 |
| LTDLRLSCVY | 45 | 1783 | 613 |
| FTSKVRKYRY | 64 | 6677 | 52 |
| YSDVRKYRYY | 19 | 849 | 794 |
| FYSKVSEFRP | | 79 | 18453 |
| FYSRIRELRF | | 83 | 12598 |
| PYAVCRVCLF | | 407 | 5226 |
| ITEYRHYNY | 114 | 625 | 418 |
| ISDYRHYNY | 16 | 45 | 455 |
| ITEYRHYQY | 90 | 1030 | 526 |
| ISDYRHYQY | 13 | 37 | 382 |
| LTDLLIRCY | 13 | 6857 | 5515 |
| KTDQRSEVY | 84 | 200429 | 1174 |
| AYRDLCIVY | | 7117 | 66 |
| KYYSKISEY | | 702 | 1.3 |
| KEYSKISEF | | 73339 | 306 |
| RYHNIRGRW | | 122644 | 15 |
| RFHINIRGRF | | 346 | 0.69 |
| AYKDLFVVY | | 639 | 1.3 |
| LFVVYRDSF | | 919 | 18 |
| RYRNIAGHY | | 138 | 0.93 |
| RFHMAGITF | | 635 | 1.4 |
| VYGTTLEKF | | 75267 | 220 |
| AYADLTVVY | | 136 | 9.3 |
| AFADLTVVF | | 779 | 137 |
| RYLSKISEY | | 4247 | 1.1 |
| RYHNISGRW | | 104884 | 13 |
| AYKDLCIVY | | 5205 | 29 |
| RYHSIAGQY | | 544 | 1.4 |
| RFHSIAGQF | | 481 | 1.2 |
| KYLFTDLRI | | 78575 | 339 |
| KFLFTDLRF | | 44 | 152 |
| LYTDLR1VY | | 4.8 | 2.1 |
| LFTDLRIVF | | 164 | 2649 |
| RFLSKISEF | | 40103 | 201 |
| EYRHYQYSF | | 13707 | 430 |
| RYHMMGRW | | 106990 | 7.1 |
| RFEINTMGRF | | 174 | 1.3 |
| NFACTELKF | | 46 | 6826 |
| PYAVCRVCF | | 5602 | 316 |
| LYYSKVRKY | | 1452 | 28 |
| VYADLRIVY | | 8.2 | 8.3 |
| VFADLRIVF | | 87 | 24062 |
| NYSLYGDTF | | 20945 | 64 |
| RFHNISGRF | | 572 | 2.8 |
| FTDLTIVY | 16 | 1275 | 39043 |
| FTDLRIVY | 26 | 813 | 8060 |
| TLEKLTNTGLY | 174 | | |
| LTDIEITCVY | 33 | | |
| LTDVSIACVY | 57 | | |
| ITDIILECVY | 187 | | |
| KTDQRSEVY | 41 | | |
| FTDLTIVY | 34 | | |
| YSDIIRELRYY | 20 | | |
| YTKVSEFRWY | 204 | | |
| FTSRIRELRY | 25 | | |
| FTSKVRKYRY | 37 | | |
| ISDYRHYNY | 28 | | |
| ISEYRHYQY | 40 | | |
| ISDYRHIYQY | 28 | | |
| EYRHIYCYSLY | 125 | 198 | 3.7 |
| BYRHYNYSLY | 111027 | 956 | 12 |
| LTDLLILRCY | 64 | | |
| ETRHYCYSLY | 43 | 755 | 10 |
| EYDHYCYSLY | 110081 | 799 | 77 |
| KTRYYDYSVY | 2957 | 87841 | 0.71 |
| KYDYYDYSVY | 186339 | 5749 | 11 |
| ETRHYNYSLY | 445 | 5464 | 29 |
| BYDHYNYSLY | 11251 | 777 | 93 |
| PTLKEYVLDLY | 195 | 805 | 408 |
| HTDTPTLHEY | 20 | 1509 | 54 |
| RTETPTLQDY | 11 | 1987 | 239 |
| BTDPVDLLCY | 6.4 | 4110 | 52640 |
| QTEQATSNYY | 11 | 9576 | 500 |
| ATDNYYIVTY | 7.4 | 1918 | 65 |
| LTEYVLDLY | 6.0 | 941 | 81 |
| QTEQATSNY | 14 | 119081 | 3247 |
| RQAKQHTCY | >135135 | 155246 | 108 |
| RTAKQHTCY | 5647 | 130343 | 346 |
| HTDTPTLHEY | 30 | | |
| RTETPTLQDY | 40 | | |
| PTLKEYVLDLY | 426 | | |
| LTEYVLDLY | 8.0 | | |
| QAEQATSNY | 132 | | |
| ATSNYYIVTY | 428 | | |
| ATDNYYIVTY | 19 | | |
| RVLPPNWKY | | | 3.0 |
| RLAHEVGWKY | | | 3.8 |
| AYKKQFSQY | | | 5.3 |
| AADNPPAQY | 9.2 | | |
| RSGPGPGNYLY | 172 | 11270 | 6.3 |
| RSDGPGPGVLY | 12 | 13162 | 12 |
| RSDSGPGPGLY | 3.3 | 11856 | 4.2 |
| RSDSVGPGPGY | 23 | 31193 | 33 |
| SLFVSNHAY | | | 1.1 |
| RWGLLLALL | | 61253 | 300 |
| YTGPGPGVY | 2.7 | 2015 | 6.4 |
| YTAGPGPGY | 7.0 | 28 | 755 |
| TQDLVQEKY | 57 | 33304 | 3796 |
| TQGPGPGKY | 4192 | 36746 | 3.2 |
| TQDGPGPGY | 381 | 37093 | 541 |
| EVGPGPGLY | 50 | 18183 | 45 |
| EVDGPGPGY | 29 | 25775 | 5766 |
| IYGPGPGLIF | | 58 | 6845 |
| RISGVDRYY | | | 3.0 |
| IMVLSFLF | | 111 | 30000 |
| ALFQEYQCY | >42016 | 149 | 1032 |
| LSEYYDXD1Y | 11 | 1647 | 489 |
| FQAAESNERY | 8958 | 1780 | 372 |
| ELEASISGKY | 142 | 21934 | 463 |
| FVSSIFISFY | 118 | 22 | 84 |
| KVSDETWNY | 435 | 230 | 1.9 |
| IMNIILMTLY | 150 | 1.7 | 1.8 |
| LIENELMNY | 412 | 3936 | 169 |
| NVDQQNIDMY | 47 | 22173 | 79057 |
| SSFFMNRFY | 239 | 36 | 7.5 |
| QAAESNERY | 353 | 24281 | 3011 |
| LEASISGKY | 57792 | 17824 | 87 |
| NLALLYGEY | 275 | 138 | 102 |
| SSPLENNFY | 117 | 389 | 73 |
| QNADKNFLY | 3811 | 24 | 663 |
| VSSIFISFY | 144 | 1800 | 55 |
| SYKSSKRDKF | | 12594 | 88 |
| RYQDPQNYEL | | 79717 | 189 |
| DFFLKSKFNI | | 47714 | 491 |
| NYMKIIVINHL | | 45443 | 110 |
| TYKKKNNHI | | 21642 | 162 |
| SFFMNRFYI | | 200 | 1022 |
| FYITTRYKY | | 9.6 | 7.5 |
| KYTNIFTNFI | | 25475 | 55 |
| TWKPTIFLL | | 21155 | 306 |
| KYNYFIIIFF | | 319 | 2.7 |
| HFFTWGTMF | | 4.0 | 220 |
| RMTSLKINEL | | 40270 | 14 |
| YYNNFNNNY | | 19 | 34 |
| GTDEXRNXY | 0.67 | | |
| ETDXXXDRSEY | 2.0 | | |
| FTDVNSXXRY | 0.20 | | |
| VXDPYNXKY | 2.3 | | |
| VADKVHXMY | 2.4 | | |
| ETXXPDWSY | 11 | | |
| XTHNXVDXY | 1.4 | | |

TABLE 13

HLA-A2 SUPERTYPE

| | | | | | | |
|---|---|---|---|---|---|---|
| | SEQ ID | | | | | |
| Sequence | NO. | AA | Organism | Protein | Position | Analog |

| | | | | | |
|---|---|---|---|---|---|
| FPFKYAAAV | 9 | Artificial | | | A |
| | | sequence | | | |
| AMAKAAAAV | 9 | Artificial | | | PolyA |
| | | sequence | | | |
| AMAKAAAAL | 9 | Artificial | | | PolyA |
| | | sequence | | | |
| AMAKAAAAT | 9 | Artificial | | | PolyA |
| | | sequence | | | |
| AXAKAAAAL | 9 | Artificial | | | PolyA |
| | | sequence | | | |
| FVYGGSKTSL | 10 | EBNA | | 508 | |
| ILGPGPGL | 8 | Flu | M1 | 59 | A |
| GILGFVFTL | 9 | Flu | M1 | 58 | |
| GLIYNRMGAV | 10 | FluA | M1 | 129 | |
| VLMEWLKTRPI | 11 | FluA | M1 | 41 | |
| FLPSDYFPSV | 10 | HBV | Core | 18 | A |
| FLGPGPGPSV | 10 | HBV | core | 18 | A |
| FLPGPGPGSV | 10 | HBV | core | 18 | A |
| FLPSGPGPGV | 10 | HBV | core | 18 | A |
| WLGPGPGFV | 9 | HBV | env | 335 | A |
| WLSGPGPGV | 9 | HBV | env | 335 | A |
| GVLGWSPQv | 9 | HBV | env | 62 | A |
| PVLPIFFCV | 9 | HBV | env | 377 | A |
| VVQAGFFLV | 9 | HBV | env | 177 | A |
| FLLAQFTSAI | 10 | HBV | Pol | 503 | |
| YLLTLWKAGI | 10 | HBV | pol | 147 | |
| YLGPGPGAGI | 10 | HBV | pol | 147 | A |
| YLLGPGPGGI | 10 | HBV | pol | 147 | A |
| YLLTGPGPGI | 10 | HBV | pol | 147 | A |
| HVYSHPIIV | 9 | HBV | pol | 1076 | A |
| FVLSLGIHV | 9 | HBV | pol | 562 | A |
| YVDDVVLGV | 9 | HBV | pol | 538 | A |
| IVRGTSFVYV | 10 | HBV | pol | 773 | A |
| SLGPGPGIAV | 10 | HIV | env | 814 | A |
| SLLGPGPGAV | 10 | HIV | env | 814 | A |
| SLLNGPGPGV | 10 | HIV | env | 814 | A |
| KITPLCVTL | 9 | HIV | Env | 134 | A |
| KLTPLCVTM | 9 | HIV | Env | 134 | A |
| KLTPLCVPL | 9 | HIV | Env | 134 | A |
| KLTPLCVSL | 9 | HIV | Env | 134 | A |
| KLTPLCITL | 9 | HIV | Env | 134 | A |
| QLTPLCVTL | 9 | HIV | Env | 134 | A |
| KLTPRCVTL | 9 | HIV | Env | 134 | A |
| ELTPLCVTL | 9 | HIV | Env | 134 | A |
| QMTFLCVQM | 9 | HIV | Env | 134 | A |
| KMTFLCVQM | 9 | HIV | Env | 134 | A |
| KLTPLCVAL | 9 | HIV | Env | 134 | A |
| KLTPFCVTL | 9 | HIV | Env | 134 | A |
| SLYNTVATL | 9 | HIV | GAG | 77 | |
| VLAEAMSQT | 9 | HIV | Gag | 386 | A |
| VLAEAMSQA | 9 | HIV | Gag | 386 | A |
| VLAEAMSQI | 9 | HIV | Gag | 386 | A |
| ILAEAMSQV | 9 | HIV | Gag | 386 | A |
| VLAEAMSKV | 9 | HIV | Gag | 386 | A |
| VLAEAMSHA | 9 | HIV | Gag | 386 | A |
| ILAEAMSQA | 9 | HIV | Gag | 386 | A |
| VLAEAMSRA | 9 | HIV | Gag | 386 | A |
| VLAEAMATA | 9 | HIV | Gag | 386 | A |
| ILAEAMASA | 9 | HIV | Gag | 386 | A |
| MTHNPPIPV | 9 | HIV | Gag | 271 | A |
| MTNNPPVPV | 9 | HIV | Gag | 271 | A |
| MTSNPPIPV | 9 | HIV | Gag | 271 | A |
| MTSNPPVPV | 9 | HIV | Gag | 271 | A |
| MTSDPPIPV | 9 | HIV | Gag | 271 | A |
| MTGNPPIPV | 9 | HIV | Gag | 271 | A |
| MTGNPPVPV | 9 | HIV | Gag | 271 | A |
| MTGNPAIPV | 9 | HIV | Gag | 271 | A |
| MTGNPSIPV | 9 | HIV | Gag | 271 | A |
| MTANPPVPV | 9 | HIV | Gag | 271 | A |
| SLYNTVATL | 9 | hiv | gag | 77 | |
| QAHONISRA | 9 | HIV | gp160 | 332 | |
| FLKEKGGV | 8 | HIV | NEF | 117 | A |
| GLGAVSRDL | 9 | HIV | NEF | 45 | A |
| GLITSSNTA | 9 | HIV | NEF | 62 | A |
| ALEEEEVGFPV | 11 | HIV | NEF | 83 | A |
| FLKEKGGLEGV | 11 | HIV | NEF | 117 | A |
| FLKEKGGLDGV | 11 | HIV | NEF | 117 | A |
| GLIYSKKRQEV | 11 | HIV | NEF | 173 | A |
| LLYSKKRQEI | 10 | HIV | NEF | 174 | A |
| LLYSKKRQEIL | 11 | HIV | NEF | 174 | A |
| RLDILDLWV | 9 | HIV | NEF | 182 | A |
| EILDLWVYHV | 10 | HIV | NEF | 185 | A |
| ILDLWVYHV | 9 | HIV | NEF | 186 | A |
| ILDLWVYNV | 9 | HIV | NEF | 186 | A |
| WLNYTPGPGT | 10 | HIV | NEF | 204 | A |
| WQNYTPGPGV | 10 | HIV | NEF | 204 | A |
| WLNYTPGPGI | 10 | HIV | NEF | 204 | A |
| YLPGPGIRYPL | 11 | HIV | NEF | 207 | A |
| YTPGPGIRYPV | 11 | HIV | NEF | 207 | A |
| LLFGWCFKL | 9 | HIV | NEF | 221 | A |
| LTFGWCFKV | 9 | HIV | NEF | 221 | A |
| LLFGWCFKLV | 10 | HIV | NEF | 221 | A |
| FGVRPQVPL | 9 | HIV | nef | 84 | A |
| FTVRPQVPL | 9 | HIV | nef | 84 | A |
| FSVRPQVPL | 9 | HIV | nef | 84 | A |
| YLKEPVHGV | 9 | HIV | pol | 476 | A |
| FLKEPVHGV | 9 | HIV | pol | 476 | |
| PVPLQLPPV | 9 | HIV | REV | 74 | A |
| LQLPPLERV | 9 | HIV | REV | 77 | A |
| LLLPPLERLTL | 11 | HIV | REV | 77 | A |
| LQLPPLERLTV | 11 | HIV | REV | 77 | A |
| ILWQVDRM | 8 | HIV | VIF | 9 | A |
| KLGSLQYL | 8 | HIV | VIF | 146 | A |
| KVGSLQYV | 8 | HIV | VIF | 146 | A |
| TLHDLCQAV | 9 | HPV | E6 | 11 | A |
| TLQDIVLHL | 9 | HPV | E7 | 7 | |
| TLGPGPGHL | 9 | HPV | E7 | 7 | A |
| TLQGPGPGL | 9 | HPV | E7 | 7 | A |
| TLSFVCPWCV | 10 | HPV | E7 | 94 | A |
| TLSFVCPWCA | 10 | HPV18 | E7 | 93 | |
| RTLHDLCQA | 9 | HPV33 | E6 | 10 | |
| TLHDLCQAL | 9 | HPV33 | E6 | 11 | |
| YLSGADLNL | 9 | Human | GEA | 605 | A |
| YLEPGPVTA | 9 | Human | gp100 | 280 | |
| LLDGTATLRL | 10 | Human | gp100 | 457 | |
| KVYGLSAFV | 9 | Human | Her2/neu | 369 | A |
| IISAVVAIL | 9 | Human | Her2/neu | 654 | A |
| ILSAVVGIL | 9 | Human | Her2/neu | 654 | A |
| IISAVVGFL | 9 | Human | Her2/neu | 654 | A |
| IISAVVGIV | 9 | Human | Her2/neu | 654 | A |
| KISAVVGIL | 9 | Human | Her2/neu | 369 | A |
| KIFAVVGIL | 9 | Human | Her2/neu | 369 | A |
| KIFASVAIL | 9 | Human | Her2/neu | 369 | A |
| ELVSEFSRV | 9 | Human | Her2/neu | 971 | A |
| VLVHPQWVV | 9 | Human | Kallikrein2 | 53 | A |
| VLVHPQWVLTV | 11 | Human | Kallikrein2 | 53 | A |
| DLMLLRLSEPV | 11 | Human | Kallikrein2 | 120 | A |
| PLVCNGVLQGV | 11 | Human | Kallikrein2 | 216 | A |
| VLVHPQWVLTV | 11 | Human | Kallikrein2 | 53 | A |
| PLVGNGVLQGV | 11 | Human | Kallikrein2 | 216 | A |
| QLGPGPLMEV | 11 | Human | MAGE3 | 159 | A |
| QLVGPGPGMEV | 11 | Human | MAGE3 | 159 | A |
| QLVFGPGIGEV | 11 | Human | MAGE3 | 159 | A |
| QLVFGGPGPGV | 11 | Human | MAGE3 | 159 | A |
| ALGIGILTV | 9 | Human | MART1 | 27 | A |
| AMGIGILTV | 9 | Human | MART1 | 27 | A |
| LLWQPIPV | 8 | Human | PAP | 136 | |
| LLGPGPGV | 8 | Human | PAP | 136 | A |
| VLAKELKFVTL | 11 | Human | PAP | 30 | |
| VLGPGPGFVTL | 11 | Human | PAP | 30 | A |
| VLAGPGPGVTL | 11 | Human | PAP | 30 | A |
| VLAKGPGPGTL | 11 | Human | PAP | 30 | A |
| VLAKEGPGPGL | 11 | Human | PAP | 30 | A |
| TLMSAMTNV | 9 | Human | PAP | 112 | A |
| ILYSAHDTTV | 10 | Human | PAP | 384 | A |
| IVYSAHDTTV | 10 | Human | PAP | 284 | A |
| VTAKELKFV | 9 | Human | PAP | 30 | A |
| ITYSAHDTTV | 10 | Human | PAP | 284 | A |
| SLSLGFLFV | 9 | Human | PAP | | |
| SLSLGFLFLV | 10 | Human | PAP | | |
| LLALFPPEGV | 10 | Human | PAP | | |
| LVALFPPEGV | 10 | Human | PAP | | |
| ALFPPEGVSV | 10 | Human | PAP | | |
| GLHGQDLFGV | 10 | Human | PAP | | |
| LLPPYASCHV | 10 | Human | PAP | | |
| LLWQPIPVHV | 10 | Human | PAP | | |
| MLLRLSEPV | 9 | Human | PSA | 118 | A |
| ALGTTCYV | 8 | Human | PSA | 143 | A |
| VLRLFVCFLI | 10 | Pf | | 2 | |
| FLIFHFFLFL | 10 | Pf | | 9 | |
| LIFHFFLFLL | 10 | Pf | | 10 | |
| FLFLLYILFL | 10 | Pf | | 15 | |
| RLPVIGSFLV | 10 | Pf | | 32 | |
| VICSFLVFLV | 10 | Pf | | 35 | |
| FLVFLVFSNV | 10 | Pf | | 39 | |
| MMIMIKFMGV | 10 | Pf | | 62 | |
| FLLYILFLV | 9 | Pf | | 17 | |
| VICSFLVFL | 9 | Pf | | 35 | |
| ATYGIIVPV | 9 | Pf | | 159 | |
| KIYKIIIWI | 9 | Pf | | 9 | |
| YMIKKLLKI | 9 | Pf | | 23 | |
| LMTLYQIQV | 9 | Pf | | 42 | |
| FMGVIYIMI | 9 | Pf | | 68 | |
| FMNRFYITT | 9 | Pf | | 312 | |
| YQDPQNYEL | 9 | Pf | | 22 | |
| KTWKPTWL | 9 | Pf | | 134 | |
| LLNESNIFL | 9 | Pf | | 142 | |
| FTHFFTWGT | 9 | Pf | | 220 | |
| VLFLQMMNV | 9 | Pf | | 180 | |
| NQMIFVSSI | 9 | Pf | | 251 | |
| MIFVSSIFI | 9 | Pf | | 253 | |
| SIFISFYLI | 9 | Pf | | 258 | |
| RLFEESLGI | 9 | Pf | | 293 | |
| ALWGFFPVL | 9 | Unknown | A2 | | A |
| | | | allo- | | |
| | | | epitope | | |
| SVYDFFVWL | 9 | | TRP2 | 180 | |
| FAPGFFPYL | 9 | | | | |
| QLFEDKYAL | 9 | | | | |
| MLLSVPLLL | 9 | | | | |

TABLE 14
HLA-A2 SUPERTYPE

| | | | | | | |
|---|---|---|---|---|---|---|
| | SEQ | | | | | |
| | ID | | | | | |
| Sequence | NO. | A*0201 | A*0202 | A*0203 | A*0206 | A*6802 |

| | | | | | |
|---|---|---|---|---|---|
| FPFKYAAAV | | | | | 92 |
| AMAKAAAAV | 181 | 196 | 6.7 | 1485 | 177 |
| AMAKAAAAL | 413 | 123 | 3.7 | 18500 | 320 |
| AMAKAAAAT | 15143 | 12413 | 84 | 37000 | >26666.67 |
| AXAKAAAAL | >50000 | 469 | 3300 | 37000 | >11428.57 |
| FVYGGSKTSL | 296 | | | | |
| ILGPGPGL | 672 | 45 | 530 | 1262 | 56099 |
| GILGFVFTL | 1.0 | 10 | 236 | 2.1 | 1395 |
| GLIYNRMGAV | 317 | | | | |
| VLMEWLKTRPI | 464 | | | | |
| FLPSDYFPSV | 8.5 | 3.3 | 3.2 | 2.2 | 276 |
| FLGPGPGPSV | 17 | 0.80 | 2.5 | 55 | 286 |
| FLPGPGPGSV | 98 | 18 | 4.0 | 665 | 332 |
| FLPSGPGPGV | 21 | 1.2 | 3.4 | 64 | 40 |
| WLGPGPGFV | 171 | 4.1 | 2.2 | 530 | 293 |
| WLSGPGPGV | 220 | 2.5 | 12 | 885 | 24 |
| GVLGWSPQV | 22 | 157 | 389 | 28 | 9428 |
| PVLPIFFCV | 8.7 | 3136 | 14286 | 22 | 1814 |
| VVQAGFFLV | 440 | 79 | 2503 | 81 | 617 |
| FLLAQFTSAI | 65 | 1.9 | 4.8 | 148 | 533 |
| YLLTLWKAGI | 20 | 19 | 20 | 40 | 1388 |
| YLGPGPGAGI | 161 | 1.0 | 4.2 | 548 | 315 |
| YLLGPGPGGI | 180 | 12 | 3.3 | 89 | 2064 |
| YLLTGPGPGI | 42 | 15 | 59 | 60 | 5678 |
| HVYSHPIIV | 150 | 1923 | 14 | 1199 | 123 |
| FVLSLGIHV | 45 | 399 | 2817 | 131 | 112 |
| YVDDVVLGV | 18 | 14 | 70 | 16 | 354 |
| IYRGTSFVYV | 50000 | 5301 | 69 | 5398 | 1217 |
| SLGPGPGIAV | 1131 | 5.3 | 11 | 917 | 281 |
| SLLGPGPGAV | 95 | 17 | 2.6 | 642 | 795 |
| SLLNGPGPGV | 65 | 3.8 | 14 | 63 | 45 |
| KITPLCVTL | 461 | 36 | 528 | 59 | 883 |
| KLTPLCVTM | 340 | 3.6 | 143 | 197 | 6288 |
| KLTPLCVPL | 15 | 0.25 | 297 | 135 | 67 |
| KLTPLCVSL | 67 | 2.4 | 240 | 16 | 5947 |
| KLTPLCITL | 1.7 | 0.27 | 23 | 1.7 | 9155 |
| QLTPLCVTL | 64 | 1.5 | 57 | 368 | 933 |
| KLTPRCVTL | 597 | 150 | 20 | 1554 | >63492.06 |
| ELTPLCVTL | 7190 | 38 | 231 | 1919 | 32 |
| QMTFLCVQM | 3153 | 40 | 1127 | 232 | 1297 |
| KMTFLCVQM | 1793 | 22 | 525 | 100 | 8744 |
| KLTPLCVAL | 209 | 2.3 | 54 | 11 | 13009 |
| KLTPFCVTL | 87 | 0.37 | 28 | 78 | 11814 |
| SLYNTVATL | 290 | 6573 | 68 | 37000 20000 | |
| VLAEAMSQT | 290 | 2.2 | 0.65 | 236 | 447 |
| VLAEAMSQA | 24 | 1.1 | 0.30 | 9.6 | 271 |
| VLAEAMSQI | 71 | 0.15 | 0.87 | 70 | 207 |
| ILAEAMSQV | 38 | 1.1 | 1.1 | 101 | 34 |
| VLAEAMSKV | 230 | 1.8 | 1.4 | 93 | 329 |
| VLAEAMSHA | 149 | 1.7 | 1.2 | 121 | 431 |
| ILAEAMSQA | 29 | 1.0 | 1.1 | 8.6 | 253 |
| VLAEAMSRA | 127 | 0.88 | 1.0 | 20 | 229 |
| VLAEAMATA | 6.7 | 1.4 | 0.73 | 8.6 | 33 |
| ILAEAMASA | 22 | 0.72 | 0.82 | 6.8 | 343 |
| MTHNPPIPV | 167 | 119 | 1.4 | 158 | 1.4 |
| MTNNPPVPV | 86 | 18 | 0.42 | 287 | 309 |
| MTSNPPIPV | 53 | 16 | 0.39 | 250 | 3.8 |
| MTSNPPVPV | 22 | 29 | 0.80 | 81 | 1.1 |
| MTSDPPIPV | 107 | 13 | 0.45 | 587 | 2.5 |
| MTGNPPIPV | 125 | 11 | 0.74 | 79 | 7.8 |
| MTGNPPVPV | 2021 | 158 | 23 | 35 | 0.84 |
| MTGNPAIPV | 1200 | 24 | 10 | 213 | 0.48 |
| MTGNPSIPV | 16 | 1.1 | 0.43 | 257 | 0.57 |
| MTANPPVPV | 20 | 5.0 | 0.62 | 134 | 4.0 |
| SLYNTVATL | 367 | 79 | 19 | 15072 | 247113 |
| QAHCNISRA | 338 | | | | |
| FLKEKGGV | 13327 | 653 | 267 | >14341.09 | >19464.72 |
| GLGAVSRDL | 18679 | 436 | 1733 | >10393.26 | >16666.67 |
| GLITSSNTA | 5800 | 102 | 64 | 7865 | >14311.27 |
| ALEEEEVGFPV | 2420 | 487 | 15744 | 2988 | >13793.1 |
| FLKEKGGLEGV | 322 | 3.5 | 6.8 | 739 | 1252 |
| FLKEKGGLDGV | 332 | 3.7 | 11 | 3207 | 3807 |
| GLIYSKKRQEV | 8971 | 57 | 152 | >8564.81 | >14260.25 |
| LLYSKKRQEI | 80687 | 382 | 152 | >9438.78 | >15686.27 |
| LLYSKKRQEIL | >38167.94 | 282 | 1569 | >8564.81 | >14260.25 |
| RLDILDLWV | 43 | 615 | 1639 | 2635 | >17777.78 |
| EILDLWVYHV | 496 | 569 | 1865 | 2229 | 163 |
| ILDLWVTYHV | 17 | 30 | 156 | 145 | 7414 |
| ILDLWVYNV | 40 | 30 | 201 | 135 | 5814 |
| WLNYTPGPGT | 547 | 124 | 231 | >31623.93 | 11808 |
| WQNYTPGPGV | 1175 | 114 | 230 | 223 | 11993 |
| WLNYTPGPGI | 135 | 4.6 | 46 | >31623.93 | 1196 |
| YLPGPGIRYPL | 1026 | 20 | 1583 | 3497 | 782 |
| YTPGPGIRYPV | 7764 | 1985 | 11126 | 1112 | 9.2 |
| LLFGWCFKL | 18 | 4.1 | 198 | 340 | 1084 |
| LTFGWCFKV | 15 | 33 | 1168 | 187 | 9.7 |
| LLFGWCFKLV | 658 | 84 | 114 | 1669 | 3276 |
| FGVRPQVPL | | | | | 321 |
| FTVRPQVPL | | | | | 13 |
| FSVRPQVPL | | | | | 52 |
| YLKEPVHGV | 54 | 0.65 | 1.9 | 212 | 63 |
| FLKEPVHGV | 44 | 0.28 | 1.9 | 140 | 135 |
| PVPLQLPPV | 10047 | >7337.88 | 12595 | 81 | >15625 |
| LQLPPLERV | 7951 | 7705 | 13517 | 203 | 1786 |
| LLLPPLERLTL | 34 | 2607 | 9010 | 45 | >12779.55 |
| LQLPPLERLTV | 159 | 4545 | 6270 | 52 | >61068.7 |
| ILWQVDRM | 1745 | 67 | 2998 | 11332 | >19464.72 |
| KLGSLQYL | 1862 | 14 | 298 | 9010 | >19464.72 |
| KVGSLQYV | 1650 | 441 | 703 | 1904 | 17480 |
| TLHDLCQAV | 331 | 17 | 15 | 10585 | 2809 |
| TLQDIVLHL | 22 | 4.4 | 46 | 781 | 5088 |
| TLGPGPGHL | 14974 | 35 | 66 | 12144 | 27910 |
| TLQGPGPGL | 6248 | 62 | 951 | 9121 | 3809 |
| TLSFVGPWGV | 786 | 123 | 370 | 4357 | 388 |
| TLSFVCPWCA | 1611 | 221 | 521 | 27321 | 13228 |
| RTLHDLCQA | 8121 | 34 | 678 | 96 | 61604 |
| TLHDLCQAL | 1404 | 2.7 | 40 | 2182 | 70390 |
| YLSGAIDLNL | 36 | 4.9 | 9.2 | 1605 | 51227 |
| YLEPGPVTA | 466 | 10 | 27 | 20720 | >470588.24 |
| LLDGTATLRL | 180 | 1.9 | 201 | 841 | >421052.63 |
| KVYGLSAFV | 33 | 1.8 | 11 | 69 | 110 |
| IISAVVAIL | 1127 | 8.0 | 45 | 1440 | 148 |
| ILSAVVGLL | 1464 | 1.9 | 21 | 2539 | 11854 |
| IISAVVGFL | 747 | 1.0 | 4.8 | 234 | 77 |
| IISAVVGIV | 712 | 15 | 20 | 958 | 390 |
| KISAVVGIL | 6238 | 42 | 60 | 1752 | 4952 |
| KIFAVVGIL | 3957 | 38 | 34 | 1539 | 6659 |
| KIFASVAIL | 1062 | 16 | 21 | 1068 | 363 |
| ELVSEFSRV | 8178 | 969 | 53 | 197 | 23 |
| VLVHPQWVV | 464 | 65 | 1988 | 3224 | 14606 |
| VLVHPQWVLTV | 11 | 1.7 | 3.0 | 13 | 3288 |
| DLMLLRLSEPV | 69 | 66 | 32 | 118 | 2078 |
| PLVCNGVLQGV | 91 | 424 | 36 | 212 | 3532 |
| VLVHPQWVLTV | 11 | 1.5 | 16 | 31 | 8889 |
| PLVCNGVLQGV | 26 | 126 | 19 | 264 | 4211 |
| QLGPGPGLMEV | 194 | 9.4 | 29 | 481 | 648 |
| QLVGPGPGMEV | 865 | 17 | 19 | 919 | 223 |
| QLVFGPGPGEV | 2944 | 106 | 50 | 4067 | 447 |
| QLVFGGPGPGV | 2153 | 96 | 242 | 3207 | 1318 |
| ALGIGILTV | 11 | | | | |
| AMGIGILTV | 15 | | | | |
| LLWQPIPV | 137 | 2445 | 9.9 | 4251 | 32939 |
| LLGPGPGV | 25 | 49 | 123 | 93 | 5620 |
| VLAKELKFVTL | 1298 | 23 | 194 | 5170 | 15664 |
| VLGPGPGFVTL | 1528 | 13 | 63 | 4766 | 42136 |
| VLAGPGPGVTL | 1118 | 2.4 | 94 | 7200 | 2645 |
| VLAKGPGPGTL | 11256 | 26 | 344 | 11450 | >170212.77 |
| VLAKEGPGPGL | 1890 | 6.9 | 37 | 59024 | 50993 |
| TLMSAMTNV | 636 | 14 | 35 | 2188 | 484 |
| ILYSAHDTTV | 397 | 1.1 | 13 | 1480 | 6285 |
| IVYSAHDTTV | 7643 | 91 | 627 | 356 | 737 |
| VTAKELKFV | 7143 | 2688 | 40 | 137 | 26667 |
| ITYSAHDTTV | 4167 | 115 | 238 | 154 | 82 |
| SLSLGFLFV | 77 | 25 | 21 | 93 | 26667 |
| SLSLGFLFLV | 1.9 | 3.9 | 17 | 42 | 348 |
| LLALFPPEGV | 5.0 | 0.73 | 1.6 | 148 | 163 |
| LVALFPPEGV | 156 | 17 | 4.8 | 463 | 28 |
| ALFPPEGVSV | 15 | 1.1 | 18 | 119 | 4444 |
| GLHGQDLFGV | 12 | 2.3 | 3.1 | 18 | >80000 |
| LLPPYASCHV | 88 | 15 | 16 | 97 | 5333 |
| LLWQPIPVTIV | 25 | 1.8 | 18 | 285 | 62 |
| MLLRLSEPV | 47 | 29 | 48 | 689 | 433 |
| ALGTTGYV | 93 | 6.7 | 12 | 292 | 28284 |
| VLRLFVCFLI | 2744 | 2112 | 299 | 68226 | 45639 |
| FLIFHFFLFL | 161 | 174 | 2087 | 288 | 475 |
| LIFHFFLFLL | 200 | 1468 | 3167 | 1562 | 460 |
| FLFLLYILFL | 2834 | 172 | 2012 | 2113 | 8248 |
| RLPVICSFLV | 12 | 2.5 | 33 | 19 | 9176 |
| VICSFLVFLV | 167 | 415 | 2916 | 197 | 1949 |
| FLVFLVFSNV | 269 | 212 | 35 | 232 | 5393 |
| MMIMIKFMGV | 123 | 19 | 25 | 109 | 39 |
| FLLYILFLV | 346 | 279 | 3091 | 1801 | 6981 |
| VICSFLVFL | 184 | 19 | 2331 | 236 | 4800 |
| ATYGIIVPV | 3.2 | 2.0 | 2.8 | 5.0 | 21 |
| KIYKIIIWI | 157 | 1179 | 638 | 101 | 2198 |
| YMIKKLLKI | 105 | 4.6 | 4.7 | 93 | 63127 |
| LMTLYQIQV | 14 | 1.6 | 20 | 615 | 1276 |
| FMGVIYIMI | 13 | 2.1 | 26 | 98 | 14501 |
| FMNRFYITT | 101 | 18 | 13 | 996 | 6543 |
| YQDPQNYEL | 79 | 18 | 441 | 52 | 166775 |
| KTWKPTIFL | 135 | 1242 | 7487 | 76 | 3617 |
| LLNESNIFL | 43 | 2.5 | 24 | 143 | 4484 |
| FIHFFTWGT | 80 | 4.7 | 64 | 60 | 383 |
| VLFLQMMNV | 31 | 1.8 | 2.7 | 9.5 | 323 |
| NQMIFVSSI | 250 | 21 | 3.6 | 14 | 198 |
| MTFVSSIFI | 85 | 18 | 83 | 114 | 5.2 |
| SIFISFYLI | 289 | 35 | 1416 | 43 | 18 |
| RLFEESLGI | 26 | 1.9 | 5.5 | 68 | 418 |
| ALWGFFPVL | 3.6 | 0.74 | 3.7 | 15 | 1503 |
| SVYDFFVWL | 36 | 169 | 226 | 10 | 0.86 |
| FAPGFFPYL | 48 | 0.85 | 44 | 2.3 | 7.6 |
| QLFEDKYAL | 646 | 1.8 | 380 | 2009 | 2982 |
| MLLSVPLLL | 9.0 | 79 | 41 | 8.4 | 24607 |

TABLE 15

HLA-A3 SUPERTYPE

| | | | | | | |
|---|---|---|---|---|---|---|
| | SEQ ID | | | | | |
| Sequence | NO. | AA | Organism | Protein | Position | Analog |

| | | | | | |
|---|---|---|---|---|---|
| ALNAAAAAK | 9 | Artificial | | | Poly |
| | | sequence | | | |
| ALAAGAAAK | 9 | Artificial | | | Poly |
| | | sequence | | | |
| ALQAAAAAK | 9 | Artificial | | | Poly |
| | | sequence | | | |
| STGPGPGVVRR | 11 | HBV | core | 141 | A |
| STLGPGPGVRR | 11 | HBV | core | 141 | A |
| STLPGPGPGRR | 11 | HBV | core | 141 | A |
| STLPEGPGPGR | 11 | HBV | core | 141 | A |
| QAGFFLLTR | 9 | HBV | ENV | 179 | |
| RVHFASPLH | 9 | HBV | POL | 818 | |
| AAYAAQGYK | 9 | HCV | II | 1247 | |
| KSKFGYGAK | 9 | HGV | II | 2551 | |
| PAAYAAQGYK | 10 | HCV | II | 1246 | |
| RMYVGGVEH | 9 | HCV | IV | 635 | |
| SQLSAPSLK | 9 | HCV | IV | 2209 | |
| TSCGNTLTCY | 10 | HCV | NS5 | 2740 | |
| VTGPGPGPVWK | 11 | HIV | env | 48 | A |
| VTVGPGPGVWK | 11 | HIV | env | 48 | A |
| VTVYGPGPGWK | 11 | HIV | env | 48 | A |
| VTVYYGPGPGK | 11 | HIV | env | 48 | A |
| PVRPQVPLR | 9 | HIV | NEF | 95 | |
| HGAITSSNTK | 10 | HIV | NEF | 61 | A |
| AVDLSFFLK | 9 | HIV | NEF | 111 | A |
| DVSHFLKEK | 9 | HIV | NEF | 113 | A |
| GVLDGLIYSK | 10 | HIV | NEF | 124 | A |
| GVDGLIYSK | 9 | HIV | NEF | 125 | A |
| EILDLWVYK | 9 | HIV | NEF | 185 | A |
| ILDLWVYK | 8 | HIV | NEF | 186 | A |
| RVPLTFGWCFK | 11 | HIV | NEF | 216 | A |
| QVYTPGPGTR | 10 | HIV | NEF | 205 | A |
| AVGPGPGLK | 9 | HIV | nef | 84 | A |
| AVDGPGPGK | 9 | HIV | nef | 84 | A |
| QMGPGPGNFK | 10 | HIV | pol | 1432 | A |
| QMAGPGPGFK | 10 | HIV | pol | 1432 | A |
| QMAVGPGPGK | 10 | HIV | pol | 1432 | A |
| TVGPGPGPEK | 10 | HIV | pol | 935 | A |
| TVQGPGPGEK | 10 | HIV | pol | 935 | A |
| TVQPGPGPGK | 10 | HIV | pol | 935 | A |
| VAIKIGGQLK | 10 | HIV | Pol | 98 | A |
| VTVKIGGQLK | 10 | HIV | Pol | 98 | A |
| VTIKVGGQLK | 10 | HIV | Pol | 98 | A |
| VTIRIGGQLK | 10 | HIV | Pol | 98 | A |
| VTVRIGGQLK | 10 | HIV | Pol | 98 | A |
| VTVKVGGQLK | 10 | HIV | Pol | 98 | A |
| VTIRVGGQLK | 10 | HIV | Pol | 98 | A |
| VTVRVGGQLK | 10 | HIV | Pol | 98 | A |
| VTVKIGGQLR | 10 | HIV | Pol | 98 | A |
| VTIRIGGQLR | 10 | HIV | Pol | 98 | A |
| VTIKLGGQIR | 10 | HIV | Pol | 98 | A |
| VSIKVGGQIK | 10 | HIV | Pol | 98 | A |
| VSIRVGGQIK | 10 | HIV | Pol | 98 | A |
| VTVKIEGQLK | 10 | HIV | Pol | 98 | A |
| VTIKIEGQLK | 10 | HIV | Pol | 98 | A |
| VTVKIEGQLR | 10 | HIV | Pol | 98 | A |
| VSIRVGGQTK | 10 | HIV | Pol | 98 | A |
| VSIRVGGQTR | 10 | HIV | Pol | 98 | A |
| VTVRIGGMQK | 10 | HIV | Pol | 98 | A |
| ITVKIGKEVR | 10 | HIV | Pol | 98 | A |
| GTRQARRNK | 9 | HIV | REV | 36 | A |
| GTRQARRNRK | 10 | HIV | REV | 36 | A |
| GTRQARRNRK | 11 | HIV | REV | 36 | A |
| GTRQTRKNRK | 9 | HIV | REV | 37 | A |
| GTRQTRKNRK | 10 | HIV | REV | 37 | A |
| GTRQTRKNRRK | 11 | HIV | REV | 37 | A |
| RVRRRRWRAR | 10 | HIV | REV | 43 | A |
| KVRRRRWRAR | 10 | HIV | REV | 43 | A |
| LTISYGRK | 8 | HIV | TAT | 46 | A |
| KTLGISYGR | 9 | HIV | TAT | 44 | A |
| LTISYGRKK | 9 | HIV | TAT | 46 | A |
| GTSYGRIKR | 9 | HIV | TAT | 47 | A |
| GTGISYGRK | 9 | HIV | TAT | 45 | A |
| KTLGISYGRK | 10 | HIV | TAT | 44 | A |
| LTISYGRKKR | 10 | HIV | TAT | 46 | A |
| KTLGISYGRKK | 11 | HIV | TAT | 44 | A |
| TVCNNCYGK | 9 | HIV | TAT | 23 | A |
| LVISYGRKKRR | 11 | HIV | TAT | 46 | A |
| ISYGRKKRRQK | 11 | HIV | TAT | 48 | A |
| ETGPSGQPCK | 10 | HIV | TAT | 101 | A |
| KVGPGGYPRR | 10 | HIV | TAT | 101 | A |
| KAGPGGYPRK | 10 | HIV | TAT | 101 | A |
| KVGPGGYPRRK | 11 | HIV | TAT | 101 | A |
| AVPGGYPRR | 9 | HIV | TAT | 102 | A |
| AVPGGYPRRK | 10 | HIV | TAT | 102 | A |
| KVGSLQYLK | 9 | HIV | VIF | 146 | A |
| ETVRHFPR | 8 | HIV | VPR | 29 | A |
| AACHKCIDFY | 10 | HPV | E6 | 63 | |
| LLIRCLRGQK | 10 | HPV | E6 | 101 | |
| KISEYRHYNY | 10 | HPV | E6 | 72 | |
| AVCRVCLLFY | 10 | HPV | E6 | 64 | |
| FAFTDLTIVY | 10 | HPV | E6 | 45 | |
| FAFADLTVVY | 10 | HPV | E6 | 45 | |
| RFLSKISEYR | 10 | HPV | E6 | 68 | |
| ILIRCIIGQR | 10 | HPV | E6 | 99 | |
| RTAMFQDPQER | 11 | HPV | E6 | 5 | |
| AMFQDPQERPR | 11 | HPV | E6 | 7 | |
| MFQDPQERPRK | 11 | HPV | E6 | 8 | |
| DLLIRCINGQK | 11 | HPV | E6 | 105 | |
| RFEDPTRRPYK | 11 | HPV | E6 | 3 | |
| ELTEVFEFAFK | 11 | HPV | E6 | 40 | |
| GLYNLLIRGLR | 11 | HPV | E6 | 97 | |
| NLLIRCLRCQK | 11 | HPV | E6 | 100 | |
| EVLEESVHEIR | 11 | HPV | E6 | 17 | |
| EVYKFLFTDLR | 11 | HPV | E6 | 41 | |
| FLFTDLRIVYR | 11 | HPV | E6 | 45 | |
| EVLEIPLIDLR | 11 | HPV | E6 | 20 | |
| DLRLSCVYCKK | 11 | HPV | E6 | 28 | |
| EVYNFACTELK | 11 | HPV | E6 | 44 | |
| RVCLLFYSKVR | 11 | HPV | E6 | 67 | |
| LLFYSKVRKYR | 11 | HPV | E6 | 70 | |
| QLCDLLIRCYR | 11 | HPV | E6 | 98 | |
| TLEQTVKK | 8 | HPV | E6 | 87 | |
| ATRDLCIVYR | 10 | HPV | E6 | 53 | A |
| AFRDLCIVYK | 10 | HPV | E6 | 53 | A |
| ATCDKCLKFY | 10 | HPV | E6 | 68 | A |
| AVCDKCLKFR | 10 | HPV | E6 | 68 | A |
| KLYSKISEYR | 10 | HPV | E6 | 75 | A |
| KFYSKISEYK | 10 | HPV | E6 | 75 | A |
| KESEYRHYCY | 10 | HPV | E6 | 79 | A |
| KISEYRHYCR | 10 | HPV | E6 | 79 | A |
| LFIRCINCQK | 10 | HPV | E6 | 106 | A |
| LLIRCINCQR | 10 | HPV | E6 | 106 | A |
| KVRFHNTRGR | 10 | HPV | E6 | 129 | A |
| KQRFHNIRGK | 10 | HPV | E6 | 129 | A |
| WFGRCMSCCR | 10 | HPV | E6 | 139 | A |
| WTGRCMSCCK | 10 | HPV | E6 | 139 | A |
| MTCCRSSRTR | 10 | HPV | E6 | 144 | A |
| MSCCRSSRTK | 10 | HPV | E6 | 144 | A |
| STCRSSRTRR | 10 | HPV | E6 | 145 | A |
| SCCRSSRTRK | 10 | HPV | E6 | 145 | A |
| DIEITCVYCR | 10 | HPV | E6 | 27 | A |
| FTFKDLFVVY | 10 | HPV | E6 | 47 | A |
| FAFKDLFVVK | 10 | HPV | E6 | 47 | A |
| AVKDLFVVYR | 10 | HPV | E6 | 48 | A |
| AFKDLFVVYK | 10 | HPV | E6 | 48 | A |
| FVVYRDSIPK | 10 | HPV | E6 | 53 | A |
| DTTPHAACHK | 10 | HPV | E6 | 58 | A |
| DSIPHAACHR | 10 | HPV | E6 | 58 | A |
| KFIDFYSRIR | 10 | HPV | E6 | 67 | A |
| DTVYGDTLEK | 10 | HPV | E6 | 83 | A |
| DSVYGDTLER | 10 | HPV | E6 | 83 | A |
| LFIRCLRCQK | 10 | HPV | E6 | 101 | A |
| LLIRCLRCQR | 10 | HPV | E6 | 101 | A |
| RVHNIAGHYR | 10 | HPV | E6 | 126 | A |
| RFHNIAGHYK | 10 | HPV | E6 | 126 | A |
| RTQCHSCCNR | 10 | HPV | E6 | 135 | A |
| RGQCHSCCNX | 10 | HPV | E6 | 135 | A |
| ATTDLTIVYR | 10 | HPV | E6 | 46 | A |
| AFTDLTIVYK | 10 | HPV | E6 | 46 | A |
| RLYSKVSEFR | 10 | HPV | E6 | 68 | A |
| RFYSKVSEFK | 10 | HPV | E6 | 68 | A |
| KFSEFRWYRY | 10 | HPV | E6 | 72 | A |
| KVSEFRWYRR | 10 | HPV | E6 | 72 | A |
| YFVYGTTLEK | 10 | HPV | E6 | 81 | A |
| YSVYGTTLER | 10 | HPV | E6 | 81 | A |
| GTTLEKLTNR | 10 | HPV | E6 | 85 | A |
| LVIRCITCQR | 10 | HPV | E6 | 99 | A |
| LLIRCITCQK | 10 | HPV | E6 | 99 | A |
| WVGRCIACWR | 10 | HPV | E6 | 132 | A |
| WTGRCIACWK | 10 | HPV | E6 | 132 | A |
| RTIACWRRPR | 10 | HPV | E6 | 135 | A |
| RCIACWRRPK | 10 | HPV | E6 | 135 | A |
| AVADLTVVYR | 10 | HPV | E6 | 46 | A |
| AFADLTVVYK | 10 | HPV | E6 | 46 | A |
| RVLSKISEYR | 10 | HPV | E6 | 68 | A |
| RFLSKISEYK | 10 | HPV | E6 | 68 | A |
| KFSEYRHYNY | 10 | HPV | E6 | 72 | A |
| KISEYRHYNR | 10 | HPV | E6 | 72 | A |
| ITIRCIICQR | 10 | HPV | E6 | 99 | A |
| ILIRCIICQK | 10 | HPV | E6 | 99 | A |
| WVGRCAACWR | 10 | HPV | E6 | 132 | A |
| WAGRCAACWK | 10 | HPV | E6 | 132 | A |
| CFACWRSRRR | 10 | HPV | E6 | 136 | A |
| DTSIACVYCK | 10 | HPV | E6 | 27 | A |
| DVSIACVYCR | 10 | HPV | E6 | 27 | A |
| CVYCKATLEK | 10 | HPV | E6 | 32 | A |
| RFEVYQFAFK | 10 | HPV | E6 | 41 | A |
| RTEVYQFAFR | 10 | HPV | E6 | 41 | A |
| AVKDLCIVYR | 10 | HPV | E6 | 48 | A |
| AFKDLCIVYK | 10 | HPV | E6 | 48 | A |
| ATCHKGIDFY | 10 | HPV | E6 | 63 | A |
| AAGHKCIDFK | 10 | HPV | E6 | 63 | A |
| NIVYGETLEK | 10 | HPV | E6 | 83 | A |
| NSVYGETLER | 10 | HPV | E6 | 83 | A |
| LSIRCLRGQK | 10 | HPV | E6 | 101 | A |
| LLIRCLRCQY | 10 | HPV | E6 | 101 | A |
| RVHSIAGQYR | 10 | HPV | E6 | 126 | A |
| RFHSIAGQYK | 10 | HPV | E6 | 126 | A |
| LVTDLRIVYR | 10 | HPV | E6 | 46 | A |
| LFTDLRIVYK | 10 | HPV | E6 | 46 | A |
| CTMGLRFLSK | 10 | HPV | E6 | 63 | A |
| CIMCLRFLSR | 10 | HPV | E6 | 63 | A |
| RLLSKISEYR | 10 | HPV | E6 | 68 | A |
| RFLSKISEYY | 10 | HPV | E6 | 68 | A |
| SFYGKTLEER | 10 | HPV | E6 | 82 | A |
| SLYGKTLEEK | 10 | HPV | E6 | 82 | A |
| WFGRCSECWR | 10 | HPV | E6 | 132 | A |
| WTGRCSECWK | 10 | HPV | E6 | 132 | A |
| AFGRVCLLFY | 10 | HPV | E6 | 64 | A |
| AVCRVCLLFR | 10 | HPV | E6 | 64 | A |
| CFLFYSKVRK | 10 | HPV | E6 | 69 | A |
| CLLFYSKVRR | 10 | HPV | E6 | 69 | A |
| LVYSKVRKYR | 10 | HPV | E6 | 71 | A |
| LFYSKVRKYK | 10 | HPV | E6 | 71 | A |
| GTTLESITKK | 10 | HPV | E6 | 88 | A |
| WVGSCLGCWR | 10 | HPV | E6 | 135 | A |
| WTGSCLGGWK | 10 | HPV | E6 | 135 | A |
| VVADLRIVYR | 10 | HPV | E6 | 46 | A |
| VFADLRIVYK | 10 | HPV | E6 | 46 | A |
| RTLSKISEYR | 10 | HPV | E6 | 68 | A |
| RLLSKISEYK | 10 | HPV | E6 | 68 | A |
| KVSEYRHYNY | 10 | HPV | E6 | 72 | A |
| KISEYRHYNK | 10 | HPV | E6 | 72 | A |
| IVIRCIICQR | 10 | HPV | E6 | 99 | A |
| WLGRCAVCWR | 10 | HPV | E6 | 132 | A |
| WTGRCAVCWK | 10 | HPV | E6 | 132 | A |
| YYVCDKCLK | 9 | HPV | E6 | 67 | A |
| YAVCDKCLR | 9 | HPV | E6 | 67 | A |
| SVCRSSRTR | 9 | HPV | E6 | 145 | A |
| SCCRSSRTK | 9 | HPV | E6 | 145 | A |
| SLPHAACHK | 9 | HPV | E6 | 59 | A |
| SIPHAACHR | 9 | HPV | E6 | 59 | A |
| FVDLTIVYR | 9 | HPV | E6 | 47 | A |
| FTDLTIVYK | 9 | HPV | E6 | 47 | A |
| SFYGTTLEK | 9 | HPV | E6 | 82 | A |
| SVYGITLER | 9 | HPV | E6 | 82 | A |
| TFLEKLTNK | 9 | HPV | E6 | 86 | A |
| TFLEKLTNR | 9 | HPV | E6 | 86 | A |
| ETNPFGIGK | 9 | HPV | E6 | 56 | A |
| EGNPFGIGR | 9 | HPV | E6 | 56 | A |
| NTLEQTVKR | 9 | HPV | E6 | 86 | A |
| ALCWRSRRR | 9 | EPY | E6 | 137 | A |
| AACWRSRRK | 9 | HPV | E6 | 137 | A |
| VSIACVYCR | 9 | HPV | E6 | 28 | A |
| SIACVYCKK | 9 | HPV | E6 | 29 | A |
| ILYRDCIAY | 9 | HPV | E6 | 54 | A |
| IVYRDGIAR | 9 | HPV | E6 | 54 | A |
| GTAYAAGHK | 9 | HPV | E6 | 59 | A |
| CIAYAACHR | 9 | HPV | E6 | 59 | A |
| SEYGETLEK | 9 | HPV | E6 | 84 | A |
| SVYGETLER | 9 | HPV | E6 | 84 | A |
| LIRCLRCQR | 9 | HPV | E6 | 102 | A |
| RTQCVQCKK | 9 | HPV | E6 | 27 | A |
| RLQCVQCKR | 9 | HPV | E6 | 27 | A |
| KFLEERVKK | 9 | HPV | E6 | 86 | A |
| KTLEERVKR | 9 | HPV | E6 | 86 | A |
| NVMGRWTGR | 9 | HPV | E6 | 127 | A |
| NIMGRWTGK | 9 | HPV | E6 | 127 | A |
| LTYRDDFPY | 9 | HPV | E6 | 55 | A |
| LVYRDDFPK | 9 | HPV | E6 | 55 | A |
| RFCLLFYSK | 9 | HPV | E6 | 67 | A |
| RVCLLFYSR | 9 | HPV | E6 | 67 | A |
| LTFYSKVRK | 9 | HPV | E6 | 70 | A |
| LLFYSKVRR | 9 | HPV | E6 | 70 | A |
| ATLESITKR | 9 | HPV | E6 | 89 | A |
| KVLCDLLIR | 9 | HPV | E6 | 97 | A |
| KQLCDLLIK | 9 | HPV | E6 | 97 | A |
| TFVHEIELK | 9 | HPV | E6 | 21 | A |
| TSVHEIELR | 9 | HPV | E6 | 21 | A |
| YTFVFADLR | 9 | HPV | E6 | 43 | A |
| DFLEQTLKK | 9 | HPV | E6 | 86 | A |
| DTLEQTLKR | 9 | HPV | E6 | 86 | A |
| LVRCIICQR | 9 | HPV | E6 | 100 | A |
| LIRCIICQK | 9 | HPV | E6 | 100 | A |
| RVAVGWRPR | 9 | HPV | E6 | 135 | A |
| RCAVCWRPK | 9 | HPV | E6 | 135 | A |
| AFCWRPRRR | 9 | HPV | E6 | 137 | A |
| AVCWRPRRK | 9 | HPV | E6 | 137 | A |
| LSFVCPWCA | 9 | HPV | E7 | 94 | |
| TFCCKCDSTLR | 11 | HPV | E7 | 56 | |
| LVVESSADDLR | 11 | HPV | E7 | 74 | |
| TLQVVCPGCAR | 11 | HPV | E7 | 88 | |
| YLIHVPGCECK | 11 | HPV | E7 | 59 | |
| FVVQLDIQSTK | 11 | HPV | E7 | 70 | |
| HTCNTTVR | 8 | HPV | E7 | 59 | |
| GLVCPICSQK | 10 | HPV | E7 | 88 | A |
| GFNIHQHLPAR | 10 | HPV | E7 | 43 | A |
| GVNHQHLPAK | 10 | HPV | E7 | 43 | A |
| NVVTFCGQCK | 10 | HPV | E7 | 53 | A |
| NIVTFCCQGR | 10 | HPV | E7 | 53 | A |
| GVSHAQLPAK | 10 | HPV | E7 | 44 | A |
| LIHVPCGECR | 10 | HPV | E7 | 60 | A |
| AVLQDIVLH | 9 | HPV | E7 | 6 | A |
| ATLQDIVLK | 9 | HPV | E7 | 6 | A |
| GVNHQHLPK | 9 | HPV | E7 | 43 | A |
| HVMLGMGCK | 9 | HPV | E7 | 59 | A |
| HTMLCMCGR | 9 | HPV | E7 | 59 | A |
| LSFVCPWCR | 9 | HPV | E7 | 94 | A |
| AQPATADYK | 9 | HPV | E7 | 45 | A |
| VVHAQLPAR | 9 | HPV | E7 | 45 | A |
| VSHAQLPAK | 9 | HPV | E7 | 45 | A |
| QLARQAKQH | 9 | HPV | E7 | 48 | A |
| KQHTCYLIR | 9 | HPV | E7 | 54 | A |
| VTLDIQSTK | 9 | HPV | E7 | 72 | A |
| VQLDIQSTR | 9 | HPV | E7 | 72 | A |
| SLGPGPGTK | 9 | Human | MAGE1 | 96 | A |
| SLFGPGPGK | 9 | Human | MAGE1 | 96 | A |
| LVGPGPGK | 8 | Human | MAGE2 116 | A | |
| KMFLQLAK | 8 | Human | p53 | 132 | |
| KMGPGPGK | 8 | Human | p53 | 132 | A |
| KQENWYSLKX | 10 | Pf | CSP | 58 | |
| GVGPGPGLK | 9 | Pf | LSA1 | 105 | A |
| GVSGPGPGK | 9 | Pf | LSA1 | 105 | A |
| FLLYILFLVK | 10 | Pf | | 17 | |
| LVFSNVLCFR | 10 | Pf | | 43 | |
| SSFDIKSEVK | 10 | Pf | | 116 | |
| TLYQTQVMKR | 10 | Pf | | 44 | |
| KQVQMMIMIK | 10 | Pf | | 58 | |
| GVIYIMNSK | 10 | Pf | | 70 | |
| ELFDKDTFFK | 10 | Pf | | 158 | |
| ALERLLSLKK | 10 | Pf | | 50 | |
| KILIKITPVTK | 10 | Pf | | 109 | |
| RLPLLPKTWK | 10 | Pf | | 128 | |
| SQVSNSDSYK | 10 | Pf | | 161 | |
| QQNQESKIMK | 10 | Pf | | 197 | |
| IIALLIIPPK | 10 | Pf | | 249 | |
| SSPLFNNFYK | 10 | Pf | | 14 | |
| FLYLLNKIKNK | 10 | Pf | | 151 | |
| LQMMNVNLQK | 10 | Pf | | 183 | |
| LTNHLTNTPK | 10 | Pf | | 195 | |
| IFISFYLINK | 10 | Pf | | 259 | |
| RLFEESLGIR | 10 | Pf | | 293 | |
| LLYILFLVK | 9 | Pf | | 18 | |
| KSMLKELTK | 9 | Pf | | 129 | |
| PVLTSLFNK | 9 | Pf | | 166 | |
| KTMNNYMIK | 9 | Pf | | 18 | |
| LFDKDTFFK | 9 | Pf | | 159 | |
| YLFNQHLKK | 9 | Pf | | 287 | |
| MQSSFFMNR | 9 | Pf | | 307 | |
| RFYITTRYK | 9 | Pf | | 315 | |
| TTRYKYLNK | 9 | Pf | | 319 | |
| AVIFTPIYY | 9 | Pf | | 34 | |
| ALERLLSLK | 9 | Pf | | 50 | |
| SISGKYDIK | 9 | Pf | | 85 | |
| EQRLPLLPK | 9 | Pf | | 126 | |
| IALLIIPPK | 9 | Pf | | 250 | |
| PVVCSMEYK | 9 | Pf | | 270 | |
| VVCSMEYKK | 9 | Pf | | 271 | |
| FSYDLRLNK | 9 | Pf | | 308 | |
| HLNIPIGFK | 9 | Pf | | 323 | |
| PLFNNFYKR | 9 | Pf | | 16 | |
| YQNFQNADK | 9 | Pf | | 141 | |
| QMMNVNLQK | 9 | Pf | | 184 | |
| AVSEIQNNK | 9 | Pf | | 222 | |
| GTMYILLKK | 9 | Pf | | 236 | |
| FISFYLINK | 9 | Pf | | 260 | |
| YLINKHWQR | 9 | Pf | | 264 | |
| ALKISQLQK | 9 | Pf | | 273 | |
| KINSNFLLK | 9 | Pf | | 282 | |
| AAMXDPTTFK | 10 | Unknown | Naturally | A | |
| | | | processed | | |
| GTMTTSXYK | 9 | Unknown | Naturally | A | |
| | | | processed | | |
| SXXPAXFQK | 9 | Unknown | Naturally | A | |
| | | | processed | | |
| ATAGDGXXEXRK | 12 | Unknown | Naturally | A | |
| | | | processed | | |

TABLE 16

HLA-A3 SUPERTYPE

| | | | | | |
|---|---|---|---|---|---|
| Sequence | A*0301 | A*1101 | A*3101 | A*3301 | A*6801 |

| | | | | | | |
|---|---|---|---|---|---|---|
| ALNAAAAAK | 74 | 21 | 10954 | >72500 | 80000 | |
| ALAAGAAAK | 19 | 37 | | | | |
| ALQAAAAAK | 57 | 65 | 51962 | >72500 | >80000 | |
| STGPGPGVVRR | 18695 | 367 | 95 | 5983 | 5.8 | |
| STLGPGPGVRR | 892 | 19 | 42 | 670 | 3.8 | |
| STLPGPGPGRR | 297 | 19 | 61 | 1893 | 25 | |
| STLPEGPGPGR | 325 | 26 | 28 | 822 | 30 | |
| QAGFFLLTR | 10138 | 1678 | 302 | 182 | 5.3 | |
| RVHFASPLH | 12 | 60 | 572 | >12288 | 1.36 | 7620 |
| AAYAAQGYK | 18 | 18 | 1175 | 14074 | 34 | |
| KSKFGYGAK | 36 | 596 | 116 | >122881.36 | >7626.31 | |
| PAAYAAQGYK | 950 | 456 | 20314 | >110687.02 | 666 | |
| RMYVGGVEH | 3.8 | 274 | 162 | >122881.36 | >28776.98 | |
| SQLSAPSLK | 306 | 25 | 1276 | >122881.36 | 3845 | |
| TSCGNTLTCY | >36666.67 | 5.0 | | | | |
| VTGPGPGPVWK | 2900 | 24 | 12964 | >102836.88 | 425 | |
| VTVGPGPGVWK | 174 | 2.7 | 2731 | 75360 | 21 | |
| VTVYGPGPGWK | 1151 | 18 | >8995.5 | >102836.88 | 206 | |
| VTVYYGPGPGK | 310 | 24 | 9720 | 101830 | 30 | |
| PVRPQVPLR | >10901.88 | 16112 | 332 | 3439 | 7012 | |
| HGAITSSNTK | 2837 | 344 | >16143.5 | >22924.9 | 1235 | |
| AVDLSFFLK | 226 | 23 | 6207 | >27831.09 | 4038 | |
| DVSHFLKEK | >9298.39 | 5645 >17839.44 | 232 | 135 | | |
| GVLDGLIYSK | 1080 | 21 | 6007 | >25151.78 | 831 | |
| GVDGLIYSK | 10089 | 47 | >17664.38 | >29652.35 | 5100 | |
| EILDLWVYK | 1032 | 64 | >5774.78 | 288 | 93 | |
| ILDLWVYK | 1265 | 320 | 13680 | 30096 | 12092 | |
| RVPLTFGWGFK | 69 | 30 | 102 | 26651 | 571 | |
| QVYTPGPGTR | 1249 | 852 | 1764 | 3334 | 273 | |
| AVGPGPGLK | 18 | 3.6 | 128 | 75754 | 444 | |
| AVDGPGPGK | 179 | 19 | 36837 | >112403.1 | 2132 | |
| QMGPGPGNFK | 49 | 22 | 2682 | 100771 | 63 | |
| QMAGPGPGFK | 9.4 | 6.2 | 667 | 4784 | 30 | |
| QMAVGPGPGK | 33 | 16 | 5961 | 86676 | 22 | |
| TVGPGPGPEK | 115 | 17 | 10140 | 98177 | 23 | |
| TVQGPGPGEK | 218 | 3.4 | 9874 | 103379 | 195 | |
| TVQPGPGPGK | 41 | 2.5 | 1335 | 68584 | 28 | |
| VAIKIGGQLK | 2593 | 151 | 46875 | 51222 | 123 | |
| VTVKIGGQLK | 296 | 61 | 24385 | 104757 | 147 | |
| VTIKVGGQLK | 188 | 59 | 6061 | 47647 | 127 | |
| VTIRIGGQLK | 51 | 14 | 4458 | 65764 | 25 | |
| VTVRIGGQLK | 226 | 15 | 5380 | 40344 | 49 | |
| VTVKVGGQLK | 206 | 54 | 21484 | 46182 | 104 | |
| VTIRVGGQLK | 43 | 13 | 3591 | 86086 | 28 | |
| VTVRVGGQLK | 216 | 19 | 8238 | >72319.2 | 141 | |
| VTVKIGGQLR | 19185 | 194 | 417 | 3833 | 52 | |
| VTIRIGGQLR | 3192 | 23 | 61 | 1352 | 16 | |
| VTIKLGGQIIR | 43252 | 219 | 590 | 12965 | 104 | |
| VSIKVGGQLK | 1921 | 86 | 57069 | >72319.2 | 2026 | |
| VSIRVGGQIK | 642 | 91 | 50677 | >61702.13 | 1960 | |
| VTVKTEGQLK | 647 | 23 | 4616 | 64604 | 30 | |
| VTIKIEGQLK | 361 | 69 | 5077 | 58024 | 27 | |
| VTVKIEGQLR | 35612 | 143 | 394 | 4057 | 146 | |
| VSIRVGGQTK | 341 | 21 | 29949 | 38958 | 290 | |
| VSIRVGGQTR | 18531 | 241 | 466 | 8595 | 288 | |
| VTVRIGGMQK | 54 | 13 | 2583 | 44425 | 155 | |
| ITVKIGKEVR | >69182.39 | 12904 | 5057 | 24985 | 154 | |
| GTRQARRNK | 67 | 749 | 9713 | 45966 | 59708 | |
| GTRQARRNRK | 100 | 634 | 3800 | >42335.77 | 7788 | |
| GTRQARRNRRK | 404 | 2596 | 7774 | >24308.47 | 9104 | |
| GTRQTRKNK | 198 | 3104 | 13373 | >29713.11 | 18657 | |
| GTRQTRKNRK | 129 | 1082 | 2485 | 60183 | 5998 | |
| GTRQTRKNRRK | 478 | 4184 | 4008 | >24308.47 | >17167.38 | |
| RVRRRRWRAR | 2443 | >16759.78 | 265 | 3758 | >36866.36 | |
| KVRRRRWRAR | 327 | >20905.92 | 342 | 3243 | 15501 | |
| LTISYGRK | 988 | 708 | 27068 | 38162 | 482 | |
| KTLGISYGR | 53 | 9.8 | 21 | 502 | 36 | |
| LTISYGRKK | 584 | 69 | 13918 | 59654 | 63 | |
| GTSYGRKKR | 9965 | 5916 | 225 | 21588 | 5778 | |
| GTGISYGRK | 480 | 77 | 58102 | >43740.57 | 7407 | |
| KTLGISYGRK | 36 | 79 | 841 | 42378 | 1629 | |
| LTISYGRKKR | 7161 | 1229 | 71 | 2515 | 33 | |
| KTLGISYGRKK | 52 | 285 | 91 | 23401 | 647 | |
| TVCNNCYGK | 9920 | 267 | 8793 | 28481 | 876 | |
| LVISYGRKKRR | >11702.13 | 8669 | 562 | 267 | 4662 | |
| ISYGRXKRRQK | 48 | 2807 | 3147 | >20000 | 4428 | |
| ETGPSGQPGK | >14569.54 | 3501 | >22500 | >17813.27 | 50 | |
| KVGPGGYPRR | 2268 | 487 | 250 | 7904 | 721 | |
| KAGPGGYPRK | 62 | 43 | 10734 | >17813.27 | 5555 | |
| KVGPGGYPRRK | 70 | 87 | 775 | >5063.73 | 921 | |
| AVPGGYPRR | 3012 | 1215 | 1349 | 3453 | 109 | |
| AVPGGYPRRK | 819 | 60 | 39974 | >5570.5 | 846 | |
| KVGSLQYLK | 482 | 70 | 2104 | >43740.57 | 4200 | |
| ETVRHFPR | >13513.51 | 4183 | 1000 | 81 | 86 | |
| AACHXCIDFY | 18824 | 261 | 20643 | >116465.86 | 32548 | |
| LLTRCLRCQK | 437 | 170 | 6612 | 28936 | 78 | |
| KISEYRHYNY | 42 | 112 | 1426 | 35341 | 25077 | |
| AVCRVGLLFY | 77 | 21 | 1978 | 4520 | 1302 | |
| FAFTDLTIVY | 40343 | 21161 | 42065 | 131202 | 346 | |
| FAFADLTVVY | 18592 | 5866 | 23676 | 26768 | 402 | |
| RFLSKISEYR | 1640 | 18468 | 33 | 436 | 172 | |
| ILIRCIICQR | 8550 | 5012 | 377 | 2480 | 537 | |
| RTAMFQDPQER | 1478 | 103 | 49 | 3459 | 19 | |
| AMFQDPQERPR | 1718 | 886 | 45 | 1787 | 1478 | |
| MFQDPQERPRK | 15493 | 8571 | 604 | 419 | 16729 | |
| DLLIRCINCQK | 2923 | 935 | 4884 | 29 | 263 | |
| RFEDPTRRPYK | 169 | 432 | 53 | 1758 | 7338 | |
| ELTEVFEFAFK | 8966 | 582 | 25205 | 1733 | 15 | |
| GLYNLLIRCLR | 1268 | 1568 | 250 | 401 | 1624 | |
| NLLIRGLRCQK | 1565 | 854 | 3140 | 397 | 1480 | |
| EVLEESVHEIR | >45643.15 | >20202.02 | 31037 | 212 | 240 | |
| EVYKFLFTDLR | 31240 | 602 | 759 | 4.3 | 11 | |
| FLFTDLRIVYR | 672 | 227 | 58 | 21 | 1.4 | |
| EVLEIPLIDLR | >47008.55 | 16638 | 36427 | 72 | 27 | |
| DLRLSCVYCKK | 3644 | 1907 | 17023 | 109 | 3002 | |
| EVYNFACTELK | 1622 | 117 | 484 | 5.9 | 2.7 | |
| RVCLLFYSKVR | 771 | 190 | 221 | 1061 | 1267 | |
| LLFYSKVRKYR | 28 | 94 | 7.0 | 11 | 15 | |
| QLCDLLIRGYR | 1240 | 700 | 450 | 106 | 489 | |
| TLEQTVKK | 4766 | 203 | >100000 | >75324.68 | 21400 | |
| ATRDLCIVYR | 237 | 156 | 4.7 | 44 | 28 | |
| AFRDLCIVYK | 31 | 15 | 10 | 132 | 57 | |
| ATGDKCLKFY | 194 | 17 | 491 | 18080 | 4562 | |
| AVCDKCLKFR | 77 | 15 | 11 | 45 | 34 | |
| KLYSKISEYR | 5.4 | 168 | 6.4 | 28 | 91 | |
| KFYSKISEYK | 7.6 | 674 | 27 | 329 | 208 | |
| KFSEYRHYCY | 5092 | 7485 | 308 | 49397 | 14571 | |
| KISEYRHYCR | 486 | 688 | 25 | 833 | 1488 | |
| LFIRCINCQK | 2880 | 702 | 52 | 42 | 56 | |
| LLIRGINCQR | 2818 | 686 | 30 | 50 | 14 | |
| KVRFHNIRGR | 39 | 8632 | 27 | 4500 | 3979 | |
| KQRFHNIRGK | 55 | 1953 | 573 | 35208 | 22879 | |
| WFGRCMSCCR | 16071 | 10690 | 288 | 98 | 303 | |
| WTGRCMSCCK | 6687 | 841 | 6496 | 15191 | 118 | |
| MTCCRSSRTR | 3825 | 933 | 410 | 601 | 2.2 | |
| MSCGRSSRTK | 352 | 169 | 2333 | 6916 | 12 | |
| STCRSSRTRR | 2989 | 118 | 152 | 1020 | 312 | |
| SCCRSSRTRK | 326 | 3272 | 5592 | 20916 | 8777 | |
| DIEITCVYCR | 2014 | 826 | 3780 | 448 | 422 | |
| FTFKDLFVVY | 14364 | 1208 | 10757 | 2725 | 62 | |
| FAFKDLFVVK | 783 | 71 | 525 | 1066 | 3.6 | |
| AVKDLFVVYR | 1728 | 91 | 3.1 | 9.1 | 3.3 | |
| AFKDLFVVYX | 3256 | 211 | 32 | 93 | 576 | |
| FVVYRDSIPK | 265 | 81 | 6216 | 146 | 30 | |
| DTIPHAACHK | 2366 | 701 | 1763 | 9.3 | 23 | |
| DSIPHAACHR | 2772 | 853 | 357 | 2.2 | 27 | |
| KFIDFYSRIR | 8891 | 9008 | 3.3 | 677 | 2551 | |
| DTVYGDTLEK | 50 | 15 | 28754 | 55090 | 31 | |
| DSVYGDTLER | 292 | 23 | 485 | 891 | 28 | |
| LFIRCLRCQK | 3390 | 1533 | 218 | 77 | 200 | |
| LLIRCLRCQR | 3360 | 1396 | 28 | 75 | 13 | |
| RVHNIAGHYR | 30 | 21 | 22 | 114 | 18 | |
| RFHNIAGHYK | 25 | 22 | 2.6 | 80 | 23 | |
| RTQCHSCCNR | 338 | 20 | 22 | 132 | 161 | |
| RGQCHSCCNK | 6135 | 113 | 425 | 37669 | 20340 | |
| ATTDLTIVYR | 247 | 10 | 34 | 1739 | 14 | |
| AFTDLTIVYK | 701 | 112 | 3952 | 9380 | 215 | |
| RLYSKVSEFR | 6.4 | 131 | 24 | 690 | 73 | |
| RFYSKVSEFK | 27 | 521 | 30 | 4452 | 547 | |
| KFSEFRWYRY | 4750 | 1595 | 34 | 856 | 12811 | |
| KVSEFRWYRR | 266 | 16 | 2.8 | 159 | 30 | |
| YFVYGTTLEK | 204 | 62 | 2167 | 15740 | 53 | |
| YSVYGTTLER | 430 | 96 | 2136 | 6903 | 19 | |
| GTTLEKLTNR | 3604 | 1720 | 382 | 706 | 2946 | |
| LVIRCITCQR | 2222 | 255 | 54 | 135 | 14 | |
| LLIRCITCQK | 291 | 120 | 3009 | 2165 | 40 | |
| WVGRGIACWR | 6227 | 1391 | 85 | 13 | 9.7 | |
| WTGRCIACWK | 2633 | 55 | 3078 | 169 | 24 | |
| RTLACWRRPR | 40 | 63 | 3.2 | 95 | 51 | |
| RCIACWRRPK | 1535 | 1476 | 292 | 176 | 1655 | |
| AVADLTVVYR | 489 | 11 | 31 | 892 | 7.3 | |
| AFADLTVVYK | 2365 | 107 | 1113 | 13557 | 50 | |
| RVLSKISEYR | 34 | 84 | 24 | 197 | 136 | |
| RFLSKISEYK | 31 | 287 | 42 | 10237 | 112 | |
| KFSEYRHYNY | 5819 | 5521 | 286 | 18351 | 1798 | |
| KISEYRHYNR | 58 | 140 | 17 | 161 | 1579 | |
| ITIRCIICQR | 488 | 93 | 50 | 123 | 12 | |
| ILIRCIICQK | 192 | 78 | 1383 | 1423 | 165 | |
| WVGRCAACWR | 2757 | 3973 | 360 | 24 | 19 | |
| WAGRCAACWK | 4662 | 583 | 23311 | 1491 | 50 | |
| CFACWRSRRR | 23542 | 7164 | 578 | 165 | 10206 | |
| DTSIACVYCK | 2936 | 89 | 5385 | 1968 | 216 | |
| DVSIACVYCR | 2814 | 217 | 406 | 487 | 658 | |
| CVYCKATLEK | 418 | 653 | 5307 | 17928 | 862 | |
| RFEVYQFAFK | 38 | 611 | 179 | 2867 | 2443 | |
| RTEVYQFAFR | 217 | 78 | 12 | 142 | 147 | |
| AVKDLCIVYR | 841 | 66 | 7.3 | 8.0 | 6.5 | |
| AFKDLCIVYK | 856 | 47 | 39 | 263 | 378 | |
| ATGHKCIDFY | 133 | 7.4 | 1164 | 12691 | 1386 | |
| AAGHKCIDFK | 118 | 20 | 437 | 53733 | 414 | |
| NLVYGETLEK | 846 | 143 | 761 | 121 | 87 | |
| NSVYGETLER | 150 | 25 | 163 | 1333 | 18 | |
| LSIRGLRGQK | 245 | 14 | 100 | 1135 | 17 | |
| LLIRGLRCQY | 727 | 452 | 2894 | 2430 | 254 | |
| RVHSIAGQYR | 31 | 34 | 7.6 | 812 | 28 | |
| RFHSIAGQYK | 17 | 43 | 1.3 | 629 | 83 | |
| LVTDLRIVYR | 3869 | 648 | 20 | 150 | 6.8 | |
| LFTDLRIVYK | 628 | 263 | 258 | 149 | 277 | |
| CTMCLRFLSK | 1002 | 226 | 6274 | 3945 | 429 | |
| CIMCLRFLSR | 41 | 101 | 167 | 83 | 155 | |
| RLLSKISEYR | 5.2 | 662 | 7.7 | 108 | 21 | |
| RFLSKISEYY | 1702 | 25535 | 14 | 41096 | 3999 | |
| SFYGKTLEER | 642 | 205 | 17 | 66 | 42 | |
| SLYGKTLEEK | 7.9 | 6.8 | 1044 | 6516 | 29 | |
| WFGRGSECWR | 1788 | 1569 | 20 | 5.5 | 26 | |
| WTGRCSECWK | 2492 | 26 | 3323 | 720 | 22 | |
| AFCRVCLLFY | 509 | 272 | 1777 | 1202 | 173 | |
| AVCRVCLLFR | 20 | 1.8 | 2.1 | 64 | 21 | |
| CFLFYSKVRK | 125 | 96 | 81 | 315 | 172 | |
| CLLFYSKVRR | 417 | 204 | 159 | 386 | 242 | |
| LVYSKVRKYR | 320 | 619 | 17 | 49 | 31 | |
| LFYSKVRXYK | 680 | 2582 | 18 | 30 | 1976 | |
| GTTLESITKK | 622 | 108 | 85182 | 132509 | 10147 | |
| WVGSCLGCWR | 48682 | 5520 | 20 | 15 | 9.3 | |
| WTGSCLGGWK | 7705 | 6.9 | 18344 | 2980 | 3.7 | |
| VVADLRIVYR | 513 | 18 | 41 | 101 | 16 | |
| VFADLRIVYK | 2086 | 127 | 402 | 200 | 273 | |
| RTLSKISEYR | 77 | 100 | 52 | 189 | 133 | |
| RLLSKISEYK | 15 | 65 | 158 | 40019 | 429 | |
| KVSEYRTYNY | 349 | 110 | 1791 | 70859 | 3498 | |
| KISEYRHYNK | 29 | 18 | 397 | 24827 | 15565 | |
| IVIRCIICQR | 984 | 217 | 52 | 529 | 28 | |
| WLGRCAVCWR | 2330 | 3002 | 356 | 40 | 112 | |
| WTGRGAVGWK | 1261 | 131 | 4176 | 3403 | 29 | |
| YVVCDKCLK | 3282 | 643 | 8.5 | 165 | 1289 | |
| YAVCDKGLR | 458 | 194 | 4261 | 26582 | 16034 | |
| SVCRSSRTR | 323 | 97 | 249 | 547 | 17 | |
| SCCRSSRTK | 21 | 3.9 | 51 | 5227 | 4.2 | |
| SLPHAACHK | 32 | 66 | 219 | 1186 | 654 | |
| SIPHAACHR | 1053 | 352 | 236 | 253 | 181 | |
| FVDLTIVYR | 29674 | 5312 | 2384 | 430 | 138 | |
| FTDLTIVYK | 557 | 16 | 24170 | 18477 | 143 | |
| SFYGTTLEK | 34 | 15 | 517 | 3385 | 498 | |
| SVYGTTLER | 28 | 6.4 | 133 | 454 | 21 | |
| TFLEKLTNK | 6839 | 815 | 451 | 148 | 918 | |
| TTLEKLTNR | 1993 | 817 | 42 | 37 | 101 | |
| ETNPFGICK | 9585 | 100 | 29103 | 804 | 14 | |
| EGNPFGICR | 11467 | 10372 | 5123 | 344 | 82 | |
| NTLEQTVKR | 20380 | 1151 | 2273 | 18 | 8.6 | |
| ALGWRSRRR | 959 | 9748 | 72 | 1289 | 7416 | |
| AACWRSRRK | 75 | 770 | 3022 | 45341 | 12877 | |
| VSIACVYGR | 3236 | 143 | 42 | 1347 | 185 | |
| SIACVYCKK | 271 | 83 | 9114 | 19632 | 96 | |
| ILYRDCIAY | 261 | 1832 | 53232 | 44670 | >19607.84 | |
| IVYRDCIAR | 465 | 106 | 27 | 325 | 64 | |
| CTAYAACHK | 726 | 196 | 2956 | 771 | 167 | |
| CIAYAACHR | 3625 | 1905 | 502 | 115 | 262 | |
| SEYGETLEK | 288 | 108 | 947 | 885 | 1074 | |
| SVYGETLER | 44 | 11 | 235 | 160 | 17 | |
| LIRCLRCQR | 21335 | 12648 | 695 | 810 | 200 | |
| RTQCVQGKK | 234 | 20 | 127 | 8147 | 3066 | |
| RLQCVQCKR | 2535 | 6081 | 65 | 1829 | 11479 | |
| KFLEERVKK | 5344 | 2229 | 30 | 9740 | 17674 | |
| KTLEERVKR | 1957 | 159 | 37 | 1360 | 17685 | |
| NVMGRWTGR | 3884 | 794 | 40 | 18 | 20 | |
| NIMGRWTGK | 52 | 54 | 3274 | 86 | 173 | |
| LTYRDDFPY | 8265 | 82 | >71146.25 | 20186 | 1529 | |
| LVYRDDFPK | 317 | 13 | 3009 | 1970 | 130 | |
| RFCLLFYSK | 1156 | 484 | 83 | 450 | 232 | |
| RVCLLFYSR | 439 | 111 | 51 | 2176 | 689 | |
| LTFYSKVRK | 3.8 | 8.0 | 87 | 3382 | 13 | |
| LLFYSKVRR | 56 | 73 | 38 | 276 | 11 | |
| ATLESITKR | 1437 | 16 | 100 | 851 | 188 | |
| KVLCDLLIR | 363 | 169 | 66 | 5896 | 9053 | |
| KQLCDLLIK | 226 | 65 | 340 | 46426 | 11897 | |
| TFVHEIELK | 4431 | 217 | 8412 | 4130 | 172 | |
| TSVHEIELR | >64327.49 | 872 | 1039 | 5948 | 12 | |
| YTFVFADLR | 3633 | 8.1 | 20 | 6.6 | 2.9 | |
| DFLEQTLKK | >57591.62 | 18809 | 34365 | 174 | 14376 | |
| DTLEQTLKR | 31347 | 12909 | 38127 | 9.2 | 110 | |
| LVRCIICQR | 677 | 358 | 59 | 109 | 201 | |
| LTRCIICQK | 445 | 252 | 639 | 834 | 285 | |
| RVAVGWRPR | 5.3 | 8.5 | 7.0 | 102 | 33 | |
| RCAVCWRPK | 285 | 340 | 382 | 131 | 1297 | |
| AFCWRPRRR | 273 | 17907 | 60 | 75 | 1087 | |
| AVCWRPRRK | 34 | 101 | 263 | 7950 | 1810 | |
| LSFVCPWCA | 38337 | 10864 | 4289 | 4603 | 341 | |
| TFCCKGDSTLR | 21772 | 8043 | 332 | 91 | 260 | |
| LVYESSADDLR | >47008.55 | 2170 | 26410 | 5624 | 28 | |
| TLQVVCPGCAR | 20997 | 1395 | 67 | 63 | 147 | |
| YLIHVPCCECK | 1748 | 1534 | 33044 | 8066 | 177 | |
| FVVQLDIQSTK | 3682 | 853 | 48593 | 31350 | 2.7 | |
| HTCNTIYR | 4862 | 1792 | 726 | 4490 | 25 | |
| GLVCPICSQK | 428 | 814 | 45293 | 70317 | 3568 | |
| GFNHQHLPAR | >46610.17 | 27889 | 173 | 5572 | 34617 | |
| GVNHQHLPAK | 42 | 11 | 3337 | 76239 | 9347 | |
| NVVTFCGQCK | 790 | 303 | 4757 | 87 | 13 | |
| NIVTFGCQGR | 1507 | 1070 | 2731 | 766 | 93 | |
| GVSHAQLPAK | 42 | 12 | 36011 | >74935.4 | 20590 | |
| LIHVPCGECR | 5326 | 5925 | 385 | 387 | 228 | |
| AVLQDIVLH | 1922 | 101 | 6307 | 25776 | 27035 | |
| ATLQDTVLK | 37 | 8.6 | 65 | 17121 | 3231 | |
| GVNHQHLPK | 26 | 7.7 | 353 | 15615 | 1192 | |
| HVMLCMCCK | 282 | 79 | 772 | 825 | 99 | |
| HTMLCMCCR | 405 | 92 | 11 | 14 | 24 | |
| LSFVCPWCR | 31676 | 200 | 47 | 231 | 152 | |
| AQPATADYK | 3500 | 109 | 10413 | 58871 | 24173 | |
| VVHAQLPAR | 423 | 127 | 3.4 | 12 | 201 | |
| VSHAQLPAK | 378 | 9.5 | 46 | 1401 | 13502 | |
| QLARQAKQH | 8423 | 6862 | 945 | 1665 | 243 | |
| KQHTCYLIR | 135 | 213 | 13 | 2275 | 12177 | |
| VTLDIQSTK | 78 | 13 | 2046 | 1954 | 237 | |
| VQLDIQSTR | 15105 | 2917 | 162 | 4588 | 10341 | |
| SLGPGPGTK | 7.8 | 5.8 | 4392 | 152133 | 3517 | |
| SLFGPGPGK | 3.4 | 2.3 | 1085 | 82275 | 36 | |
| LVGPGPGK | 1004 | 291 | 23907 | >125541.13 | 598 | |
| KMFLQLAK | 45 | 62 | 677 | >125541.13 | 8384 | |
| KMGPGPGK | 84 | 242 | 1144 | 106362 | 4156 | |
| KQENWYSLKK | 608 | 178 | 6327 | >136150.23 | 4794 | |
| GVGPGPGLK | 47 | 4.0 | 1367 | >111538.46 | 3972 | |
| GVSGPGPGK | 13 | 5.8 | >11221.95 | >111538.46 | 209 | |
| FLLYILFLVK | 446 | 1431 | 54496 | 3254 | 2266 | |
| LVFSNVLCFR | 120 | 19 | 33 | 19 | 7.7 | |
| SSFDIKSEVK | 1900 | 19 | 19829 | 70344 | 31 | |
| TLYQIQVMKR | 361 | 164 | 397 | 558 | 90 | |
| KQVQMMIMIK | 264 | 112 | 4627 | 1231 | 2247 | |
| GVIYIMIISK | 777 | 18 | 18811 | 1567 | 1134 | |
| ELFDKDTFFK | 144 | 109 | 3676 | 13 | 3.6 | |
| ALERLLSLKK | 147 | 822 | 33559 | 18255 | 22391 | |
| KILIKIPVTK | 13 | 60 | 1661 | 24992 | 19571 | |
| RLPLLPKTWK | 11 | 67 | 340 | 11392 | 2889 | |
| SQVSNSDSYK | 1656 | 83 | 24559 | >17448.86 | 1384 | |
| QQNQESKIMK | 3469 | 77 | 28120 | >17448.86 | 21310 | |
| HALLIIPPK | 30 | 5.3 | 23822 | 8426 | 82 | |
| SSPLFNNFYK | 100 | 0.7 | 1608 | 1728 | 6.3 | |
| FLYLLNKKNK | 177 | 475 | 4313 | 780 | 155 | |
| LQMMNVNLQK | 25 | 7.2 | 435 | 1113 | 320 | |
| LTNHLTNTPK | 11 | 5.9 | 62 | 373 | 10 | |
| IFISEYLINK | 1987 | 1056 | 462 | 394 | 363 | |
| RLFEESLGIR | 64 | 1096 | 297 | 788 | 409 | |
| LLYILFLVK | 13 | 207 | 90687 | 13261 | 5545 | |
| KSMLKELIK | 189 | 151 | 450 | >46548.96 | >37037.04 | |
| PVLTSLFNK | 1949 | 25 | 5107 | 18271 | 29928 | |
| KTMNNYMIK | 17 | 5.5 | 24 | 12743 | 29 | |
| LFDKDTFFK | 931 | 167 | 5706 | 1189 | 101 | |
| YLFNQHIKK | 14 | 7.8 | 4919 | 7974 | 14 | |
| MQSSFFMNR | 13 | 1.1 | 29 | 75 | 3.8 | |
| RFYITTRYK | 1.9 | 67 | 15 | 98 | 17468 | |
| TTRYKYLNK | 117 | 848 | 416 | 652 | 2565 | |
| AVIFTPIYY | 25 | 9.5 | 42321 | 10068 | 1352 | |
| ALERLLSLK | 233 | 369 | 3433 | 12786 | 13708 | |
| SISGKYDIK | 2086 | 50 | 28249 | 12437 | 1745 | |
| EQRLPLLPK | 1088 | 765 | 423 | 987 | 1911 | |
| IALLIIPPK | 1241 | 108 | 2926 | 1404 | 1965 | |
| PVVCSMEYK | 1940 | 80 | 330791 | 22608 | 414 | |
| VVCSMEYKK | 443 | 54 | 891 | 14328 | 167 | |
| FSYDLRLNK | 29 | 4.9 | 461 | 1264 | 15 | |
| HLNIPIGFK | 2.3 | 1.3 | 183 | 97 | 2.8 | |
| PLFNNFYKR | 2635 | 1890 | 520 | 1258 | 132 | |
| YONFQNADK | 2712 | 177 | 44698 | >18447.84 | 19830 | |
| QMMNVNLQK | 20 | 7.0 | 504 | 6649 | 243 | |
| AVSEIQNNK | 25 | 11 | 1429 | 25449 | 14 | |
| GTMYILLKK | 2.2 | 1.2 | 29 | 8453 | 3.1 | |
| FISFYLINK | 19 | 9.0 | 2192 | 1456 | 18 | |
| YLTNKHWQR | 1034 | 676 | 4.4 | 7.7 | 3.7 | |
| ALKISQLQK | 15 | 96 | 3203 | 23800 | >54794.52 | |
| KIINSNFLLK | 17 | 6.4 | 68 | 47740 | 2737 | |
| AAMXDPTTFK | 50 | 7.2 | | | | |
| GTMTTSXYK | 4.0 | 4.5 | | | | |
| SXXPAXFQK | 14 | 2.0 | | | | |
| ATAGDGXXEXRK | 184 | 19 | | | | |

TABLE 17

HLA-A24 SUPERTYPE

| | | | | | | |
|---|---|---|---|---|---|---|
| | SEQ | | | | | |
| | ID | | | | | |
| Sequence | NO. | AA | Organism | Protein | Position | Analog |

| | | | | | |
|---|---|---|---|---|---|
| AYGPGPGKF | 9 | Artificial | Consensus | | A |
| | | sequence | | | |
| AYIGPGPGF | 9 | Artificial | Consensus | | A |
| | | sequence | | | |
| AYAAAAAAL | 9 | Artificial | | | Poly |
| | | sequence | | | |
| AYSSWMYSY | 9 | EBV | EBNA3 | 176 | |
| DLLDTASALY | 10 | HBV | Core | 419 | |
| WFHISCLTF | 9 | HBV | NUC | 102 | |
| KYTSFPWL | 8 | HBV | pol | 745 | |
| FAAPFTQCGY | 10 | HBV | pol | 631 | |
| SYQHFRKLLL | 10 | HBV | POL | 4 | |
| LYSHPIILGF | 10 | HBV | POL | 492 | |
| MSTTDLEAY | 9 | HBV | X | 103 | |
| MYVGDLCGSVF | 11 | HCV | E1 | 275 | |
| MYGPGPGGSVF | 11 | HCV | E1 | 275 | A |
| MYVGPGPGSVF | 11 | HCV | E1 | 275 | A |
| MYVGGPGPGVF | 11 | HCV | E1 | 275 | A |
| MYVGDGPGPGF | 11 | HCV | E1 | 275 | A |
| VMGSSYGF | 8 | HCV | NS5 | 2639 | |
| EVDGVRLHRY | 10 | HCV | NS5 | 2129 | |
| KYSKSSIVGW | 10 | HIV | NEF | 4 | A |
| KWSKSSIVGF | 10 | HIV | NEF | 4 | A |
| FFLKEKGGF | 9 | HIV | NEF | 116 | A |
| IYSKKRQEF | 9 | HIV | NEF | 175 | A |
| IYSKKRQEIF | 10 | HIV | NEF | 175 | A |
| LYVYHTQGYF | 10 | HIV | NEF | 190 | A |
| VYHTQGYFPDF | 11 | HIV | NEF | 192 | A |
| RYPLTFGW | 8 | HIV | NEF | 216 | |
| RYPLTFGF | 8 | HIV | NEF | 216 | A |
| RFPLTFGF | 8 | HIV | NEF | 216 | A |
| TYGWCFKL | 8 | HIV | NEF | 222 | A |
| TFGWCFKF | 8 | HIV | NEF | 222 | A |
| LYVYHTQGY | 9 | HIV | NEF | 190 | A |
| NYTPGPGIRF | 10 | HIV | NEF | 206 | A |
| QYPPLERLTL | 10 | HIV | REV | 78 | A |
| QLPPLERLTF | 10 | HIV | REV | 78 | A |
| KYGSLQYLAL | 10 | HIV | VIF | 146 | A |
| LSKISEYRHY | 10 | HPV | E6 | 70 | |
| ISEYRHYNY | 9 | HPV | E6 | 73 | |
| RFHNIRGRW | 9 | HPV | E6 | 131 | |
| RFLSKISEY | 9 | HPV | E6 | 68 | |
| RFHNISGRW | 9 | HPV | E6 | 124 | |
| VYDFAFRDLCI | 11 | HPV | E6 | 49 | |
| PYAVCDKCLKF | 11 | HPV | E6 | 66 | |
| QYNKPLCDLLI | 11 | HPV | E6 | 98 | |
| PFGICKLCLRF | 11 | HPV | E6 | 59 | |
| VYQFAFKDLCI | 11 | HPV | E6 | 44 | |
| AYAACHKCIDF | 11 | HPV | E6 | 61 | |
| VYKFLFTDLRI | 11 | HPV | E6 | 42 | |
| PYGVCIMCLRF | 11 | HPV | E6 | 59 | |
| PYAVCRVCLLF | 11 | HPV | E6 | 62 | |
| VYDFVFADLRI | 11 | HPV | E6 | 42 | |
| QYNKPLGDLF | 10 | HPV | E6 | 98 | A |
| VYEFAFKDLF | 10 | HPV | E6 | 44 | A |
| FYSKVSEFRF | 10 | HPV | E6 | 69 | A |
| VYREGNPFGF | 10 | HPV | E6 | 53 | A |
| FYSRIRELRF | 10 | HPV | E6 | 71 | A |
| PYAVCRVCLF | 10 | HPV | E6 | 62 | A |
| FYSKVRKYRF | 10 | HPV | E6 | 72 | A |
| LYGDTLEQTF | 10 | HPV | E6 | 83 | A |
| VYDFAFRDF | 9 | HPV | E6 | 49 | A |
| AYRDLCIVY | 9 | HPV | E6 | 53 | A |
| AFRDLC1VF | 9 | HPV | E6 | 53 | A |
| PYAVGDKCF | 9 | HPV | E6 | 66 | A |
| KYYSKISEY | 9 | HPV | E6 | 75 | A |
| KEYSKISEF | 9 | HPV | E6 | 75 | A |
| CYSLYGTTF | 9 | HPV | E6 | 87 | A |
| RYHNIRGRW | 9 | HPV | E6 | 131 | A |
| RFHNIRGRF | 9 | HPV | E6 | 131 | A |
| VYCKTVLEF | 9 | HPV | E6 | 33 | A |
| AYKDLFVVY | 9 | HPV | E6 | 48 | A |
| AFKDLFVVF | 9 | HPV | E6 | 48 | A |
| LYVVYRDSI | 9 | HPV | E6 | 52 | A |
| LFVVYRDSF | 9 | HPV | E6 | 52 | A |
| RYHNIAGHY | 9 | HPV | E6 | 126 | A |
| RFHNIAGHF | 9 | HPV | E6 | 126 | A |
| VYGTFLEKF | 9 | HPV | E6 | 83 | A |
| AYADLTVVY | 9 | HPV | E6 | 46 | A |
| AFADLTVVF | 9 | HPV | E6 | 46 | A |
| RYLSKISEY | 9 | HPV | E6 | 68 | A |
| NYSVYGNTF | 9 | HPV | E6 | 80 | A |
| RYHNISGRW | 9 | HPV | E6 | 124 | A |
| AYKDLCIVY | 9 | HPV | E6 | 48 | A |
| AFKDLCIVF | 9 | HPV | E6 | 48 | A |
| AYAAGHKCF | 9 | HPV | E6 | 61 | A |
| VYGETLEKF | 9 | HPV | E6 | 85 | A |
| RYHSIAGQY | 9 | HPV | E6 | 126 | A |
| RFHSIAGQF | 9 | HPV | E6 | 126 | A |
| KYLFTDLRI | 9 | HPV | E6 | 44 | A |
| KFLFTDLRF | 9 | HPV | E6 | 44 | A |
| LYTDLRIVY | 9 | HPV | E6 | 46 | A |
| LFTDLRIVF | 9 | HPV | E6 | 46 | A |
| PYGVCIMCF | 9 | HPV | E6 | 59 | A |
| RFLSKISEF | 9 | HPV | E6 | 68 | A |
| EYRHYQYSF | 9 | HPV | E6 | 75 | A |
| RYHNIMGRW | 9 | HPV | E6 | 124 | A |
| RFHNIMGRF | 9 | HPV | E6 | 124 | A |
| VYNFACTEF | 9 | HPV | E6 | 45 | A |
| NYACTELKL | 9 | HPV | E6 | 47 | A |
| NFACTELKF | 9 | HPV | E6 | 47 | A |
| PYAVCRVCF | 9 | HPV | E6 | 62 | A |
| LYYSKVRKY | 9 | HPV | E6 | 71 | A |
| LFYSKVRKF | 9 | HPV | E6 | 71 | A |
| VYDFVFADF | 9 | HPV | E6 | 42 | A |
| VYADLRIVY | 9 | HPV | E6 | 46 | A |
| VFADLRIVF | 9 | HPV | E6 | 46 | A |
| NYSLYGDTF | 9 | HPV | E6 | 80 | A |
| RFHNISGRF | 9 | HPV | E6 | 124 A | |
| LYNLLIRCF | 9 | HPV | E6 | 98 | A |
| FYSKYSEF | 8 | HPV | E6 | 69 | |
| VYREGNPF | 8 | HPV | E6 | 53 | |
| VFEFAFKDLF | 10 | HPV | E6 | 44 | |
| EYRHYCYSLY | 10 | HPV | E6 | 82 | |
| EYRHYNYSLY | 10 | HPV | E6 | 75 | |
| ETRHYCYSLY | 10 | HPV | E6 | 82 | A |
| EYDHYGYSLY | 10 | HPV | E6 | 82 | A |
| KTRYYDYSVY | 10 | HPV | E6 | 78 | A |
| KYDYYDYSVY | 10 | HPV | E6 | 78 | A |
| ETRHYNYSLY | 10 | HPV | E6 | 75 | A |
| EYDHYNYSLY | 10 | HPV | E6 | 75 | A |
| TYCCKCDSTL | 10 | HPV | E7 | 56 | A |
| TFCCKGDSTF | 10 | HPV | E7 | 56 | A |
| TYCHSCDSTF | 10 | HPV | E7 | 58 | A |
| CYTCGTTVRF | 10 | HPV | E7 | 59 | A |
| LYPEPTDLF | 9 | HPV | E7 | 15 | A |
| NYYIVTCCF | 9 | HPV | E7 | 52 | A |
| LFLNTLSF | 8 | HPV | E7 | 89 | |
| LFLSTLSF | 8 | HPV | E7 | 90 | |
| RVLPPNATKY | 9 | Human | 40s ribo | 132 | |
| | | | prot S13 | | |
| RLAHEVGWKY | 10 | Human | 60s ribo | 139 | |
| | | | prot L13A | | |
| AYKKQFSQY | 9 | Human | 60s ribo | 217 | |
| | | | prot L5 | | |
| KTKDIVNGL | 9 | Human | Factin | 235 | |
| | | | capping | | |
| | | | protein | | |
| SLFVSNHAY | 9 | Human | fructose | 355 | |
| | | | biphosphate- | | |
| | | | aldolase | | |
| TYGPGPGSLSF | 11 | Human | Her2/neu | 63 | A |
| TYLGPGPGLSF | 11 | Human | Her2/neu | 63 | A |
| TYLPGPGPGSF | 11 | Human | Her2/neu | 63 | A |
| TYLPTGPGPGF | 11 | Human | Her2/neu | 63 | A |
| RWGLLLALL | 9 | Human | Her2/neu | 8 | |
| PYVSRLLGI | 9 | Human | Her2/neu | 780 | |
| TYLPTNASL | 9 | Human | Her2/neu | 63 | |
| IYGPGPGLIF | 10 | Human | MAGE3 | 195 | A |
| IYPGPGPGIF | 10 | Human | MAGE3 | 195 | A |
| IYPKGPGPGF | 10 | Human | MAGE3 | 195 | A |
| RISGVDRYY | 9 | Human | NADH | 53 | |
| | | | ubiqoxido- | | |
| | | | reductase | | |
| LYSACFWWL | 9 | Human | OA1 | 194 | |
| LYSACFWWF | 9 | Human | OA1 | 194 | A |
| TYSVSFDSLF | 10 | Human | PSM | 624 | |
| TYGPGPGSLF | 10 | Human | PSM | 624 | A |
| TYSGPGPGLF | 10 | Human | PSM | 624 | A |
| TYSVGPGPGF | 10 | Human | PSM | 624 | A |
| AYPNVSAKI | 9 | Lysteria | listerio- | 196 | |
| | | | lysin | | |
| AYGPGPGKI | 9 | Lysteria | listerio- | 196 | A |
| | | | lysin | | |
| IMVLSFLF | 8 | Pf | CSP | 427 | |
| YYGKQENW | 8 | Pf | CSP | 55 | |
| VFNVVNSSI | 9 | Pf | CSP | 416 | |
| ALFQEYQCY | 9 | Pf | CSP | 18 | |
| LYNTEKGRHPF | 11 | Pf | EXP | 100 | |
| YFILVNLL | 8 | Pf | LSA | 10 | |
| KFFDKDKEL | 9 | Pf | LSA | 76 | |
| KFIKSLFHI | 9 | Pf | LSA | 1876 | |
| YFILVNLLIF | 10 | Pf | LSA | 10 | |
| FYFILVNLLIF | 11 | Pf | LSA | 9 | |
| SFYFILVNLLI | 11 | Pf | LSA | 8 | |
| VFLIFFDLF | 9 | Pf | SSP2 | 13 | |
| LYLLMDCSGSI | 11 | Pf | SSP2 | 49 | |
| KVSDEIWNY | 9 | Pf | | 182 | |
| SYKSSKRDKF | 10 | Pf | | 225 | |
| RYQDPQNYEL | 10 | Pf | | 21 | |
| DFFLKSKFNI | 10 | Pf | | 3 | |
| IFHFFLFLL | 9 | Pf | | 11 | |
| VFLVFSNVL | 9 | Pf | | 41 | |
| TYGIIVPVL | 9 | Pf | | 160 | |
| NYMKIMNHL | 9 | Pf | | 34 | |
| TYKKKNNHI | 9 | Pf | | 264 | |
| VYYNILIVL | 9 | Pf | | 277 | |
| LYYLFNQHI | 9 | Pf | | 285 | |
| SFFMNRFYI | 9 | Pf | | 310 | |
| FYITTRYKY | 9 | Pf | | 316 | |
| KYINFINFI | 9 | Pf | | 328 | |
| KYEALIKLL | 9 | Pf | | 380 | |
| IYYFDGNSW | 9 | Pf | | 40 | |
| VYRHCEYIL | 9 | Pf | | 94 | |
| TWKPTIFLL | 9 | Pf | | 135 | |
| SYKVNCINF | 9 | Pf | | 168 | |
| KYNYFIHEF | 9 | Pf | | 216 | |
| NYFIHFFTW | 9 | Pf | | 218 | |
| HEFTWGTMF | 9 | Pf | | 222 | |
| MFVPKYFEL | 9 | Pf | | 229 | |
| IYTIIQDQL | 9 | Pf | | 295 | |
| FFLKSKFNI | 9 | Pf | | 4 | |
| RMTSLKNEL | 9 | Pf | | 61 | |
| YYNNFNNNY | 9 | Pf | | 77 | |
| YYNKSTEKL | 9 | Pf | | 87 | |
| EYEPTANLL | 9 | Pf | | 109 | |
| VYXKHPVSX | 9 | Unknown | Naturally | | A |
| | | | processed | | |
| TYGNXTVTV | 9 | Unknown | Naturally | | A |
| | | | processed | | |
| KYPDRVVPX | 9 | Unknown | Naturally | | A |
| | | | processed | | |
| VYVXSXVTX | 9 | Unknown | Naturally | | A |
| | | | processed | | |
| DAQXXXNTX | 9 | Unknown | Naturally | | A |
| | | | processed | | |
| KYQAVTTTL | 9 | Unknown | Tumor p198 | 197 | |
| KYGPGPGTTTL | 11 | Unknown | Tumor p198 | 197 | A |
| KYQGPGPGTTL | 11 | Unknown | Tumor p198 | 197 | A |

TABLE 18

HLA-A24 SUPERTYPE

| | | | | |
|---|---|---|---|---|
| Sequence | A*2402 | A*2301 | A*2902 | A*3002 |

| | | | | |
|---|---|---|---|---|
| AYGPGPGKF | 2.4 | 9.7 | 44854 | 3.2 |
| AYIGPGPGF | 217 | 12 | 15887 | 5728 |
| AYAAAAAAL | 443 | | | |
| AYSSWMYSY | | 21 | | 4.9 |
| DLLDTASAL | | | 74 | 37 |
| Y | | | | |
| WFHISCLTF | 204 | 11 | 95 | 75094 |
| KYTSFPWL | 208 | 177 | >172413.79 | 346 |
| FAAPFTQCG | | | 461 | 1364 |
| Y | | | | |
| SYQHFRKLL | 418 | 39 | 28 | 3768 |
| L | | | | |
| LYSHPIILG | 2.6 | 5.4 | 109 | 1116 |
| F | | | | |
| MSTTDLEAY | | | 2565 | 396 |
| MYVGDLCGS | 26 | 0.91 | 612 | 1460 |
| VF | | | | |
| MYGPGPGGS | 35 | 5.4 | 48442 | 31980 |
| VF | | | | |
| MYVGPGPGS | 35 | 4.4 | 1527 | 28177 |
| VF | | | | |
| MYVGGPGPG | 381 | 85 | 89 | 2870 |
| VF | | | | |
| MYVGDGPGP | 90 | 11 | 8656 | 39608 |
| GF | | | | |
| VMGSSYGF | 36 | 159 | 145 | 41967 |
| EVDGVRLHR | | | 14940 | 113 |
| Y | | | | |
| KYSKSSIVG | 4061 | 491 | >69444.44 | >34482.76 |
| W | | | | |
| KWSKSSIVG | 1674 | 84 | >56179.78 | 30367 |
| F | | | | |
| FFLKIEKGG | 3456 | 655 | 3015 | 141 |
| F | | | | |
| IYSKKRQEP | 306 | 421 | 29353 | 727 |
| IYSKKRQEI | 238 | 360 | >131578.95 | 21001 |
| F | | | | |
| LYVYHTQGY | 38 | 23 | 1696 | 1222 |
| F | | | | |
| VYHTQGYFP | 149 | 68 | 14923 | >22556.39 |
| DF | | | | |
| RYPLTFGW | 127 | 3836 | 13889 | 6251 |
| RYPLTFGF | 3.3 | 6.4 | 9704 | 6328 |
| RFPLTFGF | 178 | 124 | 12759 | 13472 |
| TYGWCFKL | 2181 | 333 | 25658 | >8042.9 |
| TFGWCFKF | 3424 | 462 | 4449 | >10135.14 |
| LYVYHTQGY | 7140 | 6088 | 216 | 258 |
| NYTPGPGIR | 483 | 37 | 8334 | >9646.3 |
| F | | | | |
| QYPPLERLT | 211 | 22 | >11520.74 | >9646.3 |
| L | | | | |
| QLPPLERLT | 2507 | 338 | >37313.43 | >36585.37 |
| F | | | | |
| KYGSLQYLA | 2800 | 147 | >69444.44 | 6957 |
| L | | | | |
| LSKISEYRH | >93023.26 | >23671.5 | 55190 | 186 |
| Y | | | | |
| ISEYRHYNY | 125794 | >23557.69 | 1329 | 32 |
| RFHNIIRGR | 53237 | 11416 | 18 | 58 |
| W | | | | |
| RFLSKISEY | 472 | 121 | 34623 | 23 |
| RFRNISGRW | >80536.91 | 22871 | 174 | 37 |
| VYDFAFRDL | 44 | 8.9 | 62242 | 35724 |
| CI | | | | |
| PYAVCDKCL | 99 | 8.1 | 118249 | >60000 |
| KF | | | | |
| QYNKPLCDL | 303 | 36 | >166666.67 | 6680 |
| LI | | | | |
| PFGICKLCL | 137 | 19 | 1249 | 32803 |
| RF | | | | |
| VYQFAFKDL | 30 | 1.9 | 49276 | 3477 |
| CI | | | | |
| AYAACHKCI | 91 | 14 | 1264 | 4699 |
| DF | | | | |
| VYKFLFTDL | 37 | 14 | 30216 | 1865 |
| RI | | | | |
| PYGVCLMCL | 380 | 100 | 69 | 43722 |
| RF | | | | |
| PYAVCRVCL | 226 | 150 | 2711 | 53351 |
| LF | | | | |
| VYDFVFADL | 47 | 8.0 | 8904 | 7585 |
| RI | | | | |
| QYNKPLCDL | 115 | 21 | 7658 | 525 |
| F | | | | |
| VYEFAFKDL | 15 | 1.7 | 1973 | 2038 |
| F | | | | |
| FYSKVSEFR | 7.1 | 2.2 | 79 | 18453 |
| F | | | | |
| VYREGNPFG | 197 | 91 | 11120 | 21947 |
| F | | | | |
| FYSRIRELR | 11 | 1.6 | 83 | 12598 |
| F | | | | |
| PYAVCRVCL | 12 | 4.5 | 407 | 5226 |
| F | | | | |
| FYSKVRKYR | 18 | 13 | 3042 | 1232 |
| F | | | | |
| LYGDTLEQT | 91 | 24 | 40871 | 42025 |
| F | | | | |
| YYIDFAFRD | 9.6 | 19 | 47381 | 8490 |
| F | | | | |
| AYRDLCIVY | 2094 | 1479 | 7117 | 66 |
| AFRDLCIVF | 1005 | 369 | 6722 | 3305 |
| PYAVCDKCF | 216 | 183 | 122025 | 9884 |
| KYYSKISEY | 10951 | 2165 | 702 | 1.3 |
| KEYSKISEF | 174 | 138 | 73339 | 306 |
| CYSLYGTTF | 28 | 11 | 2088 | 7823 |
| RYHNIRGRW | 145 | 14 | 122644 | 15 |
| RFHNIRGRF | 29 | 2.4 | 346 | 0.69 |
| VYCKTVLEF | 50 | 4.7 | 610 | 1139 |
| AYKDLFVVY | 1549 | 905 | 639 | 1.3 |
| AFKDLFVVF | 294 | 6.8 | 3051 | 829 |
| LYVVYRDSI | 982 | 242 | 148359 | 3483 |
| LFVVYRDSF | 268 | 134 | 919 | 18 |
| RYITNIAGH | 1227 | 195 | 138 | 0.93 |
| Y | | | | |
| RFHNIAGIT | 37 | 17 | 635 | 1.4 |
| F | | | | |
| VYGTTLEKF | 19 | 13 | 75267 | 220 |
| AYADLTVVY | 369 | 1384 | 136 | 9.3 |
| AFADLTVVF | 203 | 30 | 779 | 137 |
| RYLSKISEY | 142 | 98 | 4247 | 1.1 |
| NYSVYGNTF | 28 | 29 | 9121 | 2559 |
| RYHNISGRW | 47 | 15 | 104884 | 13 |
| AYKDLCLVY | 33798 | 3036 | 5205 | 29 |
| AFKDLCIVF | 284 | 16 | 5846 | 2305 |
| AYAACHKCF | 200 | 159 | 10972 | 3393 |
| VYGETLEKE | 45 | 14 | 91902 | 20009 |
| RYHSIAGQY | 3170 | 1904 | 544 | 1.4 |
| RFHSIAGQF | 28 | 2.9 | 481 | 1.2 |
| KYLFTDLRI | 108 | 1.9 | 78575 | 339 |
| KFLFTDLRF | 12 | 0.74 | 44 | 152 |
| LYTDLRIVY | 1986 | 1216 | 4.8 | 2.1 |
| LFTDLRIVF | 169 | 2.6 | 164 | 2649 |
| PYGVCIMCF | 190 | 147 | 144402 | 38850 |
| RFLSKISEF | 58 | 2.5 | 40103 | 201 |
| EYRHYQYSF | 21 | 2.3 | 13707 | 430 |
| RYHNIMGRW | 29 | 12 | 106990 | 7.1 |
| RFHNIMGRF | 39 | 2.6 | 174 | 1.3 |
| VYNFACTEF | 14 | 2.1 | 774 | 784 |
| NYACTELKL | 1741 | 131 | 77844 | 49107 |
| NFACTELKF | 211 | 13 | 46 | 6826 |
| PYAVCRVCF | 429 | 257 | 5602 | 316 |
| LYYSKVRKY | 21942 | 2735 | 1452 | 28 |
| LFYSKVRKF | 2008 | 277 | 11172 | 632 |
| VYDFVFADF | 9.9 | 2.2 | 1230 | 3961 |
| VYADLRIVY | 28 | 122 | 8.2 | 8.3 |
| VFADLRIVF | 23 | 2.5 | 87 | 24062 |
| NYSLYGDTF | 6.4 | 142 | 20945 | 64 |
| RFHNISGRF | 34 | 5.5 | 572 | 2.8 |
| LYNLLIIRC | 47 | 15 | 17958 | 2255 |
| F | | | | |
| FYSKVSEF | 21 | 18 | 3774 | 66667 |
| VYREQNPE | 554 | 147 | 10001 | 65970 |
| VFEFAFKDL | 400 | | | |
| F | | | | |
| EYRHYCYSL | | | 198 | 3.7 |
| Y | | | | |
| EYRIIYNYS | | | 956 | 12 |
| LY | | | | |
| ETRHYCYSL | | | 755 | 10 |
| Y | | | | |
| EYDHYCYSL | | | 799 | 77 |
| Y | | | | |
| KTRYYDYSV | | | 87841 | 0.71 |
| Y | | | | |
| KYDYYDYSV | | | 5749 | 11 |
| Y | | | | |
| ETRHYNYSL | | | 5464 | 29 |
| Y | | | | |
| EYDHYNYSL | | | 777 | 93 |
| Y | | | | |
| TYCCKCDST | 206 | 30 | 145803 | 16588 |
| L | | | | |
| TFCCKCDST | 25 | 14 | 501 | 1167 |
| F | | | | |
| TYCHSCDST | 14 | 2.9 | 5236 | 3580 |
| F | | | | |
| CYTCGTTVR | 41 | 18 | 7744 | 38331 |
| F | | | | |
| LYPEPTDLF | 38 | 17 | 1150 | 30732 |
| NYYIVTCCF | 27 | 12 | 2675 | 8398 |
| LFLNTLSF | 587 | 104 | 1013 | 118217 |
| LFLSTLSF | 2283 | 160 | 1034 | >75000 |
| RVLPPNWKY | | >49000 | | 3.0 |
| RLAHEVGWK | | 4631 | | 3.8 |
| Y | | | | |
| AYKKQFSQY | | 10669 | | 5.3 |
| KTKDIVNGL | | >49000 | | 164 |
| SLFVSNHAY | | 30295 | | 1.1 |
| TYGPGPGSL | 7.1 | 1.7 | 9853 | 47246 |
| SF | | | | |
| TYLGPGPGL | 23 | 0.65 | 600 | 26889 |
| SF | | | | |
| TYLPGPGPG | 8.8 | 2.2 | 56183 | 7275 |
| SF | | | | |
| TYLPTGPGP | 39 | 8.6 | 56574 | 32985 |
| GF | | | | |
| RWGLLLALL | 106 | 100 | 61253 | 300 |
| PYVSRLLGI | 11 | 18 | 200160 | 65465 |
| TYLPTNASL | 141 | 7.8 | 106153 | 8244 |
| IYGPGPGLI | 7.4 | 8.0 | 58 | 6845 |
| IF | | | | |
| IYPGPGPGI | 58 | 12 | 18659 | 17959 |
| F | | | | |
| IYPKGPGPG | 7.5 | 4.9 | 53603 | 61283 |
| F | | | | |
| RISGVDRYY | | >49000 | | 3.0 |
| LYSACFWWL | 28 | | | |
| LYSACFWWF | 28 | | | |
| TYSVSFDSL | 10 | 12 | 521 | 5218 |
| F | | | | |
| TYGPGPGSL | 3.9 | 8.7 | 7228 | 10871 |
| F | | | | |
| TYSGPGPGL | 50 | 92 | 7726 | 3461 |
| F | | | | |
| TYSVGPGPG | 332 | 340 | 120913 | 55200 |
| F | | | | |
| AYPNVSAKI | 14 | 45 | 56905 | 4456 |
| AYGPGPGKI | 36 | 169 | >156250 | 5427 |
| IMVLSFLF | 469 | 7.5 | 111 | 30000 |
| YYGKQENW | 85 | 951 | >50000 | >30000 |
| VFNVVNSSI | 403 | 35 | 24001 | 15737 |
| ALFQEYQCY | | | 149 | 1032 |
| LYNTEKGRH | 175 | 1947 | >50000 | >30000 |
| PF | | | | |
| YFILVNLL | 96 | 82 | 4050 | 30000 |
| KLFFDKDKE | 269 | >49000 | >50000 | 3012 |
| L | | | | |
| KIFIKSLFH | 4.1 | 2.0 | >50000 | 3495 |
| I | | | | |
| YFILVNLLI | 577 | 12 | 764 | 3388 |
| IF | | | | |
| FYFILVNLL | 599 | 50 | 902 | 9826 |
| IF | | | | |
| SFYFILVNL | 229 | 35 | 3066 | 2096 |
| LI | | | | |
| VFLTFFDLF | 40 | 12 | 1510 | 13554 |
| LYLLMDCSG | 154 | 10 | 5893 | 1469 |
| SI | | | | |
| KVSDETWNY | 52169 | >11980.44 | 230 | 1.9 |
| SYKSSKRDK | 256 | 797 | 12594 | 88 |
| F | | | | |
| RYQDPQNYE | 212 | 124 | 79717 | 189 |
| L | | | | |
| DFFLKSKFN | 1648 | 304 | 47714 | 491 |
| I | | | | |
| IIFHFFLFL | 208 | 80 | 1405 | 837 |
| L | | | | |
| VFLVFSNVL | 26 | 4.9 | 33675 | 37689 |
| TYGIIVPVL | 248 | 20 | 30056 | 1519 |
| NYMKIMNIH | 16 | 1.7 | 45443 | 110 |
| L | | | | |
| TYIKKKNNH | 30 | 81 | 21642 | 162 |
| I | | | | |
| VYYNILIVL | 265 | 52 | >192307.69 | 1127 |
| LYYLFNQHI | 33 | 1.4 | 20130 | 11035 |
| SFFMNKFYI | 172 | 11 | 200 | 1022 |
| FYITTRYKY | 350 | 11 | 9.6 | 7.5 |
| KYINFIINF | 11 | 0.72 | 25475 | 55 |
| I | | | | |
| KYEALIIKL | 2856 | 484 | 17296 | 16098 |
| L | | | | |
| IYYFDGNSW | 80 | 6.1 | 3101 | 3025 |
| VYRHCEYIL | 2200 | 64 | 117851 | 3326 |
| TWKPTIIFL | 148 | 11 | 21155 | 306 |
| L | | | | |
| SYKVNCINF | 27 | 15 | 2535 | 572 |
| KYNYFIHFF | 2.5 | 0.49 | 319 | 2.7 |
| NYFIHFFTW | 9.3 | 1.3 | 9774 | 3020 |
| HFFTWGTMF | 83 | 5.7 | 4.0 | 220 |
| MFVPKYFEL | 266 | 11 | 2560 | 8560 |
| IYTIIQDQL | 72 | 45 | >37313.43 14124 | |
| FFLKSKFNI | 1434 | 49 | 43105 >83333.33 | |
| RMTSLKINE | 12711 | 1807 | 40270 | 14 |
| L | | | | |
| YYNNFNNNY | 817 | 126 | 19 | 34 |
| YYNKSTEKL | 109 | 106 | 55636 | 21751 |
| EYEPTANLL | 127 | 44 | >37313.43 | >26086.96 |
| VYXKITPVS | 4.3 | | | |
| X | | | | |
| TYGNXTVTV | 26 | | | |
| KYPDRVVPX | 224 | | | |
| VYVXSXVTX | 5.3 | | | |
| DAQXXXNTX | 5.9 | | | |
| KYQAVTTTL | 22 | 16 | >156250 | 625 |
| KYGPGPGTT | 103 | 130 | 9180 | 7056 |
| TL | | | | |
| KYQGPGPGT | 543 | 438 | 74453 | 5999 |
| TL | | | | |

TABLE 19

HLA-B7 SUPERTYPE

| | | | | | | |
|---|---|---|---|---|---|---|
| | SEQ | | | | | |
| Sequence | ID NO. | AA | Organism | Protein | Position | Analog |

| | | | | | |
|---|---|---|---|---|---|
| APGPGPGLL | 9 | Artificial | Consensus | | A |
| | | sequence | | | |
| APRGPGPGL | 9 | Artificial | Consensus | | A |
| | | sequence | | | |
| QPRAPIRPI | 9 | EBNA | | 881 | |
| YPLHEQHGM | 9 | EBNA | | 458 | |
| CPTVQASKL | 9 | HBV | NUC | 14 | |
| SPTYKAFL | 8 | HBV | pol | 659 | |
| SPGPGFGL | 8 | HBV | pol | 659 | A |
| TPAGPGPGVF | 10 | HBV | pol | 354 | A |
| TPARGPGPGF | 10 | HBV | pol | 354 | A |
| TPTGWGLM | 9 | HBV | POL | 691 | |
| APCNFFTSA | 9 | HBV | X | 146 | |
| GPGHKARVI | 9 | HIV | GAG | 390 | A |
| RPQVPLRPMTI | 11 | HIV | NEF | 98 | A |
| FPVRPQVPI | 9 | HIV | NEF | 94 | A |
| RPQVPLRPI | 9 | HIV | NEF | 98 | A |
| RPQVPLRPMTI | 11 | HIV | NEF | 98 | A |
| YPLTFGWCI | 9 | HIV | NEF | 217 | A |
| FPLTFGWCI | 9 | HIV | NEF | 217 | A |
| FPLTFGWCFKI | 11 | HIV | NEF | 217 | A |
| FPVRPQVPL | 9 | HIV | nef | 94 | |
| FPGPGPGPL | 9 | HIV | nef | 94 | A |
| FPVGPGPGL | 9 | HIV | nef | 94 | A |
| GPKVKQWPI | 9 | HIV | POL | 197 | A |
| LPPLERLTI | 9 | HIV | REV | 79 | A |
| CPEEKQRHL | 9 | HPV | E6 | 118 | |
| VPGPGPGL | 8 | Human | Her2/neu | 884 | A |
| RPGPGPGVSEF | 11 | Human | Her2/neu | 966 | A |
| RIPRGPGPGSEF | 11 | Human | Her2/neu | 966 | A |
| RPRFGPGPGEF | 11 | Human | Her2/neu | 966 | A |
| RPRFRGPGPGF | 11 | Human | Her2/neu | 966 | A |
| APGPGPGAAPA | 11 | Human | p53 | 76 | A |
| APAGPGPGAPA | 11 | Human | p53 | 76 | A |
| APAAGPGPGPA | 11 | Human | p53 | 76 | A |
| APAAPGPGPGA | 11 | Human | p53 | 76 | A |
| RPRGDNFAV | 9 | Pf | SSP2 | 305 | |
| RPGPGPGAV | 9 | Pf | SSP2 | 305 | A |
| RPRGPGPGV | 9 | Pf | SSP2 | 305 | A |
| APRTVALTAL | 10 | Unknown | Naturally | | |
| | | | procesed | | |
| APGPGPGTAL | 10 | Unknown | Naturally | | A |
| | | | procesed | | |
| APRGPGPGAL | 10 | Unknown | Naturally | | A |
| | | | procesed | | |
| APRTGPGPGL | 10 | Unknown | Naturally | | A |
| | | | procesed | | |
| XVXDNATEY | 9 | Unknown | Naturally | | A |
| | | | procesed | | |
| LGFVFTLTV | 9 | unknown | | | |

TABLE 20

HLA-B7 SUPERTYPE

| | | | | | | |
|---|---|---|---|---|---|---|
| | SEQ | | | | | |
| Sequence | ID NO. | B*0702 | B*3501 | B*5101 | B*5301 | B*5401 |

| | | | | | |
|---|---|---|---|---|---|
| APGPGPGLL | 299 | 7481 | 1614 | 18117 | 15613 |
| APRGPGPGL | 4.9 | 974 | 633 | 19779 | 1120 |
| QPRAPIRPI | 6770 | >72000 | >55000 | 12 | >100000 |
| YPLHEQHGM | >55000 | 20785 | >55000 | 10 | >100000 |
| CPTVQASKL | 3247 | 645 | 448 | 1861 | 21643 |
| SPTYKAFL | 109 | 31169 | 4665 | 54879 | 58651 |
| SPGPGPGL | 173 | 2337 | 3535 | 25607 | 53272 |
| TPAGPGPGVF | 334 | 374 | 296 | 2629 | 351 |
| TPAIRGPGPGF | 144 | 1678 | 2418 | 2742 | 31768 |
| TPTGWGLAI | 76 | 5145 | 103 | 1343 | 172 |
| APCNFFTSA | 43 | 8087 | 1045 | >22409.64 | 0.61 |
| GPGHKARVI | 1686 | >72000 | >55000 | 2.2 | >50000 |
| RPQVPLRPMTI | 47009 | >18997.36 | 8081 | 21518 | 129 |
| FPVRPQVPI | 94 | 124 | 39 | 222 | 9.1 |
| RPQVPLRPI | 367 | >23225.81 | >9001.64 | 85335 | 1215 |
| RPQVPLRPMTI | 140 | 10455 | 5045 | 21538 | >15128.59 |
| YPLTFGWCI | 54283 | 1378 | 153 | 154 | 79 |
| FPLTFGWCI | 47951 | 164 | 63 | 36 | 14 |
| FPLTFGWCFKI | 52567 | 4991 | 590 | 188 | 105 |
| FPVRPQVPL | 17 | 3.8 | 18 | 49 | 21 |
| FPGPGPGPL | 1584 | 426 | 2330 | 21036 | 29900 |
| FPVGPGPGL | 106 | 14 | 138 | 32 | 246 |
| GPKVKQWPI | 5500 | >72000 | >55000 | 2.3 | >50000 |
| LPPLERLTI | 24398 | 13399 | 359 | 2624 | 11243 |
| CPEEKQRHL | 10 | >52554.74 | >35483.87 | >109411.76 | >76923.08 |
| VPGPGPGL | 1517 | 447 | 537 | 4094 | 46405 |
| RPGPGPGVSEF | 119 | 18115 | 16774 | 20988 | 3360 |
| RPRGPGPGSEF | 11 | 24871 | >14824.8 | 19336 | 2745 |
| RPRFGPGPGEF | 14 | >30901.29 | >14824.8 | 76844 | 15470 |
| RPRFRGPGPGF | 9.7 | >30901.29 | >14824.8 | 49682 | 60095 |
| APGPGPGAAPA | 1112 | 1252 | 1317 | 4366 | 361 |
| APAGPGPGAPA | 161 | >28915.66 | 11947 | >39743.59 | 43 |
| APAAGPGPGPA | 173 | 12845 | 12470 | 28574 | 204 |
| APAAPGPGPGA | 811 | 3484 | 15814 | >39240.51 | 158 |
| RPRGDNFAV | 12 | 20386 | 1681 | >46268.66 | 212 |
| RPGPGPGAV | 23 | 48487 | 2899 | >46268.66 | 1891 |
| RPRGPGPGV | 11 | 2368 | 52 | 34831 | 47 |
| APRTVALTAL | 12 | 4351 | 14601 | 61596 | 16804 |
| APGPGPGTAL | 81 | 16315 | 16462 | >43661.97 | 35965 |
| APRGPGPGAL | 11 | 23381 | 12732 | >43661.97 | 1665 |
| APRTGPGPGL | 15 | 1414 | 1559 | 22012 | 2043 |
| XVXDNATEY | >55000 | 444 | | | >100000 |
| LGFVFTLTV | 849 | >72000 | 27500 | >93000 | 464 |

TABLE 21

HLA-B44 SUPERTYPE

| | | | | | | |
|---|---|---|---|---|---|---|
| | SEQ | | | | | |
| | ID | | | | | |
| Sequence | NO. | AA | Organism | Protein | Position | Analog |

| | | | | | |
|---|---|---|---|---|---|
| SEAAYAKKI | 9 | Artificial | pool | | A |
| | | sequence | consensus | | |
| GEFPYKAAA | 9 | Artificial | pool | | A |
| | | sequence | consensus | | |
| SEAPYKAIL | 9 | Artificial | pool | | A |
| | | sequence | consensus | | |
| SEAPKYAIL | 9 | Artificial | pool | | A |
| | | sequence | consensus | | |
| AIEFKYIAAV | 9 | Artificial | pool | | A |
| | | sequence | consensus | | |
| AEIPYLAKY | 9 | Artificial | pool | | A |
| | | sequence | consensus | | |
| AEIPKLAYF | 9 | Artificial | pool | | A |
| | | sequence | consensus | | |
| FPFDYAAAF | 9 | Artificial | | | A |
| | | sequence | | | |
| FPFKYKAAF | 9 | Artificial | | | A |
| | | sequence | | | |
| FPFKYAKAF | 9 | Artificial | | | A |
| | | sequence | | | |
| FPFKYAAAF | 9 | Artificial | | | A |
| | | sequence | | | |
| FAFKYAAAF | 9 | Artificial | | | A |
| | | sequence | | | |
| FQFKYAAAF | 9 | Artificial | | | A |
| | | sequence | | | |
| FDFKYAAAF | 9 | Artificial | | | A |
| | | sequence | | | |
| SENDRYRLL | 9 | EBV | BZLF1 | 209 | A |
| IEDPPYNSL | 9 | EBV | lmp2 | 200 | A |
| YEANGNLI | 8 | Flu | HA | 259 | A |
| YBDLRVLSF | 9 | Flu | NP | 338 | A |
| SDYEGRLI | 8 | Flu | NP | 50 | |
| GEISPYPSL | 9 | Flu | NS1 | 158 | A |
| MDIDPYKEF | 9 | HBV | NUC | 30 | |
| LDKGIKPY | 8 | HBV | POL | 125 | |
| ADLMGYIPL | 9 | HCV | core | 131 | |
| LDPYARVAI | 9 | HCV | NS5b | 2663 | A |
| AENLWVTVY | 9 | HIV | gp120 | 1 | |
| KBNLWVTVY | 9 | HIV | gp120 | 1 | A |
| AEKLWVTVY | 9 | HIV | gp120 | 1 | A |
| AENKWVTVY | 9 | HIV | gp120 | 1 | A |
| AENLKVTVY | 9 | HIV | gp120 | 1 | A |
| AENLWKTVY | 9 | HIV | gp120 | 1 | A |
| AENLWVKVY | 9 | HIV | gp120 | 1 | A |
| AENLWVTKY | 9 | HIV | gp120 | 1 | A |
| AENLWVTYK | 9 | HIV | gp120 | 1 | A |
| FENLWVTVY | 9 | HIV | gp120 | 1 | A |
| VENLWVTVY | 9 | HIV | gp120 | 1 | A |
| PENLWVTVY | 9 | HIV | gp120 | 1 | A |
| NENLWVTVY | 9 | HIV | gp120 | 1 | A |
| DENLWYTVY | 9 | HIV | gp120 | 1 | A |
| TENLWVTVY | 9 | HIV | gp120 | 1 | A |
| YENLWVTVY | 9 | HIV | gp120 | 1 | A |
| ATNLWVTVY | 9 | HIV | gp120 | 1 | A |
| AEFLWVTVY | 9 | HIV | gp120 | 1 | A |
| AEVLWVTVY | 9 | HIV | gp120 | 1 | A |
| AEPLWVTVY | 9 | HIV | gp120 | 1 | A |
| ABDLWVTVY | 9 | HIV | gp120 | 1 | A |
| AENLWVTVY | 9 | HIV | gp120 | 1 | |
| AETLWVTVY | 9 | HIV | gp120 | 1 | A |
| AENFWVTVY | 9 | HIV | gp120 | 1 | A |
| ABNVWVTVY | 9 | HIV | gp120 | 1 | A |
| AENPWVTVY | 9 | HIV | gp120 | 1 | A |
| AENDWVTVY | 9 | HIV | gp120 | 1 | A |
| AENNWVTVY | 9 | HIV | gp120 | 1 | A |
| AENTWVTVY | 9 | HIV | gp120 | 1 | A |
| AENLFVTVY | 9 | HIV | gp120 | 1 | A |
| ABNLVVTVY | 9 | HIV | gp120 | 1 | A |
| AENLPVTVY | 9 | HIV | gp120 | 1 | A |
| AENLDVTVY | 9 | HIV | gp120 | 1 | A |
| AENLNVTVY | 9 | HIV | gp120 | 1 | A |
| AENLTVTVY | 9 | HIV | gp120 | 1 | A |
| AENLWFTVY | 9 | HIV | gp120 | 1 | A |
| AENLWLTVY | 9 | HIV | gp120 | 1 | A |
| AENLWPTVY | 9 | HIV | gp120 | 1 | A |
| AENLWDTVY | 9 | HIV | gp120 | 1 | A |
| AENLWNTVY | 9 | HIV | gp120 | 1 | A |
| AENLWTTVY | 9 | HIV | gp120 | 1 | A |
| AENLWVFVY | 9 | HIV | gp120 | 1 | A |
| AENLWVVVY | 9 | HIV | gp120 | 1 | A |
| AENLWVPVY | 9 | HIV | gp120 | 1 | A |
| AENLWVDVY | 9 | HIV | gp120 | 1 | A |
| AENLWVNVY | 9 | HIV | gp120 | 1 | A |
| AENLWVSVY | 9 | HIV | gp120 | 1 | A |
| AENLWVTFY | 9 | HIV | gp120 | 1 | A |
| AENLWVTLY | 9 | HIV | gp120 | 1 | A |
| AENLWVTPY | 9 | HIV | gp120 | 1 | A |
| AENLWVTDY | 9 | HIV | gp120 | 1 | A |
| AENLWVTNY | 9 | HIV | gp120 | 1 | A |
| ABNLWVTTY | 9 | HIV | gp120 | 1 | A |
| AENLWVTVA | 9 | HIV | gp120 | 1 | A |
| AENLWVTVG | 9 | HIV | gp120 | 1 | A |
| AENLWVTVE | 9 | HIV | gp120 | 1 | A |
| AENLWVTVF | 9 | HIV | gp120 | 1 | A |
| AENLWVTVG | 9 | HIV | gp120 | 1 | A |
| AENLWVTVH | 9 | HIV | gp120 | 1 | A |
| AENLWVTVI | 9 | HIV | gp120 | 1 | A |
| AENLWVTVL | 9 | HIV | gp120 | 1 | A |
| AENLWVTVM | 9 | HIV | gp120 | 1 | A |
| AENLWVTVN | 9 | HIV | gp120 | 1 | A |
| AENLWVTVP | 9 | HIV | gp120 | 1 | A |
| AENLWVTVQ | 9 | HIV | gp120 | 1 | A |
| AENLWVTVR | 9 | HIV | gp120 | 1 | A |
| AENLWVTVS | 9 | HIV | gp120 | 1 | A |
| AENLWVTVT | 9 | HIV | gp120 | 1 | A |
| AENLWVTVV | 9 | HIV | gp120 | 1 | A |
| AENLWVTVW | 9 | HIV | gp120 | 1 | A |
| AENLWVTVY | 9 | HIV | gp120 | 1 | |
| ABNLYVTVF | 9 | HIV | gp120 | 1 | A |
| TEPAAVGVGAV | 11 | HIV | NEF | 33 | |
| AEPAAEGV | 8 | HIV | NEF | 34 | |
| AEPAAEGVGA | 10 | HIV | NEF | 34 | |
| ABPAAEGVGAV | 11 | HIV | NEF | 34 | |
| QEEEEVGFPV | 10 | HIV | NEF | 84 | |
| EEEEVGFPV | 9 | HIV | NEF | 86 | |
| EEEVGFPV | 8 | HIV | NEF | 87 | |
| EEVGFPVRPQV | 11 | HIV | NEF | 88 | |
| DEEVGFPV | 8 | HIV | NEF | 89 | |
| KBKGGLDGL | 9 | HIV | NEF | 120 | |
| KBKGGLDGLI | 10 | HIV | NEF | 120 | |
| QEILDLWV | 8 | HIV | NEF | 184 | |
| QEILDLWVY | 9 | HIV | NEF | 184 | |
| AETFYYDGA | 9 | HIV | POL | 629 | |
| EEKPRTLHDL | 10 | HPV | E6 | 6 | |
| NEILIRCII | 9 | HPV | E6 | 97 | |
| QEKKRHVDL | 9 | HPV | E6 | 113 | |
| AEGKEVLL | 8 | Human | CEA | 46 | |
| QELFIPNL | 8 | Human | CEA | 282 | |
| QELFISNI | 8 | Human | CEA | 460 | |
| TEKNSGLY | 8 | Human | CEA | 468 | |
| AELPKPSI | 8 | Human | CEA | 498 | |
| PEAQNTTY | 8 | Human | CEA | 525 | |
| IESTPFNVA | 9 | Human | CEA | 38 | |
| AEGKEVLLL | 9 | Human | CEA | 46 | |
| EEATGQFRV | 9 | Human | CEA | 132 | |
| VEDKDAVAF | 9 | Human | CEA | 157 | |
| CEPETQDAT | 9 | Human | CEA | 167 | |
| PETQDATYL | 9 | Human | CEA | 169 | |
| CETQNPVSA | 9 | Human | CEA | 215 | |
| QELFIIPNIT | 9 | Human | GEA | 282 | |
| AEPPKPFIT | 9 | Human | CEA | 320 | |
| VEDEDAVAL | 9 | Human | CEA | 335 | |
| CEPEIQNTT | 9 | Human | CEA | 345 | |
| PEIQNTTYL | 9 | Human | CEA | 347 | |
| YECGIQNEL | 9 | Human | CEA | 391 | |
| QELFISNIT | 9 | Human | CEA | 460 | |
| TEKNSQLYT | 9 | Human | CEA | 468 | |
| AEGKIEVLLLV | 10 | Human | CEA | 46 | |
| KEVLLLVHNL | 10 | Human | CEA | 49 | |
| GERVDGNRQI | 10 | Human | CEA | 70 | |
| REIIYPNASL | 10 | Human | CEA | 98 | |
| NEEATGQFRV | 10 | Human | CEA | 131 | |
| EEATGQFRVY | 10 | Human | CEA | 132 | |
| GENLNLSCHA | 10 | Human | CEA | 252 | |
| QELFIIPNITV | 10 | Human | CEA | 282 | |
| CEPEIQNTTY | 10 | Human | CEA | 345 | |
| PEIQNTIFYLW | 10 | Human | CEA | 347 | |
| CEPEAQNTTY | 10 | Human | CEA | 523 | |
| PEAQNTTYLW | 10 | Human | CEA | 525 | |
| MESPSAPPHRW | 11 | Human | CEA | 1 | |
| IESTPFNVAEG | 11 | Human | CEA | 38 | |
| GERVDGNRQII | 11 | Human | CEA | 70 | |
| REIIYPNASLL | 11 | Human | CEA | 98 | |
| NEEATGQFRVY | 11 | Human | CEA | 131 | |
| CEPETQDATYL | 11 | Human | CEA | 167 | |
| GENLNLSCHAA | 11 | Human | CEA | 252 | |
| CEPEIQNTTYL | 11 | Human | CEA | 345 | |
| PEIQNTTYLWW | 11 | Human | CEA | 347 | |
| YECGIQNELSV | 11 | Human | CEA | 391 | |
| NELSVDHSDPV | 11 | Human | CEA | 397 | |
| GEPEAQNTTYL | 11 | Human | CEA | 523 | |
| PEAQNTTYLWW | 11 | Human | CEA | 525 | |
| PEIQNTTYLWWV | 12 | Human | CEA | 347 | |
| PEAQNTTYLWWV | 12 | Human | CEA | 525 | |
| CEPEIQNTTYLWW | 13 | Human | CEA | 345 | |
| AEMGKGSFKY | 10 | Human | elong. | 48 | |
| | | | Factor Tu | | |
| SEDCQSL | 7 | Human | Her2/neu | 209 | |
| REVRAVT | 7 | Human | Her2/neu | 351 | |
| FETLEEI | 7 | Human | Her2/neu | 400 | |
| TELVEPL | 7 | Human | Her2/neu | 694 | |
| SECRPRF | 7 | Human | Her2/neu | 963 | |
| PETHLDML | 8 | Human | Her2/neu | 39 | |
| QEVQGYVL | 8 | Human | Her2/neu | 78 | |
| RELQLRSL | 8 | Human | Her2/neu | 138 | |
| CELHCPAL | 8 | Human | Her2/neu | 264 | |
| LEEITGYL | 8 | Human | Her2/neu | 403 | |
| EEITGYLY | 8 | Human | Her2/neu | 404 | |
| DECVGEGL | 8 | Human | Her2/neu | 502 | |
| AEQRASPL | 8 | Human | Her2/neu | 644 | |
| KBILDEAY | 8 | Human | Her2/neu | 765 | |
| EEAPRSPL | 8 | Human | Her2/neu | 1068 | |
| SEDPTVPL | 8 | Human | Her2/neu | 1113 | |
| MELAALCRW | 9 | Human | Her2/neu | 1 | |
| QEVQGYVLI | 9 | Human | Her2/neu | 78 | |
| FEDNYALAV | 9 | Human | Her2/neu | 108 | |
| RELQLRSLT | 9 | Human | Her2/neu | 138 | |
| TEILKGGVL | 9 | Human | Her2/neu | 146 | |
| HEQCAAGCT | 9 | Human | Her2/neu | 237 | |
| GELHCPALV | 9 | Human | Her2/neu | 264 | |
| FESMPNPEG | 9 | Human | Her2/neu | 279 | |
| QEVTAEDGT | 9 | Human | Her2/neu | 320 | |
| CEKCSKPCA | 9 | Human | Her2/neu | 331 | |
| MEHLREVRA | 9 | Human | Her2/neu | 347 | |
| REVRAVTSA | 9 | Human | Her2/neu | 351 | |
| QEFAGCKKI | 9 | Human | Her2/neu | 362 | |
| EEITGYLYI | 9 | Human | Her2/neu | 404 | |
| RELGSGLAL | 9 | Human | Her2/neu | 459 | |
| GEGLACHQL | 9 | Human | Her2/neu | 506 | |
| QEGVEECRV | 9 | Human | Her2/neu | 538 | |
| VEECRVLQG | 9 | Human | Her2/neu | 541 | |
| EECRVLQGL | 9 | Human | Her2/neu | 542 | |
| AEQRASPLT | 9 | Human | Her2/neu | 644 | |
| QETELVEPL | 9 | Human | Her2/neu | 692 | |
| VEPLTPSGA | 9 | Human | Her2/neu | 697 | |
| TELRKVKVL | 9 | Human | Her2/neu | 718 | |
| GENVKIPVA | 9 | Human | Her2/neu | 743 | |
| KBILDEAYV | 9 | Human | Her2/neu | 765 | |
| DEAYVMAGV | 9 | Human | Her2/neu | 769 | |
| DETEYHADG | 9 | Human | Her2/neu | 873 | |
| LESILRRRF | 9 | Human | Her2/neu | 891 | |
| GERLPQPPI | 9 | Human | Her2/neu | 938 | |
| LEDDDMGDL | 9 | Human | Her2/neu | 1009 | |
| EEYLVPQQG | 9 | Human | Her2/neu | 1021 | |
| EEEAPRSPL | 9 | Human | Her2/neu | 1067 | |
| EEAPRSPLA | 9 | Human | Her2/neu | 1068 | |
| SEQAGSDVF | 9 | Human | Her2/neu | 1078 | |
| PEYVNQPDV | 9 | Human | Her2/neu | 1137 | |
| PEYLTPQGG | 9 | Human | Her2/neu | 1194 | |
| PERGAPPST | 9 | Human | Her2/neu | 1228 | |
| ABNPEYLGL | 9 | Human | Her2/neu | 1243 | |
| MELAALCRWG | 10 | Human | Her2/neu | 1 | |
| LELTYLPTNA | 10 | Human | Her2/neu | 60 | |
| QEVQGYVLIA | 10 | Human | Her2/neu | 78 | |
| FEDNYALAVL | 10 | Human | Her2/neu | 108 | |
| TEILKGGVLI | 10 | Human | Her2/neu | 146 | |
| GESSEDCQSL | 10 | Human | Her2/neu | 206 | |
| SEDCQSLTRT | 10 | Human | Her2/neu | 209 | |
| CELHCPALVT | 10 | Human | Her2/neu | 264 | |
| MEHLREVRAV | 10 | Human | Her2/neu | 347 | |
| QEFAGCKKIF | 10 | Human | Her2/neu | 362 | |
| FETLEEITGY | 10 | Human | Her2/neu | 400 | |
| LEEITGYLYI | 10 | Human | Her2/neu | 403 | |
| RELGSGLALI | 10 | Human | Her2/neu | 459 | |
| PEDECVGEGL | 10 | Human | Her2/neu | 500 | |
| QEGVEECRVL | 10 | Human | Her2/neu | 538 | |
| YEECRVLQGL | 10 | Human | Her2/neu | 541 | |
| REYVNARHCL | 10 | Human | Her2/neu | 552 | |
| PECQPQNGSV | 10 | Human | Her2/neu | 565 | |
| EEGACQPCPI | 10 | Human | Her2/neu | 619 | |
| QETELVIEPLT | 10 | Human | Her2/neu | 692 | |
| VEPLTPSGAM | 10 | Human | Her2/neu | 697 | |
| KBTELRKVKV | 10 | Human | Her2/neu | 716 | |
| TELRKVKVLG | 10 | Human | Her2/neu | 718 | |
| GENVKIPVAI | 10 | Human | Her2/neu | 743 | |
| KEILDEAYVM | 10 | Human | Her2/neu | 765 | |
| DEAYVMAGVG | 10 | Human | Her2/neu | 769 | |
| DETEYHADGG | 10 | Human | Her2/neu | 873 | |
| TEYHADGGKV | 10 | Human | Her2/neu | 875 | |
| LESILRRRFT | 10 | Human | Her2/neu | 891 | |
| REIPDLLEKG | 10 | Human | Her2/neu | 929 | |
| SECRPRFREL | 10 | Human | Her2/neu | 963 | |
| RELVSEFSRM | 10 | Human | Her2/neu | 970 | |
| NEDLGPASPL | 10 | Human | Her2/neu | 991 | |
| AEEYLVPQQG | 10 | Human | Her2/neu | 1020 | |
| EEYLVPQQGF | 10 | Human | Her2/neu | 1021 | |
| SEEEAPRSPL | 10 | Human | Her2/neu | 1066 | |
| EEEAPRSPLA | 10 | Human | Her2/neu | 1067 | |
| SETDGYVAPL | 10 | Human | Her2/neu | 1122 | |
| PERGAPPSTF | 10 | Human | Her2/neu | 1228 | |
| PEYLGLDVPV | 10 | Human | Her2/neu | 1246 | |
| MELAALCRWGL | 11 | Human | Her2/neu | 1 | |
| PETHLDMLRHL | 11 | Human | Her2/neu | 39 | |
| RELQLRSLTEI | 11 | Human | Her2/neu | 138 | |
| GESSEDCQSLT | 11 | Human | Her2/neu | 206 | |
| SEDCQSLTRTV | 11 | Human | Her2/neu | 209 | |
| GELHCPALVTY | 11 | Human | Her2/neu | 264 | |
| FESMPNPEGRY | 11 | Human | Her2/neu | 279 | |
| CEKCSKPCARV | 11 | Human | Her2/neu | 331 | |
| MEHLREVRAVT | 11 | Human | Her2/neu | 347 | |
| REVRAVTSANI | 11 | Human | Her2/neu | 351 | |
| QEFAGCKKIFG | 11 | Human | Her2/neu | 362 | |
| FEThEEITGYL | 11 | Human | Her2/neu | 400 | |
| EEITGYLYISA | 11 | Human | Her2/neu | 404 | |
| GEGLACHQLCA | 11 | Human | Her2/neu | 506 | |
| DEEGACQPCPI | 11 | Human | Her2/neu | 618 | |
| AEQRASPLTSI | 11 | Human | Her2/neu | 644 | |
| TELVEPLTPSG | 11 | Human | Her2/neu | 694 | |
| KBTELRKVKVL | 11 | Human | Her2/neu | 716 | |
| KEILDEAYVMA | 11 | Human | Her2/neu | 765 | |
| LEDVRLVHRDL | 11 | Human | Her2/neu | 836 | |
| WELMTFGAKPY | 11 | Human | Her2/neu | 913 | |
| GERLPQPPICT | 11 | Human | Her2/neu | 938 | |
| SEGRPRFRBLV | 11 | Human | Her2/neu | 963 | |
| RELVSEFSRMA | 11 | Human | Her2/neu | 970 | |
| AEEYLVPQQGF | 11 | Human | Her2/neu | 1020 | |
| EEYLVPQQGFF | 11 | Human | Her2/neu | 1021 | |
| SEEEAPRSPLA | 11 | Human | Her2/neu | 1066 | |
| SEGAGSDVFDG | 11 | Human | Her2/neu | 1078 | |
| SETDGYVAPLT | 11 | Human | Her2/neu | 1122 | |
| REGPLPAARPA | 11 | Human | Her2/neu | 1153 | |
| VENPEYLTPQG | 11 | Human | Her2/neu | 1191 | |
| PEYLTPQGGAA | 11 | Human | Her2/neu | 1194 | |
| AENPEYLGLDV | 11 | Human | Her2/neu | 1243 | |
| LELTYLPTNASL | 12 | Human | Her2/neu | 60 | |
| RELQLRSLTEIL | 12 | Human | Her2/neu | 138 | |
| PEGRYTFGASCV | 12 | Human | Her2/neu | 285 | |
| LEEITGYLYISA | 12 | Human | Her2/neu | 403 | |
| EEITGYLYISAW | 12 | Human | Her2/neu | 404 | |
| PEADQCVACAHY | 12 | Human | Her2/neu | 579 | |
| TELVEPLTPSGA | 12 | Human | Her2/neu | 694 | |
| TEYHADGGKVPI | 12 | Human | Her2/neu | 875 | |
| GERLPQPPIGTI | 12 | Human | Her2/neu | 938 | |
| AEEYLVPQQGFF | 12 | Human | Her2/neu | 1020 | |
| PEGRYTFGASCVT | 13 | Human | Her2/neu | 285 | |
| CEKCSKPCARVCY | 13 | Human | Her2/neu | 331 | |
| MEHLREVRAVTSA | 13 | Human | Her2/neu | 347 | |
| DECVGEGLAGHQL | 13 | Human | Her2/neu | 502 | |
| PECQPQNGSVTCF | 13 | Human | Her2/neu | 565 | |
| RBNTSPKANKEIL | 13 | Human | Her2/neu | 756 | |
| REIPDLLEKGERL | 13 | Human | Her2/neu | 929 | |
| SEFSRMARDPQRF | 13 | Human | Her2/neu | 974 | |
| SEGAGSDVFDGDL | 13 | Human | Her2/neu | 1078 | |
| GEFGGYGSV | 9 | Human | Histactranf | 127 | A |
| LWQLNGRLEYTLKDR | 15 | Human | IFN-B | 21 | A |
| SEFQAAI | 7 | Human | MAGE2 | 103 | |
| SEYLQLV | 7 | Human | MAGE2 | 155 | |
| WEELSML | 7 | Human | MAGE2 | 222 | |
| GEPHISY | 7 | Human | MAGE2 | 295 | |
| LEARGEAL | 8 | Human | MAGE2 | 16 | |
| QEEEGPRM | 8 | Human | MAGE2 | 90 | |
| EEEGPRMF | 8 | Human | MAGE2 | 91 | |
| VELVEELL | 8 | Human | MAGE2 | 114 | |
| AEMLESVL | 8 | Human | MAGE2 | 133 | |
| SEYLQLVF | 8 | Human | MAGE2 | 155 | |
| EEKIWEEL | 8 | Human | MAGE2 | 218 | |
| LEARGEALG | 9 | Human | MAGE2 | 16 | |
| GEALGLVGA | 9 | Human | MAGE2 | 20 | |
| QEEEGPRMF | 9 | Human | MAGE2 | 90 | |
| VELVHFLLL | 9 | Human | MAGE2 | 114 | |
| REPVTKAEM | 9 | Human | MAGE2 | 127 | |
| SEYLQLVFG | 9 | Human | MAGE2 | 155 | |
| PEEKIWEEL | 9 | Human | MAGE2 | 217 | |
| EELSMLEVF | 9 | Human | MAGE2 | 223 | |
| FEGREDSVF | 9 | Human | MAGE2 | 231 | |
| YEFLWGPRA | 9 | Human | MAGE2 | 269 | |
| EEGLEARGEA | 10 | Human | MAGE2 | 13 | |
| LEARGEALGL | 10 | Human | MAGE2 | 16 | |
| VEVTLGEVPA | 10 | Human | MAGE2 | 46 | |
| EEGPRMFPDL | 10 | Human | MAGE2 | 92 | |
| REPVTKAEML | 10 | Human | MAGE2 | 127 | |
| SEYLQLVFGI | 10 | Human | MAGE2 | 155 | |
| VEVVPISHLY | 10 | Human | MAGE2 | 167 | |
| EEKIWEELSM | 10 | Human | MAGE2 | 218 | |
| WEELSMLEVF | 10 | Human | MAGE2 | 222 | |
| FEGREDSVFA | 10 | Human | MAGE2 | 231 | |
| QENYLEYRQV | 10 | Human | MAGE2 | 252 | |
| YEFLWGPRAL | 10 | Human | MAGE2 | 269 | |
| GEPHISYPPL | 10 | Human | MAGE2 | 295 | |
| EEGLEARGEAL | 11 | Human | MAGE2 | 13 | |
| LEARGEALGLV | 11 | Human | MAGE2 | 16 | |
| GEALGLVGAQA | 11 | Human | MAGE2 | 20 | |
| EEQQTASSSST | 11 | Human | MAGE2 | 34 | |
| VEVTLGEVPAA | 11 | Human | MAGE2 | 46 | |
| EEEGPRMFPDL | 11 | Human | MAGE2 | 91 | |
| SEFQAAISRKM | 11 | Human | MAGE2 | 103 | |
| VELVHFLLLKY | 11 | Human | MAGE2 | 114 | |
| LESVLRNCQDF | 11 | Human | MAGE2 | 136 | |
| VEVVPISHLYI | 11 | Human | MAGE2 | 167 | |
| IEGDCAPEEKI | 11 | Human | MAGE2 | 211 | |
| EEKIWEELSML | 11 | Human | MAGE2 | 218 | |
| EELSMLEVFEG | 11 | Human | MAGE2 | 223 | |
| LEVEEGREDSV | 11 | Human | MAGE2 | 228 | |
| YEFLWGPRALI | 11 | Human | MAGE2 | 269 | |
| EEQQTASSSSTL | 12 | Human | MAGE2 | 34 | |
| QEEEGPRMFPDL | 12 | Human | MAGE2 | 90 | |
| SEFQAAISRKMV | 12 | Human | MAGE2 | 103 | |
| LESVLRNCQDFF | 12 | Human | MAGE2 | 136 | |
| VEVYPISHLYIL | 12 | Human | MAGE2 | 167 | |
| EEGLEARGEALGL | 13 | Human | MAGE2 | 13 | |
| LEARGEALGLVGA | 13 | Human | MAGE2 | 16 | |
| LESEFQAAISRKM | 13 | Human | MAGE2 | 101 | |
| REPVTKAEMLESV | 13 | Human | MAGE2 | 127 | |
| SEYLQLVFGIEVV | 13 | Human | MAGE2 | 155 | |
| IEVVEVVPISHLY | 13 | Human | MAGE2 | 164 | |
| VEVVPISHLYILV | 13 | Human | MAGE2 | 167 | |
| MEVDPIGHLY | 10 | Human | MAGE3 | 167 | |
| EEEGPSTF | 8 | Human | MAGE3 | 91 | |
| AELVHFLL | 8 | Human | MAGE3 | 114 | |
| FEGREDSI | 8 | Human | MAGE3 | 231 | |
| QEAASSSST | 9 | Human | MAGE3 | 36 | |
| AELVHFLLL | 9 | Human | MAGE3 | 114 | |
| AEMLGSVVG | 9 | Human | MAGE3 | 133 | |
| EELSVLEVF | 9 | Human | MAGE3 | 223 | |
| FEGREDSIL | 9 | Human | MAGE3 | 231 | |
| QEAASSSSTL | 10 | Human | MAGE3 | 36 | |
| EEGPSTFPDL | 10 | Human | MAGE3 | 92 | |
| IELMEVDPIG | 10 | Human | MAGE3 | 164 | |
| MEVDPIGHLY | 10 | Human | MAGE3 | 167 | |
| EEKIWEELSV | 10 | Human | MAGE3 | 218 | |
| WEELSVLEVF | 10 | Human | MAGE3 | 222 | |
| FEGREDSILG | 10 | Human | MAGE3 | 231 | |
| EEEGPSTFPDL | 11 | Human | MAGE3 | 91 | |
| AELVHFLLLKY | 11 | Human | MAGE3 | 114 | |
| MEVDPIGHLYI | 11 | Human | MAGE3 | 167 | |
| REGDCAPEEKI | 11 | Human | MAGE3 | 211 | |
| EEKIWEELSVL | 11 | Human | MAGE3 | 218 | |
| LEVFEGREDSI | 11 | Human | MAGE3 | 228 | |
| RERFEMF | 7 | Human | p53 | 335 | |
| LEDSSGNL | 8 | Human | p53 | 257 | |
| GEYFTLQI | 8 | Human | p53 | 325 | |
| VEPPLSQET | 9 | Human | p53 | 10 | |
| PENNVLSPL | 9 | Human | p53 | 27 | |
| DEAPRMPEA | 9 | Human | p53 | 61 | |
| HBRCSDSDG | 9 | Human | p53 | 179 | |
| VEGNLRVEY | 9 | Human | p53 | 197 | |
| VEYLDDRNT | 9 | Human | p53 | 203 | |
| LEDSSGNLL | 9 | Human | p53 | 257 | |
| RELNEALEL | 9 | Human | p53 | 342 | |
| NEALELKDA | 9 | Human | p53 | 345 | |
| LELKDAQAG | 9 | Human | p53 | 348 | |
| MEEIPQSDPSV | 10 | Human | p53 | 1 | |
| VEPPLSQETF | 10 | Human | p53 | 10 | |
| QETFSDLWKL | 10 | Human | p53 | 16 | |
| IEQWFTEDPG | 10 | Human | p53 | 50 | |
| DEAPRMPEAA | 10 | Human | p53 | 61 | |
| HERCSDSDGL | 10 | Human | p53 | 179 | |
| VEGNLRVEYL | 10 | Human | p53 | 197 | |
| VEYLDDRNTF | 10 | Human | p53 | 203 | |
| PEVGSDCTTI | 10 | Human | p53 | 223 | |
| LEDSSGNLLG | 10 | Human | p53 | 257 | |
| FEVRVCAGPG | 10 | Human | p53 | 270 | |
| TEEENLRXKG | 10 | Human | p53 | 284 | |
| GEPHHELPPG | 10 | Human | p53 | 293 | |
| GEYETLQIRG | 10 | Human | p53 | 325 | |
| RERFEMFREL | 10 | Human | p53 | 335 | |
| FEMFRELNEA | 10 | Human | p53 | 338 | |
| QETFSDLWKLL | 11 | Human | p53 | 16 | |
| HERCSDSDGLA | 11 | Human | p53 | 179 | |
| YEPPEVGSDCT | 11 | Human | p53 | 220 | |
| HELPPGSTKRA | 11 | Human | p53 | 297 | |
| FEMFRELNEAL | 11 | Human | p53 | 338 | |
| NEALELKDAQA | 11 | Human | p53 | 345 | |
| TEDPGPDEAPRM | 12 | Human | p53 | 55 | |
| GEPHHELPPGST | 12 | Human | p53 | 293 | |
| DEAPRMPEAAPPV | 13 | Human | p53 | 61 | |
| VVPPEVGSDCTTI | 13 | Human | p53 | 220 | |
| RERRDNYV | 8 | Human | unknown | | |
| SEIDLILGY | 9 | Human | unknown | | |
| AEIPTRVNY | 9 | Human | unknown | | |
| AEMGKFKFSY | 10 | Human | unknown | | |
| DEIGVIDLY | 9 | Human | unknown | | |
| AEMGKFKYSF | 10 | Human | unknown | | A |
| SEAIHTFQY | 9 | Human | unknown | | |
| SEAIYTFQF | 9 | Human | unknown | | A |
| AEGIVTGQY | 9 | Human | unknown | | |
| HETTYNSI | 8 | Mouse | beta actin | 275 | A |
| GELSYLNV | 8 | Mouse | cathepsin D | 255 | |
| YEDTGKTI | 8 | Mouse | p40 phox RNA | 245 | |
| YENDIEKKI | 9 | Pf | CSP | 375 | |

TABLE 22
HLA-B44 SUPERTYPE

| | | | | | | | |
|---|---|---|---|---|---|---|---|
| | SEQ | | | | | | |
| | ID | | | | | | |
| Sequence | NO. | B*1801 | B*4001 | B*4002 | B*4402 | B*4403 | B*4501 |

| | | | | | | |
|---|---|---|---|---|---|---|
| SEAAYAKKI | 8609 | 308 | 129 | 1685 | 61 | 287 |
| GEFPYKAAA | 286 | 170 | 3.9 | 746 | 2537 | 11 |
| SEAPYKAIL | 2258 | 29 | 8.8 | 440 | 170 | 262 |
| SEAPKYAIL | 2263 | 113 | 7.8 | 762 | 2260 | 479 |
| AEFKY1AAV | 48 | 2.8 | 6.5 | 28 | 21 | 4.9 |
| AEIPYLAKY | 116 | 7258 | 3159 | 44 | 30 | 668 |
| AEIPKLAYF | 1641 | 57 | 5.6 | 229 | 57 | 608 |
| FPFDYAAAF | 141 | | | | | |
| FPFKYKAAF | 155 | | | | | |
| FPFKYAKAF | 86 | | | | | |
| FPFKYAAAF | 16 | | | | | |
| FAFKYAAAF | 95 | | | | | |
| FQFKYAAAF | 22 | | | | | |
| FDFKYAAAF | 187 | | | | | |
| SENDRYRIL | 18281 | 271 | 23 | 183 | 164 | 1073 |
| IEDPPYNSL | 35457 | 16 | 688 | 15833 | 40075 | 18697 |
| YEANGNLI | 191 | 7.9 | 7.0 | 516 | 3085 | 10342 |
| YEDLRVLSF | 20 | 67 | 71 | 24 | 212 | 18697 |
| SDYEGRLI | >24800 | 27150 | 86 | 851 | 228 | 10469 |
| GEISPYPSL | 19361 | 24 | 1.8 | 3564 | 293 | 115 |
| MDIDPYKEF | 169477 | 3700 | 382 | 21744 | 1949 | 2615 |
| LDKGIKPY | >100000 | 17884 | 468 | >43192.49 | 19311 | 23609 |
| ADLMGYIPL | >7616.71 | 959 | 4.7 | >21395.35 | 10292 | >49000 |
| LDPYARVAI | >24409.45 | >88888.89 | 372 | >41628.96 | >39766.08 | >49000 |
| AENLWVTVY | 155 | 1053 | 547 | 522 | 284 | 200 |
| KENLWVTVY | 184 | 2738 | 373 | 308 | 306 | 6215 |
| AEKLWVTVY | 286 | 18278 | 306 | 168 | 287 | 219 |
| AENKWVTVY | 781 | 11303 | 534 | 294 | 540 | 297 |
| AENLKVTVY | 138 | 7746 | 1075 | 253 | 487 | 9624 |
| AENLWKTVY | 913 | 850 | 406 | 139 | 383 | 245 |
| AENLWVKVY | 2735 | 1482 | 1696 | 708 | 105 | 132 |
| AENLWVTKY | 511 | 1010 | 1998 | 355 | 1064 | 201 |
| AENLWVTVK | 29464 | 853 | 2004 | 6305 | 2133 | 186 |
| FENLWVTVY | 59 | 943 | 1336 | 4179 | 1312 | 21403 |
| VENLWVTVY | 25 | 5499 | 5586 | 13454 | 4856 | 15654 |
| PENLWVTVY | 190 | >72727.27 | >154545.45 | >167272.73 | >425000 | >49000 |
| NENLWVTVY | 38 | >72727.27 | 11774 | 453 | 224 | 1668 |
| DENLWVTVY | 26 | >72727.27 | 41098 | 4589 | 988 | 49000 |
| TENLWVTVY | 14 | 14040 | 1415 | 291 | 364 | 5296 |
| YENLWVTVY | 29 | 552 | 324 | 640 | 369 | 10701 |
| ATNLWVTVY | 17615 | 487 | >154545.45 | 8912 | >43037.97 | >49000 |
| AEFLWVTVY | 131 | 183 | 240 | 1013 | 156 | 472 |
| AEVLWVTVY | 142 | 1549 | 436 | 1520 | 390 | 1244 |
| AEPLWVTVY | 310 | 1727 | 2484 | 1322 | 96 | 1384 |
| AEDLWVTVY | 354 | 423 | 3521 | 2329 | 469 | 1845 |
| AENLWVTVY | 122 | 1581 | 552 | 308 | 132 | 301 |
| AETLWVTVY | 199 | 1052 | 198 | 501 | 221 | 774 |
| AENFWVTVY | 182 | 1394 | 542 | 171 | 268 | 289 |
| AENVWVTVY | 262 | 2238 | 386 | 1112 | 744 | 737 |
| AENPWVTVY | 27 | 843 | 224 | 18 | 53 | 202 |
| AENDWVTVY | 324 | 954 | 742 | 96 | 165 | 365 |
| AENNWVTVY | 167 | 1161 | 357 | 214 | 162 | 99 |
| AENTWVTVY | 213 | 1451 | 1793 | 386 | 166 | 442 |
| AENLFVTVY | 29 | 970 | 334 | 357 | 125 | 232 |
| AENLVVTVY | 62 | 876 | 1344 | 1030 | 203 | 718 |
| AENLPVTVY | 20 | 205 | 566 | 356 | 126 | 246 |
| AENLDVTVY | 517 | 220 | 12081 | 673 | 340 | 1291 |
| AENLNVTVY | 198 | 564 | 3544 | 447 | 358 | 2445 |
| AENLTVTVY | 153 | 689 | 1269 | 327 | 208 | 793 |
| AENLWFTVY | 360 | 699 | 668 | 227 | 62 | 90 |
| AENLWLTVY | 666 | 1702 | 884 | 647 | 226 | 227 |
| ABNLWPTVY | 661 | 690 | 688 | 157 | 50 | 116 |
| AENLWDTVY | 775 | 1145 | 2090 | 414 | 68 | 263 |
| AENLWNTVY | 336 | 1338 | 957 | 66 | 81 | 257 |
| AENLWTTVY | 196 | 246 | 625 | 51 | 50 | 118 |
| AENLWVFVY | 242 | 857 | 375 | 348 | 310 | 237 |
| ABNLWVVVY | 326 | 2728 | 1688 | 599 | 632 | 468 |
| ABNLWVPVY | 303 | 175 | 183 | 96 | 47 | 106 |
| AENLWVDVY | 415 | 700 | 3440 | 334 | 92 | 242 |
| AENLWVNVY | 317 | 1156 | 952 | 159 | 76 | 266 |
| AENLWVSVY | 232 | 1251 | 1347 | 351 | 178 | 292 |
| AENLWVTFY | 1299 | 1201 | 295 | 124 | 222 | 347 |
| AENLWVTLY | 392 | 463 | 731 | 199 | 119 | 349 |
| AENLWVTPY | 41 | 274 | 189 | 127 | 44 | 122 |
| AENLWVTDY | 1001 | 930 | 1208 | 191 | 103 | 328 |
| AENLWVTNY | 730 | 865 | 948 | 149 | 74 | 215 |
| AENLWVTTY | 28 | 280 | 191 | 37 | 26 | 48 |
| ABNLWVTVA | 9689 | 557 | 4.8 | 1543 | 296 | 9.1 |
| AENLWVTVC | 178026 | 157 | 1425 | 5593 | 2267 | 146 |
| AENLWVTVE | >258333.33 | 3888 | 1362 | 8910 | 2573 | 246 |
| AENLWVTVF | 365 | 162 | 20 | 346 | 162 | 262 |
| AENLWVTVG | 39743 | 861 | 47 | 1812 | 245 | 35 |
| AENLWVTVH | 16516 | 493 | 151 | 966 | 387 | 120 |
| AENLWVTVI | 11224 | 14 | 7.3 | 237 | 88 | 54 |
| AENLWVTVL | 6198 | 14 | 13 | 68 | 208 | 114 |
| AENLWVTVM | 508 | 13 | 6.1 | 195 | 35 | 50 |
| AENLWVTVN | 129167 | 6701 | 481 | 2623 | 414 | 169 |
| AENLWVTVP | 38441 | 9711 | 339 | 7715 | 2473 | 187 |
| AENLWVTVQ | 49640 | 522 | 85 | 1223 | 188 | 100 |
| AENLWVTVR | 32979 | 1246 | 1744 | 4857 | 1474 | 233 |
| AENLWVTVS | 25726 | 2163 | 103 | 4221 | 417 | 34 |
| AENLWVTVT | 12331 | 947 | 7.8 | 2696 | 343 | 10 |
| AENLWVTVV | 10709 | 84 | 19 | 5757 | 1432 | 35 |
| AENLWVTVW | 22610 | 1304 | 135 | 423 | 324 | 204 |
| AENLWVTVY | 51 | 1358 | 90 | 66 | 43 | 68 |
| AENLYVTVF | 61 | 17 | 3.1 | 39 | 47 | 69 |
| TEPAAVGVGAV | >8115.18 | 930 | 391 | 1938 | 459 | 8235 |
| ABPAAEGV | >8115.18 | 2070 | 2675 | >22604.42 | 402 | 6590 |
| AEPAAEGVGA | >8115.18 | 4116 | 1655 | >22604.42 | >11447.81 | 104 |
| AEPAAEGVGAV | >8611.11 | 20364 | 242 | >23896.1 | >11447.81 | 1499 |
| QEEEEVGFPV | >8611.11 | 13117 | 2596 | 15203 | >11447.81 | 86 |
| EEEEVGFPV | 3691 | 3340 | 417 | 7440 | 10313 | 37 |
| EEEVGFPV | 427 | 9578 | 2605 | 6372 | >10461.54 | 227 |
| EEVGFPVRPQV | >22794.12 | 9905 | 108 | 23777 | 6553 | 808 |
| DEEVGFPV | 7.1 | >32000 | 4260 | 9305 | >10461.54 | 916 |
| KEKGGLDGL | >22794.12 | 55 | 174 | >81415.93 | >10461.54 | 9926 |
| KEKGGLDGLI | >22794.12 | 843 | 233 | 14726 | 3626 | 9986 |
| QEILDLWV | >22794.12 | 142 | 1717 | >81415.93 | 5919 5504 | |
| QEILDLWVY | 52 | 740 | 4522 | 264 | 172 | 6261 |
| AETFYVDGA | >6709.96 | 21630 | 1923 | >21198.16 | 6924 | 38 |
| EEKPRTLHDL | >81578.95 | 36208 | 34027 | 15236 | 30010 | 419 |
| NEILIRCII | 5672 | 291 | 59 | 2722 | 258 | 3248 |
| QEKKRHVDL | 7.3 | 15984 | 63093 | 443 | 211 | 12613 |
| AEGKEVLL | 11455 | 1311 | 5303 | 17268 | 129 | 14165 |
| QELFIPNI | 127 | 5815 | 147 | 752 | 8.5 | 1319 |
| QELFISNI | 889 | 6396 | 1175 | 2282 | 70 | 1172 |
| TEKNSGLY | 211 | 9851 | 7117 | 1868 | 605 | 10248 |
| AELPKPSI | 7423 | 6697 | 131 | 1164 | 19 | 2608 |
| PEAQNTTY | 149 | 2594 | 2437 | 2204 | 76 | 3255 |
| IESTPFNVA | 69 | 1234 | 66 | 18749 | 0.97 | 15 |
| AEGKEVLLL | 1080 | 72 | 147 | 178 | 1.7 | 199 |
| EEATGQFRV | 805 | 5563 | 470 | 1691 | 95 | 18 |
| VEDKDAVAF | 94 | 121 | 1583 | 1661 | 1443 | 21204 |
| CEPETQDAT | 4009 | 3646 | 410 | 23421 | 50 | 97 |
| PETQDATYL | 9473 | 1240 | 33745 | >34586.47 | 301 | 13430 |
| CETQNPVSA | 73 | 7016 | 261 | 20023 | 10.0 | 15 |
| QELFIPNIT | 125 | 4361 | 172 | 1217 | 3.0 | 18 |
| AEPPKPFIT | 12850 | 7067 | 7170 | >34586.47 | 232 | 1813 |
| VEDEDAVAL | 840 | 11 | 2665 | 30667 | 51 | 27810 |
| CEPEIQNTT | 6889 | 5709 | 3081 | 31834 | 120 | 2732 |
| PEIQNTTYL | 923 | 138 | 2786 | 16816 | 231 | 1825 |
| YECGIQNEL | 82 | 71 | 53 | 452 | 5.3 | 855 |
| QELFISNIT | 530 | 6571 | 58 | 2334 | 3.9 | 80 |
| TEKNSGLYT | 1113 | 7522 | 3195 10097 | 101 | 1963 | |
| AEGKEVLLLV | 5135 | 1019 | 408 | 479 | 8.6 | 994 |
| KEVLLLVHNL | 893 | 3.1 | 4.4 | 414 | 2.3 | 2512 |
| GERVDGNRQI | 9395 | 1933 | 369 | 3900 | 13 | 19464 |
| REIIYPNASL | 741 | 2.3 | 7.5 | 374 | 1.7 | 954 |
| NEEATGQFRV | 998 | 29086 | 22678 | 4365 | 471 | 405 |
| EEATGQFRVY | 64 | >33333.33 | 55956 | 29 | 1041 | 1374 |
| GENLNLSCHA | 14373 | 1341 | 357 | 8610 | 5.3 | 271 |
| QELFIPNITV | 81 | 121 | 27 | 93 | 2.6 | 14 |
| CEPEIQNTTY | 1459 | >10322.58 | 35697 | 49 | 14596 | 43739 |
| PEIQNTTYLW | 819 | 3301 | 9423 | 13 | 6173 | 10011 |
| CEPEAQNTTY | 9525 | >12903.23 | >48571.43 | 61 | >4268.68 | 17330 |
| PEAQNTTYLW | 17082 | >9248.55 | >12592.59 | 27 | 21243 | >28654.97 |
| MESPSAPPHRW | 12 | 943 | 1915 | 5.3 | 41 | 359 |
| IESTPFNVAEG | 87 | 1074 | 352 | 89 | 8.7 | 84 |
| GERVDGNRQII | 764 | 278 | 18 | 871 | 1.3 | 27084 |
| REIIYPNASLL | 1788 | 2.4 | 12 | 57 | 0.38 | 1777 |
| NEEATGQFRVY | 7.7 | 3252 | 999 | 9.6 | 69 | 3986 |
| CEPETQDATYL | 831 | 311 | 3388 | 398 | 807 | 62150 |
| GENLNLSCHAA | 7838 | 4557 | 63 | 1907 | 9.0 | 32 |
| CEPEIQNITYL | 129 | 287 | 1603 | 1245 | 60 | 11981 |
| PEIQNTTYLWW | 172 | 749 | 1045 | 17 | 227 | 1365 |
| YECGIQNELSV | 9.2 | 33 | 26 | 1714 | 0.46 | 155 |
| NELSVDHSDPV | 49 | 2554 | 1128 | 1615 | 38 | 78 |
| CEPEAQNTTYL | 962 | 2184 | 11723 | 3419 | 131 | 2450 |
| PEAQNTTYLWW | 147 | 2096 | 3090 | 121 | 79 | 2005 |
| PBIQNTTYLWWV | 644 | 1808 | 1539 | 481 | 93 | 994 |
| PEAQNTTYLWWV | 20 | 1694 | 646 | 5.1 | 3.3 | |
| CEPEIQNTTYLWW | 84 | 858 | 3168 | 7.9 | 409 | 1243 |
| AEMGKGSFKY | 1618 | 6427 | 3820 | 112 | 90 | 305 |
| SEDCQSL | 18245 | 2691 | 14258 | 8248 | 431 | 19225 |
| REVRAVT | 8564 | 3136 | 725 | 31615 | 29 | 23544 |
| FETLEBI | 1518 | 7621 | 2110 | 42991 | 69 | 67957 |
| TELVEPL | 162 | 14164 | 1258 | 8854 | 66 | >148484.85 |
| SECRPRF | 926 | 18181 | 1157 | 852 | 48 | 8856 |
| PETHLDML | 1954 | 8387 | 6118 | >17523.81 | 83 | 20257 |
| QEVQGYVL | 3.4 | 28 | 5.0 | 1210 | 0.92 | 33 |
| RELQLRSL | 42 | 49 | 5.9 | 2025 | 0.62 | 1372 |
| CELHCPAL | 150 | 871 | 259 | 4361 | 39 | 30089 |
| LEEITGYL | 242 | 830 | 1805 | 5913 | 403 | 35502 |
| EEITGYLY | 20 | 5713 | 1223 | 11 | 83 | 238 |
| DEGVGEGL | 49 | 4864 | 481 | 938 | 34 | 14244 |
| AEQRASPL | 16 | 73 | 13 | 211 | 0.38 | 120 |
| KEILDEAY | 82 | 921 | 430 | 1081 | 74 | 2646 |
| EEAPRSPL | 1191 | 3489 | 1611 | 1593 | 171 | 1926 |
| SEDPTVPL | 103 | 71 | 161 | 12267 | 2.0 | 308 |
| MELAALCRW | 7.0 | 4833 | 138 | 16 | 9.9 | 1183 |
| QEVQGYVLI | 77 | 206 | 39 | 30 | 0.50 | 96 |
| FEDNYALAV | 12 | 34 | 5.1 | 13470 | 0.17 | 131 |
| RELQLRSLT | 638 | 316 | 13 | 465 | 0.20 | 162 |
| TEILKGGVL | 125 | 30 | 14 | 1377 | 0.28 | 2480 |
| HEQCAAGCT | 1995 | 42164 | 7377 | 19048 | 178 | 2974 |
| CELHCPALV | 136 | 4805 | 319 | 2308 | 52 | 1110 |
| FESMPNPEG | 6068 | 30237 | 59 | 16458 | 14 | 155 |
| QEVTAEDGT | 5207 | 31081 | 3122 | 7886 | 66 | 1843 |
| CEKCSKPCA | 3740 | 27386 | 2703 19957 | 342 | 8007 | |
| MEHLREVRA | 233 | 44754 | 386 | 38 | 3.2 | 19 |
| REVRAVTSA | 626 | 427 | 0.71 | 3160 | 0.18 | 9.3 |
| QEFAGCKKI | 1120 | 736 | 131 | 81 | 44 | 2684 |
| EEITGYLYI | 86 | 906 | 916 | 12 | 121 | 94 |
| RELGSGLAL | 359 | 3.7 | 0.85 | 457 | 0.97 | 2262 |
| GEGLACHQL | 13766 | 187 | 88 | 112 | 11 | 340 |
| QECVEECRV | 15799 | 8755 | 1664 | 7150 | 210 | 4542 |
| VEECRVLQG | 1528 | 8947 | 7622 | 14202 | 305 | 20142 |
| EEGRVLQGL | 890 | 7076 | 2029 | 717 | 434 | 1185 |
| AEQRASPLT | 346 | 874 | 183 | 103 | 1.8 | 10 |
| QETELVEPL | 12 | 62 | 85 | 681 | 3.5 | 1232 |
| VEPLTPSGA | 7321 | >9638.55 | 11 | 8516 | 191 | 17037 |
| TELRKVKVL | 1514 | 4698 | 54 | 2128 | 2.5 | 14147 |
| GENVKIPVA | 10755 | 14510 | 7.5 | 20309 | 2.7 | 7.0 |
| KEILDEAYV | 1358 | 62 | 146 | 6466 | 8.4 | 42 |
| DEAYVMAGV | 58 | 5327 | 1245 | 8006 | 138 | 161 |
| DETEYHADG | 159 | >11940.3 | >65384.62 | >24403.18 | 1397 | 13353 |
| LESILRRRF | 29 | >11940.3 | 3475 | 4.7 | 101 | 12918 |
| GBRLPQPPI | 62 | 71 | 15 | 63 | 1.1 | 15 |
| LEDDDMGDL | 191 | 556 | 351 | 947 | 900 | 6251 |
| EEYLVPQQG | 66 | 10344 | 136 | 651 | 126 | 131 |
| EEEAPRSPL | 902 | 4490 | 2881 | 342 | 362 | 307 |
| EBAPRSPLA | 486 | 10707 | 4900 | 180 | 294 | 4.5 |
| SEGAGSDVF | 74 | 5627 | 6525 | 69 | 192 | 6960 |
| PEYVNQPDV | 831 | 3437 | 1581 | 1109 | 48 | 2536 |
| PEYLTPQGG | 1456 | 18951 | 13860 | 6532 | 284 | 18990 |
| PERGAPPST | 385 | 4744 | 7679 | 1116 | 178 | 7767 |
| AENPEYLGL | 17 | 81 | 271 | 44 | 2.5 | 155 |
| MELAALCRWG | 102 | 8684 | 1840 | 5.7 | 135 | 408 |
| LELTYLPTNA | 332 | 325 | 10.4 | 6428 | 3.1 | 24 |
| QEVQGYVL1A | 61 | 772 | 64 | 1871 | 15 | 11 |
| FEDNYALAVL | 321 | 6.2 | 48 | 2844 | 3.8 | 3095 |
| TEILKGGVLI | 1021 | 241 | 294 | 24 | 21 | 7600 |
| GESSEDCQSL | 138636 | 8.1 | 23 | 427 | 5.1 | 2491 |
| SEDGQSLTRT | 335 | 8550 | 11529 | 518 | 2857 | 4726 |
| GELHCPALVT | 80 | >9248.55 | 65 | 933 | 18 | 477 |
| MEHLREVRAV | 72 | 20684 | 160 | 180 | 13 | 140 |
| QEFAGCKKIF | 53 | 3686 | 12 | 4.0 | 3.6 | 115 |
| FETLEEITGY | 671 | 53363 | 36302 | 262 | 1679 | >28488.37 |
| LEEITGYLYI | 143 | 914 | 2996 | 222 | 143 | 1488 |
| RELGSGLALI | 4810 | 22 | 4.4 | 32 | 0.78 | 173 |
| PEDECVGEQL | 1257 | 278 | 257 | 6331 | 49 | 24019 |
| QECVEECRVL | 315 | 444 | 399 | 606 | 22 | 2863 |
| VEEGRVLQGL | 270 | 227 | 5815 | 237 | 189 | 16094 |
| REYVNARHCL | 1327 | 39 | 4.8 | 106 | 0.97 | 126 |
| PECQPQNGSV | 7962 | 35957 | 20374 | 12964 | 472 | >28488.37 |
| EEGACQPCPI | 119 | 40113 | 340 | 52 | 80 | 401 |
| QETELVEPLT | 15 | 293 | 338 | 1619 | 13 | 288 |
| VEPLTPSGAM | 4649 | 1667 | 584 | 4368 | 108 | 20167 |
| KETELRKVKV | 11925 | 26700 | 68 | 2936 | 4.5 | 1603 |
| TELRKVKVLG | 721 | 20312 | 601 | 3650 | 14 | 12816 |
| GENVKIPVAI | 563 | 314 | 28 | 230 | 6.7 | 198 |
| KEILDEAYVM | 0.14 | 10 | 153 | 35 | 7.5 | 234 |
| DEAYVMAGVG | 122 | 203 | 154 | 4033 | 4102 | 218 |
| DETEYHADGG | 613 | 45291 | 16801 | 3891 | 269 | 29025 |
| TEYHADGGKV | 239 | 5246 | 2003 | 2911 | 15 | 1571 |
| LESILRRRFT | 82 | 28476 | 1189 | 34 | 87 | 2251 |
| REIPDLLEKG | 649 | 4493 | 814 | 1270 | 13 | 1977 |
| SEGRPRFREL | 80 | 307 | 18 | 11 | 0.20 | 25 |
| RELVSEFSRM | 9.1 | 28 | 4.3 | 33 | 0.12 | 1726 |
| NEDLGPASPL | 107 | 281 | 150 | 40 | 6.0 | 231 |
| AEEYLVPQQG | 723 | 66699 | 24424 | 417 | 479 | 127 |
| EEYLVPQQGF | 2.1 | 26569 | 2551 | 6.9 | 11 | 73 |
| SEEEAPRSPL | 151 | 155 | 217 | 37 | 8.4 | 84 |
| EEEAPRSPLA | 6611 | 49549 | 38943 | 425 | 960 | 14 |
| SETDGYVAPL | 94 | 214 | 184 | 386 | 2.4 | 302 |
| PERGAPPSTF | 1062 | 14884 | 3437 | 6871 | 208 | 15700 |
| PEYLGLDVPV | 613 | 352 | 35 | 1371 | 1.7 | 610 |
| MELAALCRWGL | 6.4 | 24 | 30 | 17 | 0.92 | 116 |
| PETHLDMLRHL | 1322 | 700 | 2971 | 11534 | 70 | 4329 |
| RELQLRSLTEI | 261 | 2.8 | 3.7 | 125 | 0.99 | 269 |
| GESSEDCQSLT | 742 | 48 | 180 | 14386 | 40 | 2158 |
| SEDCQSLTRTV | 101 | 4322 | 311 | 943 | 21 | 10 |
| CELHCPALVTY | 12 | 3469 | 3198 | 140 | 89 | 2779 |
| FESMPNPEGRY | 74 | 3666 | 3533 | 59 | 70 | 1394 |
| CEKCSKPCARV | 1167 | 4103 | 2079 | 9594 | 101 | 1561 |
| MEHLREVRAVT | 1064 | 3614 | 2207 | 795 | 111 | 74 |
| REVRAVTSANI | 4491 | 17 | 30 | 1680 | 1.8 | 421 |
| QEFAGCKKIFG | 211 | 314 | 477 | 37 | 2.1 | 138 |
| FETLEEITGYL | 133 | 78 | 649 | 7490 | 42 | 2200 |
| EEITGYLYISA | 0.94 | 1440 | 52 | 4.5 | 2.1 | 0.9 |
| GBGLACHQLCA | 62 | 39 | 97 | 159 | 2.7 | 196 |
| DEEGACQPCPI | 451 | 5517 | 7293 | 968 | 438 | 1323 |
| AEQRASPLTSI | 467 | 19 | 58 | 5.1 | 2.5 | 11 |
| TELVEPLTPSG | 601 | 2978 | 3703 | >21052.63 | 269 | 14079 |
| KETELRKVKVL | 9529 | 2973 | 1868 | 7136 | 71 | 12237 |
| KEILDEAYVMA | 731 | 252 | 95 | 11514 | 64 | 123 |
| LEDVRLVHRDL | 729 | 325 | 641 | 818 | 59 | 2382 |
| WELMTFGAKPY | 13 | 509 | 778 | 24 | 75 | 1216 |
| GERLPQPPICT | 12486 | 24270 | 23 | 9094 | 3.9 | 15 |
| SECRPRFRBLV | 1996 | 3673 | 121 | 927 | 18 | 118 |
| RELVSEFSRMA | 168 | 389 | 143 | 2613 | 3.5 | 32 |
| AEEYLVPQQGF | 125 | 584 | 1831 | 21 | 99 | 268 |
| EEYLVPQQGFF | 94 | 4291 | 1695 | 78 | 168 | 154 |
| SEEEAPRSPLA | 1318 | 3604 | 5110 | 8550 | 158 | 27 |
| SEGAGSDVFDG | 928 | 3751 | 5695 | 374 | 286 | 3008 |
| SETDGYVAPLT | 66 | 125 | 224 | 1225 | 2.2 | 45 |
| REGPLPAARIPA | 157 | 543 | 78 | 32906 | 4.2 | 347 |
| VENPEYLTPQG | 8386 | 56393 | 42593 | 17337 | 11 | 4188 |
| PEYLTPQGGAA | 1724 | 41026 | 200 | >17829.46 | 354 | 1382 |
| AENPEYLGLDV | 11934 | 28 | 139 | 69 | 3.0 | 24 |
| LELTYLPTNASL | 12 | 25 | 102 | 386 | 6.8 | 11 |
| RELQLRSLTEIL | 5954 | 151 | 600 | 3778 | 1.1 | 1371 |
| PEGRYTFGASCV | 4071 | 2.9 4.4 | 778 | 116 | | |
| LEEITGYLYISA | 209 | 28 | 31 | 263 | 18 | 694 |
| EEITGYLYISAW | 746 | 478 | 1800 | 252 | 1492 | |
| PEADQCVACAHY | 901 | 4050 | 5127 | 213 | 463 | |
| TELVEPLTPSGA | 236 | 2059 | 59 | 2132 | 206 | |
| TEYHADGGKVPI | 680 | 22 | 4.4 | 2177 | 61 | |
| GERLPQPPICTI | 17769 | 162 | 3.9 | 292 | 2.5 | |
| AEEYLVPQQGFF | 144 | 228 | 45 | 16 | 13 | |
| PEGRYTFGASCVT | 5228 | 3793 | 737 | 1419 | 267 | 673 |
| CEKCSKPCARVCY | 701 | >53333.33 | 406 | 302 | 44 | 1315 |
| MEHLREVRAVTSA | 70 | 669 | 72 | 144 | 18 | 12 |
| DECVGEGLACHQL | 464 | 2635 | 3668 | 2544 | 212 | 2063 |
| PECQPQNGSVTGF | 6293 | 381 | 5338 | 3564 | 375 | >22374.43 |
| RENTSPKANKEIL | 7750 | 3.7 | 77 | >2540.03 | 3.9 | 1510 |
| REIPDLLEKGERL | 7636 | 40 | 136 | 3050 | 16 | 2710 |
| SEFSRMARDPQRF | 61 | 350 | 57 | 23 | 12 | 247 |
| SEGAGSDVFDGDL | 5172 | 45 | 2059 | 1303 | 711 | 2458 |
| GEFGGYGSV | 307 | 112 | 6.4 | 2335 | 534 | 40 |
| LWQLNGRLEYTLKDR | | | | | 0.11 | |
| SEFQAAI | 181 | 6830 | 779 | 2660 | 33 | 9597 |
| SEYLQLV | 1375 | 7777 | 658 | 733 | 21 | 930 |
| WEELSML | 1288 | 781 | 740 | >28482.97 | 151 | 82009 |
| GEPHISY | 8833 | 12272 | 6716 | 36116 | 272 | >33333.33 |
| LEARGEAL | 163 | 99 | 65 | 29495 | 2.9 | 31463 |
| QEEEGPRM | 298 | 11598 | 1608 | 19255 | 118 | 6730 |
| EEEGPRMF | 723 | 12281 | 32093 | 2406 | 213 | 943 |
| VELVHFLL | 5.0 | 69 | 31 | 3322 | 1.2 | 2427 |
| AEMLESVL | 968 | 14 | 31 | 327 | 0.88 | 302 |
| SEYLQLVF | 0.97 | 765 | 6.0 | 284 | 0.70 | 122 |
| EEKIWEEL | 753 | 9084 | 2599 | 98976 | 104 | 171 |
| LEARGEALG | 155 | 1161 | 3006 | 11018 | 24 | 2688 |
| GEALOLYGA | 9529 | 2832 | 34 | 6134 | 2.2 | 17 |
| QEEEGPRMF | 414 | 918 | 7747 | 237 | 409 | 2171 |
| VELVHFLLL | 71 | 79 | 31 | 579 | 3.1 | 1129 |
| REPVTKAEM | 60 | 373 | 284 | 896 | 4.5 | 832 |
| SEYLQLVFG | 18 | 8890 | 421 | 271 | 19 | 113 |
| PEEKIWEEL | 577 | 19449 | 3908 | 1029 | 235 | 17345 |
| EELSMLEVF | 1.4 | 16436 | 252 | 22 | 2.8 | 1013 |
| FEGREDSVF | 9.8 | 2366 | 348 | 221 | 13 | 3339 |
| YEFLWGPRA | 5.3 | 249 | 5.2 | 2355 | 1.1 | 241 |
| EEGLEARGEA | 1077 | 3434 | 3227 | 216 | 302 | 30 |
| LEARGEALGL | 81 | 184 | 277 | 2275 | 4.1 | 964 |
| VEVTLGEVPA | 14 | 371 | 31 | 3801 | 0.52 | 15 |
| EEGPRMFPDL | 128 | 4438 | 486 | 95 | 13 | 42 |
| REPVTKAEML | 88 | 23 | 264 | 84 | 41 | 917 |
| SEYLQLVFGI | 2.2 | 20 | 6.1 | 3.7 | 0.84 | 4.4 |
| VEVVPISHLY | 20 | 11522 | 4385 | 13 | 1225 | 4885 |
| EEKIWEELSM | 17 | 21450 | 477 | 46 | 19 | 107 |
| WEELSMLEVF | 0.14 | 463 | 30 | 15 | 15 | 290 |
| FEGRLEDSVFA | 178 | >10062.89 | 4775 | 6879 | 192 | 503 |
| QENYLEYRQV | 118 | 493 | 102 | 17 | 16 | 27 |
| YEFLWGPRAL | | 8.5 | 0.97 | 130 | 0.72 | 753 |
| GEPHISYPPL | 2612 | 7.0 | 2.9 | 1200 | 0.71 | 380 |
| EEGLEARGEAL | 179 | 300 | 578 | 2630 | 19 | 1812 |
| LEARGEALGLV | 158 | 198 | 345 | >17829.46 | 13 | 1912 |
| GEALGLVGAQA | 877 | 4293 | 52 | 3575 | 1.4 | 28 |
| EEQQTASSSST | 752 | 4040 | 41162 | 5910 | 1552 | 134 |
| VEVTLGEVPAA | 124 | 25216 | 919 | >23469.39 | 44 | 1583 |
| EEEGPRMFPDL | 1011 | 2646 | 3470 | 3273 | 131 | 209 |
| SEFQAAISRKM | 7.0 | 345 | 107 | 88 | 1.2 | 161 |
| VELVHFLLLKY | 52 | 550 | 294 | 1551 | 49 | 1790 |
| LESVLRNCQDF | 64 | 5409 | 3458 | 209 | 76 | 15241 |
| VEVVPISHLYI | 97 | 135 | 146 | 335 | 7.2 | 3788 |
| IEGDCAPEEKI | 844 | 27827 | 32058 | 2627 | 486 | 183 |
| EEKIWEELSML | 1641 | 4978 | 20625 | 1862 | 375 | 181 |
| EELSMLEVFEG | 1.5 | 24061 | 294 | 4.6 | 23 | 163 |
| LEVFEGRBDSV | 639 | 2624 | 367 >21296.3 | 46 | 29449 | |
| YEFLWGPRALI | 5.2 | 4.1 | 2.8 | 92 | 0.59 | 450 |
| EEQQTASSSSTL | 7259 | 166 | 526 | 57 | 981 | 15 |
| QEEEGPRMFPDL | 3595 | 394 | 1330 | 1643 | | 120 |
| SEFQAAISRKMV | 43 | 161 | 29 | 25 | | 21 |
| LESVLRNCQDFF | 56 | 55 | 356 | 184 | 24 | 1993 |
| VEVVPISHLYIL | 266 | 3.4 | 16 | 486 | 4.0 | 1182 |
| EEGLEARGEALGL | 10416 | 1769 | 5143 | 196 | 118 | 1673 |
| LEARGEALGLVGA | 347 | 20 | 48 | 2575 | 2.2 | 116 |
| LESEFQAAISRKM | 49 | 310 | 72 | 242 | 14 | 22 |
| REPVTKAEMLESV | 5531 | 337 | 411 | 4546 | 21 | 1507 |
| SEYLQLVFGIEVV | 9.7 | 23 | 4.5 | 144 | 5.4 | 6.6 |
| IEVVEVVPTSHLY | 79 | 162 | 245 | 52 | 125 | 106 |
| VEVVPISHLYILV | 92 | 93 | 47 | 270 | 51 | 112 |
| MEVDPIGHLY | 13 | 209 | 334 | 13 | 28 | 228 |
| EEEGPSTF | 216 | 1008 | 435 | 3933 | 27 | 1819 |
| AELVHFLL | 120 | 71 | 6.8 | 1074 | 0.16 | 452 |
| FEGREDSI | 927 | 718 | 127 | 7708 | 13 | 2291 |
| QEAASSSST | 1422 | 23469 | 1480 | 9593 | 41 | 110 |
| AELVHFLLL | 160 | 25 | 3.1 | 33 | 0.94 | 141 |
| AEMLGSVVG | 96 | 1899 | 109 | 27 | 1.6 | 11 |
| EELSVLEVF | 7.3 | 10215 | 3314 | 61 | 12 | 2120 |
| FEGREDSIL | 1091 | 51 | 439 | 1925 | 11 | >27071.82 |
| QEAASSSSTL | 171 | 49 | 47 | 56 | 13 | 287 |
| EEGPSTFPDL | 158 | 655 | 591 | 198 | 127 | 128 |
| IELMEVDPIG | 194 | 6592 | 5325 | 222 | >16306.95 | 7604 |
| MEVDPIGHLY | 15 | 617 | 625 | 11 | 99 | 169 |
| EEKIWEELSV | 73 | 8947 | 79 | 396 | 17 | 17 |
| WEELSVLEVF | 1.7 | 75 | 37 | 14 | 13 | 1701 |
| FEGREDSILG | 229 | 940 | 4361 | 8534 | 172 | 20261 |
| EEEGPSTFPDL | 935 | 431 | 2120 | 2685 | 102 | 158 |
| AELVHFLLLKY | 153 | 32 | 39 | 178 | 1.6 | 670 |
| MEVDPIGHLYI | 9.8 | 34 | 16 | 64 | 0.91 | 95 |
| REGDCAPEEKI | 973 | 2418 | 830 | 4038 | 42 | 146 |
| EEKIWEELSVL | 133 | 152 | 1255 | 1416 | 58 | 218 |
| LEVFEGREDSI | 4745 | 206 | 512 | 20963 | 69 | >31012.66 |
| RERFEME | 180 | 4079 | 1907 | 25488 | 108 | 20048 |
| LEDSSGNL | 17736 | 782 | 362 | 42791 | 211 | 15946 |
| GEYFTLQI | 7774 | 112 | 60 | 3511 | 1.0 | 261 |
| VEPPLSQET | 8302 | 17052 20508 | 3186 | 236 | 29270 | |
| PENNVLSPL | 1150 | 1261 | 718 | 11174 | 8.8 | >27071.82 |
| DEAPRMPEA | 84 | 9092 | 4577 | 6448 | 98 | 10.0 |
| HERCSDSDG | 1118 | 2367 38636 | 19328 | 208 | 13390 | |
| VEGNLRVEY | 832 | 12752 67730 | 142 | 2583 | 39059 | |
| VEYLDDRNT | 1442 | 36833 35854 | 10071 | 157 | 13503 | |
| LEDSSGNLL | 1140 | 43 | 2771 | 4656 | 43 | 26134 |
| RELNEALEL | 3000 | 15 | 30 | 525 | 1.1 | 3337 |
| NEALELKDA | 1925 | 3887 27585 | 4270 | 1582 | 129 | |
| LELKDAQAG | 451 | 18706 | 3659 | 17293 | 30 | 1989 |
| MEEPQSDPSV | 12157 | 3802 | 16536 | 1927 | 816 | 175 |
| VEPPLSQETF | 814 | >37209.3 | 21732 | 406 | 525 | >24019.61 |
| QETFSDLWKL | 736 | 199 | 255 | 39 | 14 | 901 |
| IEQWFTEDPG | 151 | 1250 | 2114 | 5595 | 142 | 197 |
| DEAPRMPEAA | 121 | 3941 | 8444 | 2594 | 1037 | 100 |
| HERCSDSDGL | 139 | 171 | 61 | 1468 | 6.0 | 1723 |
| VEGNLRVEYL | 104 | 481 | 2565 | 1963 | 22 | 15189 |
| VEYLDDRNTF | 0.94 | 501 | 37 | 32 | 1.4 | 3601 |
| PEVGSDCTTI | 611 | 4552 | 248 | 2293 | 2046 | 22487 |
| LEDSSGNLLG | 103 | 531 | 697 | 7905 | 153 | 19256 |
| FEVRVCACPG | 64 | 2043 | 4.9 | 180 | 0.76 | 1872 |
| TEEENLRKKG | 74966 | >37209.3 | 11858 | >23589.74 | 315 | 30635 |
| GEPHHELPPG | 108 | 3323 | 1888 | 11728 | 4.4 | 20 |
| GEYFTLQIRG | 108 | 88 | 19 | 2452 | 3.9 | 157 |
| RERFEMFREL | 83 | 29 | 17 | 17 | 0.34 | 422 |
| FEMFRELNEA | 127 | 3207 | 223 | 952 | 2.0 | 208 |
| QETFSDLWKLL | 4158 | 3366 | 740 | 631 | 168 | 1218 |
| HERCSDSDGLA | 1408 | 4879 | 1915 | >20956.72 | 96 | 186 |
| YEPPEVGSDCT | 16872 | 4529 | 125 | 13349 | 12712 | 16034 |
| HELPPGSTKRA | 6034 | 3974 | 3255 | 47077 | 189 | 1472 |
| FEMFRELNEAL | 475 | 17 | 8.8 | 748 | 1.1 | 1352 |
| NEALELKDAQA | 742 | 6235 | 5071 | >20956.72 | 949 | 53 |
| TEDPGPDEAPRM | 888 | 327 | 893 | 2053 | 161 | 1676 |
| GEPHHELPPGST | 6822 | 24342 | 4631 | 6581 | 252 | 169 |
| DEAPRMPEAAPPV | 427 | >48484.85 | 7258 | >2762.76 | 1376 | 19 |
| YEPPEVGSDCTTI | 8796 | 2699 | 1540 | >2740.54 | 253 | >20000 |
| RERPDNYV | >73809.52 | 71554 | 62 | >67647.06 | >34517.77 | 34648 |
| SEIDLILGY | 3.0 | 285 | 140 | 4.8 | 8.5 | 397 |
| AEIPTRVNY | 1691 | 7826 | 5443 | 333 | 23 | 1286 |
| AEMGKFKFSY | 1517 | 2941 | 622 | 146 | 28 | 283 |
| DEIGVIDLY | 11 | >114285.71 | >77272.73 | 707 | 212 | >49000 |
| AEMGKFKYSF | 155 | 113 | 3.8 | 18 | 31 | 186 |
| SEAIHTFQY | 25 | 2895 | 1802 | 18 | 16 | 1078 |
| SEAIYTFQF | 5.7 | 967 | 39 | 4.8 | 20 | 293 |
| AEGIVTGQY | 7176 | 6462 | 1528 | 255 | 12 | 418 |
| HETTYNSI | 1644 | 251 | 336 | 616 | 23959 | 6608 |
| GELSYLNV | >24800 | 4856 | 100 | 19013 | 23735 | 784 |
| YEDTGKTI | 13997 | 794 | 83 | 7911 | 2177 | 49000 |
| YENDIEKKI | 30992 | 1156 | 145 | 1725 | 371 | |

TABLE 23

HLA-DQ SUPERTYPES

| | | | | | | |
|---|---|---|---|---|---|---|
| | SEQ | | | | | |
| | ID | | | | | |
| Sequence | NO. | AA | Organism | Protein | Position | Analog |

| | | | | | |
|---|---|---|---|---|---|
| AAAKAAAAAAYAA | 13 | Artificial | | | A |
| | | sequence | | | |
| (44)YAAAAAAKAAA | 13 | Artificial | | | A |
| | | sequence | | | |
| AAFAAAKTAAAFA | 13 | Artificial | | | A |
| | | sequence | | | |
| YAAFAAAKTAAAFA | 14 | Artificial | | | A |
| | | sequence | | | |
| YAAFAAAKTAAAFA | 14 | Artificial | | | |
| | | sequence | | | |
| AHAAHAAHAAHAAHAA | 16 | HA | | | A |
| VLERYLLEAKEAENI | 15 | Human | EPO | 11 | |
| VPDTKVNFYAWKRMB | 15 | Human | EPO | 41 | |
| WKRMEVGQQAVEVWQ | 15 | Human | EPO | 51 | |
| VGQQAVEVWQGLALL | 15 | Human | EPO | 56 | |
| VEVWQGLALLSEAVL | 15 | Human | EPO | 61 | |
| GLALLSEAVLRGQAL | 15 | Human | EPO | 66 | |
| SEAVLRGQALLVNSS | 15 | Human | EPO | 71 | |
| RGQALLVNSSQPWEP | 15 | Human | EPO | 76 | |
| LQLHVDKAVSGLRSL | 15 | Human | EPO | 91 | |
| KEAISPPDAASAAPL | 15 | Human | EPO | 116 | |
| PPDAASAAPLRTITA | 15 | Human | EPO | 121 | |
| SAAPLRTITADTFRK | 15 | Human | EPO | 126 | |
| EAENITTGTAEHTSL | 15 | Human | EPO | 21 | A |
| RLFDNASLRAHRLHQ | 15 | Human | Growth | 8 | |
| | | | hormone | | |
| QLAFDTYQEFEEAYI | 15 | Human | Growth | 22 | |
| | | | hormone | | |
| ISLLLIQSWLEPVQF | 15 | Human | Growth | 78 | |
| | | | hormone | | |
| NSLVYGASDSNVYDL | 15 | Human | Growth | 99 | |
| | | | hormone | | |
| SDSNVYDLLKDLEEG | 15 | Human | Growth | 106 | |
| | | | hormone | | |
| KIFGSLAFLPESFDGDPA | 18 | Human | Her2/neu | 369 | |
| CLKDRRNFDIPEEIK | 15 | Human | IFN-B | 31 | |
| QLQQFQKEDAAVTIY | 15 | Human | IFN-B | 46 | |
| QKEDAAVTIYEMLQN | 15 | Human | IFN-B | 51 | |
| STGWNETIVENILLAN | 15 | Human | IFN-B | 76 | |
| ETIVENLLANVYHQR | 15 | Human | IFN-B | 81 | |
| KEDSHCAWTIVRVEI | 15 | Human | IFN-B | 136 | |
| MSYNLLGFLQRSSNT | 15 | Human | IFN-B | 1 | A |
| QHLCGSHLVEALYLV | 15 | Human | Insulin | 4 | |
| | | | beta | | |
| | | | chain | | |
| GSHLVEALYLVGGER | 15 | Human | Insulin | 8 | |
| | | | beta | | |
| | | | chain | | |
| GSDLVEALYLVCGER | 15 | Human | Insulin | 8 | A |
| | | | beta | | |
| | | | chain | | |
| VEALYLVGGERGFLY | 15 | Human | Insulin | 12 | A |
| | | | beta | | |
| | | | chain | | |
| VEALYLVTGERGFFY | 15 | Human | Insulin | 12 | A |
| | | | beta | | |
| | | | chain | | |
| IDVWLGGLAENFLPY | 15 | Human | thyroid | 632 | |
| | | | perox | | |
| IDVWLGGLAYNFLPY | 15 | Human | thyroid | 632 | A |
| | | | perox | | |
| IDVWLGGLALNFLPY | 15 | Human | thyroid | 632 | A |
| | | | perox | | |
| IDVWLGGLASNFLPY | 15 | Human | thyroid | 632 | A |
| | | | perox | | |
| IDVWLGGLAKNFLPY | 15 | Human | thyroid | 632 | A |
| | | | perox | | |
| IDVWLGGLADNFLPY | 15 | Human | thyroid | 632 | A |
| | | | perox | | |
| IDVYLGGLAENFLPY | 15 | Human | thyroid | 632 | A |
| | | | perox | | |
| IDVLLGGLAENFLPY | 15 | Human | thyroid | 632 | A |
| | | | perox | | |
| IDVSLGGLAENFLPY | 15 | Human | thyroid | 632 | A |
| | | | perox | | |
| IDVKLGGLAENFLPY | 15 | Human | thyroid | 632 | A |
| | | | perox | | |
| IDVDLGGLAENFLPY | 15 | Human | thyroid | 632 | A |
| | | | perox | | |
| IDVWLGGLAENYLPY | 15 | Human | thyroid | 632 | A |
| | | | perox | | |
| IDVWLGGLAENVLPY | 15 | Human | thyroid | 632 | A |
| | | | perox | | |
| IDVWLGGLAENSLPY | 15 | Human | thyroid | 632 | A |
| | | | perox | | |
| IDVWLGGLAENKLPY | 15 | Human | thyroid | 632 | A |
| | | | perox | | |
| IDVWLGGLAENDLPY | 15 | Human | thyroid | 632 | A |
| | | | perox | | |
| IYVWLGGLABNFLPY | 15 | Human | thyroid | 632 | A |
| | | | perox | | |
| ILVWLGGLAENFLPY | 15 | Human | thyroid | 632 | A |
| | | | perox | | |
| ISVWLGGLAENFLPY | 15 | Human | thyroid | 632 | A |
| | | | perox | | |
| IKVWLGGLAENFLPY | 15 | Human | thyroid | 632 | A |
| | | | perox | | |
| IEVWLGGLAENFLPY | 15 | Human | thyroid | 632 | A |
| | | | perox | | |
| IDVWLGGLABNFLPE | 15 | Human | thyroid | 632 | A |
| | | | perox | | |
| IDVWLGGLAENFLPL | 15 | Human | thyroid | 632 | A |
| | | | perox | | |
| IDVWLGGLAENFLPS | 15 | Human | thyroid | 632 | A |
| | | | perox | | |
| IDVWVLGGLAENFLPK | 15 | Human | thyroid | 632 | A |
| | | | perox | | |
| TDVWLGGLAENFLPD | 15 | Human | thyroid | 632 | A |
| | | | perox | | |
| IDVWLGGLAENFYPY | 15 | Human | thyroid | 632 | A |
| | | | perox | | |
| IDVWLGGLAENFVPY | 15 | Human | thyroid | 632 | A |
| | | | perox | | |
| IDVWLGGLAENFSPY | 15 | Human | thyroid | 632 | A |
| | | | perox | | |
| IDVWLGGLAENFKPY | 15 | Human | thyroid | 632 | A |
| | | | perox | | |
| IDVWLGGLAENFDPY | 15 | Human | thyroid | 632 | A |
| | | | perox | | |
| IDVWLGGLAEYFLPY | 15 | Human | thyroid | 632 | A |
| | | | perox | | |
| IDVWLGGLAELFLPY | 15 | Human | thyroid | 632 | A |
| | | | perox | | |
| 1DVWLGGLAESFLPY | 15 | Human | thyroid | 632 | A |
| | | | perox | | |
| IDVWLGGLAEKFLPY | 15 | Human | thyroid | 632 | A |
| | | | perox | | |
| IDVWLGGLAEDFLPY | 15 | Human | thyroid | 632 | A |
| | | | perox | | |
| IDVWLGGLAEQFLPY | 15 | Human | thyroid | 632 | A |
| | | | perox | | |
| IDVWLGGLYENFLPY | 15 | Human | thyroid | 632 | A |
| | | | perox | | |
| IDVWLGGLLENFLPY | 15 | Human | thyroid | 632 | A |
| | | | perox | | |
| IDVWLGGLSENFLPY | 15 | Human | thyroid | 632 | A |
| | | | perox | | |
| IDVWLGGLKENFLPY | 15 | Human | thyroid | 632 | A |
| | | | perox | | |
| IDVWLGGLDENFLPY | 15 | Human | thyroid | 632 | A |
| | | | perox | | |
| IDVWLGGYAENFLPY | 15 | Human | thyroid | 632 | A |
| | | | perox | | |
| IDVWLGGVAENFLPY | 15 | Human | thyroid | 632 | A |
| | | | perox | | |
| IDVWLGGSABNFLPY | 15 | Human | thyroid | 632 | A |
| | | | perox | | |
| IDVWLGGKAENFLPY | 15 | Human | thyroid | 632 | A |
| | | | perox | | |
| IDVWLGGDAENFLPY | 15 | Human | thyroid | 632 | A |
| | | | perox | | |
| IDVWLGYLAENFLPY | 15 | Human | thyroid | 632 | A |
| | | | perox | | |
| IDVWLGLLAENFLPY | 15 | Human | thyroid | 632 | A |
| | | | perox | | |
| IDVWLGSLAENFLPY | 15 | Human | thyroid | 632 | A |
| | | | perox | | |
| IDVWLGKLAENFLPY | 15 | Human | thyroid | 632 | A |
| | | | perox | | |
| IDVWLGDLABNFLPY | 15 | Human | thyroid | 632 | A |
| | | | perox | | |
| IDVWLYGLAENFLPY | 15 | Human | thyroid | 632 | A |
| | | | perox | | |
| IDVWLLGLAENFLPY | 15 | Human | thyroid | 632 | A |
| | | | perox | | |
| IDVWLSGLAENFLPY | 15 | Human | thyroid | 632 | A |
| | | | perox | | |
| TDVWLKGLAENFLPY | 15 | Human | thyroid | 632 | A |
| | | | perox | | |
| TDVWLDGLAENFLPY | 15 | Human | thyroid | 632 | A |
| | | | perox | | |
| IDVWYGGLAENFLPY | 15 | Human | thyroid | 632 | A |
| | | | perox | | |
| IDVWVGGLAENFLPY | 15 | Human | thyroid | 632 | A |
| | | | perox | | |
| IDVWSGGLAENFLPY | 15 | Human | thyroid | 632 | A |
| | | | perox | | |
| IDVWKGGLAENFLPY | 15 | Human | thyroid | 632 | A |
| | | | perox | | |
| IDVWDGGLAENFLPY | 15 | Human | thyroid | 632 | A |
| | | | perox | | |
| IDYWLGGLAENFLPY | 15 | Human | thyroid | 632 | A |
| | | | perox | | |
| IDLWLGGLAENFLPY | 15 | Human | thyroid | 632 | A |
| | | | perox | | |
| IDSWLGGLAENFLPY | 15 | Human | thyroid | 632 | A |
| | | | perox | | |
| IDKWLGGLABNFLPY | 15 | Human | thyroid | 632 | A |
| | | | perox | | |
| IDDWLGGLAENFLPY | 15 | Human | thyroid | 632 | A |
| | | | perox | | |
| IDVWLGGLABNFLYY | 15 | Human | thyroid | 632 | A |
| | | | perox | | |
| IDVWLGGLAENFLLY | 15 | Human | thyroid | 632 | A |
| | | | perox | | |
| IDVWLGGLAENFLSY | 15 | Human | thyroid | 632 | A |
| | | | perox | | |
| IDVWLGGLAENFLKY | 15 | Human | thyroid | 632 | A |
| | | | perox | | |
| IDVWLGGLAENFLDY | 15 | Human | thyroid | 632 | A |
| | | | perox | | |
| YDVWLGGLAENFLPY | 15 | Human | thyroid | 632 | A |
| | | | perox | | |
| LDVWLGGLAENFLPY | 15 | Human | thyroid | 632 | A |
| | | | perox | | |
| SDVWLGGLAENFLPY | 15 | Human | thyroid | 632 | A |
| | | | perox | | |
| KDVWLGGLAENFLPY | 15 | Human | thyroid | 632 | A |
| | | | perox | | |
| DDVWLGGLAENFLPY | 15 | Human | thyroid | 632 | A |
| | | | perox | | |

TABLE 24

HLA-DQ SUPERTYPES

| | | | | |
|---|---|---|---|---|
| | SEQ | | | |
| | ID | DQB1*030 | DQB1*030 | DQB1*020 |
| Sequence | NO. | 1 | 2 | 1 |

| | | | |
|---|---|---|---|
| AAAKAAAAAAYAA | 424 | | |
| (44)YAAAAAAKAAA | 26 | | |
| AAFAAAKTAAAFA | 49 | | |
| YAAFAAAKTAAAFA | 36 | | |
| YAAFAAAKTAAAFA | 39 | | |
| AHAAHAAHAAHAAHAA | 58 | | |
| VLERYLLEAKRAENI | 10932 | 309 | 5389 |
| VPDTKVNFYAWKRME | 730 | >46666.67 | >147058.82 |
| WKRMEVGQQAVEVWQ | 13666 | 12146 | 159 |
| VGQQAVEVWQGLALL | 1807 | 4407 | 838 |
| VEVWQGLALLSEAVL | 19 | 14 | 98 |
| GLALLSEAVLRGQAL | 107 | 16963 | 6742 |
| SEAVLRGQALLVNSS | 55 | 36395 | 9755 |
| RGQALLVNSSQPWEP | 302 | 14393 | 13362 |
| LQLHVDKAVSGLRSL | 88 | 7842 | 7590 |
| KEAISPPDAASAAPL | 458 | 960 | 7287 |
| PPDAASAAPLRTITA | 20 | 3869 | 3631 |
| SAAPLRTITADTFRK | 301 | >46666.67 | 1100 |
| EAENITTGTAEHTSL | 316 | 8300 | |
| RLFDNASLRAHRLHQ | 996 | >36206.9 | 11766 |
| QLAFDTYQEFEEAYI | >89285.71 | 673 | 35 |
| ISLLLIQSWLEPVQF | >89285.71 | 562 | 5234 |
| NSLVYGASDSNVYDL | 14164 | 8337 | 731 |
| SDSNYYDLLKDLEEG | >89285.71 | 4136 | 503 |
| KIFGSLAFLPESFDG | 320 | | |
| DPA | | | |
| CLKDRRNFDIIPEEIK | 19365 | 208 | 774 |
| QLQQFQKEDAAVTIY | 26205 | 579 | 2145 |
| QKEDAAVTIYIEMLQN | 515 | 153 | 1685 |
| STGWNRTIVENLLAN | 47081 | 5041 | 322 |
| ETIVENLLANVYHQR | >92592.59 | >75000 | 344 |
| KEDSHCAWTWRVEI | 4102 | 2123 | 465 |
| MSYNLLGFLQRSSNT | 724 | >51219.51 | |
| QHLCGSHLVEALYLV | 2553 | 8413 | 359 |
| GSHLVEALYLVCGER | >89285.71 | 2491 | 677 |
| GSDLVEALYLVCGER | >89285.71 | 806 | |
| VEALYLVCGERGFLY | 27334 | 514 | |
| VEALYLVTGERGFFY | 20021 | 564 | |
| IDVWLGGLAENFLPY | 204 | 138 | 13 |
| IDVWLGGLAYNFLPY | 85 | 358 | 63 |
| IDVWLGGLALNFLPY | 49 | 457 | 52 |
| IDVWLGGLASNFLPY | 175 | 1251 | 40 |
| IDVWLGGLAKNFLPY | 170 | 10247 | >4166.67 |
| IDVWLGGLADNFLPY | 296 | 1762 | 12 |
| IDVYLGGLAENFLPY | 161 | 186 | 30 |
| IDVLLGGLAENFLPY | 166 | 437 | 27 |
| IDVSLGGLAENFLPY | 188 | 277 | 48 |
| IDVKLGGLAENFLPY | 724 | 5511 | 41 |
| IDWDLGGLAENFLPY | 218 | 73 | 17 |
| IDVWLGGLAENYLPY | 223 | 110 | 19 |
| IDVWLGGLAENVLPY | 84 | 82 | 15 |
| IDVWLGGLAENSLPY | 116 | 125 | 25 |
| IDVWLGGLAENKIPY | 353 | 5189 | 51 |
| IDVWLGGLAENDLPY | 240 | 60 | 22 |
| IYVWLGGLAENFLPY | 170 | 237 | 13 |
| ILVWLGGLAENFLPY | 216 | 147 | 10.0 |
| ISVWLGGLAENFLPY | 132 | 286 | 18 |
| IKVWLGGLAENFLPY | 180 | 220 | 37 |
| IEVWLGGLAENFLPY | 158 | 145 | 23 |
| IDVWLGGLAENFLPF | 111 | 177 | 3.6 |
| IDVWLGGLAENFLPL | 182 | 114 | 17 |
| IDVWLGGLAENFLPS | 134 | 249 | 27 |
| IDVWLGGLAENFLPK | 261 | 231 | 23 |
| IDVWLGGLAENFLPD | 115 | 91 | 20 |
| IDVWLGGLAENFYPY | 324 | 203 | 37 |
| IDVWLGGLAENFVPY | 346 | 272 | 12 |
| IDVWLGGLAENFSPY | 131 | 193 | 47 |
| IDVWLGGLAENFKPY | 195 | 262 | 310 |
| IDVWLGGLAENFDPY | 364 | 90 | 32 |
| IDVWLGGLAEYFLPY | 151 | 88 | 14 |
| IDVWLGGLAELFLPY | 107 | 81 | 22 |
| IDVWLGGLAESFLPY | 60 | 64 | 49 |
| IDVWLGGLAEKFLPY | 68 | 112 | 66 |
| IDVWLGGLAEDFLPY | 357 | 120 | 23 |
| IDVWLGGLAEQFLPY | 167 | 123 | 9.7 |
| IDVWLGGLYENFLPY | 912 | 697 | 6.4 |
| IDVWLGGLLENFLPY | 810 | 1734 | 58 |
| IDVWLGGLSENFLPY | 242 | 1348 | 37 |
| IDVWLGGLKENFLPY | 15907 | >2800 | 25 |
| IDVWLGGLDENFLPY | >19230.77 | 637 | 18 |
| IDVWLGGYAENFLPY | 900 | 492 | 39 |
| IDVWLGGVAENFLPY | 982 | 327 | 75 |
| IDVWLGGSAENFLPY | 427 | 755 | 166 |
| IDVWLGGKAENFLPY | 517 | 633 | 398 |
| IDVWLGGDABNFLPY | 11114 | 2074 | 11 |
| IDVWLGYLAENFLPY | 15215 | 1121 | 31 |
| IDVWLGLLAENFLPY | 2986 | 180 | 39 |
| IDVWLGSLAENFLPY | 654 | 278 | 72 |
| IDVWLGKLAENFLPY | 2333 | 20023 | 81 |
| IDVWLGDLAENFLPY | >44642.86 | 370 | 18 |
| IDVWLYGLAENFLPY | 2171 | 442 | 18 |
| IDVWLLGLAENFLPY | 4903 | 455 | 47 |
| IDVWLSGLAENFLPY | 3043 | 373 | 98 |
| IDVWLKGLAENFLPY | 41667 | 1115 | 55 |
| IDVWLDGLAENFLPY | 13325 | 357 | 43 |
| IDVWYGGLAENFLPY | 375 | 224 | 43 |
| IDVWVGGLAENFLPY | 128 | 158 | 14 |
| IDVWSGGLAENFLPY | 451 | 128 | 15 |
| IDVWKGGLAENFLPY | 256 | 346 | 41 |
| IDVWDGGLAENFLPY | 2086 | 299 | 112 |
| IDYWLGGLAENFLPY | 503 | 342 | 49 |
| IDLWLGGLAENFLPY | 1292 | 661 | 25 |
| IDSWLGGLAENFLPY | 508 | 276 | 35 |
| IDKWLGGLAENFLPY | 579 | 534 | 62 |
| IDDWLGGLAENFLPY | 219 | 101 | 85 |
| IDVWLGGLAENFLYY | 341 | 387 | 154 |
| IDVWLGGLAENFLLY | 649 | 491 | 52 |
| IDVWLGGLAENFLSY | 425 | 676 | 54 |
| IDVWLGGLAENFLKY | 2266 | 995 | 111 |
| IDVWLGGLAENFLDY | 371 | 149 | 49 |
| YDVWLGGLAENFLPY | 482 | 214 | 59 |
| LDVWLGGLAENFLPY | 180 | 216 | 29 |
| SDVWLGGLAENFLPY | 154 | 232 | 19 |
| KDVWLGGLAENFLPY | 348 | 254 | 54 |
| DDVWLGGLAEKFLPY | 241 | 158 | 48 |

TABLE 25

HLA-DR SUPERTYPE

| | | | | | | |
|---|---|---|---|---|---|---|
| | SEQ | | | | | |
| | ID | | | | | |
| Sequence | NO. | AA | Organism | Protein | Position | Analog |

| | | | | | |
|---|---|---|---|---|---|
| AC- | 18 | A2 | MHC | Unknown | |
| NPTKHKWEAAHVAE | | | derived | | |
| QLAA | | | | | |
| DDYVKQYTKQYTKQ | 19 | Artificial | | | |
| NTLKK | | sequence | | | |
| AAAKAAAAAAYAA | 13 | Artificial | | | A |
| | | sequence | | | |
| AC- | 13 | Artificial | | | A |
| AAAKAAAAAAYAA | | sequence | | | |
| (20)AYA(20)A(20)A(20) | 13 | Artificial | | | A |
| K(20)A(20) | | sequence | | | |
| AC- | 13 | Artificial | | | A |
| AAAKATAAAAYAA | | sequence | | | |
| AC- | 13 | Artificial | | | A |
| AAAKAAAAAAFAA | | sequence | | | |
| AC- | 13 | Artificial | | | A |
| AAAKATAAAA(10)AA | | sequence | | | |
| AC- | 13 | Artificial | | | A |
| AAAKATAAAA(23)AA | | sequence | | | |
| AAKAAAAAAA(10)AA | 13 | Artificial | | | A |
| | | sequence | | | |
| AAYAAAATAKAAA | 13 | Artificial | | | A |
| | | sequence | | | |
| AALAAAAAAKAAA | 13 | Artificial | | | A |
| | | sequence | | | |
| AAEAAAATAKAAA | 13 | Artificial | | | A |
| | | sequence | | | |
| AAYIIAAAAKAAA | 13 | Artificial | | | A |
| | | sequence | | | |
| AAYAAAAIIKAAA | 13 | Artificial | | | A |
| | | sequence | | | |
| AFLRAAAAAAFAA | 13 | Artificial | | | A |
| | | sequence | | | |
| AFLRQAAAAAFAAY | 14 | Artificial | | | A |
| | | sequence | | | |
| AAFAAAKTAAAFA | 13 | Artificial | | | A |
| | | sequence | | | |
| YAAFAAAKTAAAFA | 14 | Artificial | | | A |
| | | sequence | | | |
| AALKATAAAAAAA | 13 | Artificial | | | A |
| | | sequence | | | |
| YAR(15)ASQTTLKAKT | 14 | Artificial | | | |
| | | sequence | | | |
| YARF(33)QTTLKAKT | 14 | Artificial | | | |
| | | sequence | | | |
| PKYFKQRILKFAT | 13 | Artificial | | | A |
| | | sequence | | | |
| PKYFKQGFLKGAT | 13 | Artificial | | | A |
| | | Sequence | | | |
| PKYGKQIDLKGAT | 13 | Artificial | | | A |
| | | sequence | | | |
| AAFFFFFGGGGGA | 13 | Artificial | | | |
| | | sequence | | | |
| AADFFFFFFFFDA | 13 | Artificial | | | |
| | | sequence | | | |
| AAKGIKIGFGIFA | 13 | Artificial | | | |
| | | sequence | | | |
| AAFIFIGGGKIKA | 13 | Artificial | | | |
| | | sequence | | | |
| AAKIFIGFFIDGA | 13 | Artificial | | | |
| | | sequence | | | |
| AAFIGFGKIKFIA | 13 | Artificial | | | |
| | | sequence | | | |
| AAKIGFGIKIGFA | 13 | Artificial | | | |
| | | sequence | | | |
| AAFKIGKFGIFFA | 13 | Artificial | | | |
| | | sequence | | | |
| AADDDDDDDDDDA | 13 | Artificial | | | |
| | | sequence | | | |
| (43)AAIGFFFFKKGIA | 14 | Artificial | | | |
| | | sequence | | | |
| (43)AAFFGIFKIGKFA | 14 | Artificial | | | |
| | | sequence | | | |
| (43)AADFGIFIDFIIA | 14 | Artificial | | | |
| | | sequence | | | |
| (43)AAIGGIFIFKKDA | 14 | Artificial | | | |
| | | sequence | | | |
| (43)AAFIGFGKIKFIA | 13 | Artificial | | | |
| | | sequence | | | |
| (43)AAKIGFGIKIGFA | 13 | Artificial | | | |
| | | sequence | | | |
| (43)AAFKIGKFGIFFA | 13 | Artificial | | | |
| | | sequence | | | |
| AAAKAAAAAAAAF | 13 | Artificial | | | |
| | | sequence | | | |
| AAAKAAAAAAAFA | 13 | Artificial | | | |
| | | sequence | | | |
| AAAKAAAAAAFAA | 13 | Artificial | | | |
| | | sequence | | | |
| AAAKAAAAPAAAA | 13 | Artificial | | | |
| | | sequence | | | |
| FAAAAAAAAAAAA | 13 | Artificial | | | |
| | | sequence | | | |
| AAAAAAAAAAAAN | 13 | Artificial | | | |
| | | sequence | | | |
| AAAAAAAAAAANA | 13 | Artificial | | | |
| | | sequence | | | |
| AAANAAAAAAAAA | 13 | Artificial | | | |
| | | sequence | | | |
| AAAAAAAAAAAAS | 13 | Artificial | | | |
| | | sequence | | | |
| AAAAASAAAAAAA | 13 | Artificial | | | |
| | | sequence | | | |
| ASAAAAAAAAAAA | 13 | Artificial | | | |
| | | sequence | | | |
| AFAAAKTAA | 9 | Artificial | | | |
| | | sequence | | | |
| YARFLALTTLRARA | 14 | Artificial | | | A |
| | | sequence | | | |
| YAR(15A)SQTTLKAKT | 14 | Artificial | | | A |
| | | sequence | | | |
| YAR(15A)RQTTLKAAA | 14 | Artificial | | | A |
| | | sequence | | | |
| (15A)RQTTLKAAA | 11 | Artificial | | | A |
| | | sequence | | | |
| (16A)RQTTLKAAA | 11 | Artificial | | | A |
| | | sequence | | | |
| (46)AAKTAAAFA | 10 | Artificial | | | |
| | | sequence | | | |
| (39)AAAATKAAA | 10 | Artificial | | | |
| | | sequence | | | |
| (52)AAAATKAAAA | 11 | Artificial | | | |
| | | sequence | | | |
| (55)AAAATKAAAA | 11 | Artificial | | | |
| | | sequence | | | |
| A(14)AAAKTAAA | 10 | Artificial | | | |
| | | sequence | | | |
| AA(14)A(35)ATKAAAA | 12 | Artificial | | | |
| | | sequence | | | |
| AA(14)AA(36)TKAAAA | 12 | Artificial | | | |
| | | sequence | | | |
| AFAAAKTAA(72) | 10 | Artificial | | | |
| | | sequence | | | |
| (49)AAAKT(64)AAA | 10 | Artificial | | | |
| | | sequence | | | |
| (49)AAAKTA(64)AA | 10 | Artificial | | | |
| | | sequence | | | |
| HQAISPRTLNGPGPGS | 20 | Artificial | | | |
| PAIF | | sequence | | | |
| YAAFAAAKTAAAFA | 14 | Artificial | | | |
| | | sequence | | | |
| TBGRCLHYTVDKSKPK | 16 | Bee Venom | | 103 | |
| AWVAWRNRCK | 0 | Chicken | HEL | 107 | |
| IVSDGNGMNAWVAWRNRC | 18 | Chicken | HEL | 98 | |
| PHHTALRQAILSWGE | 20 | DPw4 binder | | | |
| LMTLA | | | | | |
| WMYYHGQRHSDEHHH | 15 | EBV | LMP | 183 | |
| YIVMSDWTGGA | 15 | EBV | LMP | 41 | |
| AHAAHAAHAAHAAHAA | 16 | HA | | | A |
| MDIDPYKEFGATVEL | 25 | HBV | core | | |
| LSFLPSDFFP | | | | | |
| GMLPVCPLIPGSSTTS | 19 | HBV | env | 102 | |
| TGP | | | | | |
| LGFFPDHQLDPAFRANT | 17 | HBV | env | 11 | |
| GYKVLVLNPSV | 11 | HCV | NS3 | 1248 | |
| LMAFTAAVTS | 10 | HCV | NS4 | 1790 | |
| TFALWRVSAEEY | 12 | HCV | NS5 | 2079 | |
| ALWRVSAEEY | 10 | HCV | NS5 | 2081 | |
| EEYVEIRQVGDFH | 13 | HCV | NS5 | 2088 | |
| VGGVYLLPRRGPRLGV | 16 | HCV | | | |
| VGGAYLLPRRGPRLGV | 16 | HCV | | | A |
| VGGVALLPRIRGPRLGV | 16 | HCV | | | A |
| VGGVYALPRRGPRLGV | 16 | HCV | | | A |
| VGGVYLAPRRGPRLGV | 16 | HCV | | | A |
| VGGVYLLARRGPRLGV | 16 | HCV | | | A |
| VGGVYLLPARGPRLGV | 16 | HCV | | | A |
| VGGVYLLRRAGPRLGV | 16 | HCV | | | A |
| GAPLGGAARALAHGV | 15 | HCV | | | |
| GAALGGAARALAHGV | 15 | HCV | | | A |
| GAPLAGAARALAHGV | 15 | HCV | | | A |
| GAPLGAAARALAHGV | 15 | HCV | | | A |
| GAPLGGLARALAHGV | 15 | HCV | | | A |
| GAPLGGALRALAHGV | 15 | HCV | | | A |
| GAPLGGAAAALAHGV | 15 | HCV | | | A |
| GAPLGGAARLLAHGV | 15 | HCV | | | A |
| GAPLGGAARAAAHGV | 15 | HCV | | | A |
| GAPLGGAARALAAGV | 15 | HCV | | | A |
| FPDWQNYTPGPGTRY | 15 | HIV | NEF | 200 | |
| RFPLTFGWCFKLVPV | 15 | HIV | NEF | 216 | |
| RQDILDLWVYHTQGY | 15 | HIV | NEF | 182 | |
| RQEILDLWVYHTQGF | 15 | HIV | NEF | 182 | |
| LSHFLKEKGGLBGLI | 15 | HIV | NEF | 114 | |
| LSFFLKEKGGLDGLI | 15 | HIV | NEF | 114 | |
| LEPWNHPGSQPKTACT | 16 | HIV | TAT | 11 | |
| QVCFITKGLGISYGR | 15 | HIV | TAT | 38 | |
| QLGFLKKGLGISYGR | 15 | HIV | TAT | 38 | |
| PPEESFRFGEEKTRPS | 16 | HIV1 | gp | 81 | |
| CIVYRDGNPYAVCDK | 15 | HPV | E6 | 58 | |
| HYCYSLYGTTLEQQY | 15 | HPV | E6 | 85 | |
| CYSLYGTTLEQQYNK | 15 | HPV | E6 | 87 | |
| NTSLQDIEITCVYCK | 15 | HPV | E6 | 22 | |
| VFEFAFKDLFVVYRD | 15 | HPV | E6 | 44 | |
| EFAFKDLFVVYRDSI | 15 | HPV | E6 | 46 | |
| DLFVVYRDSIPHAAC | 15 | HPV | E6 | 51 | |
| FVVYRDSTPHAACHK | 15 | HPV | E6 | 53 | |
| NTGLYNLLIRCLRCQ | 15 | HPV | E6 | 95 | |
| IRCLRCQKPLNPAEK | 15 | HPV | E6 | 103 | |
| PRKLHELSSALEIPY | 15 | HPV | E6 | 9 | |
| EIPYDELRLNCVYCK | 15 | HPV | E6 | 20 | |
| TEVLDFAFTDLTIVY | 15 | HPV | E6 | 40 | |
| VLDFAFTDLTIVYRD | 15 | HPV | E6 | 42 | |
| DFAFTDLTIVYRDDT | 15 | HPV | E6 | 44 | |
| TIVYRDDTPHGVCTK | 15 | HPV | E6 | 51 | |
| WYRYSVYGTTLEKLT | 15 | HPV | E6 | 78 | |
| ETTIHNIELQCVECK | 15 | HPV | E6 | 20 | |
| SEVYDFAFADLTVVY | 15 | HPV | E6 | 40 | |
| VYDFAFADLTVVYRE | 15 | HPV | E6 | 42 | |
| DFAFADLTVVYREGN | 15 | HPV | E6 | 44 | |
| TVVYREGNPFGICKL | 15 | HPV | E6 | 51 | |
| GNPFGICKLCLRFLS | 15 | HPV | E6 | 57 | |
| NYSVYGNThEQTVKK | 15 | HPV | E6 | 80 | |
| KKPLNEILIRCIIGQ | 15 | HPV | E6 | 93 | |
| NEILIRCIIGQRPLC | 15 | HPV | E6 | 97 | |
| IRCIICQRPLCPQEK | 15 | HPV | E6 | 101 | |
| CIVYRDCIAYAACHK | 15 | HPV | E6 | 53 | |
| NTELYNLLIRCLRCQ | 15 | HPV | E6 | 95 | |
| IRCLRCQKPLNPAEK | 15 | HPV | E6 | 103 | |
| REVYKFLFTDLRIVY | 15 | HPV | E6 | 40 | |
| RIVYRDNNPYGVCIM | 15 | HPV | E6 | 51 | |
| NNPYGVCIMCLRFLS | 15 | HPV | E6 | 57 | |
| EERVKKPLSEITIRC | 15 | HPV | E6 | 89 | |
| IRCIICQTPLCPEEK | 15 | HPV | E6 | 101 | |
| EIPLIDLRLSCVYCK | 15 | HPV | E6 | 23 | |
| SCVYGKKELTRAEVY | 15 | HPV | E6 | 32 | |
| VCLLFYSKVRKYRYY | 15 | HPV | E6 | 68 | |
| YYDYSVYGATLESIT | 15 | HPV | E6 | 81 | |
| IRCYRCQSPLTPEEK | 15 | HPV | E6 | 104 | |
| VYDFVFADLRIVYRD | 15 | HPV | E6 | 42 | |
| DFVFADLRIVYRDGN | 15 | HPV | E6 | 44 | |
| RIVYRDGNPFAVCKV | 15 | HPV | E6 | 51 | |
| GNPFAVGKVCLRLLS | 15 | HPV | E6 | 57 | |
| KKCLNEILIRCIICQ | 15 | HPV | E6 | 93 | |
| NEILIRCIICQRPLC | 15 | HPV | E6 | 97 | |
| RTAMFQDPQERPRKL | 15 | HPV | E6 | 5 | |
| LFVVYRDSIPHAACH | 15 | HPV | E6 | 52 | |
| LTIVYRDDTPHGVGT | 15 | HPV | E6 | 50 | |
| LCIVYRDGIAYAACH | 15 | HPV | E6 | 52 | |
| YKFLFTDLRIVYRDN | 15 | HPV | E6 | 43 | |
| YNFACTELKLVYRDD | 15 | HPV | E6 | 46 | |
| LKLVYRDDFPYAVCR | 15 | HPV | E6 | 53 | |
| YDFVFADLRIVYRDG | 15 | HPV | E6 | 43 | |
| LRIVYRDGNPFAVCK | 15 | HPV | B6 | 50 | |
| HEYMLDLQPETTDLY | 15 | HPV | E7 | 9 | |
| TLRLCVQSTHVDIRT | 15 | HPV | E7 | 64 | |
| IRTLEDLLMGTLGIV | 15 | HPV | E7 | 76 | |
| LEDLLMGTLGIVCPT | 15 | HPV | E7 | 79 | |
| DLLMGTLGIVCPICS | 15 | HPV | E7 | 81 | |
| KATLQDIVLHLEPQN | 15 | HPV | E7 | 5 | |
| IDGVNHQHLPARRAE | 15 | HPV | E7 | 41 | |
| LRAFQQLFLNTLSFV | 15 | HPV | E7 | 83 | |
| FQQLFLNTLSFVCPW | 15 | HPV | E7 | 86 | |
| QDYVLDLQPEATDLH | 15 | HPV | E7 | 9 | |
| DIRILQELLMGSFGI | 15 | HPV | E7 | 75 | |
| IRILQELLMGSFGIV | 15 | HPV | E7 | 76 | |
| ELLMGSFGIVCPNCS | 15 | HPV | E7 | 81 | |
| KBYVLDLYPEPTDLY | 15 | HPV | E7 | 9 | |
| LRTIQQLLMGTVNIV | 15 | HPV | E7 | 76 | |
| IQQLLMGTVNIVCPT | 15 | HPV | E7 | 79 | |
| QLLMGTVNIVCPTCA | 15 | HPV | E7 | 81 | |
| RETLQEIVLHLEPQN | 15 | HPV | E7 | 5 | |
| LRTLQQLFLSTLSFV | 15 | HPV | E7 | 84 | |
| LQQLFLSTLSFVCPW | 15 | HPV | E7 | 87 | |
| KDYILDLQPETTDLH | 15 | HPV | E7 | 9 | |
| LRTLQQMLLGTLQVV | 15 | HPV | E7 | 78 | |
| LQQMLLGTLQVVCPG | 15 | HPV | E7 | 81 | |
| QMLLGTTQVVCPGCA | 15 | HPV | E7 | 83 | |
| VPTLQDVVLELTPQT | 15 | HPV | E7 | 5 | |
| LQDVVLELTPQTEID | 15 | HPV | E7 | 8 | |
| QDVVLELTPQTEIDL | 15 | HPV | E7 | 9 | |
| CKFVVQLDIQSTKED | 15 | HPV | E7 | 68 | |
| VVQLDIQSTKEDLRV | 15 | HPV | E7 | 71 | |
| DLRVVQQLLMGALTV | 15 | HPV | E7 | 82 | |
| LRVVQQLLMGALTVT | 15 | HPV | E7 | 83 | |
| VQQLLMGALTVTCPL | 15 | HPV | E7 | 86 | |
| QQLLMGALTVTCPLC | 15 | HPV | E7 | 87 | |
| QLLMGALTVTCPLCA | 15 | HPV | E7 | 88 | |
| REYILDLHPEPTDLF | 15 | HPV | E7 | 9 | |
| TCCYTCGTTVRICIN | 15 | HPV | E7 | 57 | |
| VRTLQQLLMGTCTIV | 15 | HPV | E7 | 77 | |
| LQQLLMGTCTIVCPS | 15 | HPV | E7 | 80 | |
| MLDLQPETTDLYCYE | 15 | HPV | E7 | 12 | |
| VLDLYPEPTDLYCYE | 15 | HPV | E7 | 12 | |
| LREYILDLHPEPTDL | 15 | HPV | E7 | 8 | |
| HIEFTPTRTDTYACRV | 16 | Human | B2-μglobulin | 67 | |
| LWWVNNESLPVSPRL | 15 | Human | CEA | 177 | A |
| YEEYVRFDSDVGE | 13 | Human | DRB and | | |
| | | | CD4 peptide | | |
| EEYVRFDSDVGE | 12 | Human | DRB and | | |
| | | | CD4 peptide | | |
| APPRLICDSRVLERY | 15 | Human | EPO | 1 | |
| ICDSRVLERYLLEAK | 15 | Human | EPO | 6 | |
| VLERYLLEAICEAENI | 15 | Human | EPO | 11 | |
| EHCSLNENITVPDTK | 15 | Human | EPO | 31 | |
| NENITVPDTKVNFYA | 15 | Human | EPO | 36 | |
| VPDTKVNFYAWKRME | 15 | Human | EPO | 41 | |
| XFNFYAWKRMEVGQQA | 15 | Human | EPO | 46 | |
| WKRMEVGQQAVEVWQ | 15 | Human | EPO | 51 | |
| VGQQAVEVWQGLALL | 15 | Human | EPO | 56 | |
| VEVWQGLALLSEAVL | 15 | Human | EPO | 61 | |
| GLALLSEAVLRGQAL | 15 | Human | EPO | 66 | |
| SEAVLRGQALLVNSS | 15 | Human | EPO | 71 | |
| RGQALLVNSSQPWEP | 15 | Human | EPO | 76 | |
| LVNSSQPWEPLQLHV | 15 | Human | EPO | 81 | |
| QPWEPLQLHVDKAVS | 15 | Human | EPO | 86 | |
| LQLHVDKAVSGLRSL | 15 | Human | EPO | 91 | |
| DKAVSGLRSLTTLLR | 15 | Human | EPO | 96 | |
| GLRSLTTLLRALGAQ | 15 | Human | EPO | 101 | |
| TTLLRALGAQKEAIS | 15 | Human | EPO | 106 | |
| ALGAQKEAISPPDAA | 15 | Human | EPO | 111 | |
| KEAISPPDAASAAPL | 15 | Human | EPO | 116 | |
| PPDAASAAPLRTITA | 15 | Human | EPO | 121 | |
| SAAPLRTITADTFRK | 15 | Human | EPO | 126 | |
| RTITADTFRKLFRVY | 15 | Human | EPO | 131 | |
| DTFRKLFRVYSNFLR | 15 | Human | EPO | 136 | |
| LFRVYSNFLRGKLKL | 15 | Human | EPO | 141 | |
| SNFLRGKLKLYTGEA | 15 | Human | EPO | 146 | |
| KLKLYTGEACRTGDR | 15 | Human | EPO | 152 | |
| APPRLITDSRVLERY | 15 | Human | EPO | 1 | A |
| ITDSRVLERYLLEAX | 15 | Human | EPO | 6 | A |
| EHTSLNENITVPDTK | 15 | Human | EPO | 31 | A |
| KLKLYTGEATRTGDR | 15 | Human | EPO | 152 | A |
| PQPFRPQQPYPQ | 12 | Human | gliadin | | |
| PFRPQQPYPQ | 10 | Human | gliadin | | |
| PQPFRPQQPYP | 11 | Human | gliadin | | |
| PQPFRPQQP | 9 | Human | gliadin | | |
| KQPFRPQQPYPQ | 12 | Human | gliadin | | |
| PKPFRPQQPYPQ | 12 | Human | gliadin | | |
| PQPFKPQQPYPQ | 12 | Human | gliadin | | |
| PQPFRKQQPYPQ | 12 | Human | gliadin | | |
| PQPFRPQKPYPQ | 12 | Human | gliadin | | |
| PQPFRPQQPKPQ | 12 | Human | gliadin | | |
| PQPFRPQQPYKQ | 12 | Human | gliadin | | |
| PQPFRPQQPYPK | 12 | Human | gliadin | | |
| QFLGQQQPFPPQ | 12 | Human | gliadin | | |
| FLGQQQPFPPQ | 11 | Human | gliadin | | |
| LGQQQPFPPQ | 10 | Human | gliadin | | |
| QFLGQQQPFPP | 11 | Human | gliadin | | |
| QFLGQQQPF | 9 | Human | gliadin | | |
| IRNLALQTLPAMGNVY | 16 | Human | gliadin | | |
| NLALQTLPAMCNVY | 14 | Human | gliadin | | |
| LALQTLPAMCNVY | 13 | Human | gliadin | | |
| IRNLALQTLPAM | 12 | Human | gliadin | | |
| IRNLALQTLP | 10 | Human | gliadin | | |
| EGDAFELTVSGQGGLPK | 17 | Human | gp100 | 506 | |
| ESTGMTPEKVPVSEVMGT | 18 | Human | gp100 | 370 | |
| FPTIPLSRLFDNASL | 15 | Human | Growth | | |
| | | | hormone | | |
| RLFDNASLRAHRLHQ | 15 | Human | Growth | 8 | |
| | | | hormone | | |
| LRAHRLHQLAFDTYQ | 15 | Human | Growth | 15 | |
| | | | hormone | | |
| QLAFDTYQEFEEAYI | 15 | Human | Growth | 22 | |
| | | | hormone | | |
| QEFEEAYIPKEQKYS | 15 | Human | Growth | 29 | |
| | | | hormone | | |
| IPKEQKYSFLQNPQT | 15 | Human | Growth | 36 | |
| | | | hormone | | |
| SFLQNPQTSLCFSES | 15 | Human | Growth | 43 | |
| | | | hormone | | |
| TSLCFSESIPTPSNR | 15 | Human | Growth | 50 | |
| | | | hormone | | |
| REETQQKSNLELLRI | 15 | Human | Growth | 64 | |
| | | | hormone | | |
| SNLELLRISLLLIQS | 15 | Human | Growth | 71 | |
| | | | hormone | | |
| ISLLLIQSWLEPVQF | 15 | Human | Growth | 78 | |
| | | | hormone | | |
| SWLEPVQFLRSVFAN | 15 | Human | Growth | 85 | |
| | | | hormone | | |
| FLRSVFANSLVYGAS | 15 | Human | Growth | 92 | |
| | | | hormone | | |
| NSLVYGASDSNVYDL | 15 | Human | Growth | 99 | |
| | | | hormone | | |
| SDSNVYDLLKDLEEG | 15 | Human | Growth | 106 | |
| | | | hormone | | |
| GIQTLMGRLEDGSPR | 15 | Human | Growth | 120 | |
| | | | hormone | | |
| RLEDGSPRTGQIFKQ | 15 | Human | Growth | 127 | |
| | | | hormone | | |
| RTGQIFKQTYSKFDT | 15 | Human | Growth | 134 | |
| | | | hormone | | |
| QTYSKFDTNSHNDDA | 15 | Human | Growth | 141 | |
| | | | hormone | | |
| TNSHNDDALLKNYGL | 15 | Human | Growth | 148 | |
| | | | hormone | | |
| ALLKNYGLLYCFRKD | 15 | Human | Growth | 155 | |
| | | | hormone | | |
| DMDKVETFLRIVQCR | 15 | Human | Growth | 169 | |
| | | | hormone | | |
| FLRIVQCRSVEGSCGF | 16 | Human | Growth | 176 | |
| | | | hormone | | |
| FPTIPLSRLFDNAML | 15 | Human | Growth | 1 | A |
| | | | hormone | | |
| RLFDNAMLRAHRLHQ | 15 | Human | Growth | 8 | A |
| | | | hormone | | |
| QLAFDTYQEFEQNPQ | 15 | Human | Growth | 22 | A |
| | | | hormone | | |
| SFLQNPQTSLCCFRK | 15 | Human | Growth | 43 | A |
| | | | hormone | | |
| SNLELL,RIGLLLIQS | 15 | Human | Growth | 71 | A |
| | | | hormone | | |
| ICLLLIQSWLEPVQF | 15 | Human | Growth | 78 | A |
| | | | hormone | | |
| NSLVYGASDSNIYDL | 15 | Human | Growth | 99 | A |
| | | | hormone | | |
| SDSNIYDLLKDLEEG | 15 | Human | Growth | 106 | A |
| | | | hormone | | |
| DKVETFLRIVQCCGF | 15 | Human | Growth | 169 | A |
| | | | hormone | | |
| SFLQNPQTSLTFSES | 15 | Human | Growth | 43 | A |
| | | | hormone | | |
| TSLTFSESIPTPSNR | 15 | Human | Growth | 50 | A |
| | | | hormone | | |
| ALLKNYGLLYTFRIKD | 15 | Human | Growth | 155 | A |
| | | | hormone | | |
| LLYTFRKDMDKVETF | 15 | Human | Growth | 162 | A |
| | | | hormone | | |
| DMDKVETFLRIVQTR | 15 | Human | Growth | 169 | A |
| | | | hormone | | |
| FLRIVQTRSVEGSTGF | 16 | Human | Growth | 176 | A |
| | | | hormone | | |
| HLDMLRIILYQGCQVV | 15 | Human | Her2/neu | 42 | |
| RLRIVRGTQLFEDNYAL | 17 | Human | Her2/neu | 98 | |
| GVGSPYVSRLLGICL | 15 | Human | Her2/neu | 776 | |
| TLERPKTLSPGKNGV | 15 | Human | Her2/neu | 1166 | |
| KIFGSLAFLPESFDGDPA | 18 | Human | Her2/neu | 369 | |
| ELVSBFSRMARDPQ | 14 | Human | Her2/neu | 971 | |
| GEALSTLVLNRLKVG | 15 | Human | HSP60 | 280 | |
| AYVLLSEKKISSIQS | 15 | Human | HSP60 | 242 | |
| VASLLTTAEVVVTEI | 15 | Human | HSP60 | 535 | |
| KCEFQDAYVILLSEKK | 16 | Human | HSP60 | 236 | |
| ALSTLVLNRLKVGLQ | 15 | Human | HSP60 | 282 | |
| MSYNLLGFLQRSSNG | 15 | Human | IFN-B | 1 | |
| LGFLQRSSNCQCQKL | 15 | Human | IFN-B | 6 | |
| RSSNCQCQKLLWQLN | 15 | Human | IFN-B | 11 | |
| QCQKLLWQLNGRLEY | 15 | Human | IFN-B | 16 | |
| LWQLNGRLEYCLKDR | 15 | Human | IFN-B | 21 | |
| GRLEYCLKDRRITFDI | 15 | Human | IFN-B | 26 | |
| RNFDIPEBIKQLQQF | 15 | Human | IFN-B | 36 | |
| PEEIKQLQQFQKEDA | 15 | Human | IFN-B | 41 | |
| QLQQFQKEDAAVTIY | 15 | Human | IFN-B | 46 | |
| QKEDAAVTIYEMLQN | 15 | Human | IFN-B | 51 | |
| AVTIYEMLQNIFAIF | 15 | Human | IFN-B | 56 | |
| EMLQNIFAIFRQDSS | 15 | Human | IFN-B | 61 | |
| IFAIFRQDSSSTGWN | 15 | Human | IFN-B | 66 | |
| RQDSSSTGWNETIVE | 15 | Human | IFN-B | 71 | |
| STGWNETIVENLLAN | 15 | Human | IFN-B | 76 | |
| ETIVENLLANVYHQR | 15 | Human | IFN-B | 81 | |
| NLLANVYHQRNHLKT | 15 | Human | IFN-B | 86 | |
| VYHQRNHLKTVLEEK | 15 | Human | IFN-B | 91 | |
| LEKEDFTRGKPMSSL | 15 | Human | IFN-B | 106 | |
| FTRGKRMSSLHLKRY | 15 | Human | IFN-B | 111 | |
| RMSSLHLKRYYGRIL | 15 | Human | IFN-B | 116 | |
| HLKRYYGRILHYLKA | 15 | Human | IFN-B | 121 | |
| YGRILHYLKAKEDSH | 15 | Human | IFN-B | 126 | |
| HYLKAKEDSHCAWTI | 15 | Human | IFN-B | 131 | |
| KEDSHCAWTIVRVEI | 15 | Human | IFN-B | 136 | |
| CAWTIVRVEILRNEY | 15 | Human | IFN-B | 141 | |
| VRVEILRNEYVINRL | 15 | Human | IFN-B | 146 | |
| RNFYVINRLTGYLEN | 15 | Human | IFN-B | 152 | |
| MSYNLLGFLQRSSNT | 15 | Human | IFN-B | 1 | A |
| LGFLQRSSNTQTQKL | 15 | Human | IFN-B | 6 | A |
| RSSNTQTQKLLWQLN | 15 | Human | IFN-B | 11 | A |
| QTQKLLWQLNGRLEY | 15 | Human | IFN-B | 16 | A |
| LWQLNGRLEYTLKDR | 15 | Human | IFN-B | 21 | A |
| GRLBYTLKDRRNFDI | 15 | Human | IFN-B | 26 | A |
| HYLKAKEDSHTAWTI | 15 | Human | IFN-B | 131 | A |
| KEDSHTAWTIVRVEI | 15 | Human | IFN-B | 136 | A |
| TAWTIVRVEILRNFY | 15 | Human | IFN-B | 141 | A |
| LGFLQRSSNCQSQKI | 15 | Human | IFN-B | 6 | A |
| RSSNCQSQKLLWQLN | 15 | Human | IFN-B | 11 | A |
| QSQKLLWQLNGRLEY | 15 | Human | IFN-B | 16 | A |
| GIVEQCGTSICSLYQ | 15 | Human | Insulin alpha | 1 | |
| | | | chain | | |
| TSICSLYQLENYCN | 14 | Human | Insulin alpha | 8 | |
| | | | chain | | |
| GILEQCCTSICSLYQ | 15 | Human | Insulin alpha | 1 | A |
| | | | chain | | |
| GIVEQTTTSITSLYQ | 15 | Human | Insulin alpha | 1 | A |
| | | | chain | | |
| EQTTTSITSLYQLEN | 15 | Human | Insulin alpha | 4 | A |
| | | | chain | | |
| TSICSLYQLENYCG | 14 | Human | Insulin alpha | 8 | A |
| | | | chain | | |
| TSITSLYQLENYTN | 14 | Human | Insulin alpha | 8 | A |
| | | | chain | | |
| TSITSLYQLENYTG | 14 | Human | Insulin alpha | 8 | A |
| | | | chain | | |
| GIVEQCGCGSHLVEA | 15 | Human | Insulin alpha- | | A |
| | | | beta | | |
| SLYQLENYCCGERGF | 15 | Human | Insulin alpha- | | A |
| | | | beta | | |
| CCTSICSLYQLENYCC | 16 | Human | Insulin alpha- | | A |
| | | | beta | | |
| GSHLVEALYLVCCN | 14 | Human | Insulin alpha- | | A |
| | | | beta | | |
| CCGSHLVEALYLVCC | 15 | Human | Insulin alpha- | | A |
| | | | beta | | |
| FVNQHLCGSHLVEAL | 15 | Human | Insulin beta | | |
| | | | chain | | |
| QHLCGSHLVEALYLV | 15 | Human | Insulin beta | 4 | |
| | | | chain | | |
| GSHLVEALYLVGGER | 15 | Human | Insulin beta | 8 | |
| | | | chain | | |
| VEALYLVGGERGFFY | 15 | Human | Insulin beta | 12 | |
| | | | chain | | |
| YLVCGERGFFYTPKT | 15 | Human | Insulin beta | 16 | |
| | | | chain | | |
| FVNQHLCGSDLVEAL | 15 | Human | Insulin beta | 1 | A |
| | | | chain | | |
| FVNQHLTGSHLVEAL | 15 | Human | Insulin beta | 1 | A |
| | | | chain | | |
| QHLTGSHLVEALYLV | 15 | Human | Insulin beta | 4 | A |
| | | | chain | | |
| GSHLVEALYLVTGER | 15 | Human | Insulin beta | 8 | A |
| | | | chain | | |
| VEALYLVCGERGSFY | 15 | Human | Insulin beta | 12 | A |
| | | | chain | | |
| VEALYLVCGERCIFLY | 15 | Human | Insulin beta | 12 | A |
| | | | chain | | |
| VEALYLVTGERGFFY | 15 | Human | Insulin beta | 12 | A |
| | | | chain | | |
| YLVCGERGFLYTPKT | 15 | Human | Insulin beta | 16 | A |
| | | | chain | | |
| YLVCGERGFFYTDKT | 15 | Human | Insulin beta | 16 | A |
| | | | chain | | |
| YLVCGERGFFYTKPT | 15 | Human | Insulin beta | 16 | A |
| | | | chain | | |
| YLVTGERGFFYTPKT | 15 | Human | Insulin beta | 16 | A |
| | | | chain | | |
| YLVTGERGFFYTDKT | 15 | Human | Insulin beta | 16 | A |
| | | | chain | | |
| YLVTGERGFFYTKPT | 15 | Human | Insulin beta | 16 | A |
| | | | chain | | |
| VCGERGFFYTPKTRR | 15 | Human | Insulin beta | 18 | A |
| | | | chain | | |
| VTGERGFFYTPKTRP | 15 | Human | Insulin beta | 18 | A |
| | | | chain | | |
| MWDLVLSIALSVGCT | 15 | Human | Kallikrein2 | 1 | |
| DLVLSIALSVGCTGA | 15 | Human | Kallikrein2 | 3 | |
| HPQWVLTAAHCLKKN | 15 | Human | Kallikrein2 | 56 | |
| QWVLTAAHCLKKNSQ | 15 | Human | Kallikrein2 | 58 | |
| GQRVPVSHSFPHYLY | 15 | Human | Kallikrein2 | 87 | |
| RVPVSHSFPHPLYNM | 15 | Human | Kallikrein2 | 89 | |
| PHPLYNMSLLKHQSL | 15 | Human | Kallikrein2 | 97 | |
| HPLYNMSLLKHQSLR | 15 | Human | Kallikrein2 | 98 | |
| NMSLLKHQSLRPDED | 15 | Human | Kallikrein2 | 102 | |
| SHDLMLLRLSEPAKI | 15 | Human | Kallikrein2 | 118 | |
| HDLMLLRLSEPAKIT | 15 | Human | Kallikrein2 | 119 | |
| PEEFLRPRSLQCVSL | 15 | Human | Kallikrein2 | 162 | |
| PRSLQCVSLHLLSND | 15 | Human | Kallikrein2 | 168 | |
| NGVLQGITSWGPEPC | 15 | Human | Kallikrein2 | 220 | |
| KPAVYTKVVHYRKWI | 15 | Human | Kallikrein2 | 239 | |
| LHLLSNDMCARAYSE | 15 | Human | Kallikrein2 | 176 | |
| VGNWQYFFPVIFSKA | 15 | Human | MAGE3 | 140 | |
| ESEFQAALSRKVAKL | 15 | Human | MAGE6 | 102 | |
| IGHLYIFATCLGLSYDGL | 18 | Human | MAGE6 | 172 | |
| VGNWQYFFPVIFSKAS | 31 | Human | MAGE6 | 140 | |
| DSLQLVFGIELMEVD | | | | | |
| PAYEKLSAEQSPPPY | 15 | Human | MART1 | 102 | |
| RNGYRALMDKSLHV | 23 | Human | MART1 | 51 | |
| GTQCALTRR | | | | | |
| FFKNIVTFFKNIVT | 14 | Human | MBP | | A |
| YKSAHKGFKGVDAQ | 20 | Human | MBP | 134 | |
| GTLSKI | | | | | |
| VDAQGTLSKIFKLGG | 20 | Human | MBP | 144 | |
| RDSRS | | | | | |
| AC- | 23 | Human | MBP | 1 | |
| ASQKRPSQRHGSKYL | | | | | |
| ATAST | | | | | |
| ENPVVHFFKNIVTPR | 15 | Human | MBP | 85 | |
| ENPVVAFFKNIVTPR | 15 | Human | MBP | 85 | SAAS |
| ENPVVHAFKNIVTPR | 15 | Human | MBP | 85 | SAAS |
| ENPVVHFFANIVTPR | 15 | Human | MBP | 85 | SAAS |
| ENPVVHFFKNIVTPA | 15 | Human | MBP | 85 | SAAS |
| NPVVHFFKNIVT | 12 | Human | MBP | 86 | |
| HFFKNIVTPRTPPY | 14 | Human | MBP | 90 | |
| NPVVHFFKNIVTPR | 14 | Human | MBP | 86 | |
| LPVPGVLLKEFTVSGN | 20 | Human | NY-ESO-1 | 116 | |
| ILTI | | | | | |
| WITQCFLPVFLAQPPS | 20 | Human | NY-ESO-1 | 161 | |
| GQRR | | | | | |
| DHRQLQLSISSCLQQL | 20 | Human | NY-ESO-1 | 141 | |
| SLLM | | | | | |
| YLAMPFATPMEAELA | 20 | Human | NY-ESO-1 | 91 | |
| RRSLA | | | | | |
| AAPLLLARAASLSLG | 15 | Human | PAP | 3 | |
| APLLLARAASLSLGF | 15 | Human | PAP | 4 | |
| PLLLARAASLSLGFL | 15 | Human | PAP | 5 | |
| SLSLGFLFLLFFWLD | 15 | Human | PAP | 13 | |
| LLFFWLDRSVLAKEL | 15 | Human | PAP | 21 | |
| DRSVLAKELKFVTLV | 15 | Human | PAP | 27 | |
| AKELKFVTLVFRHGD | 15 | Human | PAP | 32 | |
| RSPIDTFPTDPIKES | 15 | Human | PAP | 47 | |
| FGQLTQLGMEQHYEL | 15 | Human | PAP | 67 | |
| DRTLMSAMTNLAALF | 15 | Human | PAP | 110 | |
| MSAMTNLAALFPPEG | 15 | Human | PAP | 114 | |
| MTNLAALFPPEGVSI | 15 | Human | PAP | 117 | |
| PEGVSIWNPILLWQP | 15 | Human | PAP | 126 | |
| GVSIWNPILLWQPIP | 15 | Human | PAP | 128 | |
| WNPILLWQPIPVHTV | 15 | Human | PAP | 132 | |
| NPILLWQPIPVHTVP | 15 | Human | PAP | 133 | |
| PILLWQPIPVHTVPL | 15 | Human | PAP | 134 | |
| ILLWQPLPVHTVPLS | 15 | Human | PAP | 135 | |
| WQPIPVHTVPLSEDQ | 15 | Human | PAP | 138 | |
| LSGLHGQDLFGIWSK | 15 | Human | PAP | 194 | |
| YDPLYCESVHNFTLP | 15 | Human | PAP | 210 | |
| LPSWATEDTMTKLRE | 15 | Human | PAP | 223 | |
| LRELSELSLLSLYGI | 15 | Human | PAP | 235 | |
| LSELSLLSLYGIHKQ | 15 | Human | PAP | 238 | |
| LSLLSLYGIHKQKEK | 15 | Human | PAP | 241 | |
| KSRLQGGVLVNEILN | 15 | Human | PAP | 255 | |
| GGVLVNEILNHMKRA | 15 | Human | PAP | 260 | |
| IPSYKKLIMYSAHDT | 15 | Human | PAP | 277 | |
| YKKLIMYSAHDTTVS | 15 | Human | PAP | 280 | |
| LIMYSAHDTIWSGLQ | 15 | Human | PAP | 283 | |
| DTTVSGLQMALDVYN | 15 | Human | PAP | 290 | |
| ALDVYNGLLPPYASC | 15 | Human | PAP | 299 | |
| LDVYNGLLPPYASCH | 15 | Human | PAP | 300 | |
| YNGLLPPYASCHLTE | 15 | Human | PAP | 303 | |
| FAELVGPVIPQDWST | 15 | Human | PAP | 356 | |
| TVPLSEDQLLYLPFR | 15 | Human | PAP | 145 | |
| LTELYFEKGEYFVEM | 15 | Human | PAP | 315 | |
| GPVIPQDWSTECM1T | 15 | Human | PAP | 361 | |
| QAHSLERVCHCLGKW | 21 | Human | PLP | 130 | |
| LGHPDK | | | | | |
| WTTCQSIAFPSKTSASI | 20 | Human | PLP | 181 | |
| GSL | | | | | |
| QKGRGYRGQHQAHS | 20 | Human | PLP | 121 | |
| LERVGH | | | | | |
| AATYNFAVLKLMGR | 18 | Human | PLP | 260 | |
| GTKF | | | | | |
| VATGLGFFGVALFCG | 20 | Human | PLP | 21 | |
| GGHEA | | | | | |
| FLYGALLLAEGFYTT | 20 | Human | PLP | 81 | |
| GAVRQ | | | | | |
| SAVPVYIYFNTWTTC | 20 | Human | PLP | 171 | |
| QSIAF | | | | | |
| TLSVTWIGAAPLILS | 15 | Human | PSA | 5 | |
| SVTWIGAAPLILSRI | 15 | Human | PSA | 7 | |
| VTWIGAAPLILSRIV | 15 | Human | PSA | 8 | |
| SQPWQVLVASRGRAV | 15 | Human | PSA | 31 | |
| GRAVCGGVLVHPQWV | 15 | Human | PSA | 42 | |
| GVLVHPQWVLTAAHC | 15 | Human | PSA | 48 | |
| HPQWVLTAAHCIRNK | 15 | Human | PSA | 52 | |
| QWVLTAAHCIRNKSV | 15 | Human | PSA | 54 | |
| AHCIRNKSVILLGRH | 15 | Human | PSA | 60 | |
| SVILLGRHSLFHPED | 15 | Human | PSA | 67 | |
| VILLGRHSLFHPEDT | 15 | Human | PSA | 68 | |
| GQVFQVSHSFPHPLY | 15 | Human | PSA | 83 | |
| VFQVSHSFPHPLYDM | 15 | Human | PSA | 85 | |
| PHPLYDMSLLKNRFL | 15 | Human | PSA | 93 | |
| SHDLMLLRLSEPAEL | 15 | Human | PSA | 114 | |
| HDLMLLRLSEPAELT | 15 | Human | PSA | 115 | |
| TDAVKVMDLPTQEPA | 15 | Human | PSA | 129 | |
| LHVISNDVCAQVHPQ | 15 | Human | PSA | 172 | |
| CAQVHPQKVTKFMLC | 15 | Human | PSA | 180 | |
| GGPLVCNGVLQGITS | 15 | Human | PSA | 210 | |
| GPLVCNGVLQGITSW | 15 | Human | PSA | 211 | |
| NGVLQGITSWGSEPC | 15 | Human | PSA | 216 | |
| RPSLYTKVVHYRKWI | 15 | Human | PSA | 235 | |
| HSLFHPEDTGQVFQV | 15 | Human | PSA | 74 | |
| PRWLCAGALVLAGGF | 15 | Human | PSM | 18 | |
| LGFLFGWFIKSSNEA | 15 | Human | PSM | 35 | |
| LDELKAENIKKFLYN | 15 | Human | PSM | 62 | |
| TKKFLYNFTQIPHLA | 15 | Human | PSM | 70 | |
| KFLYNFTQIPHLAGT | 15 | Human | PSM | 72 | |
| WKEFGLDSVELAHYD | 15 | Human | PSM | 100 | |
| LAHYDVLLSYPNKTH | 15 | Human | PSM | 110 | |
| GNEIFNTSLFEPPPP | 15 | Human | PSM | 135 | |
| GKVFRGNKVKNAQLA | 15 | Human | PSM | 206 | |
| GNKVKNAQLAGAKGV | 15 | Human | PSM | 211 | |
| EYAYRRGIAEAVGLP | 15 | Human | PSM | 276 | |
| AEAVGLPSIPVHPIG | 15 | Human | PSM | 284 | |
| AVGLPSIPVHPIGYY | 15 | Human | PSM | 286 | |
| IGYYDAQKLLEKMGG | 15 | Human | PSM | 297 | |
| TGNFSTQKVKMHIHS | 15 | Human | PSM | 334 | |
| TRIYNVIGTLRGAVE | 15 | Human | PSM | 353 | |
| ERGVAYINADSSIEG | 15 | Human | PSM | 444 | |
| GVAYINADSSIEGNY | 15 | Human | PSM | 446 | |
| DSSIEGNYTLRVDCT | 15 | Human | PSM | 453 | |
| NYTLRVDCTPLMYSL | 15 | Human | PSM | 459 | |
| CrPLMYSLVHNLTKE | 15 | Human | PSM | 466 | |
| DFEVFFQRLGIASGR | 15 | Human | PSM | 520 | |
| EVFFQRLGIASGRAR | 15 | Human | PSM | 522 | |
| TNKFSGYPLYHSVYE | 15 | Human | PSM | 543 | |
| YDPMFKYHLTVAQVR | 15 | Human | PSM | 566 | |
| DPMFKYHLTVAQVRG | 15 | Human | PSM | 567 | |
| MFKYHLTVAQVRGGM | 15 | Human | PSM | 569 | |
| KYHLTVAQVRGGMVF | 15 | Human | PSM | 571 | |
| VAQVRGGMVFELANS | 15 | Human | PSM | 576 | |
| RGGMVFELANSIVLP | 15 | Human | PSM | 580 | |
| GMVFELANSIVLPFD | 15 | Human | PSM | 582 | |
| VFELANSIVLPFDCR | 15 | Human | PSM | 584 | |
| ADKIYSISMKHPQEM | 15 | Human | PSM | 608 | |
| IYSISMKHPQEMKTY | 15 | Human | PSM | 611 | |
| PQEMKTYSVSFDSLF | 15 | Human | PSM | 619 | |
| TYSVSFDSLFSAVKN | 15 | Human | PSM | 624 | |
| VLRMMNDQLMFLERA | 15 | Human | PSM | 660 | |
| LRMMNDQLMFLERAF | 15 | Human | PSM | 661 | |
| RHVIYAPSSHNKYAG | 15 | Human | PSM | 688 | |
| RQIYVAAFTVQAAAE | 15 | Human | PSM | 730 | |
| QIYVAAFTVQAAAET | 15 | Human | PSM | 731 | |
| VAAFTVQAAAETLSE | 15 | Human | PSM | 734 | |
| YISIINEDGNEIFNT | 15 | Human | PSM | 127 | |
| ISIINEDGNEIFNTS | 15 | Human | PSM | 128 | |
| EDFFKLERDMKINGS | 15 | Human | PSM | 183 | |
| FFKLERDMKINCSGK | 15 | Human | PSM | 185 | |
| GVILYSDPADYFAPG | 15 | Human | PSM | 224 | |
| GAAVVHEIVRSFGTL | 15 | Human | PSM | 391 | |
| NSRLLQERGVAYINA | 15 | Human | PSM | 438 | |
| VAYINADSSIEGNYT | 15 | Human | PSM | 447 | |
| DQLMFLERAFIDPLG | 15 | Human | PSM | 666 | |
| KSNFLNCYVSGFHPSD | 16 | Human B2- | | 19 | |
| | | μglobulin | | | |
| AC- | 18 | IEd | MHC | Unknown | |
| NPDAENWNSQFEILE | | | derived | | |
| DAA | | | | | |
| EYLILSARDVLAVVS | 15 | M. leprae | | 85 | |
| YKTIAYDEEARR | 12 | MT | | 3 | |
| GEALSTLVVNKIRGT | 15 | Mycobacteria | HSP60 | 254 | |
| PYILLVSSKVSTVKD | 15 | Mycobacteria | HSP60 | 216 | |
| EAVLEDPYILLVSSK | 15 | Mycobacteria | HSP60 | 210 | |
| IAGLFLTTEAVVADK | 15 | Mycobacteria | HSP60 | 507 | |
| ALSTLVVNKIRGTFK | 15 | Mycobacteria | HSP60 | 256 | |
| MKHTLYISFYFILVN | 15 | Pf | LSA1 | 1 | |
| KSLLSTNLPYGRTNL | Pf | SSP2 | 116 | | |
| HFFLFLLYILFLVKM | 15 | Pf | | 13 | |
| LFLLYLFLVKMINAL | 15 | Pf | | 16 | |
| ILFLVKMNALRRLPV | 15 | Pf | | 21 | |
| MNALRRLPVICSFLV | 15 | Pf | | 27 | |
| SAFLESQSMNKIGDD | 15 | Pf | | 79 | |
| LKELIKVGLPSFENL | 15 | Pf | | 132 | |
| FENLVAENVKPPKVD | 15 | Pf | | 143 | |
| PATYGIIVPVLTSLF | 15 | Pf | | 158 | |
| YGIIVPVLTSLFNKV | 15 | Pf | | 161 | |
| LLKIWKNYMKIMNHL | 15 | Pf | | 28 | |
| MTLYQIQVMKRNQKQ | 15 | Pf | | 43 | |
| QKQVQMMIMIKFMGV | 15 | Pf | | 57 | |
| MIMIKFMGVIYIMII | 15 | Pf | | 63 | |
| GVIYIMIISKKMMRK | 15 | Pf | | 70 | |
| LYYLFNQHIKKELYH | 15 | Pf | | 285 | |
| HFNMLKNKMQSSFFM | 15 | Pf | | 299 | |
| LDIYQKLYIKQEEQK | 15 | Pf | | 353 | |
| QKKYIYNLIMNTQNK | 15 | Pf | | 366 | |
| YEALIKLLPFSKRIR | 15 | Pf | | 381 | |
| ENEYATGAVRPFQAA | 15 | Pf | | 2 | |
| NYELSKKAVIFTPIY | 15 | Pf | | 27 | |
| QKILIKIPVTKNIIT | 15 | Pf | | 108 | |
| KCLVISQVSNSDSYK | 15 | Pf | | 156 | |
| SKIMKLPKLPISNGK | 15 | Pf | | 202 | |
| FIHFFTWGTMFVPKY | 15 | Pf | | 220 | |
| LCNFKKNIIALLIIP | 15 | Pf | | 242 | |
| KKNIIALLIIPPKIH | 15 | Pf | | 246 | |
| ALLIIPPKIHISIEL | 15 | Pf | | 251 | |
| SMEYKKDFLITARKP | 15 | Pf | | 274 | |
| KSKFNILSSPLFNNF | 15 | Pf | | 7 | |
| FKKLKNHVLFLQMMN | 15 | Pf | | 173 | |
| KNHVLFLQMMNVNLQ | 15 | Pf | | 177 | |
| VLFLQMMNVNLQKQL | 15 | Pf | | 180 | |
| NVNLQKQLLTNHLIN | 15 | Pf | | 187 | |
| QKQLLTNHLINTPKI | 15 | Pf | | 191 | |
| NHLINTPKIMPHHII | 15 | Pf | | 197 | |
| YILLKKILSSRFNQM | 15 | Pf | | 239 | |
| FNQMIFVSSIFISFY | 15 | Pf | | 250 | |
| KVSCKGSGYTFTAYQMH | 17 | Rheumatiod | Variable | | |
| | | vector | region | | |
| IAKVPPGPNITAEYGD | 20 | Rye grass | Lolp1 | 1 | |
| KWLD | | | | | |
| TAEYGDKWLDAKST | 20 | Rye grass | Lolp1 | 11 | |
| WYGKPT | | | | | |
| AKSTWYGKPTGAGPK | 20 | Rye grass | Lolp1 | 21 | |
| DNGGA | | | | | |
| GAGPKDNGGAGGYK | 20 | Rye grass | Lolp1 | 31 | |
| DVDKAP | | | | | |
| FNGMTGCGNTPIFKD | 20 | Rye grass | Lolp1 | 51 | |
| GRGCG | | | | | |
| PIFKDGRGCGSCFEIK | 20 | Rye grass | Lolp1 | 61 | |
| CTKP | | | | | |
| SCFEIKCTKPESCSGE | 20 | Rye grass | Lolp1 | | |
| AVTV | | | | | |
| AFGSMAKKGEEQNVR | 20 | Rye grass | LoIp1 | 111 | |
| SAGEL | | | | | |
| TPDKLTGPFTVRYTTE | 20 | Rye grass | Lolp1 | 201 | |
| GGTK | | | | | |
| VRYTTEGGTKSEVED | 20 | Rye grass | Lolp1 | 211 | |
| VIPEG | | | | | |
| TCVLGKLSQELHKLQ | 15 | Salmon | Calcitonin | 6 | |
| KLSQELHKLQTYPRT | 15 | Salmon | Calcitonin | 11 | |
| LHKLQTYPRTNTGSG | 15 | Salmon | Calcitonin | 16 | |
| KLQTYPRTNTGSGTP | 15 | Salmon | Calcitonin | 18 | |
| CCVLGKLSQELHKLQ | 15 | Salmon | Calcitonin | 7 | A |
| CSNLSTCVLGKLSQE | 15 | Salmon | Calcitonin | 1 | A |
| TSNLSTTVLGKLSQE | 15 | Salmon | Calcitonin | 1 | A |
| TTVLGKLSQELHKLQ | 15 | Salmon | Calcitonin | 6 | A |
| DIAAKYKELGY | 11 | Sperm | Myoglobin | 141 | |
| | | whale | | | |
| ALVRQGLAKVA | 11 | Staph. | Nase | 102 | |
| PATLIKAIDGDTVKLM | 20 | Staph. | Nase | 11 | |
| YKGQ | | | | | |
| TPETKHPKKGVEKYG | 20 | Staph. | Nase | 41 | |
| PEASA | | | | | |
| VEKYGPEASAFTKKM | 20 | Staph. | Nase | 51 | |
| VENAK | | | | | |
| FTKKMVENAKKIEVE | 20 | Staph. | Nase | 61 | |
| FDKGQ | | | | | |
| YIYADGKMVNEALVR | 20 | Staph. | Nase | 91 | |
| QGLAK | | | | | |
| HEQHLRKSEAQAKKE | 20 | Staph. | Nase | 121 | |
| KLNIW | | | | | |
| QAKKEKLNIWSEDNA | 19 | Staph. | Nase | 131 | |
| DSGQ | | | | | |
| YFNNFTVSFWLRVPK | 15 | TetTox | | 947 | |
| FSYFPSI | 7 | TetTox | | 593 | A |
| YSFFPSI | 7 | TetTox | | 593 | A |
| YSYFPSIR | 8 | TetTox | | 593 | A |
| DPNANPNVDPNANPN | 117 | Unknown | (MAP)= | | |
| VNANPNANPNANP(X4) | | | (T1B)4 | | |
| QKWAAVVVPS | 10 | Unknown | ClassI A2 | 242 | |
| TWQLNGEELIQDMEL | 22 | Unknown | ClassI Kb | 216 | |
| VETRPAG | | | 216-237 | | |
| PEFLEQRRAAVDTYC | 15 | Unknown | IEBs2 | | |
| STORKUSP33 | | Unknown | RAGE | | |
| DYSYLQDSDPDSFQD | 15 | Unknown | Tyrosinase | 448 | |
| DFSYLQDSDPDSFQD | 15 | Unknown | Tyrosinase | 448 | SAAS |
| QNILFSNAPLGPQFP | 15 | Unknown | Tyrosinase | 56 | SAAS |
| QNILLSNAPLVPQFP | 15 | Unknown | Tyrosinase | 56 | SAAS |
| DYSYLQDSDPDSFQD | 15 | Unknown | Tyrosinase | 448 | |
| KYVKQNTLKLAT | 11 | unknown | | | |
| P(X)KQNTLKLAT | 13 | unknown | | | A |
| EEDIEIIPIQEEEY | 14 | | CD20 | 249 | A |
| HQAISPRTLNSPAIF | 15 | | | | |
| YTDVFSLDPTFTIETT | 16 | | | | |
| YAGIRiRDGLLLRLVD | 15 | | | | A |
| LFFYRKSVWSKLQSI | 15 | | | | |
| RPIVNMDYVVGARTF | 20 | | | | |
| RREKR | | | | | |
| RPGLLGASVLGLDDI | 15 | | | | |
| LYFVKVDVTGAYDTI | 15 | | | | |
| FAGIRRDGLLLRLVD | 15 | | | | |
| AKTFLRTLVRGVPEY | 15 | | | | |
| YGAVVNLRKTVVNFP | 15 | | | | |
| GTAFVQMPAHGLFPW | 15 | | | | |
| WAGLLLDTRTLEVQS | 15 | | | | |
| RTSIRASLTFNRGFK | 15 | | | | |
| RVIKNSIRLTL | 11 | | | | |
| PVIKNSIKLRL | 11 | | | | |
| ATSTKKLHKEPATLIK | 21 | | | | |
| AIDG | | | | | |

TABLE 26
HLA-DR SUPERTYPE

| | DRB1 | DRB1 | DRB1 | DRB1 | DRB1 | DRB1 | DRB1 | DRB1 |
|---|---|---|---|---|---|---|---|---|
| Sequence | *0101 | *0301 | *0401 | *0404 | *0405 | *0701 | *0802 | *0901 |

| | | | | | | | | | |
|---|---|---|---|---|---|---|---|---|---|
| ACNPTKHKWEA | | | | >900000 | 500000 | | 25000 | | |
| AHVAEQLAA | | | | | | | | | |
| DDYVKQYTKQY | 50000 | | 160 | 500000 | | 12500 | | | |
| TKQNTLKK | | | | | | | | | |
| AAAKAAAAAAY | 833 | >900000 | 229 | 500000 | | 12500 | | | |
| AA | | | | | | | | | |
| ACAAAKAAAAA | 625 | | 348 | | | | | | |
| AYAA | | | | | | | | | |
| (20)AYA(20) | 50000 | | 250 | 500000 | | 8333 | | | |
| A(20)A | | | | | | | | | |
| (20)K(20)A | | | | | | | | | |
| (20) | | | | | | | | | |
| ACAAAKATAAA | 50000 | | 381 | | | | | | |
| AYAA | | | | | | | | | |
| ACAAAKAAAAA | 50000 | | 421 | | | | | | |
| AFAA | | | | | | | | | |
| ACAAAKATAAA | 5000 | | 444 | 500000 | | | | | |
| A(10)AA | | | | | | | | | |
| ACAAAKATAAA | 1250 | | 286 | 25000 | | | | | |
| A(23)AA | | | | | | | | | |
| AAKAAAAAAA | 2500 | | >888.89 | | | | | | |
| (10)AA | | | | | | | | | |
| AAYAAAATAKA | 3.9 | | 0.54 | 2778 | | | | | |
| AA | | | | | | | | | |
| AALAAAAAAKA | 1.9 | | 12 | 152 | | 1316 | | | |
| AA | | | | | | | | | |
| AAEAAAATAKA | 2500 | | 667 | 500000 | | | | | |
| AA | | | | | | | | | |
| AAEAAAATAKA | 50000 | | 533 | 500000 | | | | | |
| AA | | | | | | | | | |
| AAYAAAAJJKA | 1250 | | 308 | 500000 | | | | | |
| AA | | | | | | | | | |
| AFLRAAAAAAF | 50000 | | 400 | 500000 | | | | | |
| AA | | | | | | | | | |
| AFLRQAAAAAF | 2500 | | 1000 | 25000 | | | | | |
| AAY | | | | | | | | | |
| AAFAAAKTAAA | 1.3 | 1063 | 0.19 | 6.2 | | 67 | | | |
| FA | | | | | | | | | |
| YAAFAAAKTAA | 0.74 | | 0.13 | 5.0 | | 34 | | | |
| AFA | | | | | | | | | |
| AALKATAAAAA | 50000 | | 800 | 500000 | | | | | |
| AA | | | | | | | | | |
| YAR(15)ASQT | 1.5 | | 0.46 | 5.2 | | 1196 | | | |
| TLKAKT | | | | | | | | | |
| YARF(33)QTT | 50000 | | 889 | 16667 | | | | | |
| LKAKT | | | | | | | | | |
| PKYFKQRLLKF | 1667 | | 400 | 1042 | | | | | |
| AT | | | | | | | | | |
| PKYFKQGFLKG | 50000 | | 800 | 500000 | | | | | |
| AT | | | | | | | | | |
| PKYGKQIDLKG | 50000 | | 444 | 500000 | | | | | |
| AT | | | | | | | | | |
| AAFFFFFGGGG | 50000 | | 800 | 500000 | | | | | |
| GA | | | | | | | | | |
| AADFFFFFFFF | 1250 | | 286 | 500000 | | | | | |
| DA | | | | | | | | | |
| AAKGIKIGFGI | 50000 | | 471 | 500000 | | | | | |
| FA | | | | | | | | | |
| AAFIFIGGGKI | 50000 | | 195 | 500000 | | | | | |
| KA | | | | | | | | | |
| AAKIFIGFFID | 1250 | | 200 | 25000 | | | | | |
| GA | | | | | | | | | |
| AAFIGFGKIKF | 50000 | | 242 | 500000 | | | | | |
| IA | | | | | | | | | |
| AAKIGFGIKIG | 50000 | | 889 | 500000 | | | | | |
| FA | | | | | | | | | |
| AAFKIGKFGIF | 50000 | | 615 | 500000 | | | | | |
| FA | | | | | | | | | |
| AADDDDDDDDD | 50000 | | 667 | 500000 | | | | | |
| DA | | | | | | | | | |
| (43)AAIGFFF | 50000 | | 258 | 500000 | | | | | |
| FKKGIA | | | | | | | | | |
| (43)AAFFGIF | 50000 | | 381 | 500000 | | | | | |
| KIGKFA | | | | | | | | | |
| (43)AADFGIF | 50000 | | 235 | 500000 | | | | | |
| IDFHA | | | | | | | | | |
| (43)AAIGGIF | 50000 | | 800 | 500000 | | | | | |
| IFKKDA | | | | | | | | | |
| (43)AAFIGFG | 50000 | | 1000 | 500000 | | | | | |
| KIKFIA | | | | | | | | | |
| (43)AAKIGFG | 50000 | | 1000 | 500000 | | | | | |
| IKIGFA | | | | | | | | | |
| (43)AAFKIGK | 50000 | | 276 | 500000 | | | | | |
| FGIFFA | | | | | | | | | |
| AAAKAAAAAAA | >1666.67 | | >347.83 | 12500 | | | | | |
| AF | | | | | | | | | |
| AAAKAAAAAAA | 50000 | | 727 | 500000 | | | | | |
| FA | | | | | | | | | |
| AAAKAAAAAAF | 50000 | | 235 | 25000 | | | | | |
| AA | | | | | | | | | |
| AAAKAAAAFAA | 50000 | | 533 | 500000 | | | | | |
| AA | | | | | | | | | |
| FAAAAAAAAAA | 1667 | | 200 | 8333 | | | | | |
| AA | | | | | | | | | |
| AAAAAAAAAAA | 50000 | | 500 | 500000 | | | | | |
| AN | | | | | | | | | |
| AAAAAAAAAAA | 50000 | | 1000 | 500000 | | | | | |
| NA | | | | | | | | | |
| AAANAAAAAAA | 50000 | | 615 | 500000 | | | | | |
| AA | | | | | | | | | |
| AAAAAAAAAAA | 50000 | | 533 | 500000 | | | | | |
| AS | | | | | | | | | |
| AAAAASAAAAA | 50000 | | 235 | 500000 | | | | | |
| AA | | | | | | | | | |
| ASAAAAAAAAA | 50000 | | 364 | 500000 | | | | | |
| AA | | | | | | | | | |
| AFAAAKTAA | 50000 | | 571 | 500000 | | | | | |
| YARFLALTTLR | 0.98 | | 0.28 | 3.4 | | | | | |
| ARA | | | | | | | | | |
| YAR(15A)SQT | 2.4 | | 0.78 | 5.2 | | 1786 | | | |
| TLKAKT | | | | | | | | | |
| YAR(15A)RQT | 1.6 | | 0.35 | 3.8 | | 8333 | | | |
| TLKA | | | | | | | | | |
| (15A)RQTTLK | 4.2 | | 0.31 | 4.3 | | 250000 | | | |
| AAA | | | | | | | | | |
| (16A)RQTTLK | 455 | | 1.3 | 37 | | 250000 | | | |
| AAA | | | | | | | | | |
| (46)AAKTAAA | 5000 | | 571 | 1852 | | | | | |
| FA | | | | | | | | | |
| (39)AAAATKA | 3333 | | 727 | 500000 | | | | | |
| AA | | | | | | | | | |
| (52)AAAATKA | 2000 | | 242 | 2632 | | | | | |
| AAA | | | | | | | | | |
| (55)AAAATKA | 2500 | | 667 | 5556 | | | | | |
| AAA | | | | | | | | | |
| A(14)AAAKTA | 39 | | 0.45 | 54 | | 96 | | | |
| AA | | | | | | | | | |
| AA(14)A(35) | 50000 | | >500 | 500000 | | | | | |
| ATKAAAA | | | | | | | | | |
| AA(14)AA | 50000 | | 667 | 25000 | | | | | |
| (36)TKAAAA | | | | | | | | | |
| AFAAAKTAA | 5000 | | 533 | 500000 | | | | | |
| (72) | | | | | | | | | |
| (49)AAAKT | 50000 | | 667 | 500000 | | | | | |
| (64)AAA | | | | | | | | | |
| (49)AAAKTA | 50000 | | 533 | 500000 | | | | | |
| (64)AA | | | | | | | | | |
| HQAISPRTLNG | 1555 | 728464 | 12089 | 2056 | 3107 | 5081 | | | |
| PGPGSPAIF | | | | | | | | | |
| YAAFAAAKTAA | 1.9 | | 0.82 | 7.0 | | | | | |
| AFA | | | | | | | | | |
| TEGRCLHYTVD | 1667 | | 200 | 500000 | | >250000 | | | |
| KSKPK | | | | | | | | | |
| AWVAWRNRCK | 50000 | | 667 | 500000 | | >12500 | | | |
| TVSDGNGMNAW | 1250 | 18371 | 1000 | 8333 | | >8333.33 | | | |
| VAWRNRC | | | | | | | | | |
| PHHTALRQAIL | 1250 | | 166 | 1773 | | 14434 | | | |
| SWGELMTLA | | | | | | | | | |
| WMYYHGQRHSD | 50000 | >900000 | 727 | 500000 | | >250000 | | | |
| EHHH | | | | | | | | | |
| YIVMSDWTGGA | 50000 | 13416 | 222 | 500000 | | 12500 | | | |
| AHAAHAAHAAH | 263 | | 80000 | 500000 | | >250000 | | | |
| AAHAA | | | | | | | | | |
| MDIDPYKEFGA | 1563 | | 170 | | | 6609 | | | |
| TV | | | | | | | | | |
| ELLSFLPSDFF | | | | | | | | | |
| P | | | | | | | | | |
| GMLPVCPLIPG | 1250 | >900000 | 400 | 1220 | | 250000 | | | |
| SSTTSTGP | | | | | | | | | |
| LGFFPDHQLDP | 1667 | 12027 | 333 | 2941 | | 250000 | | | |
| AFRANT | | | | | | | | | |
| GYKVLVLNPSV | 16 | 72407 | 27 | 2116 | 145 | 1516 | 115 | 8789 | |
| LMAFTAAVTS | 2511 | >73952.34 | 321 | 20577 | 627 | 240 | >40562.91 | 160 | |
| TFALWRVSAEE | >5279.83 | 88348 | 342 | 569 | 72 | 927 | 1433 | 517 | |
| Y | | | | | | | | | |
| ALWRVSAEEY | >6337.14 | >76595.74 | 6543 | 6669 | >35315.99 | 7954 | 4099 | 698 | |
| EEYVEIRQVGD | >1957.71 | 74884 | >5365.53 | 11627 | 26 | 11323 | 13890 | 11154 | |
| FH | | | | | | | | | |
| VGGVYLLPRRG | 177 | 236639 | 22323 | 12756 | 2764 | 351 | | | |
| PRLGV | | | | | | | | | |
| VGGAYLLPRRG | 131 | 308534 | 26164 | 125056 | >12230.45 | 703 | | | |
| PRLGV | | | | | | | | | |
| VGGVALLPRRG | 849 | 326288 | 48233 | 23669 | >12230.45 | 61558 | | | |
| PRLGV | | | | | | | | | |
| VGGVYALPRRG | 134 | 348950 | 25750 | 30504 | >12230.45 | 749 | | | |
| PRLGV | | | | | | | | | |
| VGGVYLAPRRG | 746 | 202660 | 33672 | >116550.12 | >12230.45 | 878 | | | |
| PRLGV | | | | | | | | | |
| VGGVYLLARRG | 60 | 23276 | 485 | 4396 | 2199 | 595 | | | |
| PRLGV | | | | | | | | | |
| VGGVYLLPARG | 12 | 68070 | 3644 | 3213 | 4579 | 49 | | | |
| PRLGV | | | | | | | | | |
| VGGVYLLRRAG | 202 | 39751 | 12252 | 32330 | 6432 | 433 | | | |
| PRLGV | | | | | | | | | |
| GAPLGGAARAL | 690 | 3145 | 10408 | 19762 | >13044.97 | 10773 | | | |
| AHGV | | | | | | | | | |
| GAALGGAARAL | 1081 | 26944 | 21362 | 60600 | >13044.97 | 29786 | | | |
| AHGV | | | | | | | | | |
| GAPLAGAARAL | 588 | 2983 | 39885 | 19692 | >13044.97 | 8178 | | | |
| AHGV | | | | | | | | | |
| GAPLGAAARAL | 226 | 17703 | 10255 | 52041 | >13044.97 | 6490 | | | |
| AHGV | | | | | | | | | |
| GAPLGGLARAL | 537 | 351525 | 13941 | 6564 | >13044.97 | 66 | | | |
| AHGV | | | | | | | | | |
| GAPLGGALRAL | 68 | >486486.49 | 14977 | 977 | 1271 | 1418 | | | |
| AHGV | | | | | | | | | |
| GAPLGGAAAAL | 147 | 82088 | 5472 | 1272 | >3365.21 | 31907 | | | |
| AHGV | | | | | | | | | |
| GAPLGGAARLL | 398 | 22959 | 14984 | 21017 | >3365.21 | 57549 | | | |
| AHGV | | | | | | | | | |
| GAPLGGAARAA | 797 | 377964 | 25279 | >110132.16 | >3365.21 | 31308 | | | |
| AHGV | | | | | | | | | |
| GAPLGGAARAL | 541 | 23298 | 11270 | 16747 | >3365.21 | 7419 | | | |
| AAGV | | | | | | | | | |
| FPDWQNYTPGP | 13766 | >223880.6 | 23394 | >109170.31 | >10101.01 | 59625 | 592 | 3013 | |
| GTRF | | | | | | | | | |
| RFPLTFGWCFK | 5913 | 406579 | 316 | 21384 | 121 | 4100 | 748 | 1848 | |
| LVPV | | | | | | | | | |
| RQDILDLWVYH | 2390 | 98327 | 1202 | 1624 | 1136 | 1628 | 5039 | 1665 | |
| TQGY | | | | | | | | | |
| RQEILDLWVYH | 1050 | 10530 | 5928 | 1414 | 3362 | 3052 | 2730 | 3679 | |
| TQGF | | | | | | | | | |
| LSHFLKEKGGL | 537 | >340909.09 | 2442 | 86814 | 2114 | 13676 | 1561 | 23191 | |
| EGL | | | | | | | | | |
| LSFFLKEKGGL | 172 | >340909.09 | 1275 | >109170.31 | 983 | 19957 | 1127 | 3501 | |
| DGL | | | | | | | | | |
| LEPWNHPGSQP | >33557.05 | >328467.15 | >33333.33 | >96525.1 | >8232.24 | >72254.34 | 69223 | 34468 | |
| KTACT | | | | | | | | | |
| QVCFITKGLGI | 114 | 166744 | 1529 | 1391 | 295 | 91 | 41 | 296 | |
| SYGR | | | | | | | | | |
| QLCFLKKGLGI | 185 | 158381 | 4436 | 1613 | 443 | 3634 | 40 | 200 | |
| SYGR | | | | | | | | | |
| PPEESFRFGEE | >2500 | >900000 | 267 | 500000 | | >12500 | | | |
| KTTPS | | | | | | | | | |
| CIVYRDGNPYA | 8464 | | 147 | 1084 | 3473 | >17182.13 | | 31865 | |
| VCDK | | | | | | | | | |
| HYCYSLYGTTL | 546 | | 1127 | 9713 | 76 | 9858 | | 12359 | |
| EQQY | | | | | | | | | |
| CYSLYGTTLEQ | 1086 | | 1317 | 2836 | 71 | >9964.13 | 25989 | | |
| QYNK | | | | | | | | | |
| NTSLQDIEITC | >12106.54 | | 10930 | 6143 | 4584 | >17182.13 | | 30884 | |
| VYCK | | | | | | | | | |
| VFEFAFKDLFV | 6716 | | 1059 | 2156 | 120 | 11583 | | 16797 | |
| VYRD | | | | | | | | | |
| EFAFKDLFVVY | 8944 | | 2220 | 11721 | 33 | 3688 | | 1882 | |
| RDS | | | | | | | | | |
| DLFVVYRDSIP | 1186 | | 82 | 218 | 3591 | 5213 | | 2374 | |
| HAAC | | | | | | | | | |
| FVVYRDSIPHA | 587 | 200 | 10 | 87 | 704 | 5085 | | 2122 | |
| ACHK | | | | | | | | | |
| NTGLYNLLIRC | 127 | 13429 | 686 | 358 | 258 | 6743 | | 4759 | |
| LRCQ | | | | | | | | | |
| IRCLRCQKPLN | 7240 | | 6334 | 8464 | 1229 | 16787 | | 32024 | |
| PAEK | | | | | | | | | |
| PRKLHELSSAL | 156 | 16146 | 5276 | 694 | 80 | 103 | | 213 | |
| EIPY | | | | | | | | | |
| EIPYDELRLNC | 3299 | | 15532 | 11292 | 7321 | >35612.54 | | >39432.18 | |
| VYCK | | | | | | | | | |
| TEVLDFAFTDL | 2073 | 1542 | 185 | 1083 | 871 | 1432 | | 349 | |
| TIVY | | | | | | | | | |
| VLDFAFTDLTI | 354 | 30 | 313 | 6061 | 721 | 230 | | 252 | |
| VYRD | | | | | | | | | |
| DFAFTDLTIVY | 463 | 23 | 80 | 3373 | 40 | 725 | | 1443 | |
| RDDT | | | | | | | | | |
| TIVYRDDTPHG | 3798 | | 22 | 1269 | >9753.59 | >35612.54 | | >39144.05 | |
| VCTK | | | | | | | | | |
| WYRYSVYGTTL | 163 | 26561 | 249 | 3448 | 8.5 | 107 | | 284 | |
| EKLT | | | | | | | | | |
| ETTIHNIELQC | 3623 | | 1996 | 3327 | 6561 | >35612.54 | | >39432.18 | |
| VECK | | | | | | | | | |
| SEVYDFAFADL | 31 | 2996 | 260 | 2180 | 101 | 1850 | | 174 | |
| TVVY | | | | | | | | | |
| VYDFAFADLTV | 173 | | 119 | 5281 | 133 | 7012 | | 155 | |
| VYRE | | | | | | | | | |
| DFAFADLTVVY | 3293 | | 141 | 4948 | 60 | 1728 | | 322 | |
| REGN | | | | | | | | | |
| TVVYRBGNPFG | 168 | | 121 | 1833 | >13089.91 | 10064 | | 2407 | |
| ICKL | | | | | | | | | |
| GNPFGICKLCL | 189 | | 1227 | 2073 | 377 | 13916 | | 45631 | |
| RFLS | | | | | | | | | |
| NYSVYGNTLEQ | 14059 | | 1933 | 91506 | 822 | >14602.8 | | 47481 | |
| TVKK | | | | | | | | | |
| KKPLNEILIRC | 1363 | | 315 | 1070 | 347 | 7972 | | 13328 | |
| IICQ | | | | | | | | | |
| NEILIRCIICQ | 7945 | | 11739 | 23082 | 7704 | 16901 | | 26483 | |
| RPLC | | | | | | | | | |
| IRCIICQRPLC | 7549 | | 5960 | 23092 | 2973 | >14602.8 | | 40269 | |
| PQEK | | | | | | | | | |
| CIVYRDCIAYA | 11166 | | 928 | 8560 | 3973 | >14602.8 | | 10186 | |
| ACHK | | | | | | | | | |
| NTELYNLLIRC | 1108 | | 1366 | 1293 | 873 | >14602.8 | | 12528 | |
| LRCQ | | | | | | | | | |
| IRCLRCQKPLN | 7012 | | 6668 | 9890 | 8982 | >14602.8 | | >32271.94 | |
| PAEK | | | | | | | | | |
| REVYKFLFTDL | 8.7 | 23 | 112 | 738 | 52 | 54 | | 204 | |
| RIVY | | | | | | | | | |
| RTVYRDNNPYG | 524 | 325 | 20 | 432 | 2307 | 8307 | | 24147 | |
| VCIM | | | | | | | | | |
| NNPYGVCIMCL | 1075 | | 1378 | 2522 | 454 | 12020 | | 30895 | |
| RFLS | | | | | | | | | |
| EERVKKPLSEI | 1286 | | 11896 | 9772 | 1470 | 9454 | | 19968 | |
| TIRC | | | | | | | | | |
| IRCIICQTPLC | 10847 | | 12270 | 3812 | 1407 | 25186 | | 28062 | |
| PEEK | | | | | | | | | |
| EIPLIDLRLSC | 7610 | | 1876 | 5012 | 336 | 10468 | | 1961 | |
| VYCK | | | | | | | | | |
| SCVYCKKELTR | 6466 | | 2411 | 7510 | 465 | 8446 | | 2010 | |
| AEVY | | | | | | | | | |
| VCLLFYSKVRK | 960 | 276 | 286 | 987 | 73 | 258 | | 1798 | |
| YRYY | | | | | | | | | |
| YYDYSVYGATL | 1008 | | 186 | 9855 | 230 | 744 | | 1403 | |
| ESIT | | | | | | | | | |
| IRCYRCQSPLT | 10947 | | 13358 | 83166 | 10327 | 13356 | | >36023.05 | |
| PEEK | | | | | | | | | |
| VYDFVFADLRI | 98 | 2.2 | 475 | 5856 | 717 | 5962 | | 198 | |
| VYRD | | | | | | | | | |
| DFVFADLRJVY | 6699 | | 867 | 7197 | 133 | 9847 | | 1962 | |
| RDGN | | | | | | | | | |
| RIVYRDGNPFA | 116 | 144 | 19 | 209 | 1812 | 6638 | | 4962 | |
| VCKV | | | | | | | | | |
| GNPFAVCKVCL | 134 | 3805 | 322 | 522 | 56 | 1034 | | 29300 | |
| RLLS | | | | | | | | | |
| KKCLNEILIRC | 9357 | | 424 | 1229 | 365 | 16288 | | 3997 | |
| HCQ | | | | | | | | | |
| NEILIRCIICQ | 10992 | | 14069 | 9339 | 4621 | 18947 | | 22062 | |
| RPLC | | | | | | | | | |
| RTAMFQDPQER | 9372 | 154 | 28192 | 39014 | 7977 | 32947 | | >25346.4 | |
| PRKL | | | | | | | | | |
| LFVVYRDSIPH | 131 | 62 | 3.0 | 24 | 690 | 1998 | | 2855 | |
| AACH | | | | | | | | | |
| LTIVYRDDTPH | >15384.62 | 187 | 23 | 203 | >8593.4 | >72254.34 | | >25346.4 | |
| GVCT | | | | | | | | | |
| LCIVYRDCIAY | 996 | 1855 | 357 | 1293 | 628 | 40121 | | 10660 | |
| AACH | | | | | | | | | |
| YKFLFTDLRIV | 109 | 8.8 | 292 | 256 | 91 | 1516 | | 1255 | |
| YRDN | | | | | | | | | |
| YNFACTELKLV | 7522 | 346 | 1976 | 4246 | 3147 | 2867 | | 2084 | |
| YRDD | | | | | | | | | |
| LKLVYRDDFPY | 778 | 237 | 123 | 9269 | 830 | 28971 | | 18677 | |
| AVCR | | | | | | | | | |
| YDFVFADLRIV | 1160 | 13 | 1914 | 3264 | 829 | 21352 | | 5419 | |
| YRNG | | | | | | | | | |
| LRIVYRDGNPF | 142 | 181 | 16 | 25 | 557 | 8985 | | 14207 | |
| AVCK | | | | | | | | | |
| HEYMLDLQPET | 1377 | | 222 | 3997 | 2291 | >18559.76 | | 21277 | |
| TDLY | | | | | | | | | |
| TLRLCVQSTHV | 1517 | | 11996 | 8650 | 169 | 3257 | | 6368 | |
| DIRT | | | | | | | | | |
| IRTLEDLLMGT | 16 | 5211 | 95 | 43 | 61 | 895 | | 1718 | |
| LGIV | | | | | | | | | |
| LEDLLMGTLGI | 104 | | 1136 | 353 | 1116 | 261 | | 1994 | |
| VCP | | | | | | | | | |
| DLLMGTLGIVC | 966 | | 1324 | 984 | 639 | 963 | | 2614 | |
| PICS | | | | | | | | | |
| KATLQDIVLHL | 1204 | | 1987 | 811 | 1173 | 9094 | | 17726 | |
| EPQN | | | | | | | | | |
| IDGVNHQHLPA | 1060 | | 34272 | 165545 | >16971.86 | >18559.76 | | >39914.85 | |
| RRAE | | | | | | | | | |
| LRAFQQLFLNT | 1.5 | 648 | 7.4 | 13 | 8.3 | 75 | | 174 | |
| LSFV | | | | | | | | | |
| FQQLFLNTLSF | 118 | 1321 | 134 | 1585 | 222 | 134 | | 2062 | |
| VCPW | | | | | | | | | |
| QDYVLDLQPEA | 13441 | | 253 | 45281 | 5585 | >18559.76 | | >39914.85 | |
| TDLH | | | | | | | | | |
| DIRILQELLMG | 88 | 3252 | 166 | 290 | 552 | 1591 | | 282 | |
| SFGI | | | | | | | | | |
| IRILQELLMGS | 67 | 31840 | 724 | 710 | 1208 | 1998 | | 271 | |
| FGIV | | | | | | | | | |
| ELLMGSFGIVC | 628 | | 1078 | 8518 | 1853 | 4183 | | 949 | |
| PNCS | | | | | | | | | |
| KEYVLDLYPEP | 5949 | | 131 | 89674 | 391 | >72254.34 | | >49867.02 | |
| TDLY | | | | | | | | | |
| LRTIQQLLMGT | 13 | 23182 | 108 | 208 | 179 | 513 | | 181 | |
| VNIV | | | | | | | | | |
| IQQLLMGTVNI | 71 | 93701 | 107 | 483 | 624 | 444 | | 156 | |
| YCPT | | | | | | | | | |
| QLLMGTVNIVC | 1192 | | 2874 | 10062 | 4688 | 2947 | | 2209 | |
| PTCA | | | | | | | | | |
| RETLQEIVLHL | 1592 | | 2941 | 6583 | 829 | 25856 | | 19109 | |
| EPQN | | | | | | | | | |
| LRTLQQLFLST | 8.3 | 801 | 18 | 18 | 9.0 | 60 | | 166 | |
| LSFV | | | | | | | | | |
| LQQLFLSTLSF | 121 | 2045 | 113 | 754 | 94 | 272 | | 152 | |
| VCPW | | | | | | | | | |
| KDYILDLQPET | 6409 | | 1022 | 30309 | 2771 | >72254.34 | | >49867.02 | |
| TDLH | | | | | | | | | |
| LRTLQQMLLGT | 80 | >3750000 | 437 | 644 | 79 | 6909 | | 5077 | |
| LQVV | | | | | | | | | |
| LQQMLLGTLQV | 168 | | 1496 | 631 | 1068 | 929 | | 1692 | |
| VCPG | | | | | | | | | |
| QMLLGTLQVVC | 957 | | 2773 | 425 | 3074 | 3722 | | 2082 | |
| PGCA | | | | | | | | | |
| VPTLQDVVLEL | 16056 | | 214 | 4764 | 5409 | >35360.68 | | >30612.24 | |
| TPQT | | | | | | | | | |
| LQDVVLELTPQ | 1487 | | 101 | 1094 | 417 | 5673 | | 2180 | |
| TEID | | | | | | | | | |
| QDVVLELTPQT | 1269 | | 83 | 1537 | 53 | 2716 | | 1684 | |
| EIDL | | | | | | | | | |
| CKFVVQLDIQS | 1251 | | 196 | 1642 | 374 | 4547 | | 19282 | |
| TKED | | | | | | | | | |
| VVQLDIQSTKE | 1060 | | 11122 | 8625 | 46 | 3762 | | 13906 | |
| DLRV | | | | | | | | | |
| DLRVVQQLLMG | 8.4 | 25971 | 325 | 89 | 84 | 508 | | 1845 | |
| ALTV | | | | | | | | | |
| LRVVQQLLMGA | 5.7 | 21650 | 115 | 28 | 85 | 82 | | 204 | |
| LTVT | | | | | | | | | |
| VQQLLMGALTV | 10 | 34257 | 239 | 614 | 116 | 71 | | 180 | |
| TCPL | | | | | | | | | |
| QQLLMGALTVT | 75 | | 1142 | 1286 | 201 | 743 | | 1170 | |
| CPLC | | | | | | | | | |
| QLLMOALTVTC | 54 | >3750000 | 595 | 870 | 1019 | 389 | | 303 | |
| PLCA | | | | | | | | | |
| RBYILDLHPEP | 154 | | 132 | 9957 | 354 | 7257 | | 29316 | |
| TDLF | | | | | | | | | |
| TCCYTCGTTVR | 1230 | 19884 | 719 | 2269 | 132 | 63 | | 1374 | |
| LCIN | | | | | | | | | |
| VRTLQQLLMQT | 36 | 32360 | 322 | 39 | 114 | 1820 | | 496 | |
| CTIV | | | | | | | | | |
| LQQLLMGTCTI | 197 | | 1147 | 483 | 522 | 2098 | | 1638 | |
| VCPS | | | | | | | | | |
| MLDLQPETTDL | 10076 | 720 | 1913 | 12241 | 4249 | >72254.34 | | >32230.34 | |
| YCYE | | | | | | | | | |
| VLDLYPEPTDL | 11201 | 121 | 203 | 2193 | 212 | >72254.34 | | >32230.34 | |
| YCYE | | | | | | | | | |
| LRBYILDLHPE | 134 | 891 | 23 9235 | 968 | 21989 | | 16462 | | |
| PTDL | | | | | | | | | |
| HIEFTPTRTDT | | 50000 | 30000 | 667 | 10000 | | >12500 | | |
| YACRV | | | | | | | | | |
| LWWVNNESLPV | | 315 | | | | | | | |
| SPRL | | | | | | | | | |
| YEEYVRFDSDV | 50000 | | 400 | 500000 | | 250000 | | | |
| GE | | | | | | | | | |
| EEYVRFDSDVG | 50000 | | 216 | 500000 | | 250000 | | | |
| E | | | | | | | | | |
| APPRLICDSRV | 1374 | 6.3 | 9735 | 5794 | 7141 | 8937 | 11214 | 9348 | |
| LERY | | | | | | | | | |
| ICDSRVLERYL | 2758 | 236 | 1984 | 10984 | 11016 | 57605 | 808 | >78947.37 | |
| LEAK | | | | | | | | | |
| VLERYLLEAYE | 933 | 59010 | 2598 | 12139 | 5019 | 13067 | 3150 | 6382 | |
| AENI | | | | | | | | | |
| EHCSLNENITV | 9837 | 27481 | 2294 | 28297 | 1205 | 32375 | 6191 | >78947.37 | |
| PDTK | | | | | | | | | |
| NENITVPDTKV | >24154.59 | 4.8 | >21390.37 | 7612 | >18572.83 | 42846 | 1850 | >78947.37 | |
| NFYA | | | | | | | | | |
| VPDTKVNFYAW | 2764 | 259 | 1742 | 4131 | 1328 | 38622 | 422 | >78947.37 | |
| KRME | | | | | | | | | |
| VNFYAWKRMEV | 193 | 2871 | 10 | 291 | 15 | 40163 | 35 | 1238 | |
| GQQA | | | | | | | | | |
| WKRMEVGQQAV | 62 | 514 | 24 | 2591 | 94 | 46062 | 139 | 14696 | |
| EVWQ | | | | | | | | | |
| VGQQAVEVWQG | 161 | >174081.24 | 10294 | 6283 | 923 | 4230 | >40511.09 | >78947.37 | |
| LALL | | | | | | | | | |
| VEVWQGLALLS | 86 | 13293 | 1310 | 1357 | 79 | 6863 | 13411 | 8151 | |
| EAVL | | | | | | | | | |
| GLALLSEAVLR | 83 | 816 | 11 | 21 | 1435 | 4606 | 2000 | 15148 | |
| GQAL | | | | | | | | | |
| SEAVLRGQALL | 11 | 70855 | 2064 | 4207 | 17446 | 1087 | >63636.36 | >78947.37 | |
| VNSS | | | | | | | | | |
| RGQALLVNSSQ | 1118 | 93874 | 1697 | 1168 | 3434 | 319 | 29454 | 8450 | |
| PWEP | | | | | | | | | |
| LVNSSQPWEPL | 2178 | 26138 | >21505.3 | 13031 | 19689 | 8344 | 16920 | >78947.37 | |
| QLNV | | | | | | | | | |
| QPWEPLQLHVD | 11567 | 4862 | 1296 | 6135 | 1111 | 24157 | >63636.36 | 34819 | |
| KAVS | | | | | | | | | |
| LQLHVDKAVSG | 192 | 22 | 9.7 | 44 | 13571 | 3213 | 801 | >78947.37 | |
| LRSL | | | | | | | | | |
| DKAVSGLRSLT | 13 | 4331 | 1014 | 25 | 247 | 615 | 16375 | >78947.37 | |
| TLLR | | | | | | | | | |
| GLRSLTTLLRA | 8.5 | 2345 | 24 | 9.2 | 30 | 509 | 14 | 1136 | |
| LGAQ | | | | | | | | | |
| TTLLRALGAQK | 19 | 107164 | 339 | 199 | 103 | 4281 | 652 | 4607 | |
| EAIS | | | | | | | | | |
| ALGAQKEAISP | 194 | >204081.63 | >21505.3 | 93062 | 13015 | >71225.07 | >60214.56 | 15337 | |
| PDAA | | | | | | | | | |
| KEAISPPDAAS | 15531 | 48560 | 6590 | 4389 | 28755 | 6661 | 6391 | 5735 | |
| AAPL | | | | | | | | | |
| PPDAASAAPLR | 309 | 14900 | 566 | 68 | 1555 | 24937 | >63636.36 | 8674 | |
| TITA | | | | | | | | | |
| SAAPLRTITAD | 1166 | 1262 | 1185 | 261 | 1456 | 3646 | 28110 | 2505 | |
| TFRK | | | | | | | | | |
| RTITADTFRKL | 148 | 139 | 1042 | 928 | 1957 | 3448 | 792 | 4692 | |
| FRVY | | | | | | | | | |
| DTFRKLFRVYS | 12 | 6946 | 70 | 104 | 93 | 10 | 39 | 307 | |
| NFLR | | | | | | | | | |
| LFRVYSNFLRG | 43 | 6156 | 643 | 1816 | 1275 | 5.5 | 28 | 3508 | |
| KLKL | | | | | | | | | |
| SNFLRGKLKLY | 143 | 9583 | 2883 | 2375 | 7182 | 3783 | 1433 | 8099 | |
| TGEA | | | | | | | | | |
| KLKLYTGEACR | 122 | 18435 | 5964 | 3505 | 36294 | 8082 | 7683 | 2860 | |
| TGDR | | | | | | | | | |
| APPRLITDSRV | 10144 | 15 | 6680 | 3168 | 7765 | 629 | 26382 | 8391 | |
| LERY | | | | | | | | | |
| ITDSRVLERYL | 1571 | 6501 | 1303 | 1990 | 13339 | 7498 | 967 | >78947.37 | |
| LEAK | | | | | | | | | |
| EHTSLNENITV | 43921 | 33635 | 12379 | 2769 | 1245 | 37154 | >16333.33 | >78947.37 | |
| PDTK | | | | | | | | | |
| KLKLYTGEATR | 178 | 118459 | 15 | 3230 | 1426 | 8234 | 2008 | >78947.37 | |
| TGDR | | | | | | | | | |
| PQPFRPQQPYP | | | | | | | | | |
| Q | | | | | | | | | |
| PFRPQQPYPQ | | | | | | | | | |
| PQPFRPQQPYP | | | | | | | | | |
| PQPFRPQQP | | | | | | | | | |
| KQPFRPQQPYP | | | | | | | | | |
| Q | | | | | | | | | |
| PKPFRPQQPYP | | | | | | | | | |
| Q | | | | | | | | | |
| PQPFKPQQPYP | | | | | | | | | |
| Q | | | | | | | | | |
| PQPFRKQQPYP | | | | | | | | | |
| Q | | | | | | | | | |
| PQPFRPQKPYP | | | | | | | | | |
| Q | | | | | | | | | |
| PQPFRPQQPKP | | | | | | | | | |
| Q | | | | | | | | | |
| PQPFRPQQPYK | | | | | | | | | |
| Q | | | | | | | | | |
| PQPFRPQQPYP | | | | | | | | | |
| K | | | | | | | | | |
| QFLGQQQPFPP | | | | | | | | | |
| Q | | | | | | | | | |
| FLGQQQPFPPQ | | | | | | | | | |
| LGQQQPFPPQ | | | | | | | | | |
| QFLGQQQPFPP | | | | | | | | | |
| QFLGQQQPF | | | | | | | | | |
| IRNLALQTLPA | | | | | | | | | |
| MCNVY | | | | | | | | | |
| NLALQTLPAMC | | | | | | | | | |
| NVY | | | | | | | | | |
| LALQTLPAMCN | | | | | | | | | |
| VY | | | | | | | | | |
| IRNLALQTLPA | | | | | | | | | |
| M | | | | | | | | | |
| IRNLALQTLP | | | | | | | | | |
| EGDAFELTVSC | | | 572 | 3578 | | | | | |
| QGGLPK | | | | | | | | | |
| ESTGMTPEKVP | | >50000 | >47368.42 | 510 | >71428.57 | | >31250 | | |
| VSEVMGT | | | | | | | | | |
| FPTIPLSRLFD | 8071 | 114611 | 228 | 22 | 7210 | 3175 | 4969 | 9876 | |
| NASL | | | | | | | | | |
| RLFDNASLRAH | 89 | 97 | 77 | 2043 | 10328 | 1921 | 14985 | 23832 | |
| RLHQ | | | | | | | | | |
| LRAHRLHQLAF | 162 | 15603 | 5076 | 2197 | 10139 | 123 | 5621 | 15115 | |
| DTYQ | | | | | | | | | |
| QLAFDTYQEFE | >20491.8 | 7981 | >10738.2 | 33446 | 5399 | 2580 | >33333.33 | >59523.81 | |
| EAYI | | | | | | | | | |
| QEFEEAYIPKE | >20491.8 | >171755.73 | >21276.6 | >88339.22 | 395 | 31344 | >33333.33 | >59523.81 | |
| QKYS | | | | | | | | | |
| IPKEQKYSFLQ | 128 | 49978 | 217 | 3633 | 9.0 | 8305 | 13553 | 79800 | |
| NPQT | | | | | | | | | |
| SFLQNPQTSLC | 595 | 8617 | 6376 | 16880 | >25832.77 | 48620 | >33333.33 | 93856 | |
| FSES | | | | | | | | | |
| TSLCFSESIPT | 604 | 182762 | 48 | 229 | 852 | 1064 | >33333.33 | 4395 | |
| PSNR | | | | | | | | | |
| REETQQKSNLE | 8921 | 91054 | 9341 | 1324 | 1433 | 51179 | 22467 | 9680 | |
| LLR | | | | | | | | | |
| SNLELLRISLL | 72 | 43487 | 621 | 189 | 379 | 642 | >33333.33 | 3422 | |
| LIQS | | | | | | | | | |
| ISLLLIQSWLE | 184 | 27922 | 885 | 177 | 0.86 | 83 | >33333.33 | 6247 | |
| PVQF | | | | | | | | | |
| SWLEPVQFLRS | 11 | 167103 | 1128 | 152 | 883 | 589 | 3416 | 3998 | |
| VFAN | | | | | | | | | |
| FLRSVFANSLV | 4.3 | 15221 | 6.7 | 43 | 59 | 16 | 13436 | 15127 | |
| YGAS | | | | | | | | | |
| NSLVYGASDSN | 7313 | 81158 | 190 | 1585 | 1055 | 201 | >33333.33 | 3896 | |
| VYDL | | | | | | | | | |
| SDSNVYDLLKN | 24369 | 54982 | 11032 | >25680.53 | 95 | 182355 | >33333.33 | >59523.81 | |
| LEEG | | | | | | | | | |
| GIQTLMGRLED | 98 | >55900.62 | 11914 | 2458 | 3745 | 18952 | >33333.33 | 37821 | |
| GSPR | | | | | | | | | |
| RLEDGSPRTGQ | 15693 | 76675 | 7906 | 1729 | 22125 | 35120 | >33333.33 | >59523.81 | |
| IFKQ | | | | | | | | | |
| RTGQIFKQTYS | 1555 | 20341 | 1680 | 1831 | 40 | 46 | 16432 | 8515 | |
| KFDT | | | | | | | | | |
| QTYSKFDTNSH | 17352 | >55900.62 | 97 | 11218 | 78 | 54569 | 7726 | 31341 | |
| NDDA | | | | | | | | | |
| TNSHNDDALLK | 16457 | 26397 | 20308 | >25680.53 | 16329 | 245523 | >33333.33 | >59523.81 | |
| NYGL | | | | | | | | | |
| ALIYNYGLLYC | 137 | 9819 | 446 | 1286 | 551 | 11915 | >33333.33 | 676 | |
| FRKD | | | | | | | | | |
| DMDKVETFLRI | 1277 | 4813 | 867 | 1135 | 622 | 10484 | 1673 | 16127 | |
| VQCR | | | | | | | | | |
| FLRIVQCRSVE | | 106 | 33536 | 185 | 164 | 191 | 7199 | 7262 | 5311 |
| GSCGF | | | | | | | | | |
| FPTIPLSRLFD | 6923 | 46707 | 9458 | 175 | 923 | 5529 | 1051 | 14964 | |
| NAM | | | | | | | | | |
| RLFDNAMLRAH | 2.3 | 27 | 6289 | 1520 | 4247 | 3297 | 212 | >59523.81 | |
| RL | | | | | | | | | |
| QLAFDTYQEFE | >17985.61 | 7851 | 28586 | 47399 | 4843 | 21064 | >33333.33 | >59523.81 | |
| QNPQ | | | | | | | | | |
| SFLQNPQTSLC | 106 | 1829 | 671 | 1816 | 1230 | 7026 | 7069 | 3082 | |
| CFRK | | | | | | | | | |
| SNLELLRICLL | 731 | 61913 | 1526 | 2303 | 1112 | 1222 | 19782 | 3970 | |
| LIQS | | | | | | | | | |
| ICLLLIQSWLE | 8511 | 50874 | 11303 | 5708 | 71 | 643 | >33333.33 | >59523.81 | |
| PVQF | | | | | | | | | |
| NSLVYGASDSN | 13068 | >51428.57 | 240 | 3683 | 1229 | 297 | >33333.33 | >59523.81 | |
| IYDL | | | | | | | | | |
| SDSNIYDLLKD | >17985.61 | 124500 | 17458 | 25922 | 137 | >85034.01 | >33333.33 | 50134 | |
| LEEG | | | | | | | | | |
| DKVETFLRIVQ | 953 | 18325 | 1158 | 259 | 397 | 697 | 581 | 4080 | |
| CCGF | | | | | | | | | |
| SFLQNPQTSLT | 1191 | 2395 | 7780 | 15527 | 9558 | 6197 | >33333.33 | 17714 | |
| FSES | | | | | | | | | |
| TSLTFSESIPT | 182 | 17425 | 18 | 98 | 686 | 682 | 17602 | 2461 | |
| PSNR | | | | | | | | | |
| ALLKNYGLLYT | 19 | 5982 | 160 | 266 | 303 | 5923 | 3616 | 2628 | |
| FRKD | | | | | | | | | |
| LLYTFRKDMDK | >17985.61 | 23871 | 10623 | 17771 | 1133 | 53362 | 10448 | >59523.81 | |
| VETF | | | | | | | | | |
| DMDKVETFLRI | 1111 | 11194 | 2030 | 133 | 454 | 436 | 183 | 51511 | |
| VQTR | | | | | | | | | |
| FLRIVQTRSVE | 6.4 | 3944 | 11 | 16 | 99 | 9.8 | 445 | 778 | |
| GSTGF | | | | | | | | | |
| HLDMLRHLYQG | 304 | 37552 | 9417 | 2741 | 3593 | 27027 | 5384 | 12508 | |
| CQVV | | | | | | | | | |
| RLRIVRGTQLF | 4.8 | 11287 | 8389 | 2929 | 1024 | 12 | 6325 | 1834 | |
| EDNYAL | | | | | | | | | |
| GVGSPYVSRLL | 19 | 167949 | 1570 | 49 | 4156 | 190 | 1317 | 2614 | |
| GICL | | | | | | | | | |
| TLERPKTLSPQ | 10103 | 134367 | >22471.91 | 103285 | >28592.93 | 25988 | >75384.62 | >300000 | |
| KNGV | | | | | | | | | |
| KIFGSLAFLPE | 597 | 74162 | 1195 | 1897 | 37 | 377 | >75384.62 | 15796 | |
| SFDGDPA | | | | | | | | | |
| ELVSEFSRMAR | 201 | 1026 | 120 | 4882 | 15120 | 21259 | 4082 | 91575 | |
| DPQ | | | | | | | | | |
| GEALSTLVLNR | 719 | 11783 | 3045 | 305 | 14802 | 3191 | 192 | 20167 | |
| LKVG | | | | | | | | | |
| AYVLLSEKKIS | 78 | 136 | 943 | 359 | 9471 | 3848 | 27 | 3338 | |
| SIQS | | | | | | | | | |
| VASLLTTAEVV | 604 | 136308 | 7431 | 810 | 6517 | 369 | >118357.49 | 1955 | |
| VTE | | | | | | | | | |
| KCEFQDAYVIL | 14 | 5791 | 73 | 943 | 351 | 336 | 489 | 185 | |
| LSEKK | | | | | | | | | |
| ALSTLVLNRLK | 49 | 153 | 517 | 31 | 2167 | 647 | 4.0 | 2166 | |
| VGLQ | | | | | | | | | |
| MSYNLLGFLQR | 115 | 156715 | 366 | 1584 | 788 | 1060 | 3421 | 3646 | |
| SSNC | | | | | | | | | |
| LGFLQRSSNCQ | 437 | 112406 | 120 | 401 | 827 | 767 | 218 | 3729 | |
| CQKL | | | | | | | | | |
| RSSNCQCQKLL | 9665 | >191897.65 | 1046 | 2987 | 12652 | 9689 | 4530 | 74405 | |
| WQLN | | | | | | | | | |
| QCQKLLWQLNG | 181 | 133472 | 360 | 460 | 1004 | 3702 | 2519 | 4669 | |
| RLEY | | | | | | | | | |
| LWQLNGRLEYC | 1108 | 2356 | 816 | 8882 | 1024 | 10586 | >16333.33 | 5206 | |
| LKDR | | | | | | | | | |
| GRLEYCLKDRR | 9854 | 853 | 918 | 4155 | 3238 | 12108 | 1318 | 25159 | |
| NFDI | | | | | | | | | |
| RNFDIPEEIKQ | 6969 | 26262 | 18107 | 5375 | >114457.83 | 47893 | >144117.65 | >77319.59 | |
| LQQF | | | | | | | | | |
| PEEIKQLQQFQ | 1026 | 40154 | 1618 | 618 | 7875 | 49505 | 11908 | >77319.59 | |
| KEDA | | | | | | | | | |
| QLQQFQKEDAA | 85 | 17383 | 231 | 27473 | 1121 | 500 | 4862 | 55351 | |
| VTLY | | | | | | | | | |
| QKEDAAVTIYE | 8376 | >156521.74 | 9437 | 75877 | 785 | 45455 | >144117.65 | 5989 | |
| MLQN | | | | | | | | | |
| AVTIYEMLQNI | 17 | 23730 | 101 | 808 | 163 | 267 | 6873 | 4540 | |
| FAIF | | | | | | | | | |
| EMLQNIFAIFR | 395 | 9544 | 685 | 689 | 456 | 3313 | 10429 | 9738 | |
| QDSS | | | | | | | | | |
| IFAIFRQDSSS | 132 | 402 | 9.6 | 71 | 118 | 1186 | 4725 | 970 | |
| TGWN | | | | | | | | | |
| RQDSSSTGWNE | >102040.82 | 38681 | 4637 | 184507 | 40847 | 36320 | 15135 | 9075 | |
| TIVE | | | | | | | | | |
| STGWNETIVEN | 21407 | >156521.74 | 1755 | 10422 | 7060 | 3960 | >144117.65 | >77319.59 | |
| LLAN | | | | | | | | | |
| ETIVENLLANV | 659 | 40053 | 789 | 802 | 326 | 21681 | >144117.65 | 8151 | |
| YHQR | | | | | | | | | |
| NLLANVYHQRN | 152 | 40328 | 1039 | 1440 | 1492 | 8000 | 453 | 4160 | |
| HLKT | | | | | | | | | |
| VYHQRNHLKTV | 617 | 3135 | 7757 | 76003 | 153 | 6180 | 2101 | >77319.59 | |
| LEEK | | | | | | | | | |
| LEKEDFTRGKR | 21965 | 50733 | >20887.79 | 3968 | 5694 | 946 | 804 | >77319.59 | |
| MSSL | | | | | | | | | |
| FTRGKRMSSLH | 13 | 3302 | 1013 | 970 | 484 | 136 | 553 | 10925 | |
| LKRY | | | | | | | | | |
| RMSSLHLKRYY | 275 | 2181 | 993 | 4793 | 34 | 283 | 277 | 14964 | |
| GRIL | | | | | | | | | |
| HLKRYYGRILH | 26 | 3709 | 135 | 666 | 86 | 214 | 237 | 2896 | |
| YLKA | | | | | | | | | |
| YGRILHYLKAK | 30 | 42429 | 2343 | 917 | 23 | 900 | 704 | 7577 | |
| EDSH | | | | | | | | | |
| HYLKAKEDSHC | 1128 | 34758 | 2064 | 12153 | 3701 | 581 | 34851 | >77319.59 | |
| AWTI | | | | | | | | | |
| KBDSHCAWTIV | 4835 | >46656.3 | 353 | 1090 | 74 | 30 | 40000 | 2937 | |
| RVEI | | | | | | | | | |
| CAWTIVRVEIL | 66 | 3561 | 158 | 640 | 135 | 746 | 43672 | 757 | |
| RNFY | | | | | | | | | |
| VRVEILRYFYV | 1.8 | 429 | 140 | 47 | 18 | 14 | 3585 | 485 | |
| INRL | | | | | | | | | |
| RNFYVINRLTG | 1.7 | 2199 | 219 | 4618 | 182 | 527 | 167 | 7600 | |
| YLRN | | | | | | | | | |
| MSYNLLGFLQR | 25 | 107838 | 1152 | 813 | 433 8867 | 900 | 8972 | | |
| SSNT | | | | | | | | | |
| LGFLQRSSNTQ | 142 | 26455 | 18 | 211 | 1068 | 420 | 939 | 1345 | |
| TQK | | | | | | | | | |
| RSSNTQTQKLL | 10515 | 44338 | 2139 | 15497 | 12590 | 27678 | 1283 | >77319.59 | |
| WQLN | | | | | | | | | |
| QTQKLLWQLNG | 32 | 3555 | 55 | 35283 | 86 | 3099 | 2042 | 2083 | |
| RLEY | | | | | | | | | |
| LWQLNGRLEYT | 698 | 511 | 757 | 16171 | 94 | 20198 | 43286 | 16619 | |
| LKDR | | | | | | | | | |
| GRLEYTLKDRR | 7252 | 30 | 3228 | 97035 | 1379 | 4961 | 4917 | >77319.59 | |
| NFDI | | | | | | | | | |
| HYLKAKEDSHT | 232 | 70237 | 553 | 10677 | 15067 | 801 | 8526 | 10140 | |
| AWTI | | | | | | | | | |
| KEDSHTAWTIV | 1909 | 44754 | 746 | 2178 | 302 | 35 | >79032.26 | 6079 | |
| RVEI | | | | | | | | | |
| TAWTIVRVEIL | 7.8 | 2997 | 44 | 84 | 115 | 29 | 57243 | 404 | |
| RYFY | | | | | | | | | |
| LGFLQRSSNCQ | 192 | 4888 | 8.1 | 93 | 228 | 305 | 405 | 13167 | |
| SQKL | | | | | | | | | |
| RSSNCQSQKLL | 2050 | 57946 | 595 | 16721 | 4010 | 8922 | 6943 | 4062 | |
| WQLN | | | | | | | | | |
| QSQKLLWQLNG | 127 | 33374 | 84 | 741 | 55 | 1166 | 991 | 5920 | |
| RLEY | | | | | | | | | |
| GIVEQCCTSIC | 11123 | 777105 | 10911 | 2995 | 17793 | >79872.2 | >10047.16 | 13855 | |
| SLYQ | | | | | | | | | |
| TSICSLYQLEN | 11391 | >154109.59 | 20462 | 3791 | 12457 | >85616.44 | >54444.44 | >63025.21 | |
| YCN | | | | | | | | | |
| GILEQCCTSIC | 11025 | >187500 | 14862 | 5106 | 15983 | 54113 | >54444.44 | 16714 | |
| SLYQ | | | | | | | | | |
| GIVEQTTTSIT | 6354 | 107486 | 121 | 115 | 818 | 788 | >54444.44 | 13304 | |
| SLYQ | | | | | | | | | |
| EQTTTSITSLY | 18953 | >143769.97 | 170 | 258 | 272 | 2230 | >54444.44 | 17381 | |
| QLEN | | | | | | | | | |
| TSICSLYQLEN | 1125 | 202253 | 8841 | 1986 | 1089 | 247525 | >54444.44 | >83333.33 | |
| YCG | | | | | | | | | |
| TSITSLYQLEN | 1253 | 81293 | 1468 | 138 | 851 | 6055 | 26791 | 9947 | |
| YTN | | | | | | | | | |
| TSITSLYQLEN | 1132 | 96727 | 1628 | 129 | 115 | 8371 | 14562 | 46268 | |
| YTG | | | | | | | | | |
| GIVEQCCCGSH | 10043 | >74750.83 | 19904 | 2892 | 6626 | 41276 | >54444.44 | >63025.21 | |
| LVEA | | | | | | | | | |
| SLYQLENYCCG | 3568 | 54469 | 7313 | 1527 | 2356 | 12308 | >54444.44 | >83333.33 | |
| ERGF | | | | | | | | | |
| CCTSICSLYQL | 11655 | 71239 | 8383 | 1604 | 629 | 35604 | >54444.44 | 29845 | |
| ENY | | | | | | | | | |
| GSHLVEALYLV | 194 | >59681.7 | 2280 | 11512 | 2509 | 302 | >54444.44 | 37166 | |
| CCN | | | | | | | | | |
| CCGSHLVEALY | 880 | >55693.07 | 10081 | 20487 | 5230 | 1822 | >54444.44 | >63025.21 | |
| LVCC | | | | | | | | | |
| FVNQHLCGSHL | 583 | >187500 | 19209 | 39746 | >20663.4 | 679 | >54444.44 | >63025.21 | |
| VEAL | | | | | | | | | |
| QHLCGSHLVEA | 170 | 48557 | 12954 | 4303 | 9825 | 86 | >54444.44 | 7422 | |
| LYLV | | | | | | | | | |
| GSHLVEALYLV | 525 | >187500 | 8292 | 1603 | 4609 | 560 | >54444.44 | 5386 | |
| CGER | | | | | | | | | |
| VEALYLVCGER | 76 | 17558 | 209 | 124 | 1044 | 3869 | 24623 | 2233 | |
| GFFY | | | | | | | | | |
| YLVCGERGFFY | 11063 | 37210 | 1439 | 22980 | 730 | 64644 | >54444.44 | 1520 | |
| TPKT | | | | | | | | | |
| FVNQHLCGSDL | 117 | >74750.83 | 19154 | 36693 | 14913 | 38662 | >54444.44 | >63025.21 | |
| VEAL | | | | | | | | | |
| FYNQHLTGSHL | 9.2 | 67240 | 858 | 14916 | 1065 | 15 | >54444.44 | 41482 | |
| VEAL | | | | | | | | | |
| QHLTGSHLVEA | 9.3 | 50338 | >16096.5 | 3952 | 7423 | 38 | >54444.44 | 42312 | |
| LYLV | | | | | | | | | |
| GSHLVEALYLV | 645 | >176470.59 | 15781 | 1693 | 14443 | 553 | >54444.44 | >63025.21 | |
| TGER | | | | | | | | | |
| VEALYLVCGER | 88 | 9972 | 833 | 194 | 6108 | 6485 | >54444.44 | 6311 | |
| GSFY | | | | | | | | | |
| VEALYLVCGER | 14 | 11587 | 167 | 31 | 1027 | 5351 | 10565 | 3063 | |
| GFLY | | | | | | | | | |
| VEALYLVTGER | 9.9 | 2011 | 60 | 23 | 2342 | 195 | 1224 | 683 | |
| GFFY | | | | | | | | | |
| YLVCGERGFLY | 155 | 2033 | >20460.3 | >38550.5 | >30134.81 | 12842 | >54444.44 | 124 | |
| TPKT | | | | | | | | | |
| YLVCGERGFFY | 17260 | 11790 | >20460.3 | >38550.5 | >30134.81 | 92272 | >54444.44 | 317 | |
| TDKT | | | | | | | | | |
| YLVCGERGFFY | 3207 | 42139 | >20460.3 | >38550.5 | >30134.81 | 969 | >54444.44 | 1673 | |
| TDKT | | | | | | | | | |
| YLVTGERGFFY | 779 | 517 | >20460.3 | >38550.5 | 30457 | 7737 | 29236 | 6295 | |
| TPKT | | | | | | | | | |
| YLVTGERGFFY | 3259 | 7326 | >20460.3 | >38550.5 | >30134.81 | 5328 | >25789.47 | 2909 | |
| TDKT | | | | | | | | | |
| YLVTGERGFFY | 1152 | 4801 | >20460.3 | >38550.5 | >30134.81 | 78 | 4304 | 195313 | |
| TKYT | | | | | | | | | |
| VCGERGFFYTP | 9622 | 1989 | >20460.3 | >38550.5 | >15103.34 | 5494 | 419 | 14379 | |
| KTRR | | | | | | | | | |
| VTGERGFFYTP | 18906 | 3018 | 7226 | 147000 | 13417 | 27824 | 9407 | >300000 | |
| KTRR | | | | | | | | | |
| MWDLVLSIALS | 205 | | 1846 | | | 3032 | 23046 | 1727 | |
| VGCT | | | | | | | | | |
| DLVLSIALSVG | 1197 | | 13038 | | | 4029 | >245000 | 2200 | |
| CTGA | | | | | | | | | |
| HPQWVLTAAHC | 22 | 1103 | 875 | | | 563 | 1693 | 822 | |
| LKKN | | | | | | | | | |
| QWVLTAAHCLK | 895 | | >40000 | | | 3402 | 98000 | 4813 | |
| KNSQ | | | | | | | | | |
| GQRVPVSHSFP | 1563 | | >40000 | | | 629 | >245000 | 102 | |
| HPLY | | | | | | | | | |
| RVPVSHSFPHP | 67 | | >16000 | | | 101 | 100021 | 97 | |
| LYNM | | | | | | | | | |
| PHPLYNMSLLK | 19079 | | 819 | | | 20691 | 3315 | 1592 | |
| HQSL | | | | | | | | | |
| HPLYNMSLLKH | 232 | 13007 | 499 | | | 1282 | 382 | 199 | |
| QSLR | | | | | | | | | |
| NMSLLKHQSLR | 3131 | | >40000 | | | 20620 | 26496 | 96825 | |
| PDED | | | | | | | | | |
| SHDLMLLRLSE | 56 | 2396 | 2244 | | | 106 | 1327 | 112 | |
| PAK | | | | | | | | | |
| HDLMLLRLSEP | 16 | 1406 | 3063 | | | 109 | 544 | 43 | |
| AKIT | | | | | | | | | |
| PEEFLRPRSLQ | 2001 | | >26666.6 | | | 5156 | 2207 | 5839 | |
| CVSL | | | | | | | | | |
| PRSLQCVSLHL | 1111 | | 16000 | | | 2217 | 6107 | 28307 | |
| LSND | | | | | | | | | |
| NGVLQGITSWG | 1093 | | 8433 | | | 2285 52234 | 50111 | | |
| PEPC | | | | | | | | | |
| KPAVYTKVVHY | 5000 | | 1433 | | | 2401 | 53 | 3677 | |
| RICWI | | | | | | | | | |
| LHLLSNDMCAR | 2104 | 938 | 4277 | | | 27685 | 50230 | 59904 | |
| AYSE | | | | | | | | | |
| VGNWQYFFPVI | 37 | | 4.1 | | | 100 | | | |
| FSKA | | | | | | | | | |
| ESEFQAALSRK | | | 579 | 29617 | | | | | |
| VAKL | | | | | | | | | |
| IGHLYIFATCL | | | >816.33 | 12199 | | | | | |
| GLSYDGL | | | | | | | | | |
| VGNWQYFFPVI | | | 654 | 3846 | | | | | |
| FSK | | | | | | | | | |
| ASDSLQLVFGI | | | | | | | | | |
| ELMEVD | | | | | | | | | |
| PAYEKLSAEQS | | | 479 | >250000 | | | | | |
| PPPY | | | | | | | | | |
| RNGYRALMDKS | | | 512 | 5779 | | | | | |
| LHVGTQCALTRR | | | | | | | | | |
| FFKNIVTFFKN | 50000 | | >666.67 | 500000 | | >12500 | | | |
| IVT | | | | | | | | | |
| YKSAHKGFKGV | 70 | >900000 | 889 | 25000 | | 108 | | | |
| DAQGTLSKI | | | | | | | | | |
| VDAQGTLSKAF | 25 | 1383 | 1600 | 314 | | 1171 | | | |
| KLGGRDSRS | | | | | | | | | |
| ACASQKRPSQR | 50000 | >900000 | 889 | 25000 | | 2362 | | | |
| HGSKYLATAST | | | | | | | | | |
| ENPVVHFFKNI | | | | | | | | | |
| VTPR | | | | | | | | | |
| ENPVVAFFKNI | | | | | | | | | |
| VTPR | | | | | | | | | |
| ENPVVHAFKNI | | | | | | | | | |
| VTPR | | | | | | | | | |
| ENPVVHFFANI | | | | | | | | | |
| VTPR | | | | | | | | | |
| ENPVVHFFKNI | | | | | | | | | |
| VTPA | | | | | | | | | |
| NPVVHFFIQII | | | | | | | | | |
| VT | | | | | | | | | |
| HFFKNIVTPRT | | | | | | | | | |
| PPY | | | | | | | | | |
| NPVVHFFKNIV | | | | | | | | | |
| TPR | | | | | | | | | |
| LPVPGVLLKEF | 57 | 15058 | 14 | 12 | 12 | 57 | | | |
| TVSGNILTI | | | | | | | | | |
| WITQCFLPVFL | 679 | 25534 | 88 | 2804 | 216 | 74162 | | | |
| AQPPSGQRR | | | | | | | | | |
| DHRQLQLSISS | 1356 | 42666 | 1322 | 210 | 725 | 736 | | | |
| CLQQLSLLM | | | | | | | | | |
| YLAMPFATPME | 46 | 46591 | 266 | 814 | 405 | 526 | | | |
| AELARRSLA | | | | | | | | | |
| AAPLLLARAAS | 6.8 | 35410 | 139 | | | 160 | 30 | 64 | |
| LSLG | | | | | | | | | |
| APLLLARAASL | 8.4 | 56250 | 202 | | | 59 | 76 | 124 | |
| SLGF | | | | | | | | | |
| PLLLARAASLS | 10 | >81818.18 | 521 | | | 162 | 37 | 58 | |
| LGFL | | | | | | | | | |
| SLSLGFLFLLF | 11417 | | 4711 | | | 22727 | >122500 | 24620 | |
| FWLD | | | | | | | | | |
| LLFFWLDRSVL | 2.9 | 6.3 | 2.6 | | | 135 | 163 | 518 | |
| AKEL | | | | | | | | | |
| DRSVLAKELKF | 705 | | 569 | | | 2016 | 15815 | 4719 | |
| VTLV | | | | | | | | | |
| AKELKFVTLVF | 787 | 30000 | 783 | | | 606 | 1953 | 2355 | |
| RHGD | | | | | | | | | |
| RSPIDTFPTDP | >50000 | | 13095 | | | >62500 | >245000 | 6124 | |
| IKES | | | | | | | | | |
| FGQLTQLGMEQ | 2259 | | 3210 | | | >62500 | 109567 | >187500 | |
| HYEL | | | | | | | | | |
| DRTLMSAMTNL | 97 | 64286 | 13 | | | 383 | 2362 | 222 | |
| AALE | | | | | | | | | |
| MSAMTNLAALF | 1757 | | 700 | | | 36084 | 73870 | >187500 | |
| PPEG | | | | | | | | | |
| MTNLAALFPPE | 24 | | >40000 | | | >125000 | 39231 | 22822 | |
| GVS | | | | | | | | | |
| PEGVSIWNPIL | 111 | | 1778 | | | 15030 | 28577 | 103096 | |
| LWQP | | | | | | | | | |
| GVSIWNPILLW | 44 | 56250 | 10328 | | | 4992 | 11008 | 3985 | |
| QPIP | | | | | | | | | |
| WNPILLWQPIP | 208 | >81818.18 | 695 | | | 521 | 115494 | 607 | |
| VHTV | | | | | | | | | |
| NPILLWQPIPV | 31 | >81818.18 | 206 | | | 41 | 12999 | 575 | |
| HTVP | | | | | | | | | |
| PILLWQPIPVH | 44 | >81818.18 | 258 | | | 46 | 21244 | 168 | |
| TVPL | | | | | | | | | |
| ILLWQPIPVHT | 45 | >81818.18 | 170 | | | 19 | 13091 | 131 | |
| VPLS | | | | | | | | | |
| WQPIPVHTVPL | 6386 | | >26666.67 | | | 159 | >81666.67 | 17518 | |
| SEDQ | | | | | | | | | |
| LSGLHGQDLFG | 148 | | >26666.67 | | | >35714.29 | >81666.67 | >125000 | |
| IWSK | | | | | | | | | |
| YDPLYCESVHN | 1597 | 16625 | 8889 | | | 838 | 30867 | 643 | |
| FTLP | | | | | | | | | |
| LPSWATBDTMT | 20274 | | 973 | | | >35714.29 | >81666.67 | >125000 | |
| KLRE | | | | | | | | | |
| LRELSELSLLS | 655 | | 371 | | | 4010 | 9368 | 1614 | |
| LYGI | | | | | | | | | |
| LSELSLLSLYG | 482 | >81818.18 | 1549 | | | 20906 | 1186 | 1450 | |
| IHKQ | | | | | | | | | |
| LSLLSLYGIHK | 656 | >81818.18 | 4444 | | | >35714.29 | 1637 | 4959 | |
| QKEK | | | | | | | | | |
| KSRLQGGVLVN | 362 | | >26666.6 | | | 2838 | >81666.67 | 5516 | |
| EILN | | | | | | | | | |
| GGVLVNEILNH | 2165 | 700 | 359 | | | 29463 | 3239 | 54411 | |
| MK | | | | | | | | | |
| IFSYKKLIMYS | 9.9 | 9728 | 510 | | | 1946 | 60 | 351 | |
| AHDT | | | | | | | | | |
| YKKLIMYSAHD | 17 | 22678 | 207 | | | 292 | 309 | 107 | |
| TTVS | | | | | | | | | |
| LIMYSAHDTTV | 4496 | | 24 | | | 731 | 24812 | 813 | |
| SGLQ | | | | | | | | | |
| DTTVSGLQMAL | 171 | | 4424 | | | 14706 | >245000 | 2876 | |
| DVYN | | | | | | | | | |
| ALDVYNGLLPP | 18 | | 485 | | | >83333.33 | 588 | 86603 | |
| YASC | | | | | | | | | |
| LDVYNGLLPPY | 15 | | 348 | | | >83333.33 | 404 | 31277 | |
| ASCH | | | | | | | | | |
| YNGLLPPYASC | 42 | | 6189 | | | >83333.33 | 14027 | 8022 | |
| HLTE | | | | | | | | | |
| FAELVGPVIPQ | 12 | | 4690 | | | 24056 | >245000 | 39472 | |
| DWST | | | | | | | | | |
| TVPLSEDQLLY | 4012 | 332 | 10755 | | | 11313 | 42162 | 37369 | |
| LPFR | | | | | | | | | |
| LTELYFEKGEY | 2249 | 592 | 8051 | | | 13062 | 18841 | 26949 | |
| FVEM | | | | | | | | | |
| GPVIPQDWSTE | | 52098 | | | | | | | |
| CMTT | | | | | | | | | |
| QAHSLERVCHC | 50000 | | 667 | 500000 | | >250000 | | | |
| LGKWLGHPDK | | | | | | | | | |
| WTTCQSIAFPS | | 17308 | 22 | | | | | | |
| KTSASIGSL | | | | | | | | | |
| QKGRGYRGQHQ | | >47368.42 | 88 | | | | | | |
| AHSLERVCH | | | | | | | | | |
| AATYNFAVLKL | | >52941.18 | 533 | | | | | | |
| MGRGTKF | | | | | | | | | |
| VATGLCFFGVA | | >112500 | 351 | | | | | | |
| LFCGCGHEA | | | | | | | | | |
| FLYGALLLAEG | | | | | | | | | |
| FYTTGAVRQ | | | | | | | | | |
| SAVPVYIYFNT | | | | | | | | | |
| WTTCQSIAF | | | | | | | | | |
| TLSVTWIGAAP | 3.1 | >81818.18 | 7273 | | | 16 | 840 | 5.4 | |
| LILS | | | | | | | | | |
| SVTWIGAAPLI | 4.1 | >81818.18 | 3152 | | | 83 | 139 | 30 | |
| LSPI | | | | | | | | | |
| VTWIGAAPLIL | 8.1 | >81818.18 | 8000 | | | 195 | 731 | 82 | |
| SRIV | | | | | | | | | |
| SQPWQVLVASR | 66 | >81818.18 | 7628 | | | 385 | 386 | 621 | |
| GRAV | | | | | | | | | |
| GRAVCGGVLVH | 386 | | >26666.6 | | | 3582 | >245000 | 8069 | |
| PQWV | | | | | | | | | |
| GVLVHPQWVLT | 87 | 21320 | 67 | | | 153 | 1931 | 365 | |
| AAHC | | | | | | | | | |
| HPQWVLTAAHC | 13 | 3632 | 1621 | | | 283 | 1305 | 107 | |
| IRNK | | | | | | | | | |
| QWVLTAAHCIR | 50 | | 19403 | | | 214 | 2598 | 967 | |
| NKSV | | | | | | | | | |
| AHCIRNKSVIL | 578 | 29704 | 69 | | | 2573 | 104 | 715 | |
| LGRH | | | | | | | | | |
| SVILLGRHSLF | 717 | 1400 | 12649 | | | 26088 | 500 | 5216 | |
| HPED | | | | | | | | | |
| VILLGRHSLFH | 273 | 8744 | 8208 | | | 30625 | 737 | 18520 | |
| PEDT | | | | | | | | | |
| GQVFQVSHSFP | 288 | 45000 | 8.2 | | | 27 | 548 | 33 | |
| HPLY | | | | | | | | | |
| VFQVSHSFPHP | 16 | >75000 | 25 | | | 51 | 8751 | 17 | |
| LYDM | | | | | | | | | |
| PHPLYDMSLLK | 1315 | | 20787 | | | 10699 | 29813 | 12836 | |
| NRFL | | | | | | | | | |
| SHDLMLLRLSE | 532 | 6215 | 4051 | | | 58 | 3538 | 64 | |
| PAEL | | | | | | | | | |
| HDLMLLRLSEP | 62 | 2867 | 6193 | | | 152 | 3914 | 22 | |
| AELT | | | | | | | | | |
| TDAVKVMDLPT | >50000 | | >80000 | | | >41666.67 | 20875 | >107142.8 | |
| QEPA | | | | | | | | | |
| LHVISNDVCAQ | 789 | 8318 | 790 | | | 17451 | >122500 | 32671 | |
| VHPQ | | | | | | | | | |
| CAQVHPQKVTK | 10206 | | 2566 | | | 32275 | 8731 | 34893 | |
| FMLC | | | | | | | | | |
| GGPLVCNGVLQ | 3353 | | 68 | | | >35714.29 | 9334 | 16308 | |
| GITS | | | | | | | | | |
| GPLVCNGVLQG | 1724 | | 30 | | | 4893 | 4187 | 32640 | |
| ITSW | | | | | | | | | |
| NGVLQGITSWG | 945 | 24942 | 560 | | | 485 | 5874 | 819 | |
| SEPC | | | | | | | | | |
| RPSLYTKVVHY | 6041 | 53785 | 339 | | | 652 | 39 | 5484 | |
| RKWI | | | | | | | | | |
| HSLFHPEDTOQ | | 65260 | | | | | | | |
| VFQV | | | | | | | | | |
| PRWLCAGALVL | 46 | | >20000 | | | 766 | 26531 | 1439 | |
| AGGF | | | | | | | | | |
| LGFLFGWFIKS | 10 | >75000 | 1338 | | | 2261 | 1421 | 1701 | |
| SNEA | | | | | | | | | |
| LDELKAENIKK | 1136 | 1370 | 4842 | | | 7470 | 1248 | 12778 | |
| FLYN | | | | | | | | | |
| IKKFLYNFTQI | 449 | 8080 | 43 | | | 29 | 512 | 160 | |
| PHLA | | | | | | | | | |
| KFLYNFTQIPH | 340 | 13805 | 217 | | | 30 | 415 | 54 | |
| LAGT | | | | | | | | | |
| WKEFGLDSVEL | 1139 | 85 | 96 | | | 3511 | 19971 | 7052 | |
| AHYD | | | | | | | | | |
| LAHYDVLLSYP | 79 | 37533 | 1117 | | | 3617 | 415 | 1009 | |
| NK | | | | | | | | | |
| GNEIFNTSLFE | 20412 | | >20000 | | | >35714.29 | >163333.33 | 10415 | |
| PPPP | | | | | | | | | |
| GKVFRGNKVKN | 612 | | 1087 | | | 2350 | 4121 | 31277 | |
| AQLA | | | | | | | | | |
| GNKVKNAQLAG | 677 | | 13333 | | | >83333.33 | 28904 | 7882 | |
| AKGV | | | | | | | | | |
| EYAYRRGIAEA | 5.1 | | 213 | | | 70 | 596 | 67 | |
| VGLP | | | | | | | | | |
| AEAVGLPSIPV | 5.4 | | 9923 | | | 2015 | >490000 | 23102 | |
| HPIG | | | | | | | | | |
| AVGLPSTPVHP | 3.6 | | 4193 | | | 1080 | 4432 | 15377 | |
| IGYY | | | | | | | | | |
| IGYYDAQKLLE | 1923 | | 12649 | | | >83333.33 | 8236 | 47246 | |
| KMGG | | | | | | | | | |
| TGNFSTQKVKM | 11180 | | 833 | | | 9407 | 10282 | 1450 | |
| HIHS | | | | | | | | | |
| TRIYNVIGTLR | 14 | 33333 | 6.3 | | | 4806 | 70 | 2900 | |
| GAVE | | | | | | | | | |
| ERGVAYINADS | 2440 | | 6761 | | | 34021 | >163333.33 | 25516 | |
| SIEG | | | | | | | | | |
| GVAYINADSSI | 1054 | | 146 | | | 6244 | 23360 | 3048 | |
| EGNY | | | | | | | | | |
| DSSIEGNYTLR | 16667 | | 3360 | | | 14458 | >163333.33 | >187500 | |
| VDCT | | | | | | | | | |
| NYTLRVDCTPL | 6804 | 45 | 9.9 | | | 24597 | 6323 | 48412 | |
| MYSL | | | | | | | | | |
| CTPLMYSLVHN | 93 | 19437 | 245 | | | 140 | 223 | 249 | |
| LTKE | | | | | | | | | |
| DFEVFFQRLGI | 143 | | 221 | | | 21926 | 122 | 2005 | |
| ASGR | | | | | | | | | |
| EVFFQRLGIAS | 28 | >75000 | 22 | | | 5311 | 6.3 | 2976 | |
| GRAR | | | | | | | | | |
| TNKFSGYPLYH | 3402 | | 5521 | | | 30853 | 614 | 741 | |
| SVYE | | | | | | | | | |
| YDPMFKYHLTV | 9.0 | >75000 | 19 | | | 158 | 172 | 179 | |
| AQVR | | | | | | | | | |
| DPMFKYHLTVA | 5.7 | >75000 | 9.1 | | | 168 | 43 | 258 | |
| QVRG | | | | | | | | | |
| MFKYHLTVAQV | 16 | 29032 | 18 | | | 72 | 70 | 266 | |
| RGGM | | | | | | | | | |
| KYHLTVAQVRG | 137 | 33658 | 806 | | | 228 | 1519 | 5860 | |
| GMVF | | | | | | | | | |
| VAQVRGGMVFE | 228 | | 662 | | | 4449 | >98000 | 499 | |
| LANS | | | | | | | | | |
| RGGMVFELANS | 10 | 37118 | 229 | | | 41 | 8682 | 33 | |
| IYLP | | | | | | | | | |
| GMVFELANSIV | 15 | 4604 | 230 | | | 30 | 4995 | 81 | |
| LPFD | | | | | | | | | |
| VFELANSIVLP | 19 | 667 | 999 | | | 39 | 36123 | 50 | |
| FDCR | | | | | | | | | |
| ADKIYSISMKH | 22361 | | 5310 | | | 4098 | 1136 | 3512 | |
| PQEM | | | | | | | | | |
| IYSISMKHPQE | 8452 | | 16000 | | | 11573 | 1357 | 12293 | |
| MKTY | | | | | | | | | |
| PQEMKTYSVSF | 15143 | | 3024 | | | 1192 | >98000 | 1981 | |
| DSLF | | | | | | | | | |
| TYSYSFDSLFS | 219 | 101 | 73 | | | 346 | 2256 | 526 | |
| AVKN | | | | | | | | | |
| VLRMMNDQLMF | 118 | 183 | 29 | | | 17334 | 1700 | 10684 | |
| LERA | | | | | | | | | |
| LRMMNDQLMFL | 2704 | | 392 | | | 17507 | 2492 | 4601 | |
| ERAF | | | | | | | | | |
| RHVIYAPSSHN | 2174 | | 481 | | | 31250 | 11667 | 481 | |
| KYAG | | | | | | | | | |
| RQIYVAAFTVQ | 3.7 | 28347 | 1.2 | | | 292 | 36 | 91 | |
| AAAE | | | | | | | | | |
| QIYVAAFTVQA | 1.6 | 26609 | 1.6 | | | 324 | 102 | 65 | |
| AAET | | | | | | | | | |
| VAAFTVQAAAE | 14 | >75000 | 58 | | | 793 | 1420 | 127 | |
| TLSE | | | | | | | | | |
| YISIINEDGNE | 498 | 397 | 624 | | | 23719 | >122500 | 83056 | |
| IFNT | | | | | | | | | |
| ISIINEDGNEI | 507 | 559 | >12965.96 | | | >23105.36 | >122500 | >52337.75 | |
| FNTS | | | | | | | | | |
| EDFFKLERDMK | 2710 | 468 | 226 | | | 8550 | 1439 | >52337.75 | |
| INCS | | | | | | | | | |
| FFKLERDMKIN | 4419 | 121 | 483 | | | >23105.36 | 8109 | >52337.75 | |
| CSGK | | | | | | | | | |
| GVILYSDPADY | 1566 | 17 | 7508 | | | 7848 | 106291 | 2473 | |
| FAPG | | | | | | | | | |
| GAAVVHEIVRS | | 12409 | | | | | | | |
| FGTL | | | | | | | | | |
| NSRLLQERGVA | 614 | 318 | 5089 | | | 7997 | 3224 | 2616 | |
| YINA | | | | | | | | | |
| VAYINADSSIE | 4716 | 531 | 411 | | | 9745 | 105832 | 5467 | |
| GNYT | | | | | | | | | |
| DQLMFLERAFI | | >19667.83 | | | | | | | |
| DPLG | | | | | | | | | |
| KSNFLNCYVSG | 2500 | >900000 | 296 | 3125 | | 8333 | | | |
| FHPSD | | | | | | | | | |
| ACNPDAENWNS | | | | | 500000 | | >25000 | | |
| QFEILEDAA | | | | | | | | | |
| EYLILSARDVL | | | 508 | | | | | | |
| AVVS | | | | | | | | | |
| YKTIAYDEEAR | 50000 | 143 | 4000 | 500000 | | 250000 | | | |
| R | | | | | | | | | |
| GEALSTLVVNK | 292 | 29687 | 1535 | 246 | 30057 | 2325 | 383 | 40840 | |
| IRGT | | | | | | | | | |
| PYILLVSSKVS | 1.1 | 106 | 64 | 13 | 136 | 38 | 12 | 134 | |
| TVKD | | | | | | | | | |
| EAVLEDPYILL | 34 | 479 | 233 | 172 | 681 | 933 | 1666 | 15032 | |
| VSSK | | | | | | | | | |
| IAGLFLTTEAV | 6.8 | 27189 | 13 | 106 | 67 | 230 | 3893 | 409 | |
| VADK | | | | | | | | | |
| ALSTLVVNKIR | 75 | 274 | 648 | 40 | 3626 | 396 | 20 | 18035 | |
| GTFK | | | | | | | | | |
| MKHILYISFYF | 5893 | | 189 | | 3385 | 1250 | 15558 | | |
| ILVN | | | | | | | | | |
| KSLLSTNLPYG | 4226 | | 690 | | | 50000 | | | |
| RTNL | | | | | | | | | |
| HFFLFLLYILF | 337 | | 260 | | | 42443 | 19641 | | |
| LVKM | | | | | | | | | |
| LFLLYILFLVK | 1160 | | 283 | | | 4868 | 10869 | | |
| MNAL | | | | | | | | | |
| ILFLVKMNALR | 0.80 | | 5.6 | | | 56 | 19 | | |
| RLPV | | | | | | | | | |
| MNALRRLPVIC | 2.1 | | 13 | | | 488 | 265 | | |
| SFLV | | | | | | | | | |
| SAFLESQSMNK | 549 | | 113 | | | 523 | 21493 | | |
| IGDD | | | | | | | | | |
| LKELIKVGLPS | 99 | | 163 | | | 542 | 1493 | | |
| FENL | | | | | | | | | |
| FENLVAENVKP | 56 | | 2372 | | | 120215 | >25025.54 | | |
| PKVD | | | | | | | | | |
| PATYGIIVPVL | 1.03 | | 15 | | | 139 | 181 | | |
| TSLF | | | | | | | | | |
| YGIIVPVLTSL | 6.0 | | 2.0 | | | 60 | 793 | | |
| FNKV | | | | | | | | | |
| LLKIWKNYMKI | 121 | | 132 | | | 395 | 132 | | |
| MNHL | | | | | | | | | |
| MTLYQIQVMKR | 1219 | | 117 | | | 31053 | 166 | | |
| NQKQ | | | | | | | | | |
| QKQVQMMIMTK | 121 | | 213 | | | 3618 | 182 | | |
| FMGV | | | | | | | | | |
| MIMIKFMGVIY | 2905 | | 312 | | | 68040 | 66150 | | |
| IMII | | | | | | | | | |
| GVIYIMIISKK | 10 | | 22 | | | 476 | 137 | | |
| MMRK | | | | | | | | | |
| LYYLFNQHIKK | 27 | | 1324 | | | 10244 | 1771 | | |
| ELYH | | | | | | | | | |
| HFNMLKNKMQS | 12 | | 18 | | | 3225 | 185 | | |
| SFFM | | | | | | | | | |
| LDIYQKLYIKQ | 2834 | | 1492 | | | >88339.22 | 1204 | | |
| EEQK | | | | | | | | | |
| QKKYIYNLIMN | 73 | | 24 | | | 11942 | 13255 | | |
| TQNK | | | | | | | | | |
| YEALIKLLPFS | 55 | | 1839 | | | 3578 | 180 | | |
| KRIR | | | | | | | | | |
| ENEYATGAVRP | 4438 | | 281 | | | 4970 | 17329 | | |
| FQAA | | | | | | | | | |
| NYELSKKAVIF | 713 | | 536 | | | 5498 | 141 | | |
| TPIY | | | | | | | | | |
| QKILIKIPVTK | 993 | | 303 | | | 534 | 2240 | | |
| NIIT | | | | | | | | | |
| KCLVISQVSNS | 628 | | 16 | | | 46383 | 17859 | | |
| DSYK | | | | | | | | | |
| SKIMKLPKLPI | 824 | | 6485 | | | 83674 | 110 | | |
| SNGK | | | | | | | | | |
| FIHFFTWGTMF | 745 | | 273 | | | 489 | 1699 | | |
| VPKY | | | | | | | | | |
| LCNFKKNIIAL | 9.7 | | 312 | | | 423 | 21324 | | |
| LIIP | | | | | | | | | |
| KKNIIALLIIP | 13 | | 203 | | | 495 | 157 | | |
| PKIIA | | | | | | | | | |
| ALLIIPPKIHI | 648 | | 1738 | | | 8.4 | 11957 | | |
| SIEL | | | | | | | | | |
| SMEYKKDFLIT | 939 | | 24 | | | 776 | 8897 | | |
| ARKP | | | | | | | | | |
| KSKFNILSSPL | 0.80 | | 16 | | | 65 | 152 | | |
| FNNF | | | | | | | | | |
| FKKLKNHVLFL | 2.3 | | 28 | | | 11 | 695 | | |
| QMMN | | | | | | | | | |
| KNHVLFLQMMN | 12 | | 32 | | | 757 | >120098.04 | | |
| VNLQ | | | | | | | | | |
| VLFLQMMNVNL | 6.3 | | 30 | | | 8441 | 56770 | | |
| QKQL | | | | | | | | | |
| NVNLQKQLLTN | 96 | | 2460 | | | 555 | 11245 | | |
| HLIN | | | | | | | | | |
| QKQLLTNHLIN | 675 | | 228 | | | 4412 | 20984 | | |
| TPK | | | | | | | | | |
| NHLINTPKIMP | 1378 | | 4798 | | | 625 | 1296 | | |
| HHII | | | | | | | | | |
| YILLKKILSSR | 220 | | 183 | | | 8.3 | 18 | | |
| FNQM | | | | | | | | | |
| FNQMIEVSSIF | 483 | | 2091 | | | 854 | 16504 | | |
| ISFY | | | | | | | | | |
| KVSCKGSGYTF | 5000 | | 381 | | | 50000 | 2946 | | |
| TAYQMH | | | | | | | | | |
| IAKVPPGPNIT | 50000 | >30000 | >666.67 | 500000 | | >12500 | | | |
| AEYGDKWLD | | | | | | | | | |
| TAEYGDKWLDA | 50000 | >30000 | >666.67 | 16667 | | 3125 | | | |
| KSTWYGKPT | | | | | | | | | |
| AKSTWYGKPTG | 50000 | >30000 | 667 | 500000 | | >12500 | | | |
| AGPKDNGGA | | | | | | | | | |
| GAGPKDNGGAC | 50000 | >30000 | >666.67 | 500000 | | >12500 | | | |
| GYKDVDKAP | | | | | | | | | |
| FNGMTGCGNTP | 50000 | 51962 | >666.67 | 500000 | | >12500 | | | |
| IFKDGRGCG | | | | | | | | | |
| PIFKDGRGCGS | 50000 | 6784 | >666.67 | 500000 | | >12500 | | | |
| CFEIKCTKP | | | | | | | | | |
| SCFEIKCTKPE | 50000 | >900000 | >666.67 | 500000 | | 12500 | | | |
| SCSGEAVTV | | | | | | | | | |
| AFGSMAKKGEE | 50000 | >30000 | >666.67 | 50000 | | >12500 | | | |
| QNVRSAGEL | | | | | | | | | |
| TPDKLTGPFTV | 50000 | >900000 | >666.67 | 500000 | | >12500 | | | |
| RYTTEGGTK | | | | | | | | | |
| VRYTTEGGTKS | 50000 | >30000 | >666.67 | 500000 | | >12500 | | | |
| EVEDVIPEG | | | | | | | | | |
| TCVLGKLSQEL | 26 | 29529 | 14848 | 7566 | 9001 | 18653 | 7656 | 17895 | |
| HKLQ | | | | | | | | | |
| KLSQELHKLQT | 19 | 196889 | 19684 | 2076 | 12198 | 85464 | 28656 | 19129 | |
| YPRT | | | | | | | | | |
| LHKLQTYPRTN | 2118 | >205479.45 | 15182 | 9921 | >7403.08 | 40226 | 1618 | >29228.37 | |
| TGSG | | | | | | | | | |
| KLQTYPRTNTG | >10060.36 | >205479.45 | >26490.07 | 114672 | >9806.45 | >99206.35 | >51578.95 | >29228.37 | |
| SGTP | | | | | | | | | |
| CCVLGKLSQEL | 34 | 17387 | 19764 | 31253 | 5299 | 41656 | 5640 | 21704 | |
| HKLQ | | | | | | | | | |
| CSNLSTCVLQK | 296 | >205479.45 | 14339 | 28603 | 5340 | 31837 | 3516 | 7225 | |
| LSQE | | | | | | | | | |
| TSNLSTTVLGK | 298 | 86798 | 8016 | 32358 | 9280 | 31275 | 2058 | 2469 | |
| LSQE | | | | | | | | | |
| TTVLGKLSQEL | 133 | 92782 | 22449 | 36802 | >9806.45 | 26113 | 16182 | 23824 | |
| HKLQ | | | | | | | | | |
| DIAAKYKELGY | | >900000 | >470.59 | | | | | | |
| ALVRQGLAKVA | 1250 | | 190 | 500000 | | | | | |
| PATLIKAIDGD | 278 | 6429 | 296 | 3846 | | 8333 | | | |
| TVKLMYKGQ | | | | | | | | | |
| TPETKHPKKGV | >1000 | >900000 | >500 | 500000 | | 12500 | | | |
| EKYGPEASA | | | | | | | | | |
| VEKYGPEASAF | 50000 | >900000 | 1333 | 500000 | | 12500 | | | |
| TKKMVENAK | | | | | | | | | |
| FTKKMVENAKK | >1000 | 11619 | >500 | 500000 | | 8333 | | | |
| IEVEFDKGQ | | | | | | | | | |
| YIYADGKMVNE | 65 | | 500 | 4167 | | 1563 | | | |
| ALVRQGLAK | | | | | | | | | |
| HEQHLRKSEAQ | 50000 | 90000 | 80000 | 16667 | | 6250 | | | |
| AKKEKLNIW | | | | | | | | | |
| QAKKEKLNIWS | 50000 | >900000 | 364 | 3125 | | >250000 | | | |
| EDNADSGQ | | | | | | | | | |
| YFNNFTVSFWL | 50000 | | 615 | 25000 | | | | | |
| RVPK | | | | | | | | | |
| FSYFPSI | 50000 | | 889 | 16667 | | | | | |
| YSFFPSI | 50000 | | 889 | 500000 | | | | | |
| YSYFPSIR | 50000 | >900000 | 667 | 16667 | | 7217 | | | |
| DPNANPNVDPN | | | 738 | >5494.51 | | >15625 | | | |
| AN | | | | | | | | | |
| PNVNANPNANP | | | | | | | | | |
| NANP(X4) | | | | | | | | | |
| QKWAAVVVPS | 50000 | | 1000 | 50000 | | | | | |
| TWQLNGEELIQ | 50000 | | 889 | 2273 | | | | | |
| DM | | | | | | | | | |
| ELVETRPAG | | | | | | | | | |
| PEFLEQRRAAV | 5000 | | 80000 | 500000 | | 250000 | | | |
| DTYC | | | | | | | | | |
| STORKUSP33 | | | 617 | 2069 | | | | | |
| DYSYLQDSDPD | >50000 | | 189 | >500000 | >126666.67 | >250000 | >61250 | >107142.86 | |
| SFQD | | | | | | | | | |
| DFSYLQDSDPD | | | 264 | >500000 | >126666.67 | >250000 | >61250 | >107142.86 | |
| SFQD | | | | | | | | | |
| QNILFSNAPLG | | | 195 | | | | | | |
| PQFP | | | | | | | | | |
| QNILLSNAPLV | | | 538 | | | | | | |
| PQFP | | | | | | | | | |
| DYSYLQDSDPD | | | 316 | >166666.67 | | | | | |
| SFQD | | | | | | | | | |
| KYVKQNTLKLA | 9.9 | | 6.2 | 25000 | | | | | |
| T | | | | | | | | | |
| P(X)KQNTLKL | 1.7 | | | | | | | | |
| AT | | | | | | | | | |
| BEDIEIIPIQE | >9057.97 | >18549.05 | >7518.8 | 12203 | 849 | >6742.18 | | 128305 | |
| EBY | | | | | | | | | |
| QAISPRTLNS | 1961 | 298315 | 6214 | 1314 | 3450 | 39701 | 14848 | 286179 | |
| PAIF | | | | | | | | | |
| YTDVFSLDPTF | | 217 | | | | | | | |
| TIETT | | | | | | | | | |
| YAGIRRDGLLL | | 9.6 | | | | | | | |
| RLVD | | | | | | | | | |
| LFFYRKSVWSK | 19 | 30163 | 913 | 1383 | 84 | 84 | 65 | | |
| LQSI | | | | | | | | | |
| RPIVNMDYVVG | 29 | 22 | 3.1 | 21 | 812 | 346 | 748 | | |
| ARTFRREKR | | | | | | | | | |
| RPGLLGASVLG | 1789 | 35768 | 6522 | 4414 | 3183 | 506 | >61250 | | |
| LDDI | | | | | | | | | |
| LYFVKVDVTGA | 16 | 9.6 | 2.8 | 13 | 14 | 5892 | 413 | | |
| YDTI | | | | | | | | | |
| FAGIRRDGLLL | 2381 | 3.6 | 7092 | 3820 | >3365.21 | 41148 | 7650 | | |
| RLVD | | | | | | | | | |
| AKTFLRTLVRG | 104 | 54159 | 208 | 3326 | 105 | 25 | 9.2 | | |
| VPEY | | | | | | | | | |
| YGAVVNLRKTV | 13509 | 150175 | 4194 | 4531 | >95000 | 8274 | 113 | | |
| VNFP | | | | | | | | | |
| GTAFVQMPAHG | 1.6 | 37275 | 8.1 | 34 | 18 | 90 | 99 | | |
| LFPW | | | | | | | | | |
| WAGLLLDTRTL | 2016 | 22 | 49 | 323 | 1238 | 186 | >61250 | | |
| EVQS | | | | | | | | | |
| RTSIRASLTFN | 1430 | 256 | 770 | 177 | 5131 | 411 | 5475 | | |
| RGFK | | | | | | | | | |
| RVIKNSIRLTL | 3650 | 584 | 9249 | 5389 | 80682 | 2239 | 1175 | 2566 | |
| PVIKNSIKLRL | 1549 | 198 | 34245 | 14612 | 277735 | 4091 | 541 | 2851 | |
| ATSTKKLHKEP | 4.6 | 8018 | 113 | 1020 | | 2083 | | | |
| ATLIKAIDG | | | | | | | | | |

TABLE 27

HLA-DR SUPERTYPE

| | | | | | | | | |
|---|---|---|---|---|---|---|---|---|
| | SEQ | DRB1 | DRBI | DRBI | DRB3 | DRB4 | DRB5 | DRB5 |
| Sequence | ID NO. | *1101 | *1302 | *1501 | *0101 | *0101 | *0101 | *0201 |

| | | | | | | | | |
|---|---|---|---|---|---|---|---|---|
| AC- | | >33333.33 | >10000 | 200000 | 101 | | 1250 | |
| NPTKHKWEAAHVAEQ | | | | | | | | |
| LAA | | | | | | | | |
| DDYVKQYTKQYTKQN | | >1111.11 | | | >11111.11 | | 35 | |
| TLKK | | | | | | | | |
| AAAKAAAAAAYAA | | 200000 | | | 6250 | | 2857 | |
| AC- | | | | | | | | |
| AAAKAAAAAAYAA | | | | | | | | |
| (20)AYA(20)A(20) | | 200000 | | | | | 2857 | |
| A(20)K(20)A(20) | | | | | | | | |
| AC-AAAKATAAAAYAA | | | | | | | | |
| AC-AAAKAAAAAAFAA | | | | | | | | |
| AC- | | | | | | | | |
| AAAKATAAAA(10)AA | | | | | | | | |
| AC- | | | | | | | | |
| AAAKATAAAA(23)AA | | | | | | | | |
| AAKAAAAAAA(10)AA | | | | | | | | |
| AAYAAAATAKAAA | | | | | | | | |
| AALAAAAAAKAAA | | 2222 | | | | | 67 | |
| AAEAAAATAKAAA | | | | | | | | |
| AAYJJAAAAKAAA | | | | | | | | |
| AAYAAAAJJKAAA | | | | | | | | |
| AFLRAAAAAAFAA | | | | | | | | |
| AFLRQAAAAAFAAY | | | | | | | | |
| AAFAAAKTAAAFA | | 4.6 | | | 20000 | | 25 | 6.4 |
| YAAFAAAKTAAAFA | | 2.6 | | | 33333 | 30 | 9.5 | |
| AALKATAAAAAAA | | | | | | | | |
| YAR(15)ASQTTLKAK | | 3.9 | | | | | 3.6 | |
| T | | | | | | | | |
| YARF(33)QTTLKAKT | | | | | | | | |
| PKYFKQRILKFAT | | | | | | | | |
| PKYFKQGFLKGAT | | | | | | | | |
| PKYGKQIDLKGAT | | | | | | | | |
| AAFFFFFGGGGGA | | | | | | | | |
| AADFFFFFFFFDA | | | | | | | | |
| AAKGIKIOFGIFA | | | | | | | | |
| AAFIFIGGGKIKA | | | | | | | | |
| AAKIFIGFFIDGA | | | | | | | | |
| AAFIGFGKIKFIA | | | | | | | | |
| AAKIGFGIKIGFA | | | | | | | | |
| AAFKIGKFGIFFA | | | | | | | | |
| AADDDDDDDDDDA | | | | | | | | |
| (43)AAIGFFFFKKG | | | | | | | | |
| IA | | | | | | | | |
| (43)AAFFGIFKIGK | | | | | | | | |
| FA | | | | | | | | |
| (43)AADFGIFIDFI | | | | | | | | |
| IA | | | | | | | | |
| (43)AAIGGIFIFKK | | | | | | | | |
| DA | | | | | | | | |
| (43)AAFIGFGKIKF | | | | | | | | |
| IA | | | | | | | | |
| (43)AAKIGFGIKIG | | | | | | | | |
| FA | | | | | | | | |
| (43)AAFKIGKFGIFF | | | | | | | | |
| AAAAKAAAAAAAAF | | | | | | | | |
| AAAKAAAAAAAFA | | | | | | | | |
| AAAKAAAAAAFAA | | | | | | | | |
| AAAKAAAAFAAAA | | | | | | | | |
| FAAAAAAAAAAAA | | | | | | | | |
| AAAAAAAAAAAAN | | | | | | | | |
| AAAAAAAAAAANA | | | | | | | | |
| AAANAAAAAAAAA | | | | | | | | |
| AAAAAAAAAAAAS | | | | | | | | |
| AAAAASAAAAAAA | | | | | | | | |
| ASAAAAAAAAAAA | | | | | | | | |
| AFAAAKTAA | | | | | | | | |
| YARFLALTTLRARA | | | | | | | | |
| YAR(15A)SQTTLKAK | | 2.5 | | | | | 1.4 | 48 |
| T | | | | | | | | |
| YAR(15A)RQTTLKAA | | 1.2 | | | | | 0.94 | 62 |
| A | | | | | | | | |
| (15A)RQTTLKAAA | | 1.8 | | | | | 9.5 | 3095 |
| (16A)RQTTLKAAA | | 77 | | | | | 4000 | |
| (46)AAKTAAAFA | | | | | | | | |
| (39)AAAATKAAA | | | | | | | | |
| (52)AAAATKAAAA | | | | | | | | |
| (55)AAAATKAAAA | | | | | | | | |
| A(14)AAAKTAAA | | 43 | | | | | 120 | |
| AA(14)A(35)ATKAA | | | | | | | | |
| AA | | | | | | | | |
| AA(14)AA(36)TKAA | | | | | | | | |
| AA | | | | | | | | |
| AFAAAKTAA(72) | | | | | | | | |
| (49)AAAKT(64)AAA | | | | | | | | |
| (49)AAAKTA(64)AA | | | | | | | | |
| HQAISPRTLNGPGPGS | | 9875 | 638 | 5570 | | 232 | 32930 | |
| PAIF | | | | | | | | |
| YAAFAAAKTAAAFA | | | | >4347.83 | | | | |
| TEGRCLHYTVDKSKPK | | >1250 | | | 4082 | | 2857 | |
| AWYAWRNRCK | | >5000 | | | >11111.11 | | 44 | |
| IVSDGNGMNAWVAWR | | 6667 | | | >6250 | | >2222.22 | |
| NRC | | | | | | | | |
| PHHTALRQAILSWGEL | | 3116 | | 5.3 | | 48 | 261 | |
| MTLA | | | | | | | | |
| WMYYHGQRHSDEHHH | | >10000 | | | >7692.31 | | >5000 | |
| Y1VMSDWTGGA | | >6666.67 | | | >33333.33 | | >10000 | |
| AHAAHAAHAAHAAHAA | | 200000 | | | | | 200000 | |
| MD1DPYKEFGATVELL | | | 2415 | | | | | |
| SFLPSDFFP | | | | | | | | |
| GMLPVCPLIPGSSTTS | | 2500 | | | >25000 | | 200000 | |
| TGP | | | | | | | | |
| LGFFPDHQLDPAFRAN | | 6667 | | | 1449 | | 6667 | |
| TGYKVLVLNPSV | | 26 | 21 | 126 | | 995 | >11441.65 | |
| LMAFTAAVTS | | >23337.22 | >2464.79 | 1934 | | 11687 | >12586.53 | |
| TFALWRVSAEEY | | 342 | >2569.75 | >12709.5 | | >6608.93 | 25499 | |
| ALWRVSAEEY | | 243 | >6398.54 | >15268.46 | | >7930 | >35587.19 | |
| EEYVEIRQVGDFH | | 4683 | >1895.99 | 2060 | | 2063 | 9754 | |
| VGGVYLLPRRGPRIGV | | 88 | >15350.88 | 4.2 | 60753 | 19239 | 12 | |
| VGGAYLLPRRGPRLGV | | 507 | 24663 | 4.1 | >66533.6 | 37640 | 50 | |
| VGGVALLPRRGPRLGV | | 154 | >15350.88 | 8.5 | >66533.6 | 25688 | 20459 | |
| VGGVYALPRRGPRLGV | | 12 | >15350.88 | 451 | >66533.6 | 26122 | 34 | |
| VGGVYLAPRRGPRLGV | | 35 | >15350.88 | 55 | >66533.6 | >42059.46 | 76 | |
| VGGVYLLARRGPRLGV | | 6.5 | 10325 | 2.8 | 17030 4338 | 17 | | |
| VGGVYLLPARGPRLGV | | 694 | 201 | 6.5 | 18073 | 18960 | 40 | |
| VGGVYLLRRAGPRLGV | | 67 | >15350.88 | 6.2 | 91912 | 30707 | 7.9 | |
| GAPLGGAARALAHGV | | 24 | 8739 | 1615 | >70972.32 | 3959 | 11983 | |
| GAALGGAARALAHGV | | 168 | 19335 | 4483 | >70972.32 | 3509 | 25372 | |
| GAPLAGAARALAHGV | | 9.5 | 7215 | 2810 | >70972.32 | 2963 | 7688 | |
| GAPLGAAARALAHGV | | 36 | 15091 | 3920 | >70972.32 | 16533 | 4502 | |
| GAPLGGLARALAHGV | | 12 | 76 | 1805 | 123762 | 3950 | 4256 | |
| GAPLGGALRALAHGV | | 83 | 340 | 2068 | >51098.62 | 4889 | 5396 | |
| GAPLGGAAAALAHGV | | 43842 | 23810 | 7682 | >51098.62 | 31 | 12916 | |
| GAPLGGAARLLAHGV | | 80 | 29412 | 631 | >51098.62 | 2549 | 26684 | |
| GAPLGGAARAAAHGV | | 3633 | >23489.93 | >8666.67 | >51098.62 | 41441 | 42463 | |
| GAPLGGAARALAAGV | | 45 | 23179 | 5714 | >51098.62 | 3865 | 8354 | |
| FPDWQNYTPGPGTRF | | >51282.05 | >12027.49 | 35058 | | 33923 | >20533.88 | |
| RFPLTFGWCFKLVPV | | 62289 | 4797 | 514 | | 964 | >20533.88 | |
| RQDILDLWVYHTQGY | | >51282.05 | 6775 | 723 | | 1326 | 16155 | |
| RQEILDLWVYHTQGF | | 11113 | 5384 | 985 | | 1071 | >20533.88 | |
| LSHFLKEKGGLEGLI | | 9460 | >12027.49 | >39737.99 | | 18709 | >20533.88 | |
| LSFFLKEKGGLDGLI | | 614 | >12027.49 | >39737.99 | | 13214 | 15272 | |
| LEPWNHPGSQPKTACT | | >15325.67 | >11041.01 | 2665 | | 92 | 2939 | |
| QVCFITKGLGISYGR | | 31 | 92 | 3555 | | 876 | 3950 | |
| QLCFLKKGLGISYGR | | 9.5 | 88 | 4212 | | 282 | 1190 | |
| PPEESFRFGEEKTTPS | >10000 | | | >14285.71 | | >2857.14 | | |
| CIVYRDGNPYAVCDK | | >14662.76 | 1646 | 650 | | >24786.32 | >10666.67 | |
| HYCYSLYGTTLEQQY | | 12397 | >13725.49 | 4849 | | 1292 | >10666.67 | |
| CYSLYGTTLEQQYNK | | >14662.76 | >13725.49 | 5060 | | 189 | >10666.67 | |
| NTSLQDIEITCVYCK | | >14662.76 | 14857 | 678 | | 11710 | >10666.67 | |
| VFEFAFKDLFVVYRD | | 10923 | 7675 | 4871 | | 18117 | >10666.67 | |
| EFAFKDLFVVYRDSI | | 9496 | 9996 | 5355 | | 9072 | 5998 | |
| DLFVVYRDSIPHAAC | | 1163 | 11172 | 2832 | | 2676 | 10741 | |
| FVVYRDSIPHAACHK | | 1194 | 1851 | 349 | | 18144 | 2343 | |
| NTGLYNLLIRCLRCQ | | 14 | 5692 | 67 | | 222 | 598 | |
| IRCLRCQKPLNPAEK | | >14662.76 | >13725.49 | 6928 | | 611 | >10666.67 | |
| PRKLHELSSALEIPY | | 5990 | 51 | 1116 | | 1710 | >10666.67 | |
| EIPYDELRLNCVYCK | | >18001.8 | 858 | 2084 | | 9047 | >62305.3 | |
| TEVLDFAFTDLTIVY | | >18001.8 | >13059.7 | 561 | | 110 | >62305.3 | |
| VLDFAFTDLTIVYRD | | 7474 | 3102 | 645 | | 11294 | 14839 | |
| DFAFTDLTIVYRDDT | | 14334 | 5008 | 3651 | | 21621 | 675 | |
| TIVYRDDTPHGVCTK | | >18001.8 | 6280 | 5449 | | >21521.34 | >62305.3 | |
| WYRYSVYGTTLEKLT | | 1670 | 805 | 421 | | 1039 | 62 | |
| ETTIHNIELQCVECK | | >18001.8 | 6282 | 11191 | | 112 | >62305.3 | |
| SEVYDFAFADLTVVY | | >18001.8 | >13059.7 | 955 | | 1325 | 11802 | |
| VYDFAFADLTVVYRE | | >18001.8 | >13059.7 | 9446 | | 10720 | 27275 | |
| DFAFADLTVVYREGN | | >18001.8 | 9627 | 4915 | | 17973 | 39785 | |
| TVVYREGNPFGICKL | | >18001.8 | >13059.7 | 13850 | | 16200 | 48840 | |
| GNPFGICKLCLRFLS | | 1084 | 9737 | 1139 | | 196 | 6594 | |
| NYSVYGNTLEQTVKK | | >56657.22 | 8614 | 15587 | | >25108.23 | 14326 | |
| KKPLNEILIRCHCQ | | 1299 | 965 | 1870 | | 140 | 26273 | |
| NEILIRCIICQRPLC | | 20827 | 7174 | 18927 | | 883 | >29761.9 | |
| IRCIICQRPLCPQEK | | 6757 | 7295 | 25349 | | 510 | 15154 | |
| CIVYRDCIAYAACHK | | 35566 | 12898 | 3847 | | 2578 | 1912 | |
| NTELYNLLIRCLRCQ | | 259 | 5674 | 2449 | | 797 | 854 | |
| IRCLRCQKPLNPAEK | | 21581 | >9641.87 | 27591 | | 447 | 20171 | |
| REVYKFLFTDLRIVY | | 2263 | 80 | 258 | | 203 | 155 | |
| RIVYRDNNPYGVCIM | | 3446 | 119 | 821 | | 1403 | 20474 | |
| NNPYGVCIMCLRFLS | | 7786 | 4797 | 6662 | | 207 | 7258 | |
| EERVKKPLSEITIRC | | 6877 | 8919 | 132 | | 2990 | 7910 | |
| IRCIICQTPLCPEEK | | 5461 | 17444 | 9766 | | 916 | >51020.41 | |
| EIPLIDLRLSCVYCK | | 47355 | 6936 | 656 | | 861 | 16853 | |
| SCVYCKKELTRAEVY | | 569 | 23385 | 4374 | | 673 | 3197 | |
| VCLLFYSKVRKYRYY | | 326 | 309 | 61 | | 2343 | 182 | |
| YYDYSVYGATLESIT | | 9122 | 8923 | 1106 | | 32378>51020.41 | | |
| IRCYRCQSPLTPEEK | | 6645 | >14403.29 | 480 | 28659 | >51020.41 | | |
| VYDFVFADLRGVYRD | | 12168 | 79 | 855 | | 4392 | >51020.41 | |
| DFVFADLRIVYRDGN | | 6957 | 162 | 1253 | | 6709 | 8433 | |
| RIVYRDGNPFAVCKV | | 174 | 122 | 81 | | 1606 | 3148 | |
| GNPFAVCKVCLRLLS | | 296 | 7389 | 117 | | 126 | 657 | |
| KKCLNEILIRCIICQ | | 7579 | 731 | 3176 | | 257 | >9925.56 | |
| NEILIRCIICQRPLC | | 16056 | 10184 | 8177 | | 372 | >22909.51 | |
| RTAMFQDPQERPRKL | | 1034 | 17086 | 73192 | | 20481 | 7474 | |
| LFVVYRDSIPHAACH | | 1582 | 697 | 437 | | 3580 | 7854 | |
| LT1VYRDDTPHGVCT | | 15880 | 1852 | 27048 | | 16993 | >15267.18 | |
| LCIVYRDCIAYAACH | | 9886 | 5662 | 2269 | | 2881 | 9738 | |
| YKFLFTDLRIVYRDN | | 10122 | 77 | 2912 | | 1342 | 800 | |
| YNFACTELKLVYRDD | | 11615 10167 | 3082 | | 12866 | 1673 | | |
| LKLVYRDDFPYAVCR | | 698 | 699 | 1877 | | 3828 | 9156 | |
| YDFVFADLRIVYRDG | | 6540 | 8173 | 25727 | | 10907 | 11161 | |
| LRIVYRDGNPFAVCK | | 109 | 123 | 169 | | 1566 | 6820 | |
| HEYMLDLQPETTDLY | | >56179.78 | 12990 | 30895 | | 2099 | >22909.51 | |
| TLRLCVQSTHVDIRT | | 17613 | 932 | 3957 | | 243 | >22909.51 | |
| IRTLEDLLMGTLGIV | | 1156 | 789 | 2181 | | 23 | 12385 | |
| LEDLLMGTLGIVCPI | | 8514 | 1693 | 229 | | 1800 | 9475 | |
| DLLMGTLGIVCPICS | | >56179.78 | 1053 | 1427 | | 4123 | 16198 | |
| KATLQDIVLHLEPQN | | 25948 | 603 | 6968 | | 159 | >9925.56 | |
| IDGVNHQHLPARRAE | | >56179.78 | >11475.41 | >36842.11 | | 344 | 12573 | |
| LRAFQQLFLNTLSFV | | 106 | 1.01 | 20 | | 2.2 | 253 | |
| FQQLFLNTLSFVCPW | | 10311 | 9.3 | 24792 | | 309 | 17330 | |
| QDYVLDLQPEATDLH | | >11918.95 | >11475.41 | >62758.62 | | 1851 | >22909.51 | |
| DIRILQELLMGSFGI | | 18982 | 5796 | 1625 | | 16 | >55096.42 | |
| IRILQELLMGSFGIV | | 7978 | 1038 | 294 | | 17 | >55096.42 | |
| ELLMGSFGIVCPNCS | | >59171.6 | 933 | 1928 | | 206 | >55096.42 | |
| KEYVLDLYPEPTDLY | | >59171.6 | >14767.93 | 3171 | | 476 | >55096.42 | |
| LRTIQQLLMGTVNIV | | 3641 | 6.4 | 265 | | 15 | 32108 | |
| IQQLLMGTVNIVCPT | | 11062 | 9.0 | 2010 | | 166 | >55096.42 | |
| QLLMGTVNIVCPTCA | | >59171.6 | 118 | >38396.62 | | 11550 | >55096.42 | |
| RETLQEIVLHLEPQN | | 7896 | 11360 | 16220 | | 95 | >55096.42 | |
| LRTLQQLFLSTLSFV | | 208 | 55 | 29 | | 3.1 | 1994 | |
| LQQLFLSTLSFVCPW | | 11693 | 133 | 296 | | 22 | 36943 | |
| KDYILDLQPETTDLH | | >17436.79 | 23654 | >37448.56 | | 490 | >55096.42 | |
| LRTLQQMLLGTLQVV | | 907 | 616 1697 | | 88 | >46620.05 | | |
| LQQMLLGTLQVVCPG | | >31645.57 | 395 | 1266 | | 1014 | 29198 | |
| QMLLGTLQVVCPGCA | | >31645.57 | 874 | 4144 | | 258 | >31446.54 | |
| VPTLQDVVLELTPQT | | >31645.57 | 14985 | 12263 | | 1000 | >31446.54 | |
| LQDVVLELTPQTEID | | >31645.57 | 1145 | >33090.9 | | 1116 | >31446.54 | |
| QDVVLELTPQTEIDL | | >31645.57 | 10274 | >33090.9 | | 1719 | >31446.54 | |
| CKFVVQLDIQSTKED | | >31645.57 | >11437.91 | 22851 | | 301 | >31446.54 | |
| VYQLDIQSTKEDLRV | | 7353 | 708 | 5044 | | 226 | 8690 | |
| DLRVVQQLLMGALTV | | 667 | 57 | 132 | | 9.5 | 10879 | |
| LRVVQQLLMGALTVT | | 314 | 8.9 | 56 | | 7.7 | 8755 | |
| VQQLLMGALTVTCPL | | 11074 | 574 | 526 | | 204 | 7151 | |
| QQLLMGALTVTCPLC | | 7657 | 1223 | 4461 | | 1470 | >31446.54 | |
| QLLMGALTVTCPLCA | | >31645.57 | 1817 | 3761 | | 2224 | >31446.54 | |
| REYILDLHPEPTDLF | | 4152 | 13183 | >33090.9 | | 316 | >31446.54 | |
| TCCYTCGTTVRLCNG | | 8636 | 739 | 3820 | | 891 | 16033 | |
| VRTLQQLLMGTCTIV | | 1409 | 37 | 1829 | | 139 | >15267.18 | |
| LQQLLMGTCTIVCPS | | 9447 | 753 | 2441 | | 2667 | >15267.18 | |
| MLDLQPEITDLYCYE | | >15209.13 | >12027.49 | >48404.26 | | 20 | >15267.18 | |
| VLDLYPEPTDLYCYE | | >15209.13 | >12027.49 | 21591 | | 18 | >15267.18 | |
| LREYILDLHPEPTDL | | 9827 | 12365 | 10949 | | 2040 | >40404.04 | |
| HIEFTPTRTDTYACRV | | 200000 | | | >7142.86 | | 200000 | |
| LWWVNNESLPVSPRL | | | | | | | | |
| YEEYVRFDSDVGE | | 200000 | | | | | 200000 | |
| EEYVRFDSDVGE | | 200000 | | | | | 200000 | |
| APPRLICDSRVLERY | | >1111111.11 | 149 | 1384 | 1617 | 2840 | 6087 | |
| ICDSRVLERYLLEAK | | 2945 | 20402 | 85 | 16159 | 8550 | 7295 | |
| VLERYLLEAKEAENI | | 17227 | 881 | 269 | 340 | 8920 | 6714 | |
| EHCSLNENITVPDTK | | >1111111.11 | 84 | 12013 | 8307 | 52943 | 6626 | |
| NENITVPDTKVNFYA | | 17921 | 9338 | 22568 | >38167.94 | >38461.54 | 12214 | |
| VPDTKVNFYAWKRME | | 8861 | 14795 | 333 | >38167.94 | 23602 | 449 | |
| VNFYAWKRMEVGQQA | | 50 | 14798 | 1194 | 22507 | 1490 | 455 | |
| WKRMEVGQQAVEVWQ | | 512 | 159 | 1812 | >42194.09 | 238 | 4300 | |
| VGQQAVEVWQGLALL | | >17241.38 | 1313 | 12 | >38167.94 | 3901 | >7785.13 | |
| VEVWQGLALLSEAVL | | 5157 | 4473 | 58 | >38167.94 | 1334 | 13794 | |
| GLALLSEAVLRGQAL | | 2578 | 1216 | 1939 | >38167.94 | 3.5 | 105 | |
| SEAVLRGQALLVNSS | | 3484 | 7.4 | 151 | 3997 | 23 | 1057 | |
| RGQALLVNSSQPWEP | | 7698 | 3.4 | 2876 | 6165 | 1554 | 558 | |
| LVNSSQPWEPLQLHV | | >8163.27 | 504 | 2359 | 18044 | 3412 | 10039 | |
| QPWEPLQLHVDKAVS | | 8897 | 695 | 12480 | 1924 | 103 | 2929 | |
| LQLHVDKAVSGLRSL | | 910 | 53 | 2707 | 1044 | 31 | 76 | |
| DKAVSGLRSLTTLLR | | 52 | 187 | 60 | 3150 | 2006 | 104 | |
| GLRSLTTLLRALGAQ | | 3.7 | 871 | 6.2 | 12947 | 283 | 2.7 | |
| TTLLRALGAQKEAIS | | 860 | 1512 | 89 | 33256 | 251 | 21 | |
| ALQAQKEAISPPDAA | | 4212 | >12411.35 | 14216 | >91743.12 | 27294 | 3963 | |
| KEAISPPDAASAAPL | | 601 | 9272 | 1201 | 27203 | 2988 | 310 | |
| PPDAASAAPLRTITA | | 2582 | 10205 | 1267 | 10584 | 182 | 1117 | |
| SAAPLRT1TADTFRK | | 3883 | 809 | 858 | 2111 | 17 | 45 | |
| RTITADTFRKLFRVY | | 166 | 95 | 35 | 672 | 1561 | 93 | |
| DTFRKLFRVYSNFLR | | 11 | 10 | 0.95 | 43687 | 1029 | 26 | |
| LFRVYSNFLRGKLKL | | 173 | 80 | 2.8 | 8981 | 2333 | 2.9 | |
| SNFLRGKLKLYTGEA | | 192 | 4730 | 30 | 4075 | 2442 | 5.7 | |
| KLKLYTGEACRTGDR | | >17241.38 | 880 | 130 | 17787 | 20089 | 636 | |
| APPRLITDSRVLERY | | 2750 | 92 | 238 | 710 | 2263 | 698 | |
| ITDSRVLERYLLEAK | | 5279 | >14705.88 | 18 | >42194.09 | 12401 | 621 | |
| EHTSLNENITVPDTK | | >408163.27 | 13 | 11082 | >42194.09 | >29029.03 | 5547 | |
| KLKLYTGEATRTGDR | | 4364 | 841 | 18 | 5298 | 14838 | 731 | |
| PQPFRPQQPYPQ | | | | | | 15 | | |
| PFRPQQPYPQ | | | | | | 42 | | |
| PQPFRPQQPYP | | | | | | 14 | | |
| PQPFRPQQP | | | | | | 19 | | |
| KQPFRPQQPYPQ | | | | | | 56 | | |
| PKPFRPQQPYPQ | | | | | | 3.4 | | |
| PQPFKPQQPYPQ | | | | | | 19 | | |
| PQPFRKQQPYPQ | | | | | | 22 | | |
| PQPFRPQKPYPQ | | | | | | 22 | | |
| PQPFRPQQPKPQ | | | | | | 325 | | |
| PQPFRPQQPYKQ | | | | | | 35 | | |
| PQPFRPQQPYPK | | | | | | 22 | | |
| QFLGQQQPFPPQ | | | | | | 2.8 | | |
| FLGQQQPFPPQ | | | | | | 31 | | |
| LGQQQPFPPQ | | | | | | 151 | | |
| QFLGQQQPFPP | | | | | | 2.3 | | |
| QFLGQQQPF | | | | | | 5.3 | | |
| IRNLALQTLPAMCNVY | | | | | | 1.9 | | |
| NLALQTLPAMCNVY | | | | | | 27 | | |
| LALQTLPAMCNVY | | | | | | 153 | | |
| IRNLALQTLPAM | | | | | | 2.0 | | |
| IRNLALQTLP | | | | | | 3.0 | | |
| EGDAFELTVSCQGGLP | | | | | | | | |
| K | | | | | | | | |
| ESTGMTPEKVPVSEVM | | | >17500 | | | >64444.44 | | |
| FPTIPLSRLFDNASL | | 30675 | 7495 | 1390 | 2585 | 194 | 5799 | |
| RLFDNASLRAHRLHQ | | 12461 | 84 | 85 | 11411 | 3210 | 557 | |
| LRAHRLHQLAFDTYQ | | 3208 | 7590 | 90 | 19811 | 2.0 | 4471 | |
| QLAFDTYQEFEEAYI | | >15384.62 | 15167 | 23166 | 595 | 11495 | >38610.04 | |
| QEFEEAYIPKEQKYS | | 12821 | >15837.1 | >15582.19 | >54554.47 | >41134.75 | 5418 | |
| IPKEQKYSFLQNPQT | | >15384.62 | 13695 | 16207 | 30572 | 55587 | 13118 | |
| SFLQNPQTSLCFSES | | >15384.62 | 190 | 6513 | 93809 | 21651 | >9647.76 | |
| TSLCFSESIPTPSNR | | >15384.62 | 99 | 1944 | 3920 | 1883 | >38610.04 | |
| RBETQQKSNLELLRI | | >15384.62 | 15709 | 9736 | >270270.27 | 52 | 25133 | |
| SNLELLRISLLLIQS | | 23669 | 196 | 59 | >91901.83 | 147 | 50110 | |
| ISLLLIQSWLEPVQF | | 2675 | 120 | 60 | 6765 | 2.5 | >9960.16 | |
| SWLEPVQFLRSVFAN | | 2715 | 4322 | 136 | >270270.27 | 291 | 4815 | |
| FLRSVFANSLVYGAS | | 973 | 5.6 | 13 | 157978 | 814 | 141 | |
| NSLVYGASDSNVYDL | | >15384.62 | 14038 | 3640 | 11769 | 1792 | >13046.31 | |
| SDSNVYDLLKDLEEG | | >15384.62 | >17857.14 | >30536.91 | 219298 | >137767.22 | >13046.31 | |
| GIQTLMGRLEDGSPR | | 4474 | 10433 | 1348 | 186220 | 2110 | 18006 | |
| RLEDGSPRTGQIFKQ | | 7896 | >17857.14 | 9106 | 18119 | 296 | 12580 | |
| RTGQIFKQTYSKFDT | | 6961 | 66 | 155 | 14736 | 201 | 64 | |
| QTYSKFDTNSHNDDA | | >15384.62 | >17857.14 | 25883 | 38715 | >137767.22 | 5787 | |
| TNSHNDDALLKNYGL | | >15384.62 | 5169 | 133 | 130378 | >137767.22 | >13046.31 | |
| ALLKNYGLLYCFRIW | | >15384.62 | 10 | 17 | 2309 | 1230 | 462 | |
| DMDKVETFLRIVQCR | | 885 | 1232 | 201 | >27322.4 | 826 | 7447 | |
| FLRIVQCRSVEGSCGF | | 2708 | 1017 | 839 | >27322.4 | 1078 | 7102 | |
| FPTIPLSRLFDNAML | | 46404 | 9313 | 2770 | 121212 | 216 | 11521 | |
| RLFDNAMLRAHRLHQ | | 267 | 738 | 18 | >270270.27 | 1628 | 58 | |
| QLAFDTYQEFEQNPQ | | >15384.62 | 19718 | >86666.67 | 738 | >32842.58 | >9510.22 | |
| SFLQNFQTSLCCFRK | | 3801 | 128 | 103 | >270270.27 | 8500 | 3739 | |
| SNLELLRICLLLIQS | | >15384.62 | 773 | 90 | 17024 | 164 | >11771.33 | |
| ICLLLIQSWLEPVQF | | >15384.62 | 954 | 1771 | 187970 | 49 | >9510.22 | |
| NSLVYGASDSNIYDL | | >15384.62 | 10854 | 971 | 31616 | 3287 | >9510.22 | |
| SDSNIYDLLKDLEEG | | >15384.62 | >16203.7 | >86666.67 | >18726.59 | 24259 | >9510.22 | |
| DKVETFLRIVQCCGF | | 1023 | 1034 | 383 | 6278 | 184 | 6350 | |
| SFLQNPQTSLTFSES | | >15384.62 | 121 | 1511 | 864 | 17824 | 12365 | |
| TSLTFSESIPTPSNR | | 22152 | 16 | 176 | >95238.1 | 3476 | >1335.38 | |
| ALLKNYGLLYTFRKD | | 1737 | 0.89 | 6.5 | 50 | 1335 | 29 | |
| LLYTFRKDMDKVETF | | 7905 | >14522.82 | 886 | 941 | 12493 | 154 | |
| DMDKVETFLRIVQTR | | 206 | 3381 | >86666.67 | 13712 | 190 | 1263 | |
| FLRIVQTRSVEGSTGF | | 143 | 1.5 | 9.8 | 27345 | 21 | 116 | |
| HLDMLRHLYQGCQVV | | 2076 | 2879 | 359 | 107066 | 163 | 7087 | |
| RLRIVRGTQLFEDNYA | | 2072 | 5.2 | 31 | 1198 | 120 | 46 | |
| L | | | | | | | | |
| GVGSPYVSRLLGICL | | 696 | 955 | 46 | 148588 | 316 | 14197 | |
| TLERPKTLSPGKNGV | | >52631.58 | 835 | 23264 | >263157.89 | 25739 | 11337 | |
| KIFGSLAFLPESFDGD | | >52631.58 | 1073 | 2264 | 43745 | 10020 | 8008 | |
| PA | | | | | | | | |
| ELVSEFSRMARDPQ | | 4573 | >71428.57 | 7891 | 15838 | 970 | 4055 | |
| GEALSTLVLNRLKVG | | 79 | 29 | 269 | | 1023 | 46 | |
| AYVLLSEKKISSIQS | | 51 | 816 | 489 | | 902 | 4517 | |
| VASLLTTAEVVVTEI | | >18674.14 | >10294.12 | >50837.99 | | >26435.73 | >119047.62 | |
| KCEFQDAYVILLSEKK | | 1078 | >10294.12 | >47643.98 | | >19594.59 | 20 | |
| ALSTLVLNRLKVGLQ | | 9.1 | 4.6 | 191 | | 17 | 3.9 | |
| MSYNLLGFLQRSSNC | | 3628 | 1190 | 89 | >42194.09 | 6503 | 710 | |
| LGFLQRSSNCQCQKL | | 6025 | 112 | 1397 | >42194.09 | 1167 | 649 | |
| RSSNCQCQKLLWQLN | | >408163.27 | 6153 | 802 | 3519 | 21 | 6981 | |
| QCQKLLWQLNGRLEY | | 1644 | 227 | 175 | 8709 | 209 | 924 | |
| LWQLNGRLEYCLKDR | | 4215 | 808 | 893 | 29028 | 15576 | 3241 | |
| GRLEYCLKDRRNFDI | | 1707 | 1240 | 940 | 5213 | 15870 | 64725 | |
| RNFD1PEEIKQLQQF | | 7326 | >15418.5 | 2036 | 23832 | 311 | 6854 | |
| PEEIKQLQQFQKEDA | | 1953 | 13325 | 1873 | >26315.79 | 215 | 675 | |
| QLQQFQKEDAAVTIY | | >408163.27 | 68 | 1724 | 348 | 1338 | 4270 | |
| QKEDAAVTIYEMLQN | | >408163.27 | 7315 | 1146 | >42194.09 | 15173 | >10482.18 | |
| AVTIYEMLQNIFAIF | | 29718 | 109 | 262 | 2828 | 1118 | 14047 | |
| EMLQNIFAIFRQDSS | | 36832 | 61 | 1718 | 726 | 164 | 3187 | |
| IFAIFRQDSSSTGWN | | 4558 | 775 | 204 | 2181 | 30 | 109290 | |
| RQDSSSTGWNETIVE | | >42553.19 | 848 | >189583.33 | 9172 | 1497 | 8650 | |
| STGWNETIVENLLAN | | 20576 | 105 | 897 | >26315.79 | 166 | 5822 | |
| ETIVENLLANVYHQR | | >42553.19 | 8.5 | 1603 | >42194.09 | 2503 | 18559 | |
| NLLANVYHQRNHLKT | | 8258 | 61 | 20 | >123456.79 | 3071 | 65 | |
| VYHQRNHLKTVLEEK | | 22002 | 1267 | 1662 | >123456.79 | 9585 | 4.7 | |
| LEKEDFTRGKRMSSL | | 698 | 25362 | 14118 | 6267 | 16057 | 4903 | |
| FTRGKRMSSLHLKRY | | 81 | 10245 | 118 | 18836 | 2027 | 84 | |
| RMSSLHLKRYYGRIL | | 1035 | 2532 | 1.3 | >26178.01 | 2255 | 491 | |
| HLKRYYGRILHYLKA | | 2721 | 868 | 0.69 | 6608 | 22 | 2.3 | |
| YORILHYLKAKBDSH | | 812 | 2783 | 16 | 454545 | 140 | 39 | |
| HYLKAKEDSHCAWTI | | >60606.06 | 11571 | 627 | 301205 | 7501 | 2632 | |
| KEDSHCAWTIVRVEI | | 9320 | 506 | 1397 | >1754385.96 | 7.9 | 4056 | |
| CAWTIVRVEILRNFY | | 4167 | 147 | 196 | 10300 | 152 | 4143 | |
| VRVEILRNFYVINRL | | 504 | 5.8 | 1.04 | 80386 | 187 | 485 | |
| RNFYVINRLTGYLRN | | 55 | 9.4 | 18 | 689 | 1249 | 5.6 | |
| MSYNLLGFLQRSSNT | | 3069 | 1334 | 6.8 | 51787 | 4660 | 9.0 | |
| LGFLQRSSNTQTQKL | | 26247 | 21 | 2331 | >1754385.96 | 1041 | 339 | |
| RSSNTQTQKLLWQLN | | >42553.19 | 169 | 2740 | 751 | 26 | 8545 | |
| QTQKLLWQLNGRLEY | | 20654 | 121 | 20 | 6582 | 88 | 417 | |
| LWQLNGRLEYTLKDR | | 6521 | 2447 | 853 | 4402 | 14310 | 6004 | |
| GRLEYTLKDRRNFDI | | 4998 | 1468 | 168 | 9901 | 21427 | 796 | |
| HYLKAKEDSHTAWTI | | >60606.06 | 2264 | 529 | 35829 | 11750 | 19617 | |
| KEDSHTAWTIVRVEI | | 7443 | 3046 | 1992 | 56205 | 18 | 575 | |
| TAWTIYRVEILRNFY | | 5052 | 72 | 242 | 14419 | 26 | 518 | |
| LGFLQRSSNCQSQKL | | 604 | 131 | 541 | >1754385.96 | 124 | 508 | |
| RSSNCQSQKLLWQLN | | >60606.06 | 1960 | 2962 | 68823 | 27 | 4077 | |
| QSQKLLWQLNGRLEY | | >60606.06 | 155 | 108 | 5609 | 166 | 402 | |
| GIVEQCCTSICSLYQ | | 7940 | 239 | 1280 | 14353 | 4245 | >37593.98 | |
| TSICSLYQLENYCN | | >10526.32 | >15021.46 | 837 | 8048 | 13496 | >40322.58 | |
| GILEQCCTSICSLYQ | | >10526.32 | 858 | 1097 | >18726.59 | 5871 | 19231 | |
| G1VEQTTTSITSLYQ | | >10526.32 | 14 | 849 | >95238.1 | 2303 | >37593.98 | |
| EQTTTSITSLYQLEN | | >10526.32 | 16949 | 1078 | >18726.59 | 29614 | 48505 | |
| TSICSLYQLENYCG | | >10526.32 | 10346 | 173 | >95238.1 | 1645 | >40322.58 | |
| TSITSLYQLENYTN | | 1095 | >17073.17 | 99 | >95238.1 | 3245 | 6048 | |
| TSITSLYQLENYTG | | 1014 | >17073.17 | 182 | 92336 | 1658 | 16073 | |
| GIVEQCCCGSHLVEA | | >10526.32 | 15347 | 237 | 14184 | 11017 | >43290.04 | |
| SLYQLENYCCGERGF | | >1111111.11 | >15909.09 | 151 | 92336 | 30978 | >43290.04 | |
| CCTSICSLYQLENYCC | | >1111111.11 | 7096 | 877 | >18726.59 | 1582 | >40650.41 | |
| GSHLVEALYLVCCN | | >1111111.11 | 3259 | 11191 | >18726.59 | 14065 | >46403.71 | |
| CCGSHLVEALYLVCC | | >10526.32 | 6027 | 12986 | >18726.59 | 11357 | >43290.04 | |
| FVNQHLCGSHLVEAL | | >1111111.11 | 10595 | 1195 | >95238.1 | 3153 | 47170 | |
| QHLCGSHLVEALYLV | | >10526.32 | 7624 | 103 | 14819 | 1480 | 32049 | |
| GSHLVEALYLVCGER | | >10526.32 | 8030 | 1350 | >18726.59 | 372 | 29283 | |
| VEALYLVCGERGFFY | | 3563 | 4403 | 181 | 4443 | 30 | 25543 | |
| YLVCGERGFFYTPKT | | >10526.32 | 9272 | 10655 | 92764 | 34450 | 95238 | |
| FVNQHLCGSDLVEAL | | >1111111.11 | 20248 | 9679 | 10031 | 24511 | >43290.04 | |
| FVNQHLTGSHLVEAL | | >10526.32 | 12413 | 799 | 94518 | 4084 | >43290.04 | |
| QHLTGSHLVEALYLV | | >10526.32 | 6862 | 184 | 4027 | 939 | 23716 | |
| GSHLVEALYLVTGER | | >10526.32 | 12185 | 1429 | 18215 | 225 | 11398 | |
| VEALYLVCGERGSFY | | >10526.32 | 4288 | 1240 | >95238.1 | 129 | 804 | |
| VEALYLVCGERGFLY | | 55402 | 1871 | 149 | 843 | 19 | 5149 | |
| VEALYLVTGERGFFY | | 4860 | 1076 | 116 | 17156 | 13 | 78 | |
| YLVCGERGFLYTPKT | | >1111111.11 | 2120 | >25633.8 | >95238.1 | 33114 | 971 | |
| YLVCGERGFFYTDKT | | >60606.06 | 1014 | >25633.8 | 616 | 48099 | >28449.5 | |
| YLVCGERGFFYTKPT | | >60606.06 | 3467 | >25633.8 | 12805 | 40379 | >28449.5 | |
| YLVTGERGFFYTPKT | | 7625 | 2100 | >25633.8 | 13737 | 20721 | >28449.5 | |
| YLVTGERGFFYTDKT | | 16849 | 17353 | >25633.8 | 359 | 30824 | >28449.5 | |
| YLVTGERGFFYTKPT | | 9341 | 17869 | >21016.17 | 9573 | 27915 | 11926 | |
| VCGERGFFYTPKTRR | | 3817 | 34669 | >25633.8 | 17416 | >30999.47 | 92 | |
| VTGERGFFYTPKTRR | | 10116 | 25362 | 2824 | 243902 | >29820.05 | 540 | |
| MWDLVLSIALSVGCT | | 81096 | 108 | 11375 | 15205 | 158 | 70711 | |
| DLVLSIALSVGCTGA | | >200000 | 98 | 18200 | >14918.69 | 459 | >100000 | |
| HPQWVLTAAHCLKKN | | 981 | 483 | 1219 | 8114 | 1106 | 11 | |
| QWVLTAAHCLKKNSQ | | 14213 | >35000 | >45500 | >14918.69 | 14395 | 382 | |
| GQRVPVSHSFPHPLY | | >200000 | 703 | 3960 | >14918.69 | 9860 | >200000 | |
| RVPVSHSFPHPLYNM | | >200000 | 377 | 5518 | >14918.69 | 9213 | 11650 | |
| PHPLYNMSLLKHQSL | | 6455 | 3307 | 3873 | >14918.69 | 49 | 1901 | |
| HPLYNMSLLKHQSLR | | 248 | 546 | 472 | >14918.69 | 8.4 | 219 | |
| NMSLLKHQSLRPDED | | 25820 | >35000 | >30333.33 | >14918.69 | 105 | >100000 | |
| SHDLMLLRLSEPAKI | | 5267 | 1.8 | 365 | 5361 | 10 | 2031 | |
| HDLMLLRLSEPAKIT | | 1147 | 0.83 | 115 | 488 | 12 | 211 | |
| PEEFLRPRSLQCVSL | | 10675 | 11667 | 3193 | >14413.38 | 117 | 57537 | |
| PRSLQCVSLHLLSND | | 11128 | 3731 | 1597 | 11650 | 544 | 46416 | |
| NGVLQGITSWGPEPC | | 32444 | >17500 | 835 | >14413.38 | 5761 | >100000 | |
| KPAVYTKVVHYRKWI | | 327 | 1947 | 401 | 7186 | 4581 | 23 | |
| LHLLSNDMCARAYSE | | 26012 | 1876 | >2367.33 | 1308 | 324 | 28817 | |
| VGNWQYFFPVIFSKA | | | | | | | | |
| ESEFQAALSRKVAKL | | | | | | | | |
| IGHLYIFATCLGLSYD | | | | | | | | |
| GL | | | | | | | | |
| VGNWQYFFPVIFSKAS | | | | | | | | |
| DSLQLVFGIELMEVD | | | | | | | | |
| PAYEKLSAEQSPPPY | | | | | | | | |
| RNGYRALMDKSLHVG | | | | | | | | |
| TQCALTRR | | | | | | | | |
| FFKNIVTFFKNIVT | | | | | | | | |
| YKSAHKGFKGVDAQG | | 2000 | | | | | 1333 | 2065 |
| TLSKI | | | | | | | | |
| VDAQGTLSKIFKLGGR | | 18 | | | 769 | | 6667 | 1152 |
| DSRS | | | | | | | | |
| AC- | | 200000 | | | | 200000 | 4561 | |
| ASQKRPSQRHGSKYLA | | | | | | | | |
| TAST | | | | | | | | |
| ENPVVHFFKNIVTPR | | | | 5.2 | | | 463 | |
| ENPVVAFFKNIVTPR | | | | 2.8 | | | 302 | |
| ENPVVHAFKNIVTPR | | | | 4.1 | | | 910 | |
| ENPVVHFFANIVTPR | | | | 2.9 | | | 6235 | |
| ENPVVHFFKIHVTPA | | | | 2.5 | | | 3333 | |
| NPVVHFFKNIVT | | | | 23 | | | 10000 | |
| HFFKNIVTPRTPPY | | | | 460 | | | 377 | |
| NPVVHFFKNIVTPR | | | | 3.7 | | | 1890 | |
| LPVPGVLLKEFTVSGN | | 216 | 52 | 84 | | 349 | 1840 | |
| ILTI | | | | | | | | |
| WITQCFLPVFLAQPPS | | 13208 | 23649 | 726 | | 688 | 286 | |
| CIQRR | | | | | | | | |
| DHRQLQLSISSCLQQL | | >98522.17 | 69 | 67 | | 532 | 63772 | |
| SLLM | | | | | | | | |
| YLAMPFATPMEAELAR | | 3754 | 2813 | 865 | | 1965 | 641 | |
| RSLA | | | | | | | | |
| AAPLLLARAASLSLG | | 100 | 3.2 | 35 | 10470 | 79 | 79 | |
| APLLLARAASLSLGF | | 322 | 12 | 91 | 13359 | 59 | 114 | |
| PLLLARAASLSLGFL | | 1255 | 12 | 118 | >9742.79 | 52 | 151 | |
| SLSLGFLFLLFFWLD | | 100000 | 639 | 11375 | 3710 | >10955.8 | 66667 | |
| LLFFWLDRSVLAKEL | | 154 | 24 | 34 | 86 | 7.5 | 134 | |
| DRSVLAKELKFVTLV | | 20966 | 4410 | 1359 | >14413.38 | 53 | 2217 | |
| AKELKFVTLVFRHGD | | 12309 | 824 | 1529 | 8563 | 51 | 24 | |
| RSPIDTFPTDPIKES | | >200000 | >35000 | 2373 | >14413.38 | 469 | 28571 | |
| FGQLTQLGMEQHYEL | | 27217 | >35000 | >22750 | >14413.38 | 543 | 100000 | |
| DRTLMSAMTNLAALF | | 2367 | 114 | 871 | 3927 | 57 | 26138 | |
| MSAMTALAALFPPEG | | >200000 | 249 | 12384 | 7158 | 1072 | 63246 | |
| MTNLAALFPPEGVSI | | 141421 | 1310 | 10370 | >8829.24 | 4606 | 141421 | |
| PEGVSIWNPILLWQP | | 30861 | 444 | 7.2 | 4624 | 107 | 22222 | |
| GVSIWNPILLWQPIP | | 10287 | 207 | 5.0 | 4428 | 492 | 523 | |
| WNPILLWQPIPVHTV | | 19640 | 2259 | 14 | >8829.24 | 81 | 100000 | |
| NPILLWQPIPVHTVP | | 599 | 250 | 4.6 | >8829.24 | 67 | 25000 | |
| PILLWQPIPVHTVPL | | 4041 | 567 | 6.9 | >8829.24 | 106 | 41491 | |
| ILLWQPIPVHTVPLS | | 2343 | 1111 | 65 | >8829.24 | 712 | 28768 | |
| WQPIPVHTVPLSEDQ | | >66666.67 | 2692 | >45500 | >8829.24 | 1228 | >100000 | |
| LSGLHGQDLFGIWSK | | 30151 | >35000 | 32173 | >8829.24 | 135 | 81650 | |
| YDPLYCESVHNFTLP | | 30151 | >35000 | 2136 | >8829.24 | 6901 | 28768 | |
| LPSWATEDTMTKIRE | | >66666.67 | >35000 | >45500 | 5973 | >11134.57 | 343 | |
| LRELSELSLLSLYGI | | 6958 | 3218 | 235 | >14956.63 | 544 | 5185 | |
| LSELSLLSLYGIHKQ | | 1657 | 1253 | 45 | >13046.31 | 79 | 7.3 | |
| LSLLSLYGIHKQKEK | | 742 | >35000 | 58 | >14956.63 | 772 | 3.4 | |
| KSRLQGGVLVNEILN | | >66666.67 | 318 | >30333.33 | >14956.63 | 713 | >100000 | |
| GGVLVNEILNHMKRA | | 255 | 49 | 576 | 8124 | 5.8 | 8.7 | |
| IPSYKKLIMYSAHDT | | 53 | 2122 | 17 | 9982 | 12 | 191 | |
| YKKLIMYSAHDTTVS | | 208 | 37 | 15 | 13224 | 5.8 | 5482 | |
| LIMYSAHDTTVSGLQ | | >66666.67 | 1752 | 184 | 6828 | 4381 | >100000 | |
| DTTVSGLQMALDVYN | | >50000 | 3500 | 1042 | 10843 | 961 | >200000 | |
| ALDVYNGLLPPYASC | | 182 | >35000 | 1091 | >14956.63 | >10090.47 | 115470 | |
| LDVYNGLLPPYASCH | | 194 | >35000 | 3035 | >14956.63 | >10918.67 | 25820 | |
| YNGLLPPYASCHLTE | | 5300 | 11667 | 252 | >14956.63 | >10918.67 | 100000 | |
| FAELVGPVIPQDWST | | >50000 | >35000 | >45500 | >14956.63 | 983 | >200000 | |
| TVPLSEDQLLYLPFR | | 26455 | 5300 | >2367.33 | 4323 | 872 | 27221 | |
| LTELYFEKGEYFVEM | | >18903.59 | 3157 | >2367.33 | 124 | 601 | 6655 | |
| GPVIPQDWSTECMTT | | | | | 20295 | 961 | | |
| QAHSLERVCHCLGKWL | | 2857 | | | | | 2500 | |
| GHPDK | | | | | | | | |
| WTTCQSIAFPSKTSAS | | 40000 | | 277 | 37450 | 505 | 400 | |
| IGSL | | | | | | | | |
| QKGRGYRGQHQAHSLE | | 30151 | | >9100 | >500000 | 17951 | 9759 | |
| RVCH | | | | | | | | |
| AATYNFAVLKLMGRGT | | 17 | | 239 | 70014 | 1218 | 18 | |
| KF | | | | | | | | |
| VATGLCFFGVALFCGC | | 33333 | | | 117851 | 193333 | | |
| GHEA | | | | | | | | |
| FLYGALLLAEGFYTTG | | | | 45 | | | 256 | |
| AVRQ | | | | | | | | |
| SAVPVYIYFNTWTTCQS | | | | 92 | | | 20000 | |
| IAF | | | | | | | | |
| TLSVTWIGAAPLILS | | 6860 | 642 | 97 | 6031 | 3506 | 31 | |
| SVTWIGAAPLILSRI | | 2196 | 420 | 147 | 13676 | 42 | 104 | |
| VTWIGAAPLILSRIV | | 1779 | 2339 | 552 | >10729.61 | 88 | 147 | |
| SQPWQVLVASRGRAV | | 135 | 32 | 11259 | >12116.81 | 7562 | 84 | |
| GRAVCGGVLVHPQWV | | >50000 | 5456 | 12888 | >12116.81 | 62 | 100000 | |
| GVLVHPQWVLTAAHC | | 263 | 2427 | 66 | >10729.61 | 6.2 | 1062 | |
| HPQWVLTAAHCIRNK | | 785 | 1170 | 6500 | 1324 | 5518 | 40 | |
| QWVLTAAHCIRNKSV | | 2169 | 2062 | 13565 | 7342 | 3802 | 35 | |
| AHCIRNKSVILLGRH | | 93 | 75 | 88 | 4752 | 8.7 | 3630 | |
| SVILLGRHSLFHPED | | 96 | 96 | 106 | 13045 | 4411 | 16116 | |
| VILLGRHSLFHPEDT | | 344 | 543 | 426 | >12116.81 | 10696 | 100000 | |
| GQVFQVSHSFPHPLY | | 103 | 146 | 2172 | 1071 | 416 | 128 | |
| VFQVSHSFPHPLYDM | | 881 | 83 | 2396 | 23433 | >12491.92 | 897 | |
| PHPLYDMSLLKNRFL | | >50000 | 11667 | 712 | >13533.63 | 7486 | 3104 | |
| SHDLMLLRISEPAEL | | 4471 | 5.8 | 1099 | 13577 | 12 | 100000 | |
| HDLMLLRLSEPAELT | | 2141 | 2.3 | 662 | 5305 | 45 | 10541 | |
| TDAVKVMDLPTQEPA | | >50000 | >35000 | >45500 | >13533.63 | 747 | >200000 | |
| LHVISNDVCAQVHPQ | | >50000 | 239 | 22750 | 1887 | 1087 | >200000 | |
| CAQVHPQKVTKFMLC | | 18490 | 2192 | 809 | >13533.63 | 604 | 1229 | |
| GGPLVCNGVLQGITS | | 1828 | 36 | 30333 | >6567.28 | 815 | 13417 | |
| GPLVCNGVLQGITSW | | 915 | 49 | 6310 | 11615 | 646 | 6537 | |
| NGVLQGITSWGSEPC | | 9724 | 775 | 258 | 8038 | 4487 | 11619 | |
| RPSLYTKVVHYRKWI | | 350 | 4183 | 717 | 2982 | 4897 | 13 | |
| HSLFHPEDTGQVFQV | | | | | 553 | 11503 | | |
| PRWLCAGALVLAGGF | | >40000 | 20207 | 15167 | 13150 | 883 | 40825 | |
| LGFLFGWFIKSSNEA | | 7303 | 10104 | 355 | 681 | 9285 | 461 | |
| LDELKAENIKKFLYN | | 324 | 597 | 414 | 548 | 788 | 150 | |
| IKKFLYNFTQIPHLA | | 137 | 27 | 305 | 477 | 96 | 658 | |
| KFLYNFTQIPHLAGT | | 91 | 221 | 227 | 10212 | 256 | 1600 | |
| WKEFGLDSVELAHYD | | 4935 | 8413 | 22750 | 829 | 5925 | 89443 | |
| LAHYDVLLSYPNKTA | | 380 | 268 | 82 | 1406 | 589 | 172 | |
| GNEIFNTSLFEPPPP | | >40000 | 2804 | >91000 | >13164.82 | 835 | >200000 | |
| GKVFRGNKVKNAQLA | | 894 | 46 | 3373 | 7591 | 7884 | 1385 | |
| GNKVKNAQLAGAKGV | | >66666.67 | >35000 | >45500 | >12462.61 | 1065 | 1218 | |
| EYAYRRGIAEAVGLP | | 2590 | 5217 | >45500 | 8773 | 6325 | 1204 | |
| AEAVGLPSIPVHPIG | | >66666.67 | 5456 | 56 | >11848.34 | 12394 | 69336 | |
| AVGLPSIPVHPIGYY | | 33333 | 1191 | 518 | >11848.34 | 5387 | 38517 | |
| IGYYDAQKLLEKMGG | | >28571.43 | 5729 | 1978 | 17305 | 13588 | 506 | |
| TGNFSTQKVKMHIHS | | 11856 | 6187 | 3745 | >11848.34 | 508 | 1927 | |
| TRIYNVIGTLRGAVE | | 45 | 1460 | 1605 | 17550 | 447 | 32 | |
| ERGVAYINADSSIEG | | >50000 | 3689 | 30333 | 6846 | 87 | 200000 | |
| GVAYINADSSIEGNY | | >40000 | 497 | 7610 | 1420 | 477 | 66667 | |
| DSSLEGNYTLRVDCT | | >50000 | 7.6 | 1202 | 576 | 1262 | 16824 | |
| NYTLRVDCTPLMYSL | | 7116 | 9.0 | 5056 | 25 | 404 | 66667 | |
| CTPLMYSLVHNLTKE | | 590 | 260 | 426 | 18348 | 58 | 36 | |
| DFEVFFQRLGIASGR | | 128 | 10069 | 10249 | 30745 | 4.2 | 3559 | |
| EVFFQRLGIASGRAR | | 31 | 17500 | 4556 | >15037.59 | 51 | 7.9 | |
| TNKFSGYPLYHSVYE | | 33333 | >35000 | 489 | >21853.15 | 12466 | 2942 | |
| YDPMFKYHLTVAQVR | | 252 | 1014 | 1348 | 8137 | 553 | 62 | |
| DPMFKYHLTVAQVRG | | 69 | 699 | 230 | 7297 | 467 | 11 | |
| MFKYHLTVAQVRGGM | | 147 | 1615 | 1198 | 3648 | 1062 | 5.8 | |
| KYHLTVAQVRGGMVF | | 859 | 193 | 1222 | >21853.15 | 3446 | 86 | |
| VAQVRGGNIVFELANS | | >50000 | 2802 | 117 | <21853.15 | 100 | 64366 | |
| RGGMVFELANSIVLP | | >50000 | 4.4 | 94 | 132 | 411 | 413 | |
| GMVFELANSIVLPFD | | >50000 | 12 | 83 | 234 | 4154 | 903 | |
| VFELANSIVLPFDCR | | 11765 | 24 | 477 | 128 | 1215 | 10815 | |
| ADKIYSISMKHPQEM | | 169 | 4957 | 8273 | >21853.15 | 3550 | 26726 | |
| IYSISMKHPQEMKTY | | 213 | >35000 | 5025 | >21853.15 | 5356 | 2588 | |
| PQEMKTYSVSFDSLF | | >50000 | 24749 | 919 | 14564 | 579 | 100000 | |
| TYSVSFDSLFSAVKN | | 5981 | 5888 | 3223 | 8547 10461 | 61 | | |
| VLRMMNDQLMFLERA | | 2353 | 130 | 127 | 98 | 88 | 85 | |
| LRMMNDQLMFLERAF | | 1833 | 1314 | 1411 | 1570 | 50 | 758 | |
| RHVIYAPSSHNKYAG | | 13363 | 8750 | 1291 | >62814.07 | 5293 | 88 | |
| RQIYVAAFTVQAAAE | | 35 | 524 | 166 | 6808 | 47 | 143 | |
| QIYVAAFTVQAAAET | | 34 | 344 | 252 | 1324 | 50 | 216 | |
| VAAFTVQAAAETLSE | | 2126 | 446 | 18200 | 2116 | 464 | 378 | |
| YISIINEDGNEIFNT | | >18903.59 | 346 | 2713 | 30 | 3705 | 72993 | |
| ISIINEDONEIENTS | | >18903.59 | 343 | 3006 | 35 | 6394 | >37807.18 | |
| EDFFKLERDMKINCS | | 10433 | 3188 | >3490.6 | 4036 | 7886 | 3494 | |
| FFKLERDMKINCSGK | | 9687 | 382 | >3490.6 | 4918 | 98 | 3796 | |
| GVILYSDPADYFAPG | | >18903.59 | 39 | 965 | 8.8 | 64 | 14168 | |
| GAAVVHEIVRSFGTL | | | | | 788 | 89 | | |
| NSRLLQERGVAYINA | | 12812 | 327 | 1229 | 3366 | 699 | 3473 | |
| VAYINADSSIEGNYT | | >18903.59 | 2147 | >3490.6 | 471 | 841 | >37807.18 | |
| DQLMFLERAFIDPLG | | | | | 17115 | 6.6 | | |
| KSNFLNCYVSGFHPSD | | 5000 | | | | | 2857 | |
| AC- | | >33333.33 | >10000 | >10000 | 1000 | | 50000 | |
| NPDAENWNSQFEILED | | | | | | | | |
| AA | | | | | | | | |
| EYLILSARDVLAVVS | | 6860 | | 2340 | | 2527 | 4154 | |
| YKTIAYDEEARR | | 200000 | | >91000 | >50000 | | 200000 | |
| GEALSTLVVNKIRGT | | 977 | 55 | 2314 | | 1514 | 108 | |
| PYILLVSSKVSTVKD | | 112 | 7.2 | 22 | | 107 | 32 | |
| EAVLEDPYILLVSSK | | 4376 | >10294.12 | >50837.99 | | >26435.73 | 357 | |
| IAGLFLTTEAVVADK | | 867 | >10294.12 | >50837.99 | | >26435.79 | 606 | |
| ALSTLVVNK1RGTFK | | 32 | 7.6 | 160 | | 214 | 38 | |
| MKHILYISFYFILVN | | 2082 | | | | | >9523.81 | |
| KSLLSTALPYGRTNL | | | | | | | | |
| HFFLFLLYILFLVKM | | | 84 | 21473 | | 1064 | 10083 | |
| LFLLYILFLVKMNAL | | | 129 | 30829 | | 1290 | 32446 | |
| ILFLVKMNALRRLPV | | | 0.13 | 1.4 | | 7.6 | 14 | |
| MNALRRLPVICSFLV | | | 15 | 36 | | 5.7 | 2557 | |
| SAFLESQSMNKIGDD | | | 52 | 18689 | | 302 | 243 | |
| LKELIKVGLPSFENL | | | 147 | 361 | | 110 | 41322 | |
| FENLVAENVKPPKVD | | | 3029 | >50837.99 | | 9297 | 62661 | |
| PATYGIIVPVLTSLF | | | 0.83 | 2557 | | 118 | 52 | |
| VGIRTPVLTSLFNKV | | | 0.30 | 223 | | 97 | 80 | |
| LLKIWKNYMKIMNHL | | | 3.7 | 6.8 | | 12 | 35 | |
| MTLYQIQVMKRNQKQ | | | 323 | 2429 | | 82 | 22 | |
| QKQVQMMIMIKFMGV | | | 17 | 363 | | 5.3 | 915 | |
| MJMIKFMGVIYIMII | | | 102 | 23611 | | 145 | 12310 | |
| GVIYIMIISKKMMRK | | | 38 | 173 | | 157 | 46 | |
| LYYLFNQHIKKELYH | | | 327 | 2861 | | 1089 | 606 | |
| HFNMLKNKMQSSFFM | | | 54 | 616 | | 934 | 60 | |
| LDIYQKLYIKQEEQK | | | 4346 | 47 | | 70 | 6958 | |
| QKKYIYNLIMNTQNK | | | 53 | 844 | | 87 | 245 | |
| YEALIKLLPFSKRIR | | | 230 | 36 | | 15 | 11 | |
| ENEYATGAVRPFQAA | | | 9302 | 3007 | | 10026 | >10303.97 | |
| NYELSKXAVIFTPIY | | | 410 | 537 | | 136 | 10581 | |
| QKILIKIPVTKNIIT | | | 332 | 3614 | | 953 | 297 | |
| KCLVISQVSNSDSYK | | | 236 | 403 | | 81 | >42553.19 | |
| SK1MKLPKLPISNGK | | | 6460 | 3570 | | 6739 | >10303.97 | |
| FIHFFTWGTMFVPKY | | | 328 | 2375 | | 387 | 9608 | |
| LCNFKKNIIALLHP | | | 16 | 29302 | | 99 | >42553.19 | |
| KKNIIALLIIPPKIH | | | 15 | 32 | | 8.2 | 143 | |
| ALLIIPPKIHISIEL | | | 162 | 1823 | | 10 | 7135 | |
| SMEYKKDFLITARKY | | | 3818 | 4610 | | 10448 | 442 | |
| KSKFNILSSPLFNNF | | | 25 | 5.9 | | 135 | 32 | |
| FKKLKNHVLFLQMMN | | | 20 | 29 | | 14 | 59 | |
| KNHVLFLQMMNVNLQ | | | 36 | 224 | | 22 | >7212.41 | |
| VLFLQMMNVNLQKQL | | | 8.6 | 8200 | | 12 | >7212.41 | |
| NVNLQKQLLTNHLIN | | | 28 | 4448 | | 354 | >7212.41 | |
| QKQLLTNHLINTPKI | | | 1.6 | 514 | | 904 | 6595 | |
| NHLINTPKIMPHHII | | | 32 | 560 | | 1632 | 8882 | |
| YILLKKILSSRFNQM | | | 1.01 | 26 | | 340 | 83 | |
| FNQMIFVSSIFISSFY | | | 33 | 3903 | | 1291 | >12484.39 | |
| KVSCKGSGYTFTAYQM | | >200000 | | | | | | |
| H | | | | | | | | |
| IAKVPPGPNITAEYGD | | 200000 | | | >20000 | 200000 | | |
| KWLD | | | | | | | | |
| TAEYGDKWLDAKSTW | | 200000 | | | >20000 | 10000 | | |
| YGKPT | | | | | | | | |
| AKSTWYGKPTGAGPKD | | 200000 | | | >20000 | 10000 | | |
| NGGA | | | | | | | | |
| GAGPKDNGGACGYKD | | 200000 | | | >20000 | 200000 | | |
| VDKAP | | | | | | | | |
| FNGMTGCGNTPIFKDG | | 200000 | | | >20000 | 200000 | | |
| RGCG | | | | | | | | |
| PIFKDGRGCGSCFEIK | | 200000 | | | >20000 | 200000 | | |
| CTKP | | | | | | | | |
| SCFEIKCTKPESCSGE | | 200000 | | | >20000 | 200000 | | |
| AVTV | | | | | | | | |
| AFGSMAKKGEEQNVRS | | 1818 | | | >33333.33 | 200000 | | |
| AGEL | | | | | | | | |
| TPDKLTGPFTVRYTTE | | 200000 | | | >25000 | 200000 | | |
| GGTK | | | | | | | | |
| VRYTTEGGTKSEVEDV | | 200000 | | | >25000 | 200000 | | |
| IPEG | | | | | | | | |
| TCVLGKLSQELHKLQ | | 1398 | >12589.93 | 2009 | >263157.89 | 163 | 3986 | |
| KLSQELHKLQTYPRT | | 2375 | >12589.93 | 287 | >263157.89 | 870 | 37 | |
| LHKLQTYPRTNTGSG | | 6091 | >12589.93 | 157 | >263157.89 | 22948 | 40 | |
| KLQTYPRTNTGSGTP | | 8210 | 987 | 520 | >263157.89 | >104693.14 | >14044.94 | |
| CCVLGKLSQELHKLQ | | 5243 | >12589.93 | 570 | >263157.89 | 346 | 5158 | |
| CSNLSTCVLGKLSQE | | 5263 | 7907 | 4538 | >263157.89 | 11756 | 5709 | |
| TSNLSTTVLGKLSQE | | 534 | 9333 | 7697 | >263157.89 | 13210 | 2529 | |
| TTVLGKLSQELHKLQ | | 3524 | 12715 | 525 | >263157.89 | 241 | 10618 | |
| DIAAKYKELGY | | >10000 | | | >25000 | | 200000 | |
| ALVRQGLAKVA | | 200000 | | | | | >10000 | |
| PATLIKAIDGDTVKLM | | >6666.67 | | | 2381 | | 3333 | |
| YKGQ | | | | | | | | |
| TPETKHPKKGVEKYGP | | >6666.67 | | | >25000 | | >4000 | |
| EASA | | | | | | | | |
| VEKYGPEASAFTKKMV | | 20000 | | | 16667 | | 34 | |
| ENAK | | | | | | | | |
| FTKKMVENAKKIEVEF | | 6667 | | | >25000 | | 1000 | |
| DKGQ | | | | | | | | |
| YIYADGKMVNEALVRQ | | >6666.67 | | | >5555.56 | | >4000 | |
| GLAK | | | | | | | | |
| HEQHLRKSEAQAKKEK | | 200000 | | | >5555.56 | | 11 | |
| LNIW | | | | | | | | |
| QAKKEKLNIWSEDNAD | | 200000 | | | >5555.56 | | 200000 | |
| SGQ | | | | | | | | |
| YFNNFTVSFWLRVPK | | | | | | | | |
| FSYFPSI | | | | | | | | |
| YSFFPSI | | | | | | | | |
| YSYFPSIR | | 20000 | | | | | >200000 | |
| DPNANPNVDPNANPNV | | >12500 | | >7583.33 | | >72500 | >2898.55 | |
| NANPNANPNANP(X4) | | | | | | | | |
| QKWAAVVVPS | | | | | | | | |
| TWQLNGEELIQDMELV | | | | | | | | |
| ETRPAG | | | | | | | | |
| PEFLEQRRAAVDTYC | | 488 | | | | | 200000 | |
| STORKUSP33 | | | | | | | | |
| DYSYLQDSDPDSFQD | | >66666.67 | >35000 | >45500 | | | >40000 | |
| DFSYLQDSDPDSFQD | | | >35000 | >91000 | | | >40000 | |
| QNILFSNAPLGPQFP | | | | | | | | |
| QNILLSNAPLVPQFP | | | | | | | | |
| DYSYLQDSDPDSFQD | | | | | | | | |
| KYVKQNTLKLAT | | | | | | | | |
| P(X)KQNTLKLAT | | | | | | | | |
| EEDIEIIFIQEEEY | | >20576.13 | | | | | 46083 | |
| HQAISPRTLNSPAIF | | 33686 | 1036 | 8106 | >83333.33 | 130 | >200000 | |
| YTDVFSLDPTFTIETT | | | | | | | | |
| YAGIRRDGLLLRLVD | | | | | | | | |
| LFFYRKSVWSKLQSI | | 12 | 121 | 20 | 5915 | 1933 | 18 | |
| RPIVNMDYVVGARTFR | | 222 | 73 | 43 | 3324 | 160 | 6.6 | |
| REKR | | | | | | | | |
| RPGLLGASVLGLDDI | | >93896.71 | 2056 | 6000 | 30212 | 22038 | >88888.89 | |
| LYFVKVDVTGAYDTI | | 221 | 79 | 9753 | 16 | 22 | 4962 | |
| FAGIRRDGLLLRIVD | | 804 | 1294 | 28 | 553 | 1670 | 1355 | |
| AKTFLRTLVRGVPEY | | 6.3 | 94 | 829 | 546 | 472 | 3484 | |
| YGAVVNLRKTVVNFP | | 89 | 11236 | 470 | 51496 | 302 | 36 | |
| GTAFVQMPAHGLFPW | | 17 | 2819 | 1.2 | 769 | 2361 | 43 | |
| WAGLLLDTRTLEVQS | | 20960 | 92 | 3468 | | 862 | >102040.82 | |
| RTSIRASLTFNRGFK | | 4807 | 49 | 497 | | 79 | 52 | |
| RVIKNSIRLTL | | 1740 | 32 | 4317 | | 143 | 8834 | |
| PVIKNSIKLRL | | 2772 | 77 | 2579 | | 198 | 1039 | |
| ATSTKKLHKEPATLIK | | >6666.67 | | | 462 | | 267 | |
| AIDG | | | | | | | | |

TABLE 28
MURINE CLASS I SUPERTYPE

| | | | | | | |
|---|---|---|---|---|---|---|
| | SEQ ID | | | | | |
| Sequence | NO. | AA | Organism | Protein | Position | Analog |

| | | | | | |
|---|---|---|---|---|---|
| SGPSNTPPEI | 10 | Adenovirus | E1A | | |
| RNPRFYNL | 8 | Artificial | Consensus | | |
| | | sequence | | | |
| QPQRGYENF | 9 | Artificial | Consensus | | A |
| | | sequence | | | |
| SEAAYAKKI | 9 | Artificial | pool | | A |
| | | sequence | consensus | | |
| AYAPAKAAI | 9 | Artificial | | | Poly |
| | | sequence | | | |
| AYAEAKAAI | 9 | Artificial | | | Poly |
| | | sequence | | | |
| AYANAKAAI | 9 | Artificial | | | Poly |
| | | sequence | | | |
| AYAGAKAAI | 9 | Artificial | | | Poly |
| | | sequence | | | |
| AYAVAKAAI | 9 | Artificial | | | Poly |
| | | sequence | | | |
| AAAAYAAM | 8 | Artificial | | | |
| | | sequence | | | |
| AAAAYAAAAM | 10 | Artificial | | | |
| | | sequence | | | |
| AAAANAAAM | 9 | Artificial | | | |
| | | sequence | | | |
| AAAAAANAAAM | 11 | Artificial | | | |
| | | sequence | | | |
| NAIVFKGL | 8 | Chicken | Ova | 176 | |
| SIINFEKL | 8 | Chicken | Ova | 257 | |
| IFYCPIAI | 8 | Chicken | Ova | 27 | |
| KVVRFDKL | 8 | Chicken | Ova | 55 | |
| VYSFSLASRL | 10 | Chicken | Ova | 96 | |
| SIINFEKL | 8 | Chicken | Ova | 257 | |
| KVVRFDKL | 8 | Chicken | Ova | 55 | |
| SENDRYRLL | 9 | EBV | BZLF1 | 209 | A |
| SFYRNLLWL | 9 | Flu | HA | 142 | |
| YEANGNLI | 8 | Flu | HA | 259 | A |
| MGLIYNRM | 8 | Flu | M1 | 128 | |
| MGYIYNRM | 8 | Flu | M1 | 128 | |
| MGIIYNRM | 8 | Flu | M1 | 128 | |
| MGLIFNRM | 8 | Flu | M1 | 128 | |
| MGLIYNRM | 8 | Flu | M1 | 128 | |
| RMIQNSLTI | 9 | Flu | NP | 55 | |
| RLIQNFLTI | 9 | FLu | NP | 55 | |
| GMRQNATEI | 9 | Flu | NP | 17 | |
| YMRVNGKWM | 9 | Flu | NP | 97 | |
| FYIQMATEL | 9 | Flu | NP | 39 | |
| FYIQMCTFL | 9 | Flu | NP | 39 | |
| AYERMANIL | 9 | Flu | NP | 218 | |
| AYQRMCNIL | 9 | Flu | NP | 218 | |
| AYERMCTIL | 9 | Flu | NP | 218 | |
| ASNENMETM | 9 | Flu | NP | 366 | |
| TYQRTRALM | 9 | Flu | NP | 147 | A |
| TYQKTRALV | 9 | Flu | NP | 147 | A |
| TYQPTRALV | 9 | Flu | NP | 147 | A |
| TYQFTRALV | 9 | Flu | NP | 147 | A |
| TYQLTRALV | 9 | Flu | NP | 147 | A |
| SDYEGRLI | 8 | Flu | NP | 50 | |
| MITQFESL | 8 | Flu | NS | 31 | |
| RTFSFQLI | 8 | Flu | NS | 114 | |
| FSVIFDRL | 8 | Flu | NS | 134 | |
| RTFSFQLI | 8 | Flu | NS1 | 114 | |
| MITQFESL | 8 | Flu | NS1 | 31 | |
| FSVIFDRL | 8 | Flu | NS2 | 134 | |
| KSSFYRNL | 8 | FluA | HA | 158 | |
| SSLPFQNI | 8 | FluA | HA | 305 | |
| MNIQFTAV | 8 | FluA | HA | 403 | |
| MNYYWTLL | 8 | FluA | HA | 244 | |
| SFYRNLLWL | 9 | FluA | HA | 160 | |
| SSLPFQNI | 8 | FluA | HA | 305 | |
| MNIQFTAV | 8 | FluA | HA | 403 | |
| MNYYWTLL | 8 | FluA | HA | 244 | |
| KSSFYRNL | 8 | FluA | HA | 158 | |
| SIIPSGPL | 8 | FluA | M1 | 13 | |
| LSYSAGAL | 8 | FluA | M1 | 117 | |
| LSYSAGAL | 8 | FluA | M1 | 117 | |
| SSISFCGV | 8 | FluA | NM | 426 | |
| TGICNQNII | 9 | FluA | NM | 46 | |
| ITYKNSTWV | 9 | FluA | NM | 54 | |
| FCGVNSDTV | 9 | FluA | NM | 430 | |
| TGICNQNII | 9 | FluA | NM | 46 | |
| FCGVNSDTV | 9 | FluA | NM | 430 | |
| ITYKNSTWV | 9 | FluA | NM | 54 | |
| SSISFGGV | 8 | FluA | NM | 426 | |
| IGRFYIQM | 8 | FluA | NP | 36 | |
| MMIWHSNL | 8 | FluA | NP | 136 | |
| ASNENMETM | 9 | FluA | NP | 366 | |
| IGRFYIQM | 8 | FluA | NP | 36 | |
| MMIWHSNL | 8 | FluA | NP | 136 | |
| FFYRYGFV | 8 | FluA | POL1 | 495 | |
| KMITQRTI | 8 | FluA | POL1 | 198 | |
| RSYLIRAL | 8 | FluA | POL1 | 215 | |
| RFYRTCKL | 8 | FluA | POL1 | 465 | |
| TALANTIEV | 9 | FluA | POL1 | 141 | |
| TALANTIEV | 9 | FluA | POL1 | 141 | |
| RSYLIRAL | 8 | FluA | POL1 | 215 | |
| RFYRTGKL | 8 | FluA | POL1 | 465 | |
| VYINTALL | 8 | FluA | POL2 | 463 | |
| VYINTALL | 8 | FluA | POL2 | 463 | |
| VYIEVLHL | 8 | FluA | POL3 | 227 | |
| VYIEVLHL | 8 | FluA | POL3 | 227 | |
| WYIPPSLRTL | 10 | GAD | | | |
| MURTAZAKDPEPTIDES | 0 | GAD65 | | 107 | |
| IYSTVASSL | 9 | HA | | 553 | |
| LYEKVKSQL | 9 | HA | | 462 | |
| LYQKVKSQL | 9 | HA | | 462 | |
| LYEKMKSQL | 9 | HA | | 462 | |
| LYEKVFSQL | 9 | HA | | 462 | |
| LYQNVGTYV | 9 | HA | | 204 | |
| MGLKFRQL | 8 | HBV | core | 122 | |
| VSYVNTNM | 8 | HBV | core | 115 | |
| SYVNTNMGL | 9 | HBV | core | 116 | |
| MGLKFRQL | 8 | HBV | core | 122 | |
| VSYVNTNM | 8 | HBV | core | 115 | |
| SYVNTNMGL | 9 | HBV | core | 116 | |
| WGPSLYSI | 8 | HBV | env | 364 | |
| ASARFSWL | 8 | HBV | env | 329 | |
| WGPSLYSIL | 9 | HBV | env | 364 | |
| TGPCRTCMT | 9 | HBV | env | 281 | |
| WYWGPSLYSI | 10 | HBV | env | 362 | |
| IPQSLDSWWTSL | 12 | HBV | env | 28 | |
| IPQSLDSYWTSL | 12 | HBV | env | 28 | A |
| ASARFSWL | 8 | HBV | env | 329 | |
| WYWGPSLYSI | 10 | HBV | env | 362 | |
| APQSLDSWWTSL | 12 | HBV | env | 28 | |
| IPQALDSWWTSL | 12 | HBV | env | 28 | A |
| IPQSLASWWTSL | 12 | HBV | env | 28 | A |
| IPQSLDAWWTSL | 12 | HBV | env | 28 | A |
| IPQSLDSAWTSL | 12 | HBV | env | 28 | A |
| IPQSLDSWWASL | 12 | HBV | env | 28 | A |
| IPQSLDSWWTAL | 12 | HBV | env | 28 | A |
| EPQSLDSWWTSL | 12 | HBV | env | 28 | A |
| IPESLDSWWTSL | 12 | HBV | env | 28 | A |
| IPQSLDEWWTSL | 12 | HBV | env | 28 | A |
| IPQSLDSWWTEL | 12 | HBV | env | 28 | A |
| RPQSLDSWWTSL | 12 | HBV | env | 28 | A |
| IPRSLDSWWTSL | 12 | HBV | env | 28 | A |
| IPQRLDSWWTSL | 12 | HBV | env | 28 | A |
| IPQSRDSWWTSL | 12 | HBV | env | 28 | A |
| IPQSLRSWWTSL | 12 | HBV | env | 28 | A |
| IPQSLDRWWTSL | 12 | HBV | env | 28 | A |
| IPQSLDSRWTSL | 12 | HBV | env | 28 | A |
| IPQSLDSWWRSL | 12 | HBV | env | 28 | A |
| IPQSLDSWWTRL | 12 | HBV | env | 28 | A |
| YPQSLDSWWTSL | 12 | HBV | env | 28 | A |
| IPYSLDSWWTSL | 12 | HBV | env | 28 | A |
| IPQYLDSWWTSL | 12 | HBV | env | 28 | A |
| IPQSLYSWWTSL | 12 | HBV | env | 28 | A |
| IPQSLDYWWTSL | 12 | HBV | env | 28 | A |
| IPQSLDSWYTSL | 12 | HBV | env | 28 | A |
| IPQSLDSWWTYL | 12 | HBV | env | 28 | A |
| IPGSLDSWWTSL | 12 | HBV | env | 28 | A |
| IPQSLDSGWTSL | 12 | HBV | env | 28 | A |
| IPQSLDSPWTSL | 12 | HBV | env | 28 | A |
| IPQSLDSWGTSL | 12 | HBV | env | 28 | A |
| IPQSLDSWPTSL | 12 | HBV | env | 28 | A |
| IPQSLDSWWTGL | 12 | HBV | env | 28 | A |
| IPQSLDSWWTPL | 12 | HBV | env | 28 | A |
| IPQVLDSWWTSL | 12 | HBV | env | 28 | A |
| IPQFLDSWWTSL | 12 | HBV | env | 28 | A |
| IPQPLDSWWTSL | 12 | HBV | env | 28 | A |
| IPQMLDSWWTSL | 12 | HBV | env | 28 | A |
| IPQILDSWWTSL | 12 | HBV | env | 28 | A |
| IPQLLDSWWTSL | 12 | HBV | env | 28 | A |
| IPQGLDSWWTSL | 12 | HBV | env | 28 | A |
| IPQTLDSWWTSL | 12 | HBV | env | 28 | A |
| IPQHLDSWWTSL | 12 | HBV | env | 28 | A |
| IPQCLDSWWTSL | 12 | HBV | env | 28 | A |
| IPQNLDSWWTSL | 12 | HBV | env | 28 | A |
| IPQQLDSWWTSL | 12 | HBV | env | 28 | A |
| IPQWLDSWWTSL | 12 | HBV | env | 28 | A |
| IPQDLDSWWTSL | 12 | HBV | env | 28 | A |
| IPQKLDSWWTSL | 12 | HBV | env | 28 | A |
| IPQSLVSWWTSL | 12 | HBV | env | 28 | A |
| IPQSLFSWWTSL | 12 | HBV | env | 28 | A |
| IPQSLPSWWTSL | 12 | HBV | env | 28 | A |
| IPQSLMSWWTSL | 12 | HBV | env | 28 | A |
| IPQSLISWWTSL | 12 | HBV | env | 28 | A |
| IPQSLLSWWTSL | 12 | HBV | env | 28 | A |
| IPQSLGSWWTSL | 12 | HBV | env | 28 | A |
| IPQSLSSWWTSL | 12 | HBV | env | 28 | A |
| IPQSLTSWWTSL | 12 | HBV | env | 28 | A |
| IPQSLHSWWTSL | 12 | HBV | env | 28 | A |
| IPQSLCSWWTSL | 12 | HBV | env | 28 | A |
| IPQSLNSWWTSL | 12 | HBV | env | 28 | A |
| IPQSLQSWWTSL | 12 | HBV | env | 28 | A |
| IPQSLWSWWTSL | 12 | HBV | env | 28 | A |
| IPQSLKSWWTSL | 12 | HBV | env | 28 | A |
| IPSLDSWWTSL | 11 | HBV | env | 28 | A |
| IPQSLDSWTSL | 11 | HBV | env | 28 | A |
| IPQSLDSWWTL | 11 | HBV | env | 28 | A |
| IPQALASWWTSL | 12 | HBV | env | 28 | A |
| IPQSLDSWWTSM | 12 | HBV | env | 28 | A |
| IPQSLDSWWTSF | 12 | HBV | env | 28 | A |
| KTPSFPNI | 8 | HBV | pol | 75 | |
| HAVEFHNL | 8 | HBV | pol | 289 | |
| VSAAFYHL | 8 | HBV | pol | 419 | |
| VIGCYGSL | 8 | HBV | pol | 588 | |
| KQYLNLYPV | 9 | HBV | pol | 668 | |
| CYGSLPQEHI | 10 | HBV | pol | 591 | |
| VSAAFYHL | 8 | HBV | pol | 419 | |
| HAVEFHNL | 8 | HBV | pol | 289 | |
| VIGCYGSL | 8 | HBV | pol | 588 | |
| KTPSFPNI | 8 | HBV | pol | 75 | |
| RPQSLDSWWTSL | 12 | HBVs | env | 28 | A |
| IPQRLDSWWTSL | 12 | HBVs | env | 28 | A |
| IPQSLRSWWTSL | 12 | HBVs | env | 28 | A |
| IPQSLDRWWTSL | 12 | HBVs | env | 28 | A |
| IPQSLDSRWTSL | 12 | HBVs | env | 28 | A |
| IPQSLDSWWRSL | 12 | HBVs | env | 28 | A |
| IPQSLDSWWTRL | 12 | HBVs | env | 28 | A |
| IPQELDSWWTSL | 12 | HBVs | env | 28 | A |
| IPQSLYSWWTSL | 12 | HBVs | env | 28 | A |
| IPQSLDSWETSL | 12 | HBVs | env | 28 | A |
| IPQSLDSWWESL | 12 | HBVs | env | 28 | A |
| VESENKVV | 8 | HCV | Entire | 2253 | |
| AGPYRAFVTI | 10 | HIV | env | 18 | A |
| RAPYRAFVTI | 10 | HIV | env | 18 | A |
| RGPYRAFVTA | 10 | HIV | env | 18 | A |
| KGPYRAFVTI | 10 | HIV | env | 18 | A |
| RGPYRAFVTK | 10 | HIV | env | 18 | A |
| RGPGRAFVTI | 10 | HIV | env | 18 | |
| RGPGRYFVTI | 10 | HIV | env | 18 | A |
| RGPGRAYVTI | 10 | HIV | env | 18 | A |
| RGPGRAFYTI | 10 | HIV | env | 18 | A |
| VESMNKEL | 8 | HIV | POL | 903 | |
| TDSQYALGI | 9 | HIV | POL | 689 | |
| RGAYRAFVTI | 10 | HIV | | 18 | A |
| RGPARAFVTI | 10 | HIV | | 18 | A |
| RGPYRAAVTI | 10 | HIV | | 18 | A |
| RGPYRAFATI | 10 | HIV | | 18 | A |
| RGPYRAFVAI | 10 | HIV | | 18 | A |
| RGKYRAFVTI | 10 | HIV | | 18 | A |
| RGPFRAFVTI | 10 | HIV | | 18 | A |
| RGPYKAFVTI | 10 | HIV | | 18 | A |
| RGPYRKFVTI | 10 | HIV | | 18 | A |
| RGPYRAYVTI | 10 | HIV | | 18 | A |
| RGPYRAFKTI | 10 | HIV | | 18 | A |
| RGPYRAFVKI | 10 | HIV | | 18 | A |
| NEILIRCII | 9 | HPV | E6 | 97 | |
| QEKKRHVDL | 9 | HPV | E6 | 113 | |
| LFVVYRDSI | 9 | HPV | E6 | 52 | |
| FYSRIRELRF | 10 | HPV | E6 | 71 | A |
| SSIEFARL | 8 | HSV | | 498 | |
| KVPRNQDWL | 9 | Human | gp100 | | |
| VYDFYVWM | 8 | Human | TRP2 | | A |
| KNKFFSYL | 8 | Human | Tyrosinase | 131 | |
| LAVLYCLL | 8 | Human | Tyrosinase | 3 | |
| YMVPFIPL | 8 | Human | Tyrosinase | 425 | |
| GQMNNGSTPM | 10 | Human | Tyrosinase | 157 | |
| IVTMFEAL | 8 | LCMV | GP | 4 | |
| ISHNFCNL | 8 | LCMV | GP | 118 | |
| GVYQFKSV | 8 | LCMV | GP | 70 | |
| HYISMGTSGL | 10 | LCMV | GP | 99 | |
| SGVENPGGYCL | 11 | LCMV | GP | 276 | |
| KAVYNFATM | 9 | LCMV | GP | 33 | |
| CMANNSHHYI | 10 | LCMV | GP | 92 | A |
| CSANNSHHYM | 10 | LCMV | GP | 92 | A |
| SMVENPGGYCL | 11 | LCMV | GP | 276 | A |
| SGVENPGGYCM | 11 | LCMV | GP | 276 | A |
| KAVYNFATM | 9 | LCMV | GP | 33 | |
| KAVYNAATM | 9 | LCMV | GP | 33 | A |
| KAVANFATM | 9 | LCMV | GP | 33 | A |
| KAVYNYATM | 9 | LCMV | GP | 33 | A |
| KAVYNFAAM | 9 | LCMV | GP | 33 | A |
| YTVKYPNL | 8 | LCMV | NP | 205 | |
| FQPQNGQFI | 9 | LCMV | NP | 396 | |
| VGLSYSQTM | 9 | LCMV | NP | 356 | |
| FQPQNGQFI | 9 | LCMV | NP | 396 | |
| FQPQNGQFIHFY | 12 | LCMV | NP | 396 | |
| RPQASGVYM | 9 | LCMV | NP | 118 | |
| RPQASQVYM | 9 | LCMV | NP | 118 | A |
| YTYKYPNL | 8 | LCMV | NP | 205 | A |
| RPQASGVYM | 9 | LCMV | NP | 118 | A |
| RPQASGVAM | 9 | LCMV | NP | 118 | A |
| RPQGSGVYM | 9 | LCMV | NP | 118 | A |
| RPNASGVYM | 9 | LCMV | NP | 118 | A |
| KAVYNFATCGI | 11 | LCMV | | | |
| KAVYNFATB | 9 | LCMV | | | |
| VYAKECTGL | 9 | Lysteria | listeriolysin | 479 | |
| YPHFMPTNL | 9 | MCMV | | 168 | |
| YPHYMPTNL | 9 | MCMV | | 168 | A |
| HETTYNSI | 8 | Mouse | beta actin | 275 | A |
| YEDTGKTI | 8 | Mouse | p40 phox | 245 | |
| | | | RNA | | |
| LGYDYSYL | 8 | Mouse | Tyrosinase | 445 | |
| SSMHNALHI | 9 | Mouse | Tyrosinase | 360 | |
| ANFSFRNTL | 9 | Mouse | Tyrosinase | 336 | |
| SYLTLAKHT | 9 | Mouse | Tyrosinase | 136 | |
| HYYVSRDTL | 9 | Mouse | Tyrosinase | 180 | |
| YYVSRDTLL | 9 | Mouse | Tyrosinase | 181 | |
| SFFSSWQII | 9 | Mouse | Tyrosinase | 267 | |
| SYMVPFIPL | 9 | Mouse | Tyrosinase | 424 | |
| PYLEQASRI | 9 | Mouse | Tyrosinase | 466 | |
| SYLTLAKHTI | 10 | Mouse | Tyrosinase | 136 | |
| HYYVSRDTLL | 10 | Mouse | Tyrosinase | 180 | |
| SQVMNLHNL | 9 | Mouse | TYRP2 | 363 | |
| YENDIEKKI | 9 | P. falciparum | CSP | 375 | |
| NEEPSDKHI | 9 | P. falciparum | CSPZ | 347 | |
| EEKHEKKHV | 9 | P. falciparum | LSA1 | 52 | |
| SYVPSAEQIL | 10 | P. yoelii | CSP | 280 | |
| RYLENGKETL | 10 | Unknown | HLA-A24 | 170 | |
| RYLKNGKETL | 10 | Unknown | HLA-Cw3 | 170 | |
| IYTQNRRAL | 9 | Unknown | P815 | 12 | |
| VYDFFVWM | 8 | Unknown | TRP2 | 181 | A |
| SVYDFFVWL | 9 | Unknown | TRP2 | 180 | |
| SVYDFYVWM | 9 | Unknown | TRP2 | 180 | A |
| ASNENMDAM | 9 | unknown | | | |
| FAPGYNPAL | 9 | unknown | | | |
| SIQFFGERAL | 10 | unknown | | | |
| SIQFFGEL | 8 | unknown | | | |
| RGYVYQGL | 8 | VSV | NP | 52 | |
| RGPRLNTL | 8 | | | | |
| HMWNFIGV | 8 | | | | |
| GGAYRLIVF | 9 | | | | |
| KYLVTRHADV | 19 | | | | |
| FSPRRNGYL | 9 | | | | |
| SHYAFSPM | 8 | | | | |
| FQPQNGQFI | 9 | | | | |

TABLE 29

MURINE CLASS I SUPERTYPE

| | | | | | | | |
|---|---|---|---|---|---|---|---|
| | SEQ | | | | | | |
| | ID | | | | | | |
| Sequence | NO. | Dd | Kb | Kd | Db | Ld | Kk |

| | | | | | | |
|---|---|---|---|---|---|---|
| SGPSNTPPEI | 18500 | >31000 | >10000 | 8.1 | | |
| RNPRFYNL | | 7.9 | | >44000 | | |
| QPQRGYENF | | | | | 319 | |
| SEAAYAKKI | | | | | | 3.9 |
| AYAPAKAAI | | | 3.5 | | | |
| AYABAKAAI | | | 50 | | | |
| AYANAKAAI | | | 60 | | | |
| AYAGAKAAI | | | 48 | | | |
| AYAVAKAAI | | | 42 | | | |
| AAAAYAAM | | 375 | | >44000 | | |
| AAAAYAAAAM | | 228 | | >44000 | | |
| AAAANAAAM | | 10960 | | 23 | | |
| AAAAAANAAAM | | 31000 | | 257 | | |
| NAIVFKGL | | 484 | | | | |
| SIINFEKL | | 3.7 | | | | |
| IFYCPIAI | | 195 | | | | |
| KVVRFDKL | | 92 | | | | |
| VYSFSLASRL | | | 303 | | | |
| SIINFEKL | >37000 | 1.5 | >10000 | 30508 | | |
| KVVRFDKL | | 37 | | | | |
| SENDRYRLL | | | | | | 13 |
| SFYRNLLWL | | | >10000 | 304 | | |
| YEANGNLI | | | | | | 0.65 |
| MGLIYNRM | | 16 | | | | |
| MGYIYNRM | | 2.3 | | | | |
| MGIIYNRM | | 14 | | | | |
| MGLIFNRM | | 21 | | | | |
| MGLIYNRM | | 9.9 | | | | |
| RMIQNSLTI | | | | 4.6 | | |
| RLIQNFLTI | | | | 40 | | |
| GMRQNATEI | | | | 81 | | |
| YMRVNGKWM | | | | 50 | | |
| FYIQMATEL | | | 0.31 | | | |
| FYIQMCTFL | | | 1.1 | | | |
| AYERMANIL | | | 233 | | | |
| AYQRMCNIL | | | 2.7 | | | |
| AYERMCTTL | | | 4.1 | | | |
| ASNENMETM | >37000 | >31000 | >10000 | 33 | | |
| TYQRTRALM | | | 69 | | | |
| TYQKTRALV | | | 44 | | | |
| TYQPTRALV | | | 17 | | | |
| TYQFTRALV | | | 371 | | | |
| TYQLTRALV | | | 110 | | | |
| SDYEGRLI | | | | | | 0.60 |
| MITQFESL | | 64 | | | | |
| RTFSFQLI | | 26 | | | | |
| FSVIFDRL | | 201 | | | | |
| RTFSFQLI | | 27 | | | | |
| MITQFESL | | 42 | | | | |
| FSVIFDRL | | 115 | | | | |
| KSSFYRNL | | 209 | | | | |
| SSLPFQNI | | 53 | | | | |
| MNIQFTAV | | 131 | | | | |
| MNYYWTLL | | 169 | | | | |
| SFYRNLLWL | | | | 46 | | |
| SSLPFQNI | | 9.5 | | | | |
| MNIQFTAV | | 26 | | | | |
| MNYYWTLL | | 56 | | | | |
| KSSFYRNL | | 117 | | | | |
| SIIPSGPL | | 393 | | | | |
| LSYSAGAL | | 60 | | | | |
| LSYSAGAL | | 31 | | | | |
| SSISFCGV | | 29 | | | | |
| TGIGNQNII | | | | 13 | | |
| ITYKNSTWV | | | | 409 | | |
| FCGVNSDTV | | | | 206 | | |
| TGICNQNII | | | | 21 | | |
| FCGVNSDTV | | | | 166 | | |
| ITYKNSTWV | | | | 276 | | |
| SSISFCGV | | 2.3 | | | | |
| IGRFYIQM | | 42 | | | | |
| MMIWHSNL | | 238 | | | | |
| ASNENMETM | | | | 41 | | |
| IGRFYIQM | | 24 | | | | |
| MMIWHSNL | | 287 | | | | |
| FFYRYGFV | | 350 | | | | |
| KMITQRTI | | 300 | | | | |
| RSYLIRAL | | 103 | | | | |
| RFYRTCKL | | 117 | | | | |
| TALANTIEV | | | | 16 | | |
| TALANTIEY | | | | 3.7 | | |
| RSYLIRAL | | 78 | | | | |
| RFYRTCKL | | 47 | | | | |
| VYINTALL | | 65 | | | | |
| VYINTALL | | 14 | | | | |
| VYIEVLHL | | 75 | | | | |
| VYIEVLHL | | 21 | | | | |
| WYIPPSLRTL | | | 96 | | | |
| MURTAZAKDPE | | | 0.96 | | | |
| PTIDES | | | | | | |
| IYSTVASSL | | | 4.1 | | | |
| LYEKVKSQL | | | 2.2 | | | |
| LYQKVKSQL | | | 2.8 | | | |
| LYBKMKSQL | | | 1.6 | | | |
| LYEKVFSQL | | | 7.4 | | | |
| LYQNVGTYV | | | 6.9 | | | |
| MGLKFRQL | | 7.4 | | | | |
| VSYVNTNM | | 60 | | | | |
| SYVNTNMGL | | | 19 | | | |
| MGLKFRQL | | 6.3 | | | | |
| VSYVNTNM | | 33 | | | | |
| SYVNTNMGL | | | 12 | | | |
| WGPSLYSI | 17 | | | | | |
| ASARFSWL | | | 323 | | | |
| WGPSLYSIL | 6.6 | | | | | |
| TGPCRTCMT | 108 | | | | | |
| WYWGPSLYSI | | | 8.3 | | | |
| IPQSLDSWWTS | | | | | 2.2 | |
| L | | | | | | |
| IPQSLDSYWTS | | | | | 2.7 | |
| L | | | | | | |
| ASARIFSWL | | 49 | | | | |
| WYWGPSLYSI | | | 16 | | | |
| APQSLDSWWTS | | | | | 15 | |
| L | | | | | | |
| IPQALDSWWTS | | | | | 6.1 | |
| L | | | | | | |
| IPQSLASWWTS | | | | | 4.2 | |
| L | | | | | | |
| IPQSLDAWWTS | | | | | 4.0 | |
| L | | | | | | |
| IPQSLDSAWTS | | | | | 13 | |
| L | | | | | | |
| IPQSLDSWWAS | | | | | 0.34 | |
| L | | | | | | |
| IPQSLDSWWTA | | | | | 134 | |
| L | | | | | | |
| EPQSLDSWWTS | | | | | 86 | |
| L | | | | | | |
| IPESLDSWWTS | | | | | 13 | |
| L | | | | | | |
| IPQSLDEWWTS | | | | | 1.9 | |
| L | | | | | | |
| IPQSLDSWWTE | | | | | 3.0 | |
| L | | | | | | |
| RPQSLDSWWTS | | | | | 60 | |
| L | | | | | | |
| IPRSLDSWWTS | | | | | 160 | |
| L | | | | | | |
| IPQRLDSWWTS | | | | | 23 | |
| L | | | | | | |
| TPQSRDSWWTS | | | | | 21 | |
| L | | | | | | |
| IPQSLRSWWTS | | | | | 12 | |
| L | | | | | | |
| IPQSLDRWWTS | | | | | 5.0 | |
| L | | | | | | |
| IPQSLDSRWTS | | | | | 47 | |
| L | | | | | | |
| IPQSLDSWWRS | | | | | 485 | |
| L | | | | | | |
| IPQSLDSWWTR | | | | | 196 | |
| L | | | | | | |
| YPQSLDSWWTS | | | | | 91 | |
| L | | | | | | |
| IPYSLDSWWTS | | | | | 0.78 | |
| L | | | | | | |
| IPQYLDSWWTS | | | | | 92 | |
| L | | | | | | |
| IPQSLYSWWTS | | | | | 4.7 | |
| L | | | | | | |
| TPQSLDYWWTS | | | | | 1.6 | |
| L | | | | | | |
| IPQSLDSWYTS | | | | | 17 | |
| L | | | | | | |
| IPQSLDSWWTY | | | | | 0.89 | |
| L | | | | | | |
| IPGSLDSWWTS | | | | | 24 | |
| L | | | | | | |
| IPQSLDSGWTS | | | | | 70 | |
| L | | | | | | |
| IPQSLDSPWTS | | | | | 19 | |
| L | | | | | | |
| IPQSLDSWGTS | | | | | 138 | |
| L | | | | | | |
| IPQSLDSWPTS | | | | | 60 | |
| L | | | | | | |
| IPQSLDSWWTG | | | | | 2.5 | |
| L | | | | | | |
| IPQSLDSWWTP | | | | | 1.2 | |
| L | | | | | | |
| IPQVLDSWWTS | | | | | 5.1 | |
| L | | | | | | |
| IPQFLDSWWTS | | | | | 4.3 | |
| L | | | | | | |
| IPQPLDSWWTS | | | | | 6.3 | |
| L | | | | | | |
| IPQMLDSWWTS | | | | | 4.1 | |
| L | | | | | | |
| IPQILDSWWTS | | | | | 12 | |
| L | | | | | | |
| IPQLLDSWWTS | | | | | 0.25 | |
| L | | | | | | |
| IPQGLDSWWTS | | | | | 2.7 | |
| L | | | | | | |
| IPQTLDSWWTS | | | | | 7.7 | |
| L | | | | | | |
| IPQHLDSWWTS | | | | | 39. | |
| L | | | | | | |
| IPQCLDSWWTS | | | | | 25 | |
| L | | | | | | |
| IPQNLDSWWTS | | | | | 12 | |
| L | | | | | | |
| IPQQLDSWWTS | | | | | 1.7 | |
| L | | | | | | |
| IPQWLDSWWTS | | | | | 3.7 | |
| L | | | | | | |
| IPQDLDSWWTS | | | | | 22 | |
| L | | | | | | |
| IPQKLDSWWTS | | | | | 9.3 | |
| L | | | | | | |
| IPQSLVSWWTS | | | | | 11 | |
| L | | | | | | |
| IPQSLFSWWTS | | | | | 11 | |
| L | | | | | | |
| IPQSLPSWWTS | | | | | 16 | |
| L | | | | | | |
| IPQSLMSWWTS | | | | | 0.95 | |
| L | | | | | | |
| IPQSLISWWTS | | | | | 17 | |
| L | | | | | | |
| IPQSLLSWWTS | | | | | 0.84 | |
| L | | | | | | |
| IPQSLGSWWTS | | | | | 2.7 | |
| L | | | | | | |
| IPQSLSSWWTS | | | | | 0.49 | |
| L | | | | | | |
| IPQSLTSWWTS | | | | | 1.7 | |
| L | | | | | | |
| IPQSLHSWWTS | | | | | 1.5 | |
| L | | | | | | |
| IPQSLCSWWTS | | | | | 1.1 | |
| L | | | | | | |
| IPQSLNSWWTS | | | | | 1.5 | |
| L | | | | | | |
| IPQSLQSWWTS | | | | | 0.81 | |
| L | | | | | | |
| IPQSLWSWWTS | | | | | 2.4 | |
| L | | | | | | |
| IPQSLKSWWTS | | | | | 1.1 | |
| L | | | | | | |
| IPSLDSWWTSL | | | | | 119 | |
| IPQSLDSWTSL | | | | | 0.22 | |
| IPQSLDSWWTL | | | | | 1.3 | |
| IPQALASWWTS | | | | | 26 | |
| L | | | | | | |
| IPQSLDSWWTS | | | | | 0.80 | |
| M | | | | | | |
| IPQSLDSWWTS | | | | | 1.9 | |
| F | | | | | | |
| KTPSFPNI | | 270 | | | | |
| HAVEFHNL | | 49 | | | | |
| VSAAFYHL | | 7.0 | | | | |
| VIGCYGSL | | 157 | | | | |
| KQYLNLYPV | | | | 3.4 | | |
| CYGSLPQEHI | | | 303 | | | |
| VSAAFYHL | | 5.2 | | | | |
| HAVEFHNL | | 158 | | | | |
| VIGCYGSL | | 63 | | | | |
| KTPSFPNI | | 155 | | | | |
| RPQSLDSWWTS | | | | | 144 | |
| L | | | | | | |
| IPQRLDSWWTS | | | | | 34 | |
| L | | | | | | |
| IPQSLRSWWTS | | | | | 11 | |
| L | | | | | | |
| IPQSLDRWWTS | | | | | 2.0 | |
| L | | | | | | |
| IPQSLDSRWTS | | | | | 2.6 | |
| L | | | | | | |
| IPQSLDSWWRS | | | | | 335 | |
| L | | | | | | |
| IPQSLDSWWTR | | | | | 27 | |
| L | | | | | | |
| IPQELDSWWTS | | | | | 18 | |
| L | | | | | | |
| IPQSLYSWWTS | | | | | 8.3 | |
| L | | | | | | |
| IPQSLDSWETS | | | | | 5.3 | |
| L | | | | | | |
| IPQSLDSWWES | | | | | 394 | |
| L | | | | | | |
| VESENKVV | | | | | | 349 |
| AGPYRAFVTI | 5.0 | | | | | |
| RAPYRAFVTI | 176 | | | | | |
| RGPYRAFVTA | 126 | | | | | |
| KGPYRAFVTI | 5.8 | | | | | |
| RGPYRAFVTK | 91 | | | | | |
| RGPGRAFVTI | 9.7 | 31000 | >10000 | 22000 | | |
| RGPGRYFVTI | 2.7 | | | | | |
| RGPGRAYVTI | 14 | | | | | |
| RGPGRAFYTI | 7.2 | | | | | |
| VESMNKEL | | | | | | 114 |
| TDSQYALGI | | | | | | 179 |
| RGAYRAFVTI | 3.4 | | | | | |
| RGPARAFVTI | 1.04 | | | | | |
| RGPYRAAVTI | 2.0 | | | | | |
| RGPYRAFATI | 2.1 | | | | | |
| RGPYRAFVAI | 1.3 | | | | | |
| RGKYRAFVTI | 67 | | | | | |
| RGPFRAFVTI | 0.78 | | | | | |
| RGPYKAFVTI | 13 | | | | | |
| RGPYRKFVTI | 3.6 | | | | | |
| RGPYRAYVTI | 2.1 | | | | | |
| RGPYRAFKTI | 2.3 | | | | | |
| RGPYRAFVKI | 3.9 | | | | | |
| NEILIRCII | | | | | | 12 |
| QEKKRHVDL | | | | | | 256 |
| LFVVYRDSI | | | 453 | | | |
| FYSRIRELRF | | | 447 | | | |
| SSIEFARL | | 1.8 | >10000 | | | |
| KVPRNQDWL | | | | 38 | | |
| VYDFYVWM | | 145 | | | | |
| KNKFFSYL | | 57 | | | | |
| LAVLYCLL | | 72 | | | | |
| YMVPFIPL | | 70 | | | | |
| GQMNNGSTPM | | | | 242 | | |
| IVTMFEAL | | 82 | | | | |
| ISHNFCNL | | 411 | | | | |
| GVYQFKSV | | 11 | | | | |
| HYISMGTSGL | | | 83 | | | |
| SGVENPGGYC | | >31000 | | 60 | | |
| L | | | | | | |
| KAVYNFATM | | | | 3.3 | | |
| CMANNSHHYI | | | | 220 | | |
| CSANNSHHYM | | | | 42 | | |
| SMVENPGGYC | | | | 154 | | |
| L | | | | | | |
| SGVENPGGYC | | | | 128 | | |
| M | | | | | | |
| KAVYNFATM | | | | 1.5 | >27000 | |
| KAVYNAATM | | | | 2.0 | >27000 | |
| KAVANFATM | | | | 1.2 | 27000 | |
| KAVYNYATM | | | | 2.1 | >27000 | |
| KAVYNFAAM | | | | 4.4 | 27000 | |
| YTVKYPNL | | 204 | | | | |
| FQPQNGQFI | | | | 6.9 | | |
| VGLSYSQTM | | 71 | | | | |
| FQPQNGQFI | | >31000 | | 4.9 | | |
| FQPQNGQFIHF | | 15500 | | 280 | | |
| Y | | | | | | |
| RPQASGVYM | | >31000 | | >44000 | 0.99 | |
| RPQASQVYM | | | | | 3.8 | |
| YTYKYPNL | | 1.8 | | | | |
| RPQASGVYM | | | | | 3.0 | |
| RPQASGVAM | | | | | 12 | |
| RPQGSGVYM | | | | | 39 | |
| RIPNASGVYM | | | | | 19 | |
| KAVYNFATCGI | | | | 29 | | |
| KAVYNFATB | | | | 7.9 | | |
| VYAKECTGL | | | 129 | | | |
| YPHFMLPTNL | | | | | 7.5 | |
| YPHYMPTNL | | | | | 9.5 | |
| HETTYNSI | | | | | | 1.8 |
| YEDTGKTI | | | | | | 0.86 |
| LGYDYSYL | | 3.4 | | | | |
| SSMHNALHI | | | | 7.6 | | |
| ANFSFRNTL | | 6.0 | | | | |
| SYLTLAKHT | | | 188 | | | |
| HYYVSRDTL | | | 43 | | | |
| YYVSRDTLL | | | 99 | | | |
| SFFSSWQII | | | 16 | | | |
| SYMVPFIPL | | | 144 | | | |
| PYLEQASRI | | | 173 | | | |
| SYLTLAKHTI | | | 4.4 | | | |
| HYYVSRDTLL | | | 167 | | | |
| SQVMNLHNL | | | | 2.3 | | |
| YENDIEKKI | | | | | | 3.8 |
| NEEPSDKHI | | | | | | 40 |
| EEKHEKKHV | | | | | | 284 |
| SYVPSAEQIL | | | 280 | | | |
| RYLENGKETL | | | 80 | | | |
| RYLKNGKETL | | | 217 | | | |
| IYTQNRRAL | | | 144 | | | |
| VYDFFVWM | | 464 | | | | |
| SVYDFFVWL | | 1.0 | | | | |
| SVYDFYVWM | | 1.2 | 3365 | | | |
| ASNENMDAM | | | | 28 | | |
| FAPGYNPAL | | | | 2.0 | | |
| SIQFFGERAL | | | | 21 | | >44000 |
| SIQFFGEL | | | | 16 | | >44000 |
| RGYVYQGL | >37000 | 2.1 | >10000 | >44000 | | |
| RGPRLNTL | 186 | | | | | |
| HMWNFIGV | | 202 | | | | |
| GGAYRLIVF | 3.5 | | | | | |
| KYLVTRHADV | | | 33 | | | |
| FSPRRNGYL | 2.7 | | | | | |
| SHYAFSPM | | 250 | | >88000 | | |
| FQPQNGQFI | | 9513 | | 17 | | |

1. A composition comprising one or more peptides from any of Tables 11-29.

2. A composition comprising nucleic acids encoding one or more peptides from any of Tables 11-29.

3. The composition of claim 1, wherein at least one of the one or more peptides is an HTL epitope.

4. The composition of claim 1, wherein at least one of the one or more peptides is a CTL epitope.

5. The composition of claim 4, further comprising an HTL epitope.

6. The composition of claim 1, further comprising a spacer molecule.

7. The composition of claim 1, further comprising a carrier.

8. The composition of claim 1, further comprising an MHC targeting sequence.

9. The composition of claim 1, further comprising a lipid.

10. The composition of claim 1, wherein the one or more peptides are incorporated as part of a liposome.

11. The composition of claim 1, wherein at least one of the one or more peptides is a heteropolymer.

12. The composition of claim 1, wherein at least one of the one or more peptides is a homopolymer.

13. The composition of claim 1, wherein at least one of the one or more peptides is a peptide from an antigen selected from the group consisting of prostate specific antigen (PSA), prostate specific membrane antigen (PSM), hepatitis B virus (HBV) antigen, hepatitis C virus (HCV) antigen, malignant melanoma antigen (MAGE), Epstein Barr virus, human immunodeficiency type-1 (HIV-1), human immunodeficiency type-2 (HIV-2), papilloma virus, Lassa virus, mycobacterium tuberculosis (MT), p53, murine p53 (mp53), CEA, HER2/neu, and tyrosine kinase related protein (TKP).

14. A pharmaceutical composition comprising an active ingredient, wherein the active ingredient comprises the composition of claim 1.

15. A vaccine composition comprising an active ingredient, wherein the active ingredient comprises the composition of claim 1.

16. The use of the composition of claim 1, wherein the composition is a prophylactic composition for the prevention of viral infection or cancer.

17. The use of the composition of claim 1, wherein the composition is a therapeutic composition for the treatment of viral infection or cancer.

18. A diagnostic reagent comprising the composition of claim 1.

19. The use according to claim 17, for the treatment of prostate cancer, hepatitis B, hepatitis C, AIDS, renal carcinoma, cervical carcinoma, lymphoma, CMV or chondyloma acuminatum.
